(19) United States
(12) Patent Application Publication
(10) Pub. No.: US 2026/0301953 A1
(43) Pub. Date: Oct. 1, 2026

(54) Non-Invasive Analysis of Electrophysiological Processes Using Imaging and Deep Learning

(71) Applicant: The Regents of the University of California, Oakland, CA (US)

(72) Inventors: Jan Christoph, San Francisco, CA (US); Jan Lebert, San Francisco, CA (US)

(21) Appl. No.: 19/481,040

(22) PCT Filed: May 10, 2024
(86) PCT No.: PCT/US2024/028939

§ 371 Date: Nov. 3, 2025

Related U.S. Application Data
(60) Provisional application No. 63/465,826, filed on May 11, 2023.
(60) Provisional application No. 63/613,466, filed on Dec. 21, 2023.

(51) Int. Cl. G16H 50/20; G16H 30/20; G16H 50/50

(52) U.S. Cl. CPC G16H 50/20; G16H 30/20; G16H 50/50

(57) ABSTRACT

This invention provides new and useful methods for accurately imaging heart rhythm disorders. Novel panoramic optical mapping methods, and recent advances in machine learning techniques, are used to create a mechano-electrical model effective in predicting electrical activity of a tissue based on motion of the tissue. Deep learning techniques for constructing three-dimensional intramural electrical wave patterns from surface data, imaging chambers for performing panoramic optical mapping methods, and deep learning techniques for motion tracking tissue via optical and/or ultrasound data are also provided. The optical mapping and deep learning techniques of the disclosure may be applied in a plurality of different ways, individually or in combination, to improve the diagnosis and treatment of heart rhythm disorders. Also provided are systems for performing the methods described herein as well as non-transitory computer readable storage media and computer products.

### STATEMENT REGARDING FEDERALLY SPONSORED RESEARCH

This invention was made with government support under Grant No. DP2HL168071 awarded by the National Institutes of Health. The government has certain rights in the invention.

### CROSS-REFERENCE TO RELATED APPLICATION

Pursuant to 35 U.S.C. § 119(e), this application claims priority to the filing dates of United States Provisional Patent Application Ser. No. 63,465,826 filed May 11, 2023, and United States Provisional Patent Application Ser. No. 63,613,466 filed Dec. 21, 2023, the disclosures of which applications are herein incorporated by reference in their entirety.

### INTRODUCTION

Heart disease is a major cause of morbidity and mortality worldwide. Understanding the origins and underlying mechanisms of heart rhythm disorders is pivotal in the planning of therapeutic interventions. Yet, despite decades of research, the driving mechanisms underlying complex heart rhythm disorders (such as, e.g., atrial or ventricular fibrillation) remain poorly understood. This is due, at least in part, to the major technological and scientific challenges inherently associated with the imaging of heart rhythm disorders. Although cardiovascular imaging has significantly progressed over the past several decades, imaging the heart's physiological processes still has major limitations.

Currently, heart rhythm disorders, including atrial fibrillation (AF) and ventricular tachycardia (VT), are routinely diagnosed using catheter-based mapping. Unfortunately, in addition to being invasive and time-consuming, catheter-based measurements typically only provide information about the heart's abnormal electrical activity on its surface. It is not presently possible to map the three-dimensional morphology of electrical waves throughout the entire heart muscle using catheter-based approaches, especially during arrhythmias such as ventricular tachycardia. However, the electrical wave phenomena associated with abnormal cardiac rhythms (e.g., arrhythmias, such as atrial arrhythmias or ventricular arrhythmias) are inherently three-dimensional phenomena evolving deep within the cardiac muscle, where they often interact with an arrhythmogenic substrate (e.g., scar tissue) in locations that are inaccessible to standard catheter mapping. Better imaging technology is greatly needed to improve diagnostics and therapies.

### SUMMARY

Thus, there is a need for improved and useful methods and systems for accurately imaging (e.g., measuring and visualizing) heart rhythm disorders. In particular, there is a need for methods and systems useful in visualizing the heart's intramural electrical activity. This invention provides such new and useful methods and systems, addressing the limitations mentioned above. To accomplish this, the invention pursues the highly unconventional approach of calculating or predicting electrical activity from the heart's motion (captured via, e.g., 4D ultrasound or MRI). To enable such an approach, the invention leverages recent advances in machine learning techniques (including, e.g., deep learning techniques), and generates high-resolution training data via novel optical mapping methods, in order to train a model to predict three-dimensional electrical wave dynamics from deformation mechanics (e.g., cardiac deformation mechanics, such as ventricular or atrial deformation mechanics). The methods and systems of the invention, e.g., as described in greater detail below, find use in a variety of applications where it is desirable to improve the diagnosis and treatment of heart rhythm disorders—or other cardiac pathophysiology.

As discussed above, conventional cardiovascular mapping techniques are invasive and do not capture the three-dimensional morphology of electrical waves in the heart. As such, existing methods of creating electrophysiological and mechanical movement cardiac data are incapable of producing training data-sets of sufficient size, quality, or heterogeneity to train a model capable linking electrophysiological cardiac tissue dynamics to mechanical cardiac tissue dynamics. In order to generate training data sufficient for the creation of a robust model effective in predicting three-dimensional electrical wave dynamics from deformation mechanics (e.g., cardiac deformation mechanics, such as ventricular or atrial deformation mechanics), novel deep learning techniques and models for constructing three-dimensional intramural electrical wave patterns from surface data, novel imaging techniques and apparatuses for fully panoramic stereoscopic imaging of contracting hearts, and novel deep learning techniques and models for motion tracking cardiac tissues via optical and/or ultrasound data are also presented in this disclosure.

The aforementioned models, apparatuses, and techniques useful for the generation of sufficient mechano-electrical model training data may be combined (e.g., via networks or iterative improvement loops) in order to enhance or refine the training data and/or the mechano-electrical model. Alternatively, as these models, chambers, and techniques each possess utility in their own right, they may be individually presented and applied, e.g., in order to improve the diagnosis and treatment of heart rhythm disorders or other cardiac pathophysiology. Accordingly, embodiments of the present invention may comprise one or more of the following aspects: 1. Deep learning-based reconstruction of electrical maps from catheter or optical mapping data including calculation of activation maps, phase maps and rotor cores. 2. Deep learning-based reconstruction of three-dimensional intramural electrical wave patterns from surface data. 3. Training data generation or model refinement via panoramic and/or stereoscopic imaging of contracting hearts. 4. Deep learning-based motion tracking of tissues via ultrasound, MRI, and/or optical data. 5. Deep learning-based reconstruction of surface or three-dimensional intramural electrical wave patterns from the motion of the heart. 6. Deep learning-based localization of myocardial fibrosis or scarring from the motion of the heart. Also provided are systems for performing the methods described herein as well as non-transitory computer readable storage media and computer products.

### BRIEF DESCRIPTION OF THE DRAWINGS

FIG. 1 provides a flow diagram for practicing methods of modeling mechanical movement patterns and electrophysiological activation patterns of a tissue in accordance with an embodiment of the disclosure.

FIG. 2 provides a flow diagram for practicing methods of modeling mechanical movement patterns and electrophysiological activation patterns of a tissue in accordance with an embodiment of the disclosure.

FIGS. 3A to 3C depict a distribution matching of in silico, ex vivo and clinical electromechanical data with sparse, heterogeneous, and out-of-distribution data to achieve generalization in accordance with an embodiment of the disclosure.

FIGS. 4A to 4B depict training data generation using electromechanical simulations in accordance with an embodiment of the disclosure.

FIGS. 5A to 5B illustrate both the ‘forward’ and the ‘inverse’ mechano-electrical problem.

FIGS. 6A to 6B provide block diagrams of a mechano-electrical model and a data set used to train the model in accordance with an embodiment of the disclosure.

FIGS. 7A to 7B illustrate a self-supervised deep learning-based approach for solving the ‘inverse’ mechano-electrical problem in accordance with an embodiment of the disclosure.

FIGS. 8A to 8C depict computer simulations used to generate training data for a mechano-electrical model in accordance with an embodiment of the disclosure.

FIGS. 9A to 9C show reconstruction of electrical excitation waves from mechanical deformation using both a deep neural network and numerical modeling.

FIGS. 10A to 10B illustrate electrical 3D activation patterns causing unique time-varying 3D contraction patterns.

FIGS. 11A to 11B provide block diagrams illustrating the use of an electrical wave dynamics model and an optical motion tracking model to train a mechano-electrical model in accordance with an embodiment of the disclosure.

FIGS. 12A to 12B provide block diagrams illustrating the use of an ultrasound motion tracking model to train a mechano-electrical model and the use of an optical motion tracking model to train an ultrasound motion tracking model in accordance with an embodiment of the disclosure.

FIGS. 13A to 13E illustrate ex vivo electromechanical imaging of arrhythmias to obtain training data for AI in accordance with an embodiment of the disclosure.

FIG. 14 depicts electromechanical high-resolution imaging of arrhythmias ex vivo using panoramic fluorescence and 4D ultrasound imaging in accordance with an embodiment of the disclosure.

FIGS. 15A to 15D show ex vivo electromechanical imaging of arrhythmias, using 16-camera high-speed panoramic fluorescence imaging, highspeed 4D ultrasound, and an imaging chamber, to obtain training data for an inverse mechano-electrical model in accordance with an embodiment of the disclosure.

FIG. 16 depicts ultrasound imaging during catheter ablation procedures in humans in accordance with an embodiment of the disclosure.

FIGS. 17A to 17B depict block diagrams of stereoscopic multi-camera optical mapping being used to train and/or validate a mechano-electrical model, and to train an optical motion tracking model in conjunction with an ultrasound motion tracking model, in accordance with an embodiment of the disclosure.

FIGS. 18A to 18B show models and experiments connected together in a training network or training loop in order to iteratively refine a mechano-electrical model in accordance with an embodiment of the disclosure.

FIGS. 19A to 19B show models and experiments connected together in a training network or training loop in order to iteratively refine a mechano-electrical model in accordance with an embodiment of the disclosure.

FIG. 20 illustrates deep learning-based reconstruction of scroll wave chaos inside a three-dimensional volume from partial observations of the dynamics on its surface in accordance with an embodiment of the disclosure.

FIGS. 21A to 21D provide surface observations and projections of three-dimensional scroll wave chaos (‘turbulent’ parameter regime) in a bulk medium in accordance with an embodiment of the disclosure.

FIG. 22 depicts observations of three-dimensional scroll waves (‘laminar’ parameter set) on the top and bottom surfaces of an opaque medium, and in the projection of the full dynamics in a transparent medium in the bulk's z-direction (depth).

FIGS. 23A to 23C provide predictions of three-dimensional ‘laminar’ scroll wave dynamics from two-dimensional observations using deep convolutional encoding-decoding neural network (U-Net) in an anisotropic excitable medium in accordance with an embodiment of the disclosure.

FIGS. 24A to 24D provide predictions of three-dimensional scroll wave dynamics (‘laminar’ parameter set) and their vortex filaments from either single- or dual-surface observations in a thick, opaque, anisotropic excitable medium using a deep learning model in accordance with an embodiment of the disclosure.

FIGS. 25A to 25C depict different electrical wave patterns as seen in the top layer, bottom layer, and projection of all layers of a three-dimensional bulk.

FIG. 26 illustrates bulk thickness and transmurality of scroll wave dynamics (‘turbulent’ scroll wave chaos).

FIGS. 27A to 27D provide predictions of (‘turbulent’) electrical scroll waves in subsurface layers of anisotropic (left) and isotropic (right) bulk tissue using U-Net in accordance with an embodiment of the disclosure.

FIGS. 28A to 28B provide predictions of electrical scroll waves within subsurface layers of a bulk-shaped ‘turbulent’ anisotropic excitable medium using U-Net in accordance with an embodiment of the disclosure.

FIGS. 29A to 29F show average reconstruction error over bulk depth in opaque or transparent excitable media with anisotropy using a U-Net model in accordance with an embodiment of the disclosure.

FIGS. 30A to 30B provide a comparison of reconstruction errors obtained with different imaging configurations in opaque anisotropic excitable medium using a U-Net model in accordance with an embodiment of the disclosure.

FIG. 31 depicts average reconstruction error per layer (depth) over time in an anisotropic excitable medium for 5 different depths using a deep learning model in accordance with an embodiment of the disclosure.

FIGS. 32A to 32B illustrate deep learning-based reconstructions on ‘laminar’ scroll wave dynamics in the presence of various noise levels in accordance with embodiments of the disclosure.

FIGS. 33A to 33B show reconstruction errors obtained with different neural network architectures for ‘turbulent’ scroll wave chaos in accordance with embodiments of the disclosure.

FIGS. 34A to 34E provide predictions of bulk thickness from surface observations of scroll wave chaos for various media using either a regression or a classification neural network in accordance with embodiments of the disclosure.

FIGS. 35A to 35C provide prediction errors at various bulk depths for isotropic excitable media using a deep learning model in accordance with an embodiment of the disclosure.

FIGS. 36A to 36B provide a comparison of deep learning-based reconstruction of scroll waves with a naive reconstruction in isotropic excitable media.

FIGS. 37A to 37D illustrate diffusion-based generative modeling of electrical wave dynamics in cardiac tissue in accordance with embodiments of the disclosure.

FIGS. 38A to 38B show diffusion-based reconstruction of scroll wave dynamics inside a three-dimensional bulk from two-dimensional observations of the dynamics on the bulk's top and bottom surface in accordance with embodiments of the disclosure.

FIGS. 39A to 39B provide transmural reconstruction error per layer number or depth with diffusion, U-Net or diffusion+U-Net models in accordance with embodiments of the disclosure.

FIGS. 40A to 40E illustrate hallucination of diffusion model during inpainting of electrical spiral wave dynamics.

FIGS. 41A to 41E illustrate diffusion-based modeling of reentrant electrical waves in heart-shaped bi-ventricular geometries in accordance with embodiments of the disclosure.

FIGS. 42A to 42C depict data-driven modeling of spiral wave dynamics using diffusion models in accordance with embodiments of the disclosure.

FIGS. 43A to 43C provide a comparison of spiral wave dynamics generated in computer simulations using a biophysical model (ground-truth) and ‘fake’ diffusion-generated spiral wave dynamics.

FIGS. 44A to 44E illustrate parameter-specific generation of spiral wave dynamics using diffusion generative modeling in accordance with embodiments of the disclosure.

FIGS. 45A to 45B show generation of 1,000 different bi-ventricular heart geometries for generation of an electromechanical training dataset consisting of tens of thousands of training samples of ventricular arrhythmias, according to an embodiment of the disclosure.

FIGS. 46A to 46B show computer simulation of ventricular deformation during reentrant arrhythmia, according to an embodiment of the disclosure.

FIGS. 47A to 47B illustrate deep learning-based prediction of electrical arrhythmia circuits from mechanical deformation in simulations of the heart's ventricles, according to embodiments of the disclosure.

FIGS. 48A to 48C depict simulated training data of focal and reentrant ventricular arrhythmias generated with electromechanical computer simulations, according to embodiments of the disclosure.

FIG. 49 shows spatial heterogeneity and scar tissue in the ventricles automatically generated for each simulation, according to embodiments of the disclosure.

FIG. 50 depicts neural network architecture and training samples for inverse mechano-electrical prediction of electrical arrhythmia circuits from ventricular deformation according to embodiments of the disclosure.

FIGS. 51A to 51B show deep learning-based predictions of focal electrical (action potential) waves from ventricular mechanical deformation.

FIGS. 52A to 52B provide electrical activation maps computed from the sequence of electrical wave patterns predicted using a deep learning-based approach, in accordance with embodiments of the disclosure.

FIGS. 53A to 531 show deep learning-based predictions of reentrant electrical (action potential) waves from ventricular motion, in accordance with embodiments of the disclosure.

FIGS. 54A to 54B provide deep learning-based prediction of electrical (action potential) waves from ventricular mechanical deformation in the presence of scar tissue, in accordance with embodiments of the disclosure.

FIGS. 55A to 55B illustrate training of a deep learning model on simulated data generated with the smoothed particle hydrodynamics (SPH) method, and application of the trained model to simulated data generated with the finite element method (FEM), in accordance with embodiments of the disclosure.

FIGS. 56A to 56C provide prediction accuracies of deep learning-based inverse reconstruction of electrical waves from ventricular deformation of the heart on synthetic simulated data with different models in accordance with embodiments of the disclosure.

FIG. 57 provides experimental results demonstrating that the prediction accuracy of a neural network of the disclosure stays the same over a wide parameter range.

FIGS. 58A to 58C illustrate optical mapping combined with numerical motion tracking for measurement of an action potential or calcium waves in moving and deforming cardiac tissues, such as an isolated heart, during voltage-sensitive optical mapping, according to embodiments.

FIG. 59 provides a flow diagram depicting the sequential order of the fully automatic numerical pre- and post-processing and tracking of optical mapping data, according to embodiments.

FIGS. 60A to 60F show typical examples of optical mapping data acquired with 4 different high-speed cameras, according to embodiments.

FIGS. 61A to 61E depict the performance of the Farneback GPU algorithm with sinus rhythm data imaged in an isolated, contracting rabbit heart, according to embodiments.

FIGS. 62A to 62E depict performance of the numerical motion tracking and motion-stabilization procedure on optical mapping data showing a rabbit heart during ventricular fibrillation, according to embodiments.

FIGS. 63A to 63D show numerical motion tracking and motion-stabilization procedures using the Farneback motion tracking algorithm with cell culture data, according to embodiments.

FIGS. 64A to 64E provide examples of sinus rhythm in a contracting mouse and rabbit heart, according to embodiments of the disclosure.

FIGS. 65A to 65B show a comparison of the processing speeds with low-resolution (128×128 pixels) voltage-sensitive optical mapping data, according to embodiments.

FIGS. 66A to 66B show a comparison of the processing speeds with 128×128, 256×256, 512×512, and 1,024×1,024-pixel video images, according to embodiments of the disclosure.

FIGS. 67A to 67C depict the accuracy of GPU-based motion tracking algorithms applied to synthetic voltage-sensitive optical mapping data, according to embodiments of the disclosure.

FIGS. 68A to 68C provide optical traces obtained from contracting heart surface during ventricular fibrillation (VF) in voltage-sensitive optical mapping recordings, according to embodiments of the disclosure.

FIGS. 69A to 69C provide a comparison of motion tracking error of 5 different GPU-accelerated motion tracking algorithms, according to embodiments.

FIGS. 70A to 70C show contrast-enhancement with different kernel diameters k for three different optical mapping recordings, according to embodiments.

FIG. 71 provides an illustration of the synthetic-to-real approach, according to embodiments of the disclosure.

FIG. 72 shows a comparison of PWC-Net architecture and a classical coarse-to-fine approach, according to embodiments.

FIG. 73 provides results on the validation dataset using PWC-Net, according to embodiments of the disclosure.

FIGS. 74A to 74B provide a tracking accuracy comparison between deep learning-based and conventional motion tracking algorithms, according to embodiments.

FIGS. 75A to 751 show seven samples of a validation dataset and predicted optical flow vector fields by a neural network according to embodiments of the disclosure.

FIGS. 76A to 76B illustrate tracking accuracy of deep learning-based motion tracking versus conventional motion tracking on experimental video data with added gaussian noise, according to embodiments.

FIGS. 77A to 77G depict an imaging setup, according to embodiments of the disclosure.

FIG. 78 shows camera poses and three-dimensional reconstruction of an isolated rabbit heart, according to embodiments.

FIGS. 79A to 79E depict an imaging configuration for panoramic voltage-sensitive optical mapping of the entire contracting ventricular surface, according to embodiments of the disclosure.

FIGS. 80A to 80B show sinus rhythm imaged in a contracting rabbit heart with voltage-sensitive multi-camera optical mapping, according to embodiments.

FIGS. 81A to 81D illustrate ventricular wall motion during sinus rhythm imaged with an optical mapping system, according to embodiments of the disclosure.

FIGS. 82A to 82C illustrate ventricular wall motion during sinus rhythm imaged with an optical mapping system, according to embodiments.

FIGS. 83A to 83D depict action potential wave fronts following a pacing stimulus, according to embodiments.

FIGS. 84A to 84F show ventricular fibrillation imaged across the entire surface of a contracting isolated rabbit heart using voltage-sensitive 3D optical mapping according to embodiments of the disclosure.

FIGS. 85A to 85C illustrate that action potential waves cause rotating mechanical motion of the ventricular surface in a fibrillating and contracting rabbit heart.

FIGS. 86A to 86C show action potential vortex waves and mechanical vortices on the three-dimensional, deforming ventricular surface during ventricular fibrillation in a rabbit heart, according to embodiments.

FIGS. 87A to 87C provide optical traces from the ventricular surface of a fibrillating, contracting rabbit heart measured with a multi-camera optical mapping system, according to embodiments of the disclosure.

FIGS. 88A to 88C present overviews of a panoramic imaging setup according to an embodiment of the disclosure.

FIG. 89 provides a schematic representation of a computer-controlled optical mapping system's electrical components, according to embodiments.

FIGS. 90A to 90C illustrate ventricular segmentation from B-mode ultrasound data, according to embodiments.

FIGS. 91A to 91C show optical flow-based motion tracking of the ventricles in 4D ultrasound data, according to embodiments.

FIG. 92 shows a particle-based representation of ventricles from 4D ultrasound data, according to embodiments of the disclosure.

FIG. 93 illustrates action potential wavefront propagation across a beating rabbit heart over time following electrical stimulation, according to embodiments.

FIG. 94 illustrates inverse mechano-electrical prediction with a neural network, according to embodiments of the disclosure.

FIG. 95 depicts how 4D ultrasound and computed three-dimensional motion vectors of tissue movement may be used to predict electrical wavefront propagation and calculate electrical activation maps, according to embodiments of the disclosure.

FIG. 96 illustrates how 3D optical surface reconstructions and electrophysiology, in combination with 3D ultrasound, is used to map a surface mesh onto ultrasound datapoints of the heart and compute three-dimensional motion vectors of tissue movement, according to embodiments of the disclosure.

### DETAILED DESCRIPTION

Methods and systems for accurately imaging heart rhythm disorders are provided. Embodiments of the methods leverage panoramic optical mapping approaches and recent advances in machine learning techniques to create (e.g., train or configure) a mechano-electrical model effective in predicting or calculating electrical activity of a tissue based on motion of the tissue (e.g., in combination with the localized characteristics of the tissue, such as fibrosis or scarring). Deep learning and differentiable simulation techniques for constructing three-dimensional intramural electrical wave patterns from surface data or partial data, imaging chambers for performing panoramic optical mapping methods, and deep learning or gradient descent-based techniques for motion tracking tissue via optical and/or ultrasound data are also provided. The optical mapping and deep learning techniques of the disclosure may be applied in a plurality of different ways, individually or in combination, to improve the diagnosis and treatment of heart rhythm disorders and other cardiac pathophysiology such as, e.g., hypertrophic cardiomyopathy, bundle branch block, long QT syndrome, and/or heart failure. Also provided are systems for performing the methods described herein as well as non-transitory computer readable storage media and computer products.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims. Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### Methods

As summarized above, methods for accurately imaging heart rhythm disorders are provided. Embodiments of the methods leverage panoramic optical mapping approaches and recent advances in machine learning techniques to create (e.g., train or configure) a mechano-electrical model effective in predicting or calculating electrical activity of a tissue based on motion of the tissue. Details of embodiments of the present invention, including the deep learning techniques and models for constructing three-dimensional intramural electrical wave patterns from surface data, the imaging chambers for performing panoramic and/or stereoscopic optical mapping methods, the deep learning techniques and models for motion tracking tissue via optical and/or ultrasound data, and the machine learning techniques and models for constructing three-dimensional intramural electrical wave patterns or tissue characteristics from the motion (i.e., mechanical movement or deformation patterns) of the heart, and aspects thereof, are described below. Also described are methods of enhancing or refining the mechano-electrical training data and/or the mechano-electrical model by combining (e.g., via networks/webs or iterative improvement loops) the aforementioned models, chambers, and techniques.

### Machine Learning Models and Experimental Setups

As described above, embodiments of the methods include methods of training an electrical wave dynamics (EWD) model for, e.g., constructing three-dimensional intramural electrical wave patterns from cardiac tissue surface data. The cardiac tissue surface data may comprise any type of electrophysiological activation patterns correlated with the electrical waves of the heart that induce the mechanical deformation of cardiac tissue (that occurs, e.g., during a heartbeat or heart pulsation). For example, the cardiac tissue surface data may comprise electrical data such as, e.g., voltage measurements or calcium concentration measurements associated with action potential (AP) waves.

The surface data may be obtained through any number of means including, e.g., through conventional catheter-based cardiac mapping approaches and/or the panoramic stereoscopic multi-camera optical mapping techniques of this disclosure described below. In some embodiments, the EWD model may be able to construct the three-dimensional (3D) intramural electrical wave patterns occurring during normal heart function (e.g., sinus rhythm) from cardiac tissue surface data. In some embodiments, the EWD model may be able to construct the 3D intramural wave patterns that occur during abnormal heart function. For example, the EWD model may be able to generate or construct 3D intramural focal wave and/or reentrant wave (e.g., scroll and/or spiral wave) dynamics from measurements of action potential waves taken at the surface of the heart. In some embodiments (e.g., of the EWD model and/or the mechano-electrical model below), the electrical wave dynamics and mechanical response to it may depend on tissue heterogeneities such as fibrosis or scarring. In some embodiments, the EWD model is able to construct or generate the 3D intramural electrical wave patterns that occur during both normal and abnormal heart function from cardiac tissue surface data. In order to train the EWD model, a training dataset sufficient for training the EWD model to perform one or more specific tasks is generated or obtained. By ‘sufficient’ in this context is meant the training data has a size, quality, and heterogeneity that enables training a model to perform one or more desired tasks with a predetermined threshold level of accuracy. In some cases, the predetermined threshold level of accuracy is calculated or determined for a trained (e.g., EWD or mechano-electrical) model in a manner that ensures less frequently occurring phenomena (e.g., abnormal heart function dynamics) are accurately reproduced or generated (e.g., from cardiac tissue surface data). In some embodiments, EWD model training datasets may comprise high-resolution in silico, ex vivo, and/or in human (e.g., clinically obtained) data.

In silico training data may be generated using computer simulations of, e.g., electrical numerical models. In some embodiments, multiple different numerical models are used to ensure sufficient heterogeneity such as, e.g., an Aliev-Panfilov model, a Fenton-Karma model, a Mitchell-Schaeffer model, and/or a Bueno-Orovio model (as described in, e.g., Example 3, below). In these cases, the parameters used to run each simulation of each model may also be varied, e.g., randomly, within a specific range of values. In some embodiments, synthetic noise is introduced into the in silico training data simulations. The synthetic noise may be configured to recreate or capture the noise phenomena occurring in the measurements of cardiac tissue surface data that are intended to be input into the trained EWD model during use (i.e., the noise naturally occurring in the surface data measurements the trained EWD model will be applied to). For example, synthetic noise that accurately captures or recreates the noise of catheter-based measurements of cardiac tissue surface data may be introduced or injected into simulated training data, e.g., when the trained EWD model is intended to construct 3D intramural electrical wave patterns from said catheter-based measurements.

In some embodiments, ex vivo training data may be generated from optical mapping experiments, such as, e.g., the panoramic/stereoscopic optical mapping experiments of this disclosure, discussed below. In some cases, ex vivo training data may be generated from mechanical movement patterns of cardiac tissue (e.g., obtained from optical mapping experiments) that are input into a mechano-electrical model of this disclosure in order to construct or generate electrical wave dynamics of the tissue. In human (e.g., clinically obtained) training data may be obtained via, e.g., conventional catheter-based cardiac mapping approaches. In some embodiments, the training dataset includes a combination of in silico, ex vivo, and clinical data sufficient to ensure that the dataset is of adequate size, quality, and heterogeneity for its intended purpose. In some embodiments, the training data input during training of the EWD model is augmented by, e.g., adding random noise to the input values, randomly rotating or flipping the data, removing a random section of the input data, scaling the data randomly, blurring the input data, and/or adding artifacts to the input data in order to ensure that the dataset is of sufficient size, quality, and heterogeneity for its intended purpose.

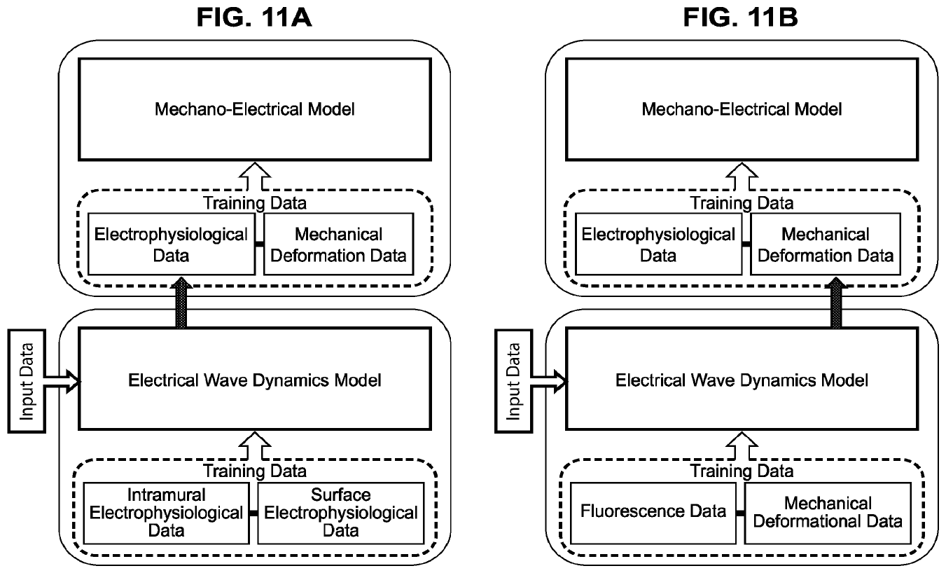

The training data may be generated based on the task the EWD model is trained to perform. In some embodiments, the EWD model may be trained to reconstruct 3D waves from 2D measurements, as discussed above. In these instances, the EWD model may be applied in order to create a training dataset for the electro-mechanical model of this disclosure from, e.g., clinical catheter-based measurements (performed in conjunction with, e.g., ultrasound or MRI measurement generation) (see, e.g., FIG. 11A). In some embodiments, the EWD model may be trained to predict future three-dimensional intramural electrical wave patterns from cardiac tissue surface data. In these embodiments, the EWD model may be used in conjunction with the mechano-electrical model in order to, e.g., predict future three-dimensional intramural electrical wave patterns from mechanical movement patterns, or to create a training dataset such that the mechano-electrical model of this disclosure may be trained to do so directly. In some cases, the EWD model may be trained (or fitted) to detect the presence of, or to localize, fibrosis or tissue scarring from cardiac tissue surface data. In some embodiments, the EWD model may be a machine learning model including an artificial neural network (NN). In some embodiments, the machine learning model is a deep learning model. In these cases, the model may be three or more layers deep, such as five or more layers deep, or ten or more, or twelve or more, or thirty or more, or fifty or more, or one hundred or more. In some cases, the EWD model is a diffusion-based generative model, or a differentiable simulation fitted to data (e.g., fitted to the training dataset discussed above).

As described above, embodiments of the methods include methods of training a motion tracking model, e.g., for tracking fluorescing tissues of an optical mapping experiments using optical measurements (e.g., an array of sequential images) (i.e., an optical motion tracking model) or for tracking tissues of in human or ex vivo experiments and procedures from ultrasound measurements (i.e., an ultrasound motion tracking model). By ‘tracking’ in this context is meant generating or determining the position and/or motion of one or more discrete areas or voxels of the tissue in relation to all the other areas/voxels of the tissue (e.g., obtaining mechanical movement patterns of the tissue).

In order to train the optical or ultrasound motion tracking model, a training dataset sufficient for training the motion tracking model to perform one or more specific tasks is generated or obtained. In some embodiments, motion tracking model training datasets may comprise high-resolution in silico, ex vivo, and/or in human (e.g., clinically obtained) data. In silico training data may be generated using computer simulations of, e.g., optical mapping experiments (such as, e.g., the panoramic optical mapping experiments of the disclosure). For example, numerical models of optical mapping experiments, in conjunction with virtual cameras, may be used in order to generate training data for the optical motion tracking models of the disclosure. Similarly, synthetic ultrasound data may be generated using numerical models of optical mapping experiments and/or using actual experimental data obtained by the panoramic optical mapping experiments of this disclosure discussed below (e.g., by adjusting in silico synthetic ultrasound data generating models to better capture real ultrasound measurements). Synthetic noise may be introduced into the motion tracking model training data similar to as was discussed for the EWD model above.

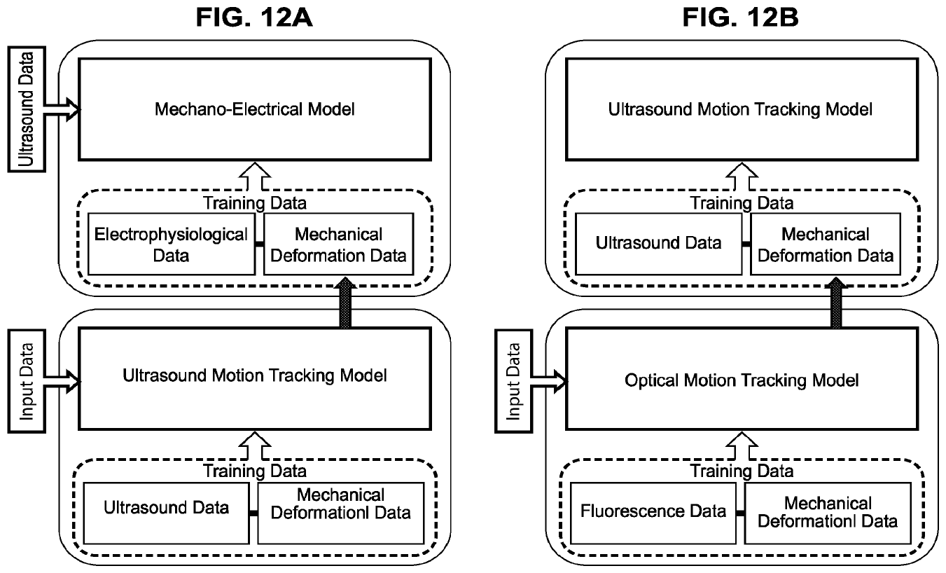

In some embodiments, the optical motion tracking model may be applied in order to create a training dataset for the mechano-electrical model of this disclosure from, e.g., optical measurements (e.g., images) obtained during optical mapping experiments such as, e.g., the panoramic optical mapping experiments of this disclosure (see, e.g., FIG. 11B). In some embodiments, the optical motion tracking model may be applied in order to create a training dataset for the ultrasound motion tracking model of this disclosure, e.g., from optical measurements coupled with ultrasound measurements obtained via the panoramic optical mapping experiments of the disclosure (see, e.g., FIG. 12B). In some instances, the ultrasound tracking model may be used in conjunction with the mechano-electrical model in order to, e.g., predict three-dimensional electrical wave dynamics from clinically obtained ultrasound data, or to create a training dataset such that the mechano-electrical model of this disclosure may be trained to do so directly (see, e.g., FIG. 12A).

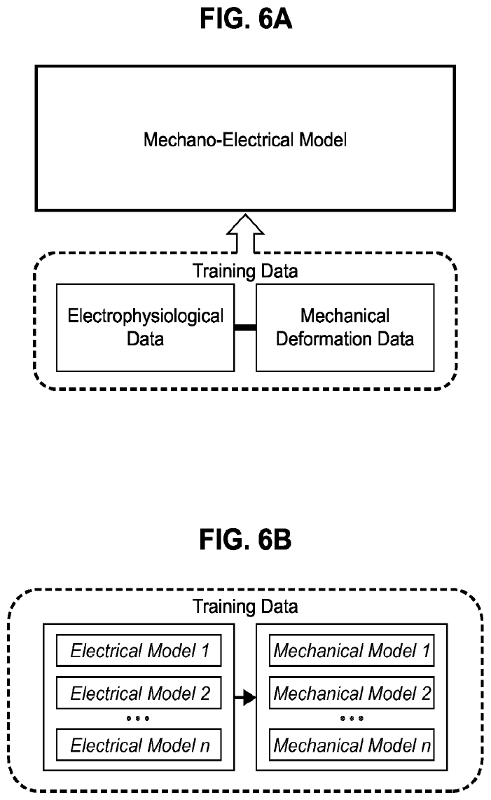

As described above, embodiments of the methods include methods of training a mechano-electrical model for, e.g., linking electrophysiological cardiac tissue dynamics to mechanical cardiac tissue dynamics (e.g., predicting three-dimensional electrophysiological activation patterns from mechanical movement patterns) (see, e.g., FIG. 6A). The mechanical dynamics data (or, i.e., mechanical movement pattern data or mechanical deformation data) may comprise any type of spatial measurement of the cardiac tissue such as, e.g., an ultrasound, MRI, or optical measurement. The electrophysiological dynamic data (or, i.e., electrophysiological activation pattern data or electrical dynamic data) may comprise any type of electrophysiological activation patterns correlated with the electrical waves of the heart that induce the mechanical deformation of cardiac tissue (that occurs, e.g., during a heartbeat or heart pulsation). For example, the cardiac tissue surface data may comprise electrical data such as, e.g., voltage measurements or calcium concentration measurements associated with action potential (AP) waves.

The mechanical movement pattern data may be obtained through any number of means including, e.g., through ultrasound, MRI, or optical measurements. The surface data may be obtained through any number of means including, e.g., through conventional catheter-based cardiac mapping approaches and/or the panoramic stereoscopic multi-camera optical mapping techniques of this disclosure, described below. In some embodiments, the mechano-electrical model may be able to construct the 3D intramural electrophysiological activation patterns occurring during normal heart function (e.g., sinus rhythm) from mechanical movement pattern data. In some embodiments, the mechano-electrical model may be able to construct the 3D intramural electrophysiological activation patterns that occur during abnormal heart function. In some embodiments, the mechano-electrical model is able to construct or generate the 3D intramural electrical wave patterns that occur during both normal and abnormal heart function, e.g., from ultrasound data. In some embodiments, the mechano-electrical model may be able to generate or construct mechanical movement pattern data, e.g., from 2D or 3D electrophysiological activation patterns of cardiac tissue.

In order to train the mechano-electrical model, a training dataset sufficient for training the mechano-electrical model to perform one or more specific tasks is generated or obtained. In some embodiments, mechano-electrical model training datasets may comprise high-resolution in silico, ex vivo, and/or in human (e.g., clinically obtained) data. In silico training data may be generated for the mechano-electrical model in a similar manner as to the training data of the EWD model discussed above. For example, to ensure sufficient heterogeneity, simulations may be run using multiple different electrical numerical models coupled to multiple different mechanical models (such as, e.g., the Holzapfel-Ogden, Goktepe-Ogden, Wang et al., and Eriksson et al. models discussed in Example 3 of the disclosure) and using multiple different computational techniques (such as, e.g., finite-difference method (FDM), finite element method (FEM), finite volume method (FVM), smoothed-particle hydrodynamics (SPH), and/or boundary-element method (BEM)) (see, e.g., FIG. 6B). The parameters used to run each simulation of each model may also be varied, e.g., randomly, within a specific range of values to ensure sufficient heterogeneity. In some embodiments, synthetic noise is introduced into the in silico training data simulations. The synthetic noise may be configured to recreate or capture the noise phenomena occurring in the measurements of the mechanical movement patterns, or electrophysiological activation patterns, that are intended to be input into the trained mechano-electrical model during use (e.g., the noise naturally occurring in ultrasound measurements the trained mechano-electrical model will be applied to).

In some embodiments, ex vivo training data may be generated from optical mapping experiments, such as, e.g., the panoramic/stereoscopic optical mapping experiments of this disclosure discussed below. In some cases, ex vivo training data may be generated from electrophysiological activation patterns on the surface of cardiac tissue (e.g., obtained from optical mapping experiments) that are input into an EWD model of this disclosure in order to construct or generate 3D electrophysiological activation pattern data of the training dataset. In some cases, ex vivo training data may be generated from optical or ultrasound measurements that are input into an optical or ultrasound motion tracking model of this disclosure in order to construct/generate mechanical movement pattern data of the training dataset. In human (e.g., clinically obtained) training data may be obtained via, e.g., conventional catheter-based cardiac mapping measurements in conjunction with ultrasound or MRI measurements. In some embodiments, in human training data may be obtained using an EWD or motion tracking model of this disclosure. In some embodiments, the training dataset includes a combination of in silico, ex vivo, and clinical data sufficient to ensure that the dataset is of a size, quality, and heterogeneity sufficient for its intended training purpose. In some embodiments, the training data input during training of the mechano-electrical model is augmented in a similar manner as was discussed for the EWD model above.

The training data may be generated based on the task the mechano-electrical model is trained to perform. In some embodiments, the mechano-electrical model may be trained to construct or generate electrophysiological activation patterns from mechanical movement patterns of a tissue that are obtained, e.g., via ultrasound. In some cases, the mechano-electrical model may be trained to identify a heart rhythm disorder from a temporally consecutive sequence of ultrasound measurements obtained from a patient. In some instances, the mechano-electrical model may be trained to predict future mechanical movement pattern data from one or more measurements of mechanical movement patterns and/or electrophysiological activation patterns. In some cases, the mechano-electrical model may be trained to predict future electrophysiological activation pattern data from one or more measurements of mechanical movement patterns and/or electrophysiological activation patterns.

In some cases, the mechano-electrical model may be trained (or fitted) to detect the presence of, or to localize, fibrosis or tissue scarring from one or more measurements of mechanical movement patterns and/or electrophysiological activation patterns. In some cases, the mechano-electrical model may be trained to compute myocardial fibrosis or scarring maps from ultrasound data and/or catheter data. In these instances, the myocardial fibrosis or scarring maps may be used for diagnosis and/or to guide treatment, e.g., in the form of ablation procedures. In some cases, the (e.g., ground truth) training data used to train the mechano-electrical model regarding tissue fibrosis and/or scarring associated tasks may be generated using one or more of: MRI with a gadolinium-based contrast agent (commonly called late gadolinium enhancement), MRI without contrast agents (called T1 mapping), and/or PET/CT with fibroblast activation protein inhibitor radiotracers.

In some embodiments, the mechano-electrical model may be used in order to, e.g., guide tissue ablation (or another tissue modification technique). For example, methods of the disclosure might include: imaging the heart of a subject using 4D ultrasound in order to generate spatio-temporal mechanical deformation data of the subject's heart during one or more contractions; inputting the mechanical deformation data into a mechano-electrical model of the disclosure, wherein the output of the model is used to locate tissue associated with an abnormal electrical wave, an abnormal electrical pathway, or scar tissue; and ablating tissue associated with the abnormal electrical wave, the abnormal electrical pathway, or the scar tissue in order to disrupt formation or propagation of the abnormal electrical wave. In some embodiments, the output of the mechano-electrical model is used to locate the origin of an abnormal electrical wave. In some instances, the electrical circuits which give rise to an abnormal electrical wave (e.g., an arrhythmia) may be extremely complex. In these cases, the mechano-electrical model may not be able to locate a single origin and instead may, e.g., identify or locate one or more tissue locations which may be ablated or otherwise modified in order to block the propagation of electrical waves and break up the abnormal pathway/abnormal electrical wave.

In some embodiments, the mechano-electrical model is trained to simulate a patient's heart from mechanical movement pattern data, electrophysiological activation pattern data, and/or patient specific data obtained from the patient. Patient or subject specific data may comprise, e.g., demographic data, genetic information, patient history data, structural imaging data, and/or quantitative medical data. In some embodiments, a trained mechano-electrical model may be configured to perform its task in real time as it receives input data. For example, a mechano-electrical model trained to identify heart rhythm disorders from a temporally consecutive sequence of ultrasound measurements may identify heart rhythm disorders in real-time as it receives ultrasound measurements or, e.g., may identify heart rhythm disorders 10 minutes or less after it receives ultrasound measurements including, e.g., 5 minutes or less, or 2 minutes or less, or 1 minute or less, or 10 seconds or less.

In some embodiments, several heart beats (e.g., heart contractions) are averaged and/or analyzed to improve temporal resolution. In some embodiments, cardiac gating based on, e.g., ECG signals is used to improve temporal resolution and/or to minimize imaging artifacts caused by cardiac motion. In some embodiments, the mechano-electrical model may input ECG signals and/or may input data from several heart beats to improve temporal resolution and/or to minimize imaging artifacts. In some embodiments, temporal resolution improvement and/or imaging artifact minimization (via, e.g., averaging/analyzing several heart beats and/or ECG signals) may be performed by a separate model (e.g., the optical or ultrasound motion tracking models of the disclosure), and the separate model may feed into the mechano-electrical model in order to improve the performance of the mechano-electrical model.

In some embodiments, the mechano-electrical model is trained to work in conjunction with a numerical model in order to simulate a heart (e.g., for drug discovery). In some embodiments, the mechano-electrical model comprises physics-informed neural networks (PINNs). In some embodiments, the mechano-electrical model may be a machine learning model including an artificial neural network (NN). In some embodiments, the machine learning model is a deep learning model. In these cases, the model may be three or more layers deep, such as five or more layers deep, or ten or more, or twelve or more, or thirty or more, or fifty or more, or one hundred or more. In some cases, the mechano-electrical model comprises attention or convolutional mechanisms. In some cases, the mechano-electrical model may be a numerical model and, e.g., the training data of the mechano-electrical model discussed above is used to fit the model or adjust the parameters of the model. For example, the mechano-electrical model may be a differentiable simulation model fitted to data (e.g., fitted to the training dataset discussed above) using, e.g., gradient descent techniques. In some embodiments of the present invention, simulations of focal and reentrant electromechanical activation dynamics in idealized bi-ventricular geometries are generated, and the generated data is used to train a mechano-electrical model (including, e.g., a neural network) to analyze ventricular deformation mechanics and, subsequently, to predict the three-dimensional electrical wave pattern(s) that caused the deformation. Accordingly, next to focal wave patterns, even complicated three-dimensional electrical scroll wave patterns can be reconstructed using the deep learning techniques of this disclosure, even if, e.g., the trained mechano-electrical model has never seen the particular arrhythmia or heart geometry and was trained on a different electrophysiological or mechanical model. Mechano-electrical models, such as deep learning models, of the present invention have the ability to generalize through training on data generated, e.g., with one computational technique (e.g., smoothed particle hydrodynamics (SPH) method) and, subsequently, being applied to data generated with a different computational technique (e.g., finite element method (FEM)). Predictions can be performed in the presence of scars and with significant heterogeneity. With adequate (i.e., sufficient) training data, embodiments of the present invention can be used to calculate intramural action potential wave patterns from imaging data of the motion of the heart muscle.

As described above, embodiments of the methods include imaging techniques and apparatuses (e.g., chambers) for fully panoramic stereoscopic imaging of contracting hearts. By ‘fully panoramic’ is meant that all 360° of the surface of the heart (apart from the surface covered, e.g., by mechanisms used to mount or stimulate the heart) is imaged, e.g., while the heart is in motion. By ‘stereoscopic’ is meant each voxel or region of the surface of the heart is imaged from multiple perspectives simultaneously, e.g., using multiple cameras (such that, e.g., stereoscopic algorithms may be applied for motion tracking the voxels/regions of the surface). The panoramic/stereoscopic optical mapping techniques of this disclosure may generate electrophysiological activation pattern data (obtained via, e.g., imaging of voltage- or calcium-sensitive dyes introduced into the heart) temporally and spatially coupled with mechanical movement pattern data (obtained via, e.g., optically imaging the surface of the heart and/or via 3D ultrasound imaging of the intramural regions of the heart). In some embodiments, the coupled electrophysiological activation pattern data and mechanical movement pattern data may be used to generate training date for, validate the performance of, and/or refine parameters of a mechano-electrical model of this disclosure.

In some embodiments, the panoramic and/or stereoscopic optical mapping methods of the disclosure are performed using an imaging apparatus including: an enclosed interior volume into which a tissue is introduced; a substrate on which the tissue is mounted; an electrode for electrically stimulating the tissue; a perfusion subsystem configured to support physiological conditions of the tissue; a plurality of light sources configured for optically illuminating the tissue; an imaging system comprising an optical imaging subsystem configured for obtaining optical images of the tissue; and a plurality of mounts configured for mounting the light sources and claims of the imaging system such that the tissue is evenly illuminated, and the imaging system obtains optical images of the tissue from a plurality of different perspectives. In some cases, the imaging system of the imaging apparatus further includes an ultrasound imaging subsystem configured for obtaining ultrasound images of the tissue. In some embodiments, the imaging apparatus further includes a plurality of electrodes configured to detect electrophysiological activation patterns of the tissue, e.g., by measuring electrocardiograms from the tissue. In some cases, the plurality of mounts are configured for mounting aspects of the imaging system such that every point of the surface of the tissue is imaged from two or more perspectives (i.e., such that stereoscopic motion tracking techniques may be used).

In some cases, the imaging apparatus comprises a rig or chamber. The chamber may have a soccer ball geometry or a truncated icosahedron shape and may include, e.g., a plurality of penta- or hexagonal surfaces. In some cases, the imaging apparatus includes a shape with angles of optical axes between two adjacent surfaces of 37.4° or 41.8°. In some embodiments, the optical imaging subsystem includes 6 or more cameras such as, e.g., 10 or more cameras, or 12 or more, or 15 or more, or 20 or more. In some cases, the angle between neighboring cameras is between 35 and 45°. In some embodiments, the optical imaging subsystem comprises a plurality of CCD or CMOS cameras. In some instances, the optical imaging subsystem is configured for measuring changes in fluorescence of aspects of the tissue such including, e.g., in response to a physiological change of the tissue. The physiological change may include a change in transmembrane potential of the tissue. In some embodiments, the ultrasound imaging subsystem is configured for obtaining ultrasound images of the tissue at extremely rates such as, e.g., a rate of 100 volumes per second or more, or 200 volumes per second or more, or 500 volumes per second or more, or 100 volumes per second or more, or 2000 volumes per second or more, or 300 volumes per second or more, or 5000 volumes per second or more, or 5200 volumes per second or more. some embodiments, the ultrasound imaging subsystem is configured for obtaining ultrasound images of the tissue with high spatial resolution.

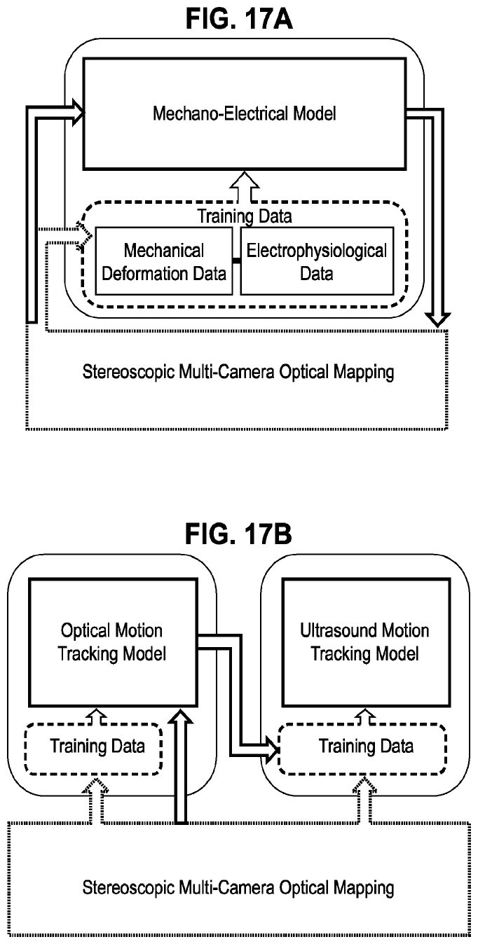

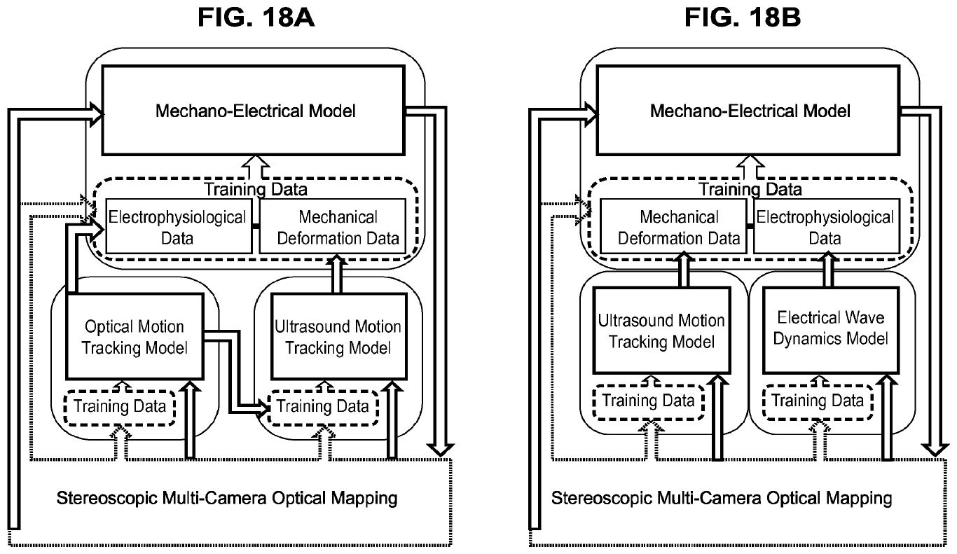

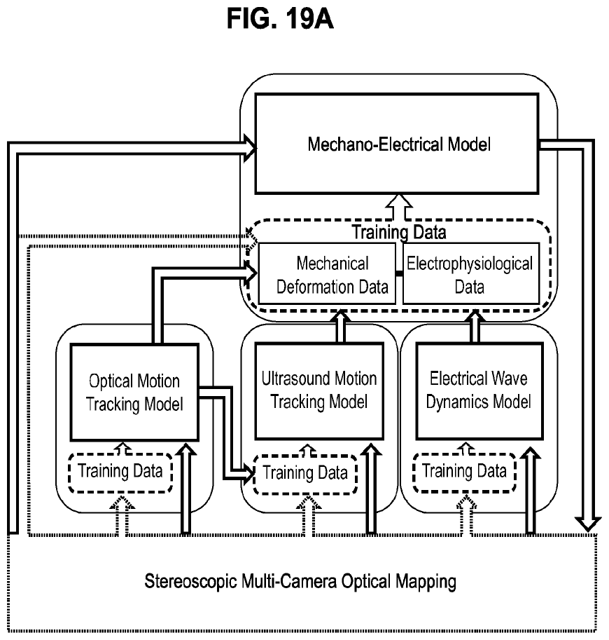

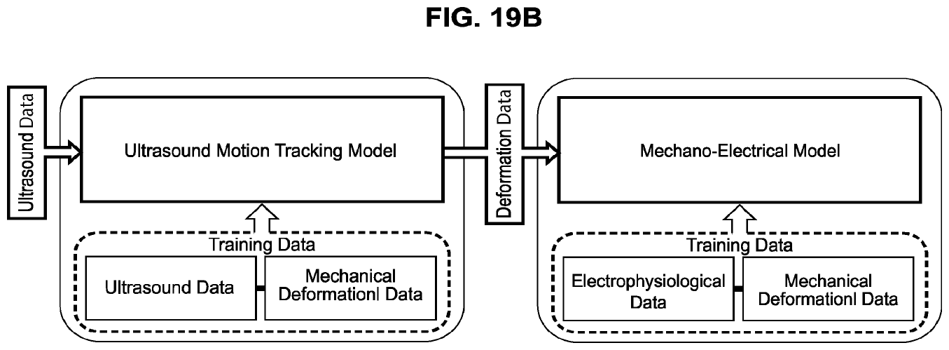

As discussed above, existing methods of creating electrophysiological and mechanical movement cardiac data are incapable of producing training data-sets of sufficient size, quality, or heterogeneity to train a mechano-electrical model of this disclosure (e.g., a model capable linking electrophysiological cardiac tissue dynamics to mechanical cardiac tissue dynamics). In order to generate training data sufficient for the creation of a robust and effective mechano-electrical model, the panoramic and/or stereoscopic optical mapping methods of the disclosure may be used (see, e.g., FIG. 17A). In this way, the panoramic/stereoscopic optical mapping methods of the disclosure may be compared to, e.g., the final piece of a puzzle enabling the creation of the mechano-electrical models of this disclosure. In some cases, the EWD models, optical motion tracking models, and ultrasound motion tracking models of the disclosure may be used to further enhance the performance of mechano-electrical models of this disclosure (see, e.g., FIGS. 18 and 19). For example, the EWD models, optical motion tracking models, and ultrasound motion tracking models, in combination with the panoramic stereoscopic optical mapping methods, may be combined via, e.g., a training network or web, or an iterative improvement loop, in order to enhance or refine mechano-electrical model training data and/or mechano-electrical model performance. Further, similar to the enabling nature of the panoramic/stereoscopic optical mapping techniques for the mechano-electrical model, the panoramic/stereoscopic optical mapping techniques discussed above may enable the creation of training data sufficient for the creation of the optical motion tracking models and/or ultrasound motion tracking models of the disclosure (see, e.g., FIG. 17B).

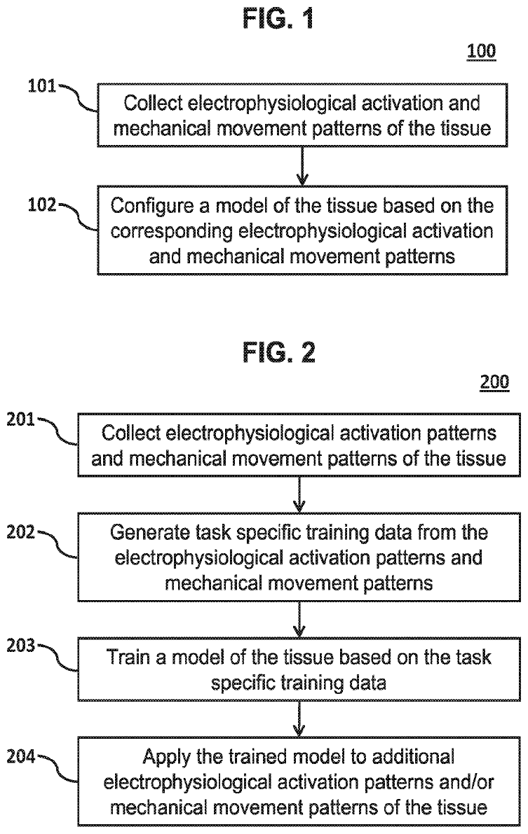

FIGS. 1 and 2 provide flow diagram for practicing methods of modeling mechanical movement patterns and electrophysiological activation patterns of a tissue in accordance with an embodiment of the disclosure.

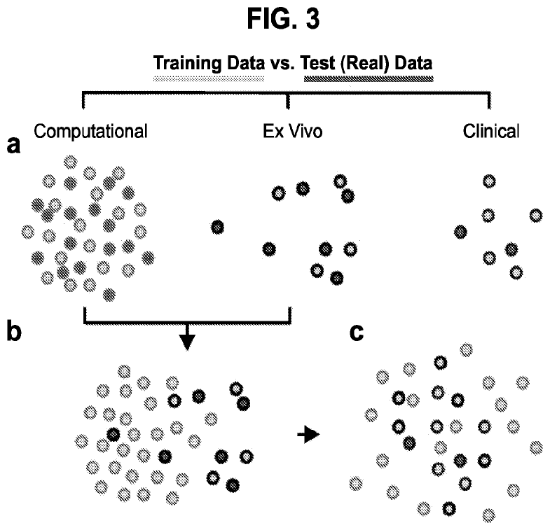

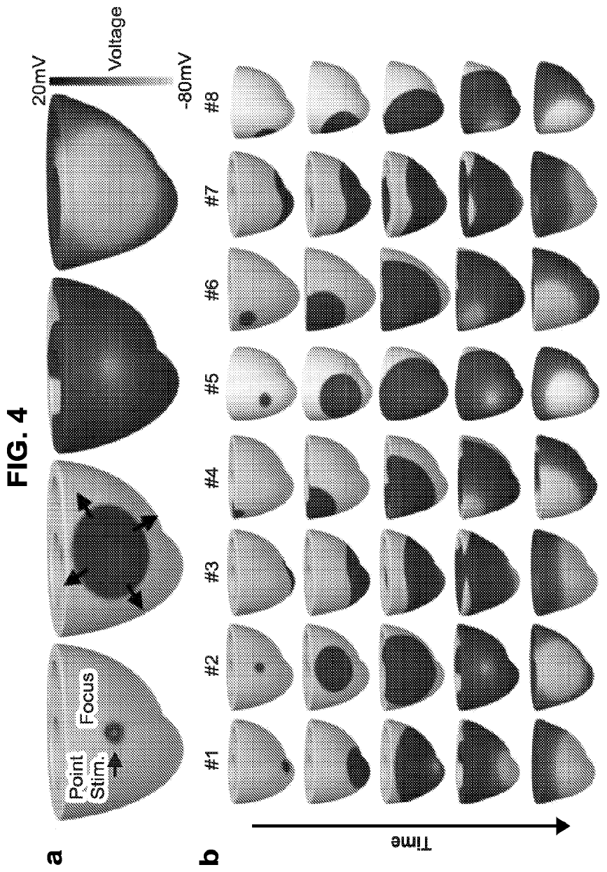

FIGS. 3A to 3C depict a distribution matching of in silico, ex vivo and clinical training data with sparse, heterogeneous, and out-of-distribution data to achieve generalization in accordance with an embodiment of the disclosure. In some cases, training data (such as, e.g., electromechanical data, electrophysiological data, fluorescence data, mechanical deformation data, and/or ultrasound data, depending on the model) may be combined based, e.g., on observations of the panoramic stereoscopic optical mapping experiments of the disclosure. The combined data set is adjusted, combined, refined, and augmented in order to generalize a model or, i.e., to enable the model to learn underlying dynamics of a phenomena rather than simply the dynamics of a specific data generating modality.

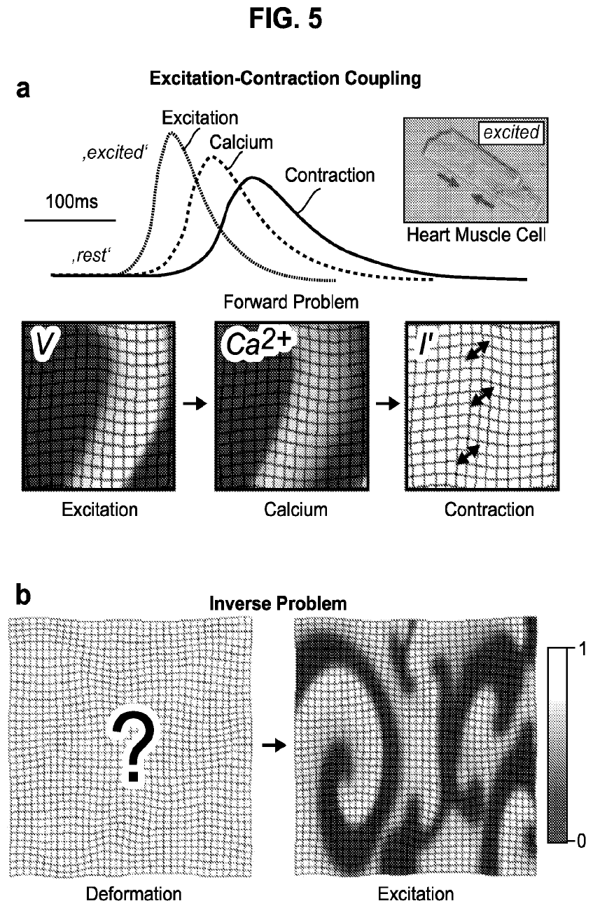

FIGS. 4A to 4B depict training data generation for the mechano-electrical model using electromechanical simulations with randomized initialization of focal or reentrant ventricular arrhythmias. The training data enables the mechano-electrical model to reconstruct cardiac excitation waves from tissue deformation. FIGS. 5A to 5B illustrate both the ‘forward’ (FIG. 5A) and the ‘inverse’ (FIG. 5B) mechano-electrical problem addressed by the mechano-electrical models of this disclosure.

FIG. 6A provides a block diagram of a mechano-electrical model and a training data set of paired electrophysiological data (including, e.g., electrophysiological activation patterns) and mechanical deformation data (including, e.g., mechanical movement patterns) used to train the mechano-electrical model. The electro-physiological data and mechanical deformation data varies depending on the function of the mechano-electrical model (i.e., the task the mechano-electrical model will be applied to perform). For example, the mechanical deformation data may comprise consecutive ultrasound measurements when training the mechano-electrical model to predict future deformation states and/or future electrical wave states from mechanical or electrophysiological wave measurements.

FIG. 6B demonstrates an example of potential in silico data that may be included in a mechano-electrical model training dataset. Multiple different electrical models (e.g., numerical electrical models and/or EWD models of this disclosure) are used to generate electrophysiological data. The electrophysiological data is then used by multiple different mechanical models (e.g., numerical mechanical models and/or motion tracking models of this disclosure) to generate mechanical deformation data. In embodiments where the mechano-electrical model is applied to ultrasound measurements, training data may be generated via an ultrasound motion tracking model used in conjunction with a numerical mechanical model. For example, the ultrasound motion tracking model may be used to generate synthetic ultrasound data from the mechanical deformation data output by the numerical mechanical model.

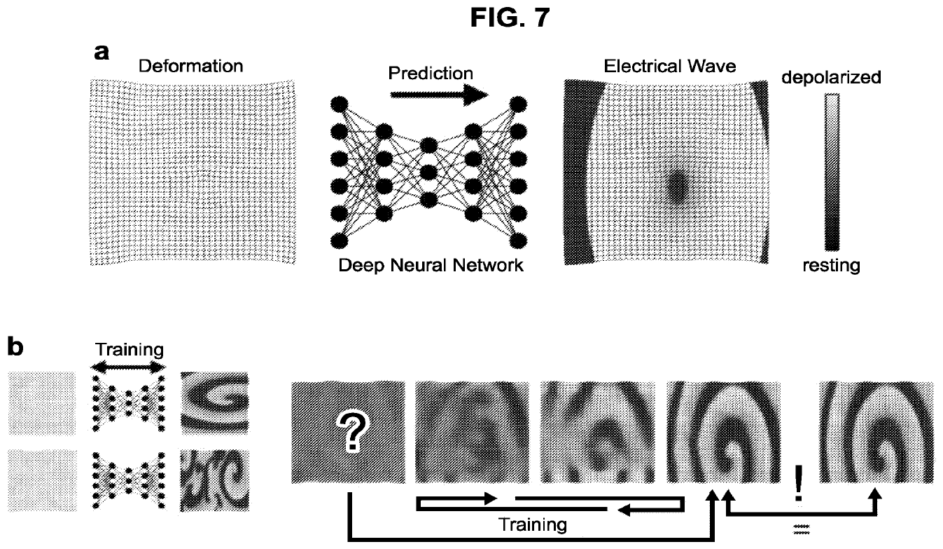

FIGS. 7A to 7B illustrate a self-supervised deep learning-based approach for solving the ‘inverse’ mechano-electrical problem in accordance with an embodiment of the disclosure. FIG. 7A depicts a deep neural network (AI) for the reconstruction/prediction of electrical excitation waves (right) from deformation (left). FIG. 7B illustrates Training with many thousands of pairs of mechanical and electrical data. The neural network memorizes many different electrical patterns, which caused corresponding deformation patterns, and can then subsequently be applied to new data.

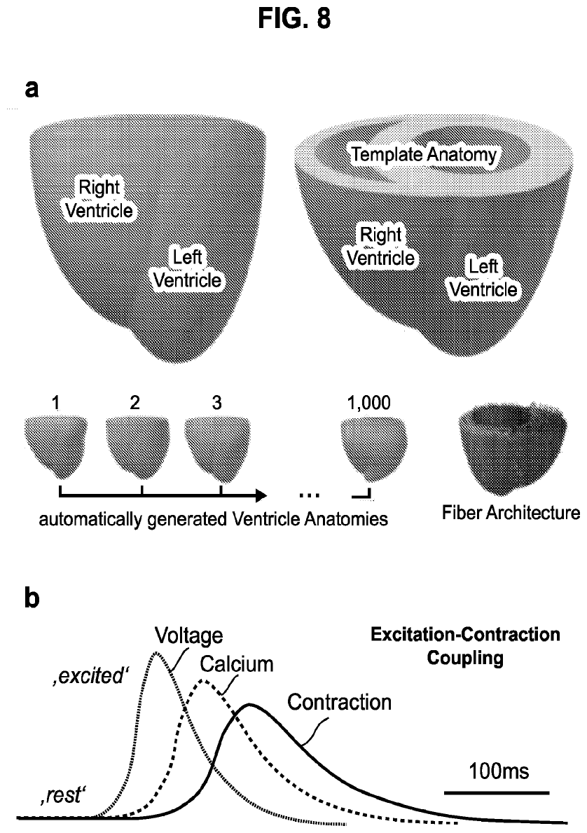

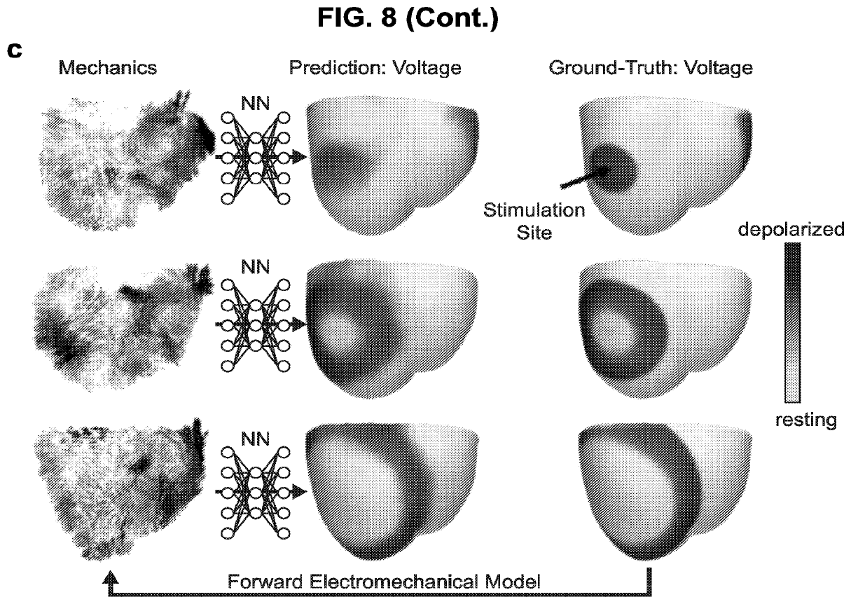

FIGS. 8A to 8C depict computer simulations used to generate training data for a mechano-electrical model in accordance with an embodiment of the disclosure. FIG. 8A shows 1,000 different ventricular geometries used to perform 50,000 unique simulations. FIG. 8B illustrates the cardiac excitation-contraction-coupling mechanism that connects electrical and mechanical dynamics in the heart. FIG. 8C shows a mechano-electrical model that may be used, e.g., to predict subsequent mechanical or electrical states from measured deformation data. Left: 3D motion of the heart. Center: Predictions of electrical waves (dark: depolarized phase of action potential) obtained with AI (NN: neural network). Right: Ground-truth generated in simulations. The ground-truth was used to compute the deformations and motion vectors on the left.

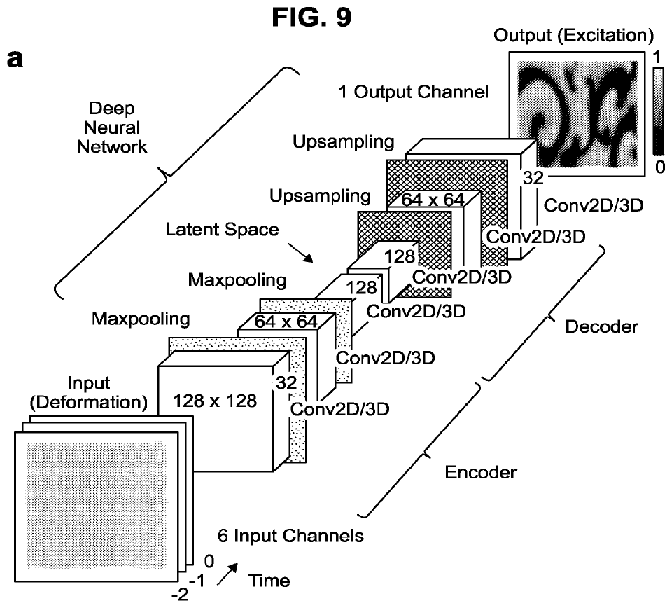

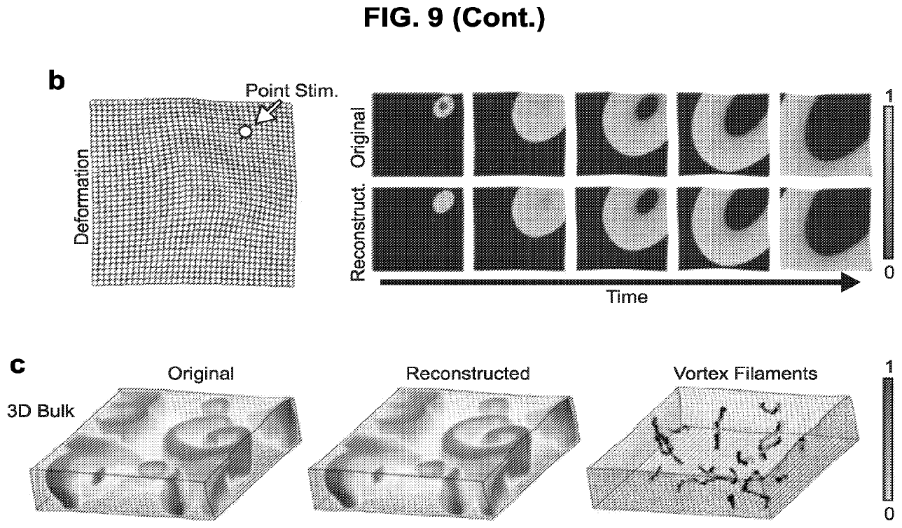

FIGS. 9A to 9C show reconstruction of electrical excitation waves from mechanical deformation using both a deep neural network and numerical modeling. FIG. 9A illustrates a deep neural network (AI) (i.e., a mechano-electrical model) for the reconstruction of electrical excitation waves from deformation. FIG. 9B provides a numerical reconstruction of focal excitation wave from deformation in 2D, the reconstruction being visually indistinguishable from the original. FIG. 9C depicts reconstruction of 3D scroll wave chaos (VF) with very good match between reconstructed (black) and original (gray) vortex filaments.

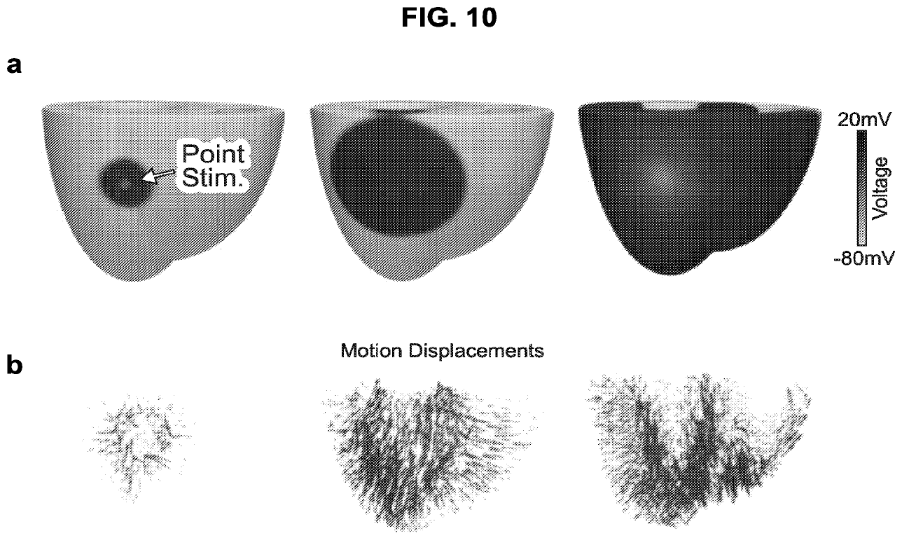

FIGS. 10A to 10B illustrate electrical 3D activation patterns causing unique time-varying 3D contraction patterns. FIG. 10A shows an electrical 3D activation pattern (action potential wave) causing FIG. 10B unique, fingerprint-like time-varying 3D contraction patterns (displacement vectors indicating motion), from which in turn a specially trained mechano-electrical model of the disclosure will be able to compute the original action potential wave.

FIG. 11A provides a block diagram illustrating the use of an electrical wave dynamics (EWD) model to train a mechano-electrical model in accordance with an embodiment of the disclosure. In these instances, the EWD model may be applied in order to create a training dataset for the electro-mechanical model of this disclosure. For example, the EWD model may be used to generate 3D electrical wave mechanics from 2D clinical catheter-based measurements, fill in missing data obtained from clinical imaging techniques, adjust experimental data, inpaint randomly deleted sections of electrical numerical model simulations, generate electrophysiological activation patterns (such as, e.g., scroll waves), etc.

FIG. 11B provides a block diagram illustrating the use of an optical motion tracking model to train a mechano-electrical model in accordance with an embodiment of the disclosure. In these instances, the optical motion tracking model may be applied in order to create a training dataset for the electro-mechanical model of this disclosure. For example, the optical motion tracking model may be applied to optical measurements (e.g., images) obtained during optical mapping experiments (such as, e.g., the panoramic optical mapping experiments of this disclosure) in order to obtain more accurate ground-truth mechanical deformation data.

FIG. 12A provides a block diagram illustrating the use of an ultrasound motion tracking model to train a mechano-electrical model in accordance with an embodiment of the disclosure. For example, clinically obtained ultrasound measurements may be input into the ultrasound tracking model in order to generate or determine the position and/or motion of a discrete area or voxel of a tissue in relation to all the other areas/voxels of the tissue (i.e., mechanical deformation data). The positions and/or motions of the areas/voxels output by the ultrasound tracking model may be coupled with electrophysiological data obtained from catheter-based measurements simultaneously generated with the ultrasound measurements in order to obtain paired electromechanical training data. In some cases, the ultrasound tracking model may be used to generate synthetic ultrasound data, e.g., from numerical mechanical model simulations (e.g., as discussed above) in order to train the mechano-electrical model to predict electrophysiological cardiac tissue dynamics (e.g., intramural electrical waves) directly from ultrasound measurements.

FIG. 12B provides a block diagram illustrating the use of an optical motion tracking model to train an ultrasound motion tracking model in accordance with an embodiment of the disclosure. In some embodiments, the optical motion tracking model may be applied in order to create a training dataset for the ultrasound motion tracking model. For example, optical measurements temporally coupled with ultrasound measurements obtained, e.g., via the panoramic optical mapping experiments of the disclosure may be used to generate ‘ground truth’ training data for the ultrasound motion tracking model.

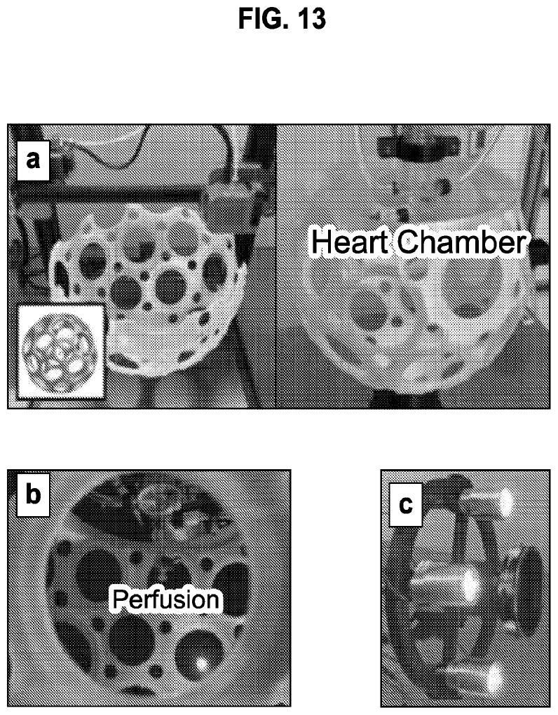

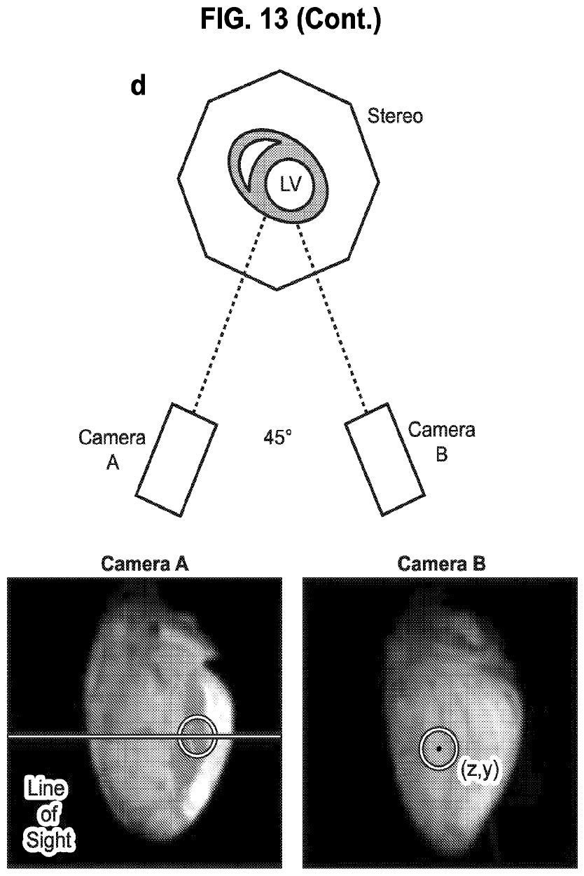

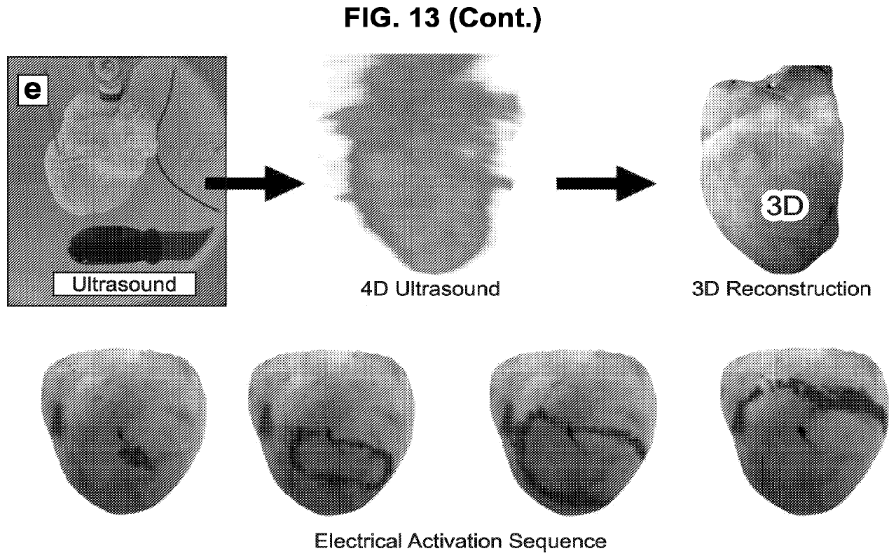

FIGS. 13A to 13E illustrate ex vivo electromechanical imaging of arrhythmias to obtain training data for AI in accordance with an embodiment of the disclosure. FIGS. 13A-13B show 3D-printed heart chamber with 16, 24 or 32 windows for multi-camera optical mapping. FIG. 13C depicts a CMOS camera with LEDs. FIG. 13D illustrates stereoscopic 3D optical mapping of ventricles. FIG. 13E provides ventricular contractions imaged using 4D ultrasound at high speeds in order to produce a 3D reconstruction of cardiac tissue (which may, e.g., then be used to predict an electrical activation sequence or overlay an imaged/observed electrical activation sequence over the 3D reconstruction).

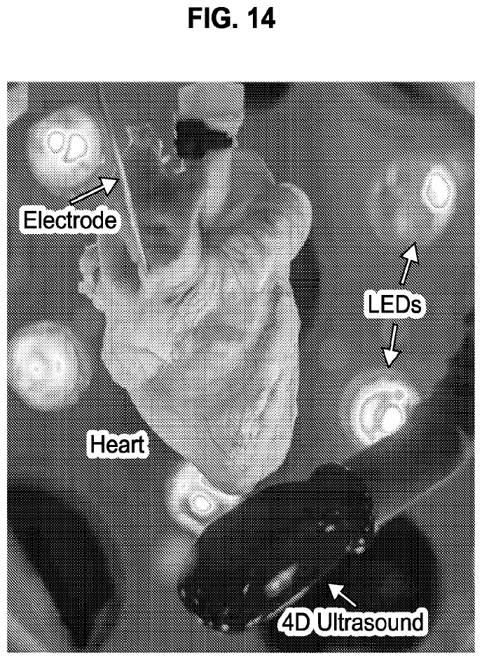

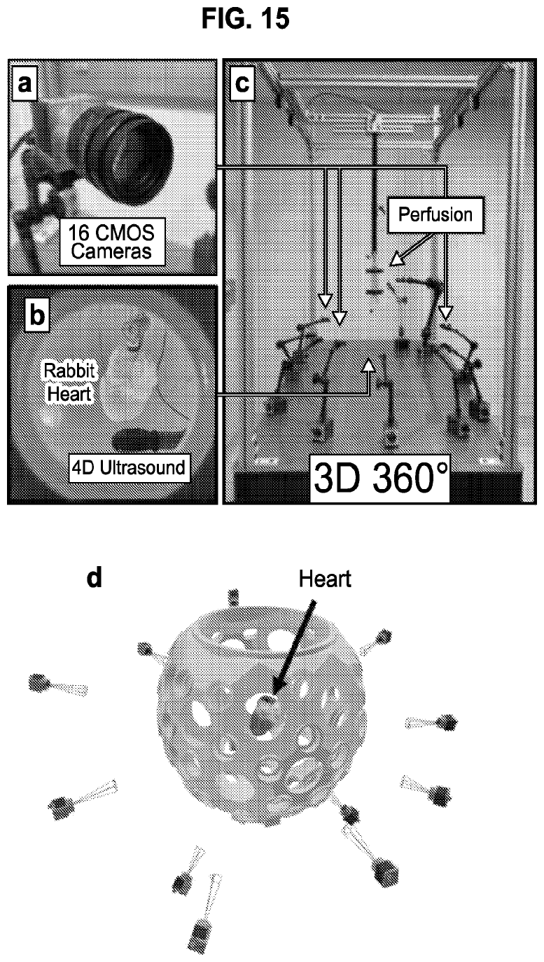

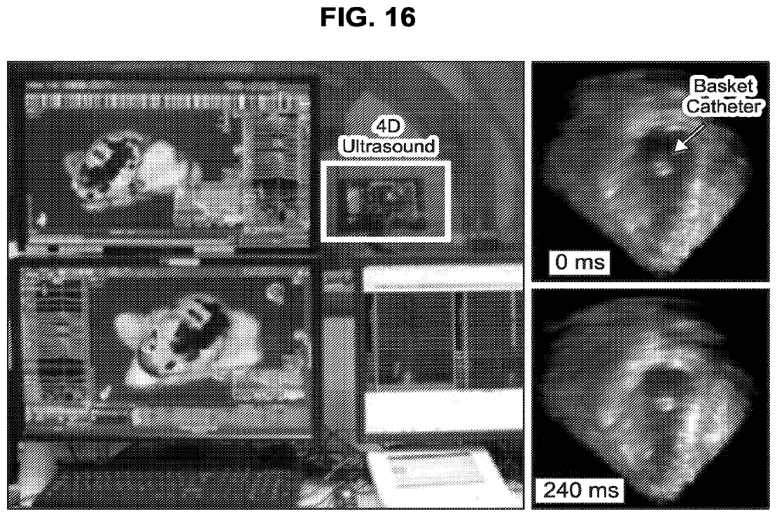

FIG. 14 depicts electromechanical high-resolution imaging of arrhythmias ex vivo using panoramic fluorescence and 4D ultrasound imaging in accordance with an embodiment of the disclosure. FIGS. 15A to 15D show ex vivo electromechanical imaging of arrhythmias, using 16-camera high-speed panoramic fluorescence imaging and highspeed 4D ultrasound, to obtain training data for an inverse mechano-electrical model in accordance with an embodiment of the disclosure. FIGS. 15A, 15C and 15D depict a 16-camera high-speed panoramic fluorescence imaging setup and FIGS. 15B, 15C and 15D illustrate highspeed 4D ultrasound to obtain training data for inverse AI (i.e., training an mechano-electrical model of the present disclosure). FIG. 16 depicts ultrasound imaging during catheter ablation procedures in humans in accordance with an embodiment of the disclosure.

FIG. 17A depicts a block diagram of stereoscopic panoramic multi-camera optical mapping being used to train and/or validate a mechano-electrical model in accordance with an embodiment of the disclosure. In some cases, stereoscopic multi-camera optical mapping is used to produce paired electrophysiological data (e.g., measurements of action potential (AP) waves or wave fronts, calcium waves or wave fronts, etc.) and mechanical deformation data (e.g., measurements of motion including, e.g., three-dimensional motion vectors of tissue movement) used to train the mechano-electrical model. In some embodiments, paired electrophysiological and mechanical deformation data (i.e., electromechanical data) is used to evaluate or validate a trained mechano-electrical model. For example, mechanical deformation data of an experimentally obtained electromechanical dataset may be input into the mechano-electrical model in order to predict electrophysiological data. The predicted electrophysiological data may then be compared with the corresponding electrophysiological data of the electromechanical dataset (i.e., the experimentally obtained electrophysiological data corresponding with the input mechanical deformation data) in order to evaluate or validate the mechano-electrical model. The evaluation of the mechano-electrical model may then be used to, e.g., alter the distribution of in silico, ex vivo and clinical training data used to train the mechano-electrical model. In some cases, the evaluation of the mechano-electrical model may be used to adapt or configure the stereoscopic panoramic multi-camera optical mapping to generate data better suited for mechano-electrical model training or refinement. For example, certain pacing regiments may be applied by a stimulation electrode of the stereoscopic panoramic multi-camera optical mapping methods based on the evaluation in order to better train the mechano-electrical model to capture, e.g., a specific electrical wave dynamic. In some embodiments, electromechanical data obtained via panoramic stereoscopic multi-camera optical mapping may be used to, e.g., adjust parameters of numerical simulations used to generate the mechano-electrical training data, adjust augmentation of the mechano-electrical training data (e.g., refine synthetic noise to better capture experimentally observed phenomena), and/or otherwise refine or curate the mechano-electrical training data.

FIG. 17B depicts a block diagram of stereoscopic multi-camera optical mapping being used to train and/or validate an optical motion tracking model in conjunction with an ultrasound motion tracking model, in accordance with an embodiment of the disclosure. In some embodiments, stereoscopic multi-camera optical mapping may be used to produce optical measurements temporally coupled with ultrasound measurements, which may be used to generate ground truth training data (comprising, e.g., mechanical deformation data paired with ultrasound data) for the ultrasound motion tracking model. The ground truth training data of the ultrasound motion tracking model may be generated using, e.g., numerical stereoscopic motion tracking techniques or the optical motion tracking model. In some embodiments, stereoscopic multi-camera optical mapping may be used to produce optical measurements temporally coupled with mechanical deformation data, which may be used to generate ground truth training data for the optical motion tracking model. The ground truth training data of the optical motion tracking model may be generated using, e.g., numerical stereoscopic motion tracking techniques. In some embodiments, the numerical (e.g., stereoscopic) motion tracking techniques are accelerated by leveraging the capabilities of modern graphics processing units (GPUs).

FIG. 18A shows an optical motion tracking model, an ultrasound motion tracking model, and panoramic stereoscopic optical mapping connected together in a training network or training loop in order to iteratively refine a mechano-electrical model in accordance with an embodiment of the disclosure. In a similar manner as described for FIG. 17A, the stereoscopic panoramic multi-camera optical mapping may be used to train and/or validate the mechano-electrical model, and in a similar manner as described for FIG. 17B, the stereoscopic panoramic multi-camera optical mapping may be used to train and/or validate the optical motion tracking model and/or the ultrasound motion tracking model. Further, in a similar manner as described for FIGS. 11B and 12B, the ultrasound motion tracking model and the optical motion tracking model may be used to train the mechano-electrical model. In some embodiments, each model achieves better performance every time the stereoscopic panoramic multi-camera optical mapping is performed, which, in turn further elevates downstream models (i.e., models wherein training is affected by a different model, e.g., the ultrasound motion tracking model relative to the optical motion tracking model in FIG. 18A), culminating in significant improvements to the mechano-electrical model each iterative performance of the stereoscopic panoramic multi-camera optical mapping. Further, although not depicted in FIG. 18A, the mechano-electrical model may be used to train the ultrasound motion tracking model and/or the optical motion tracking model. Accordingly, the training network or training loop formed by the models may include synergistic effects that improve the performance of each model relative to what could be accomplished if each model were to be evolved or refined separately.

FIG. 18B shows an ultrasound motion tracking model, an EWD model, and panoramic stereoscopic optical mapping connected together in a training network or training loop in order to iteratively refine a mechano-electrical model in accordance with an embodiment of the disclosure. In a similar manner as described for FIG. 17A, the stereoscopic/panoramic multi-camera optical mapping may be used to train and/or validate the mechano-electrical model, and in a similar manner as described for FIG. 17B, the stereoscopic/panoramic multi-camera optical mapping may be used to train and/or validate the ultrasound motion tracking model. Further, in a similar manner as described for FIGS. 11A and 12B, the ultrasound motion tracking model and the EWD motion tracking model may be used to train the mechano-electrical model. In some embodiments, the stereoscopic panoramic multi-camera optical mapping may be used to train and/or validate the EWD model. For example, a plurality of detecting/measuring electrodes may be positioned within a tissue of an optical mapping experiment such that surface electrophysiological data (obtained via, e.g., imaging voltage- or calcium-sensitive dyes) paired with intramural electrophysiological data (obtained via the plurality of measuring electrodes) may be used to generate ground truth training data for the EWD model. In some embodiments, each model achieves better performance every time the stereoscopic/panoramic multi-camera optical mapping is performed, which, in turn further elevates downstream models, culminating in significant improvements to the mechano-electrical model each iterative performance of the stereoscopic/panoramic multi-camera optical mapping. Further, although not depicted in FIG. 18B, the mechano-electrical model may be used to train the ultrasound motion tracking model and/or the EWD model. Accordingly, the training loop formed by the models may include synergistic effects that improve the performance of each model relative to what could be accomplished if each model were to be evolved or refined separately.

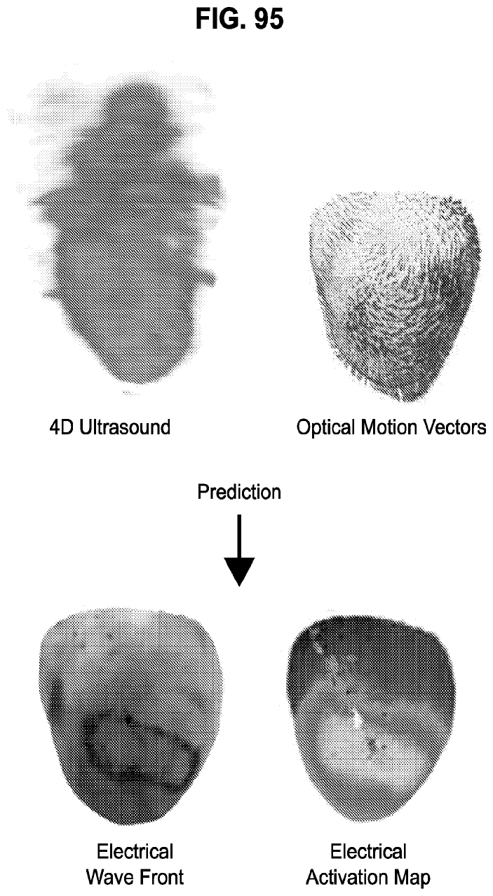

FIG. 19A shows an embodiment of a training network or training loop formed by an EWD model, an optical motion tracking models, an ultrasound motion tracking model and panoramic/stereoscopic optical mapping and used in order to iteratively refine a mechano-electrical model in accordance with an embodiment of the disclosure. Similar to as was described for FIGS. 11-12 and 17-19, the training loop formed by the models may include synergistic effects that improve the performance of each model relative to what could be accomplished if each model were to be evolved or refined separately. FIG. 19B shows an alternate configuration of the ultrasound tracking model being used with the mechano-electrical model in order to, e.g., predict three-dimensional electrical wave dynamics from clinically obtained ultrasound data. In this instance, clinically obtained ultrasound data may be input into the ultrasound tracking model in order to generate or determine the position and/or motion of each discrete area or voxel of a tissue in relation to all the other areas/voxels of the tissue. The positions and/or motions of the areas/voxels output by the ultrasound tracking model may then be input into the mechano-electrical model in order to for the mechano-electrical model to, e.g., predict three-dimensional electrophysiological activation patterns from the ultrasound data. FIG. 95 depicts how 4D ultrasound and computed three-dimensional motion vectors of tissue movement may be used in order to predict electrical wavefront propagation and calculate electrical activation maps. For example, optical flow estimation techniques or differentiable motion estimation techniques (including, e.g., the optical motion tracking models discussed above) may be applied, e.g., to compensate for tissue movement and/or to generate optical motion vectors from optically obtained data (obtained via, e.g., the panoramic stereoscopic mapping techniques of the disclosure). The mechano-electrical model may then be applied, e.g., in order to predict electrical wavefront propagation and calculate electrical activation maps from the 4D ultrasound data and/or the three-dimensional motion vectors of tissue movement. In some embodiments, the 4D ultrasound data may be used to compute three-dimensional motion vectors of tissue movement.

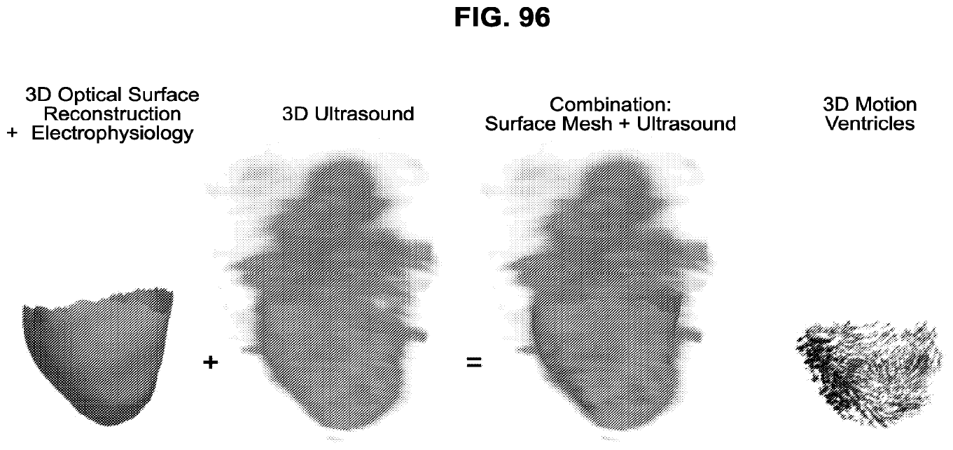

FIG. 96 illustrates how 3D optical surface reconstructions and electrophysiology, in combination with 3D ultrasound, is used to map a surface mesh onto ultrasound datapoints of the heart and compute three-dimensional motion vectors of tissue movement. In some embodiments, the 3D optical surface reconstructions, electrophysiology, and/or 3D ultrasound data may be generated, e.g., using the panoramic stereoscopic mapping techniques of the disclosure. The generated data (including, e.g., the mapped surface mesh and computed three-dimensional motion vectors) may be used in order to train a mechano-electrical model of the disclosure (or, similarly, fit a differentiable simulation mechano-electrical model of the disclosure)

### Applications and Training Schemes

### Mechano-Electrical Reconstructions Using 4D Ultrasound:

Embodiments of the present invention comprise training a machine learning model (i.e., an AI) that can recognize the electrophysiological processes underlying heart rhythm disorders based on, for example, ultrasound videos—an approach that is highly unconventional. The AI may be trained with high-resolution training data of heart rhythm disorders generated in a laboratory with intact, isolated hearts. Using unique ex vivo imaging setup, electrophysiological and tissue-mechanical phenomena can be measured at unprecedented resolutions, which are currently unattainable in humans. In other words, embodiments of the present invention relate to an approach that is comparable to the approach used by developers of self-driving cars. These developers generate training data for AI-assisted driving, collecting extensive video and other sensor data. Based on the data, the AI then learns to ‘drive a car’. Machine learning models in embodiments of the present invention learn to predict the underlying electrophysiology of cardiac arrhythmias. Trained with high-resolution data, the AI will become highly specialized in recognizing the spatio-temporal organization of heart rhythm disorders and visualizing their dynamics in 3D over time (=4D) at rapid speeds throughout the entire organ. Based on its training in a laboratory, the AI can then be applied ‘in the wild’ and visualize the same processes in human patients during ultrasound examinations.

The heart's contractions are triggered by electrophysiological waves, or action potential waves, which propagate rapidly through the heart muscle and initiate the release of intracellular calcium, which in turn is involved in the generation of active contractile stress in cardiac muscle cells via the so-called cellular ‘excitation-contraction coupling mechanism’. Traditionally, cardiologists are interested in the electrophysiological wave phenomena because they reveal potential causes of heart rhythm disorders, and their visualization can guide therapies such as catheter ablation. However, it is difficult and time-consuming to visualize the heart's electrical activity because it requires inserting electrodes into a patient's heart and measuring the activity point by point across the heart surface. This procedure can take up to several hours and is invasive. By contrast, embodiments of the present invention are based on the idea that, because the heart's contractions and deformations are triggered by electrophysiological waves, inversely these waves can be computed by analyzing the heart's contractions and deformations. The heart's motion can be visualized routinely non-invasively using high-speed three-dimensional (3D+t=4D) ultrasound. However, thus far ultrasound cannot visualize electrophysiological waves, but merely display motion. Embodiments of the present invention change that. Recent work has found i) that the heart's global electrophysiological and mechanical dynamics are highly correlated even during complex heart rhythm disorders and ii) that ultrasound can be used to visualize the mechanical fingerprints of electrophysiological vortex phenomena during cardiac fibrillation (see, e.g., Example 1 of this disclosure, provided below). Even more importantly, it has been found iii) that deep learning can accurately ‘predict’ electrophysiological wave phenomena from simulated tissue deformation at high resolutions (see, e.g., Example 3 of this disclosure, provided below), even if the deformations exhibited by the heart muscle during heart rhythm disorders are highly complex. The latter aspect is particularly exciting, because the three-dimensional electrophysiological wave phenomena, that are underlying heart rhythm disorders such as atrial or ventricular fibrillation, degenerate into complex spatio-temporal electrical chaos and cause very irregular and complex deformation patterns.

Embodiments of the present invention are able to solve this ‘inverse problem’ with computer simulations. They can do so because the machine learning models have been trained with tens of thousands of examples of heart rhythm disorders from which they learned to associate an electrical state with a given deformation. After training, machine learning models of embodiments of the present invention can predict any ‘electrical circuit’ underlying a particular rhythm from the heart's motion, even if it has never seen this individual rhythm and although the relationship between tissue deformation and electrophysiology is highly complex, the arrhythmia potentially chaotic, and in each individual case unique.

The central hypothesis underlying embodiments of the present invention is that the motion and deformation of the heart contains a wealth of information, which-if captured in a 4D measurement and analyzed properly—is unique enough to be able to compute the 4D electrophysiological dynamics causing the heart rhythm disorder. Subsequently, training a machine learning model (i.e., an AI) to learn the complex relationship between cardiac electrics and tissue mechanics from a well-curated 4D imaging dataset obtained under laboratory conditions and undergirded by clinical and simulated data, will enable the AI to predict any electrical activation pattern from any deformation in any heart. Just as AI can recognize objects, faces, speech or handwritten text and any other complex high-dimensional feature in data, embodiments of the present invention comprising a specially trained AI will also be able to recognize any heart rhythm disorder solely by observing 4D ultrasound data. Embodiments of the present invention are developed with intact isolated hearts. A first training dataset with isolated (rabbit, pig, and human) hearts using 4D ultrasound and panoramic voltage-sensitive fluorescence imaging have been developed in connection with embodiments of the present invention. Both modalities generate ex vivo training data, which can be mixed with in silico and human training data generated in computer simulations and in the clinical setting.

Embodiments of the present invention may be useful in providing diagnostics that could be performed non-invasively and in real-time (compared to 30-90 minutes) and the approach could be used complementarily to conventional electrical catheter mapping to guide catheter ablation or other forms of therapy.

### AI-Enabled 4D Ultrasound-Based Mapping of Electrical Ventricular Tachycardia Circuits

Embodiments of the present invention establish an entirely novel ultrasound-based imaging technique utilizing artificial intelligence (AI) for the non-invasive, transmural visualization and diagnosis of the 3D morphology of ventricular arrhythmias. The imaging will supersede existing mapping techniques, as it will produce 3D visualizations of heart rhythm disorders in real-time and provide novel insights into the origins and driving mechanisms of heart rhythm disorders.

Despite significant progress in the field of biomedical imaging, imaging of heart rhythm disorders remains a major technological and scientific challenge. Understanding the origins and underlying mechanisms of heart rhythm disorders is pivotal in the planning of therapeutic interventions. Currently, heart rhythm disorders, such as atrial fibrillation (AF) or ventricular tachycardia (VT), are routinely diagnosed using catheter-based mapping. Catheter-based measurements are time-consuming and typically provide information about the heart's abnormal electrical activity on its surface. However, the electrical wave phenomena associated with ventricular arrhythmias are inherently three-dimensional phenomena evolving deep within the cardiac muscle, where they often interact with an arrhythmogenic substrate (e.g. scar tissue) in locations that are inaccessible to standard catheter mapping.

Embodiments of the present invention comprise a novel AI-enabled and 4D (time-varying 3D) ultrasound-based imaging approach for the non-invasive and in-depth visualization of heart rhythm disorders. The imaging approach is based on the idea that, because the heart's contractions and deformations are triggered by electrical activity, inversely this electrical activity can be computed by analyzing the heart's deformations. The imaging approach is enabled by AI, which will learn and translate this complex relationship; embodiments of the present invention utilize high-resolution multi-modal optical mapping of voltage, calcium and strain in intact, isolated hearts, and are combined with 4D ultrasound. With this powerful methodological approach, embodiments of the present invention are able to correlate electrophysiological and mechanical tissue dynamics at high resolutions and develop an inverse computational reconstruction technique that is able to decipher the heart's complex deformations. Based on this analysis, the imaging approach will provide high-resolution numerical reconstructions of the arrhythmic electrical phenomena throughout the ventricular heart muscle.

The central hypothesis underlying embodiments of the present invention is that the motion and deformation of the heart contains a wealth of information, which can serve like a fingerprint of an arrhythmia, and which-if captured in a 4D measurement and analyzed properly—is unique enough to be able to compute the 4D morphology of the electrical dynamics causing the arrhythmia. Subsequently, training a machine learning model (i.e., an AI) to learn the complex relationship between cardiac electrics and mechanics from a well-curated 4D imaging dataset obtained ex vivo under laboratory conditions and undergirded by synthetic data generated in computer simulations, will enable the AI to predict any electrical activation pattern from any deformation in any heart. Just as AI can recognize objects, faces, speech or handwritten text and any other complex high-dimensional feature in data, embodiments of the present invention comprising a specially trained AI will also be able to recognize any arrhythmia morphology or electrical circuit solely by observing 4D ultrasound data. The AI will be able to recognize the 3D arrhythmia morphology even though arrhythmias are chaotic in nature and will be different and unique in each patient.

Background: The heart's contractions are triggered by action potential waves, which propagate rapidly from cell to cell and through the heart muscle and lead via the excitation-contraction coupling mechanism to macroscopic deformations of the heart muscle. Heart rhythm disorders are caused by abnormal electrical waves, which may degenerate into complicated, disorganized, self-sustained wave patterns that initiate irregular and persistent contractions of the heart muscle. These waves are conjectured to take on the shapes of rotating scroll waves or ‘rotors’ during reentrant ventricular arrhythmias. However, the precise origin of triggers and the three-dimensional morphology of arrhythmic electrical circuits remains often insufficiently understood, because imaging technology able to resolve the waves throughout the heart muscle does not exist. Modern ultrasound imaging can provide high-speed 4D videos of the entire heart at resolutions sufficient to resolve even the rapid, small irregular contractions during ventricular fibrillation. However, ultrasound provides solely images of the motion of the heart and does not provide electrical information.

Current State-of-the-Art: The current state-of-the-art for the imaging of heart rhythm disorders is electro-anatomic catheter-mapping, which provides measurements of electrical activity in locations sparsely distributed across the heart surface. Catheter mapping is time-consuming as the mapping needs to be performed sequentially, point-by-point to obtain high-resolution maps. Procedural times are typically 30-90 minutes to map an arrhythmia. The measurements are superficial and prone to motion-related artifacts and maps cannot distinguish epi-, endo- or intracardial signal. Intramural measurements using needle electrode catheters are performed in specialized cardiac electrophysiology laboratories to identify intramural, often isthmus-related origins of VTs in patients with ischemic cardiomyopathies (ICM) but are not routinely done in most electrophysiology laboratories because these catheters are not commercially available and require significant time and expertise to perform. An alternative imaging technique is inverse electrocardiogramhich involves recording body surface electrical potentials to calculate potentials on the heart surface. However, the technique provides indirect measurements and requires further validation. Both techniques require 3D location sensors or imaging data (e.g. CT or MRI) to create electro-anatomic maps. In the laboratory, heart rhythm disorders can be studied with optical mapping, which provides highly detailed visualizations of the heart's electrical activity but fails to image activity deep within the cardiac muscle, similarly as clinical techniques. Optical mapping is limited by low penetration depths of light in tissue and involves toxic substances and can therefore not be used in patients.

Embodiments of the present invention address a very significant challenge within cardiovascular medicine, and a long-standing problem within biomedical engineering: the non-invasive and in-depth visualization of cardiac arrhythmias. Currently, no experimental method exists that can visualize three-dimensional, transmural electrical activity of the heart non-invasively. The inventors have shown that 4D ultrasound imaging can resolve the electromechanical phenomena underlying cardiac fibrillation, and that this technique could-if combined with AI-provide high-resolution, three-dimensional visualizations of heart rhythm disorders non-invasively, in real-time and with high precision (see, e.g., Examples 3, 6 and 7 of this disclosure, provided below). The proposed 4D imaging approach is expected to supersede existing cardiac mapping approaches and could greatly advance the diagnosis of heart rhythm disorders and be used to guide therapeutics, such as catheter ablation, more reliably and effectively. For example, while simple reentrant arrhythmias (e.g. “AVNRT”, “AVRT”, atrial flutter) are easily mapped and ablated with high success, complex reentrant arrhythmias (e.g. ischemic VT, polymorphic VT, persistent AF or VF) are not easily mapped and ablated clinically. In some cases, the imaging technique could provide highly detailed live 4D visualizations of these heart rhythm disorders, while guiding more targeted, intracardial ablation procedures. Independent of its potential transformative clinical impact, embodiments of the present invention will open doors to a novel field with extraordinary potential in unraveling cardiac arrhythmia mechanisms.

High-Resolution 4D Ultrasound-based Imaging of Electromechanical Rotors during Ventricular Fibrillation (VF) Ex Vivo: The inventors have demonstrated ex vivo that it is possible to image and characterize the electrical and mechanical phenomena underlying ventricular fibrillation (VF) at unprecedented resolutions using 4D ultrasound and panoramic optical mapping. By imaging the fibrillating heart with both imaging techniques simultaneously, the inventors were able to show that the heart's mechanical strain and electrophysiological dynamics are very similar during VT, polymorphic VT and VF. More specifically, the inventors found that rotating action potential and calcium waves observed on the heart's ventricular surface cause similarly rotating strain waves, suggesting that the cellular excitation-contraction coupling mechanism can lead to highly correlated electrophysiological and mechanical phenomena on the tissue and organ scale. The experimental findings suggest that it is possible to compute electrophysiological wave phenomena from the heart's muscle deformations.

AI for Solving the ‘Inverse Mechano-Electrical Problem’: Recently it has been demonstrated in computer simulations that it is possible to cross-estimate or predict electrical excitation wave phenomena from tissue deformation that was caused by the electrical wave phenomena. In these proof-of-principle studies, two inverse numerical approaches were developed, one knowledge- or physics-based and one self-supervised deep learning-based approach, to solve the ‘inverse mechano-electrical problem’, in simulated 2D and 3D bulk-shaped tissues. While the physics-based approach employed a replicate biophysical model that assimilates mechanical observation data to drive an internal reproduction of the original electrical dynamics, the deep learning approach employed an encoder-decoder-like convolutional neural network (CNN) that was trained on many thousands of pairs of mechanical and corresponding electrical data. While both approaches can successfully compute electrical wave phenomena from tissue deformation, the inventors found that deep learning outperforms the physics-based approach (90-95% vs. 75-85% accuracy). Key to embodiments of the present invention, a self-supervised deep learning algorithm trained on a large electromechanical dataset can easily associate the underlying electrical pattern with an observed deformation pattern although their relationship is highly complex, and the dynamics are chaotic and in each individual case unique. The inventors found that a neural network can reliably predict even complicated electrical scroll wave chaos, the type of wave dynamics conjectured to underlie VF, with high precision even with measurement noise and at low spatial resolutions. The results suggest that an AI may equally be able to predict the 3D morphologies of electrical circuits during VT from the heart's rapid deformations.

4D Ultrasound Imaging during Ventricular Tachycardia (VT) in Humans: It has been demonstrated that 4D ultrasound can be applied during VT ablation procedures in human patients. It was found that with transthoracic 4D ultrasound the spatial and temporal resolutions and image quality are sufficient to resolve arrhythmic ventricular wall motion and deformation during VT, while mapping the heart's endocardial surface using basket catheters (unpublished).

### Simultaneous Imaging of Voltage, Calcium and Strain in Contracting Cardiac

Tissue using Optical Imaging and Computer Vision: Optical mapping of the heart using voltage- and/or calcium-sensitive fluorescent dyes is widely used in basic cardiovascular research. However, until recently it was not possible to perform optical mapping with contracting hearts. Instead, the heart's contractile motion had to be inhibited using pharmacological excitation-contraction uncoupling agents such as Blebbistatin. Embodiments of the present invention relate to optical and computer vision techniques, with which it is possible to measure action potential and calcium waves together with tissue strain at high spatial resolutions on the strongly contracting 3D deforming heart surface using multi-camera optical mapping systems.

Embodiments of the present invention combine computational, cardiovascular and bioengineering methods and clinical imaging. Embodiments of the present invention will consist of i) generating high-resolution in silico, ex vivo and in human electromechanical data of the heart's arrhythmic electrical and mechanical 4D dynamics to identify and characterize a universal mapping between these two dynamics and ii) developing an AI algorithm, which can translate 4D cardiac mechanics into electrophysiological activation dynamics. The algorithm will ultimately be able to predict these electrical dynamics from 4D imaging data produced with a commercial ultrasound system.

Solving the Inverse Mechano-Electrical Problem in Computer Simulations of Ventricular Arrhythmias: Using a combination of computer simulations and inverse numerical reconstruction techniques, embodiments of the present invention compute electrophysiological wave phenomena, such as action potential or calcium waves, from the 3D deformations of the ventricles of a simulated heart. Embodiments of the present invention may be used to explore first i) in simplified ventricular geometries, which deform due to focal and reentrant ventricular arrhythmias (VT) created with phenomenological models, and next ii) in more realistic ventricular and whole heart geometries and models with more complex arrhythmias (e.g. poly-morphic VT or VF) and pathophysiology created with more detailed models. Embodiments of the present invention may apply the AI-enabled inverse numerical re-construction technique to the simulated data and aims at further refining the technique.

Embodiments of the present invention may i) Provide a proof-of-concept in silico for ventricular arrhythmias to compute 3D electrophysiological morphologies of arrhythmias, as well as locate arrhythmia origins from tissue motion in real-time. ii) Perform these computations in or close to real-time2 (data rate and voxel numbers in computer simulations are comparable to ultrasound imaging) using state-of-the-art GPU (graphics processing unit) hardware. Embodiments of the present invention may be used to generate synthetic simulated training data for the training of a deep learning model (e.g. encoder-decoder-like 3D-CNN), which is consequently specialized in computing 3D electrical activation dynamics from the heart's 3D/4D tissue motion and deformation. In embodiments, it is possible to generate sufficiently large training data sets (2-4 TB, >10,000 samples) with phenomenological computational models within 3-5 days computing time. The simulations will initially exclude heterogeneity and other tissue abnormalities to focus solely on the mapping between mechanics and electrics given a particular arrhythmia morphology in an otherwise idealized situation. As the project progresses, simulations will include more intricate scenarios (e.g. scar tissue, slow conduction channels, dissociation phenomena between voltage and calcium, etc.) and the simulations will be refined to match experimental data.

High-Resolution Ex Vivo Training Data Generation and First Proof-of-Principle in Intact Excised Porcine Hearts: One of the central goals of embodiments of the present invention is to produce high-resolution optical mapping data of arrhythmic hearts in a unique ex vivo imaging environment as training or development data. Intact, isolated porcine hearts (N=50) may be imaged in a Langendorff-perfusion and/or working heart setup. Hearts may be imaged from all sides with up to 32 high-speed CMOS cameras at speeds of 500 fps, measuring voltage and/or calcium. Simultaneously, the hearts may be imaged at speeds of up to 180 volumes per second using high-speed 4D ultrasound (Siemens Acuson SC2000). The two imaging data types may be cross-registered, such that the 3D voltage- and/or calcium-sensitive optical maps can be compared with the volumetric ultrasound data showing the heart's 3D wall motion within the same coordinate system. Embodiments of the present invention may be used to generate 4D high-resolution electromechanical data of the arrhythmic heart ex vivo. Embodiments of the present invention may be used to process data in such a way that it can i) be used to create and be assimilated into biophysical computer models, ii) be used to create data-driven computer models and, ultimately, be iii) used to develop an AI-based reconstruction technique. Ultimately, embodiments of the present invention may comprise training an AI with a mix of in silico and ex vivo data and apply the AI to unseen ex vivo or in human data to predict electrical activation patterns from the mechanical deformations with the same precision and at the same high resolutions that are attainable with optical mapping. The high-resolution optical mapping measurements may be used as the ‘ground truth’ to cross-validate the estimated reconstruction results. A secondary goal will be to extrapolate missing electrical data within the volume of the heart from surface observations during polymorphic VT or VF with epi-endocardial dissociation.

First Application In Human: Embodiments of the present invention may be used to gather in human data during VT ablations (N=60). The data may be acquired together with cardiologists using transthoracic or transesophageal 4D ultrasound (Siemens Acuson SC2000 system used for ex vivo imaging is also available in the clinic) as well as electro-anatomical catheter-mapping. The in human data may be cross-registered with electrical mapping data to compare electrical and mechanical arrhythmia morphologies and to use the data for the development and training of an AI-based reconstruction approach. Embodiments of the present invention may be used: i) to explore the feasibility of the approach in a clinical setting, ii) to determine whether the in silico, ex vivo and in human data can be pooled in a general training dataset and iii) to provide a proof-of-principle in humans for VT. Embodiments of the present invention may be used to show that it is possible to reconstruct arbitrary VT morphologies from 4D ultrasound data in patients with structural or ischemic heart disease (e.g. ICM). Embodiments of the present invention may be used to determine to what extent AI that was trained either solely on in silico data or a mixture of in silico, ex vivo and/or in human data can make accurate predictions about the underlying arrhythmia circuit.

### AI-Assisted Imaging & Prediction of Cardiac Arrhythmia Origins Using 4D Ultrasound

Embodiments of the present invention establish an entirely novel ultrasound-based imaging technique utilizing artificial intelligence (AI) for the non-invasive, transmural visualization and diagnosis of heart rhythm disorders in real-time that will supersede existing imaging techniques and provide novel insights into the origins and driving mechanisms of heart rhythm disturbances. Heart disease is the major cause of morbidity and mortality worldwide. Despite significant progress in the field of biomedical imaging, imaging of heart rhythm disorders remains a major technological and scientific challenge. Understanding their origins and underlying mechanisms is pivotal in the planning of therapeutic interventions. Currently, cardiologists rely on catheter-based contact electrode measurements when diagnosing heart rhythm disorders, such as atrial fibrillation (AF) or ventricular tachycardia (VT). Catheter-based measurements are invasive, time-consuming and typically provide information about the heart's abnormal electrical activity on its endo- or epicardial surface. However, the electrophysiological wave phenomena causing heart rhythm disorders are inherently three-dimensional phenomena evolving deep within the tissue, where they often interact with an arrhythmogenic substrate (e.g. scar tissue) in locations that are inaccessible to standard catheter mapping.

Embodiments of the present invention comprise a novel AI-assisted and 4D (time-varying 3D) ultrasound-based imaging approach for the non-invasive and in-depth visualization of heart rhythm disorders. The imaging approach may be based on the idea that, because the heart's contractions and deformations are triggered by electrical activity, inversely this electrical activity can be computed by analyzing the heart's deformations even during complex arrhythmias. The imaging approach will be enabled by AI, which will learn and translate this complex relationship, 4D imaging and embodiments of the present invention's use of high-resolution multi-modal fluorescence imaging of intact, isolated hearts. With this powerful methodological approach, embodiments may be able to link electrophysiological and mechanical tissue dynamics at high resolutions and develop an AI-based inverse reconstruction technique that are able to decipher the heart's complex deformations and provide high-resolution predictions of the arrhythmic electrical phenomena throughout the entire heart muscle.

Background: The heartbeat is triggered by nonlinear waves of electrical excitation, which propagate rapidly through the heart muscle and activate the cellular contractions of heart muscle cells via the excitation-contraction coupling mechanism. Heart rhythm disorders are caused by abnormal electrical waves, which may degenerate into disorganized, self-sustained wave patterns that initiate irregular and persistent contractions of the heart muscle and underlie cardiac fibrillation. These waves are conjectured to take on the shapes of rotating spiral and scroll waves or ‘rotors’. However, their precise origin and three-dimensional morphology remains insufficiently understood, because imaging technology able to resolve the waves throughout the heart muscle's volume does not exist. Modern ultrasound imaging can provide high-speed 4D videos of the entire heart at resolutions sufficient to resolve even the rapid, small irregular contractions during fibrillation. However, ultrasound provides solely images of the motion of the heart and does not provide electrical information.

Current State-of-the-Art: The current state-of-the-art for the imaging of heart rhythm disorders is electro-anatomic catheter-mapping. Catheter-based mapping is invasive, time-consuming and provides information only from local measurements sparsely distributed across the heart surface. Even with multi-electrode catheters (e.g. multi-arm- or basket-catheters with 256 electrodes) the mapping needs to be performed sequentially, point-by-point to obtain high-resolution maps and, accordingly, the procedural times are typically 30-90 minutes to map an arrhythmia. The measurements are superficial and prone to motion-related artifacts and maps cannot distinguish epi-, endo- or intracardial signal. Intramural measurements are performed in specialized cardiac electrophysiology laboratories using needle electrode catheters to identify intramural, often isthmus-related origins of VTs in patients with ischemic cardiomyopathies (ICM) but are not routinely done in most electrophysiology laboratories because these catheters are not commercially available and require significant time and expertise to perform. An alternative imaging technique for the diagnosis of heart rhythm disorders is inverse electrocardiogramhich involves recording electrical potentials from the patient's body surface and using inverse computational techniques to calculate potentials on the heart surface. However, the technique provides indirect measurements and requires further validation. Both techniques require 3D location sensors or imaging data (e.g., CT or MRI) to create electro-anatomic maps of the abnormal electrical activity during heart rhythm disorders. In the laboratory, heart rhythm disorders can be studied using voltage-sensitive fluorescence imaging, which provides highly detailed visualizations of the heart's electrical activity but fails to image activity deep within the cardiac muscle, similarly as the techniques used in the clinical setting. Fluorescence imaging is limited by low penetration depths of light in tissue and involves toxic substances and can therefore not be used in patients.

Significance: Embodiments of the present invention address a very significant challenge within cardiovascular medicine, and a long-standing problem within biomedical engineering: the non-invasive and in-depth visualization of cardiac arrhythmias. Currently, no experimental method exists that is capable of visualizing three-dimensional, transmural electrical activity of the heart non-invasively. The inventors have shown that 4D ultrasound-based imaging can resolve the electromechanical phenomena underlying cardiac fibrillation, and that the technique could-if combined with AI-provide high-resolution, three-dimensional visualizations of heart rhythm disorders and their origins non-invasively, in real-time and with high (see, e.g., Examples 3, 6 and 7 of this disclosure, provided below). The proposed 4D imaging approach is expected to supersede existing cardiac mapping approaches and could greatly advance the diagnosis of heart rhythm disorders and be used to guide therapeutics, such as catheter ablation, more reliably and effectively. For example, while simple reentrant arrhythmias (e.g. “AVNRT”, “AVRT”, atrial flutter) are easily mapped and ablated with high success, complex reentrant arrhythmias (e.g. ischemic VT, persistent AF or VF) are not easily mapped and ablated clinically. In embodiments, imaging techniques could provide highly detailed live 4D visualizations of these heart rhythm disorders, while guiding more targeted, intracardial ablation procedures using, for instance, focused ultrasound, microwaves or electroporation. Independent of its potential transformative clinical impact, embodiments of the present invention may open doors to a novel field with extraordinary potential in unraveling the mysteries underlying heart rhythm disorders and will provide novel insights into their transmural dynamics.

High-Resolution 4D Ultrasound-based Imaging of Electromechanical Rotors during Ventricular Fibrillation (VF) Ex Vivo: The inventors have previously demonstrated in intact isolated pig hearts ex vivo that it is possible to image and characterize the electrical and mechanical phenomena underlying ventricular fibrillation (VF) at unprecedented resolutions using 4D ultrasound and panoramic fluorescence imaging (see, e.g., Examples 4-7 of this disclosure, provided below). By imaging the fibrillating heart with both imaging techniques simultaneously, the inventors showed, for the first time, that the heart's mechanical strain dynamics are very similar to the electrophysiological dynamics during VT, polymorphic VT and VF. More specifically, the inventors have found that rotating action potential and calcium waves observed on the heart's ventricular surface cause similarly rotating strain waves, suggesting that the cellular excitation-contraction coupling mechanism can lead to highly correlated electrophysiological and mechanical phenomena on the tissue and organ scale and produces ‘electro-mechanical rotors’. Using 4D ultrasound, the inventors demonstrated that the rotating electrical waves produce mechanical filament-like phase singularities, which evolve through the heart muscle and indicate the rotational centers of mechanical scroll waves, which in turn are likely produced by electrical scroll vortex waves during VF. The experimental findings are an indirect measurement of 3D electrophysiological wave phenomena and suggest that it is possible to compute electrophysiological wave phenomena from the heart's muscle deformations during arrhythmias.

Inverse AI- and Physics-based Computational Techniques for Solving the ‘Inverse Mechano-Electrical Problem’: In recent studies, the inventors have demonstrated in computer simulations that it is possible to cross-estimate or predict electrical excitation wave phenomena from tissue deformation that was caused by the electrical wave phenomena. In these proof-of-principle studies, the inventors developed two inverse numerical approaches, one knowledge- or physics-based and one deep learning-based approach, to solve the ‘inverse mechano-electrical problem’, in simulated 3D bulk-shaped tissues. While the physics-based approach employed a replicate numerical model that assimilates mechanical observation data to drive an internal reproduction of the original electrical dynamics via synchronization, the deep learning approach employed a convolutional neural network (CNN) with an encoder-decoder-like architecture that was trained on many thousands of pairs of mechanical and corresponding electrical data. While both approaches can successfully compute electrical wave phenomena from tissue deformation, the inventors found that deep learning outperforms the physics-based approach (90-95% vs. 75-85% accuracy). Key embodiments of the present invention, a self-supervised deep learning algorithm trained on a large electromechanical dataset can easily associate the underlying electrical pattern with an observed deformation pattern although their relationship is highly complex, and the dynamics are chaotic and in each individual case unique. A neural network can reliably predict even complicated electrical scroll wave chaos, the type of wave dynamics conjectured to underlie VF, with high precision even with measurement noise and at low spatial resolutions. The results suggest that an AI may equally be able to predict spatio-temporal electrical wave phenomena from the heart's rapid arrhythmic deformations imaged with 4D ultrasound, based on its vast knowledge of arrhythmic electromechanics acquired through extensive training.

4D Ultrasound Imaging during Ventricular Tachycardia (VT) in Humans: It has been demonstrated that 4D ultrasound can be applied during VT ablation procedures in human patients (N=6). It was found that with transthoracic 4D ultrasound the spatial and temporal resolutions and image quality are sufficient to resolve arrhythmic ventricular wall motion and deformation during VT, while mapping the heart's endocardial surface using basket catheters.

Simultaneous Imaging of Voltage, Calcium and Strain in Contracting Cardiac Tissue using Optical Imaging and Computer Vision: Fluorescence imaging of the heart using voltage- and/or calcium-sensitive fluorescent dyes has been widely used in basic cardiovascular research for over 30 years. However, until recently, it was not possible to image action potential or calcium waves propagating across the contracting heart surface. Instead, the heart's contractile motion had to be inhibited using pharmacological excitation-contraction uncoupling agents such as Blebbistatin. Embodiments of the present invention utilize optical and computer vision techniques, with which it is possible to measure action potential and calcium waves together with tissue strain at high spatial resolutions using multi-camera measurement systems on the strongly contracting 3D deforming heart surface.

Embodiments of the present invention comprise i) generating high-resolution in silico, ex vivo and in human electromechanical data of the heart's arrhythmic electrical and mechanical 4D dynamics to identify and characterize a universal mapping between these two dynamics and ii) developing an AI-based and/or physics-based inverse algorithm, which can translate 4D cardiac mechanics into electrophysiological activation dynamics. The algorithm will ultimately be able to predict these electrical dynamics from 4D imaging data produced with a commercially available ultrasound system.

Solving the Inverse Mechano-Electrical Problem in Computer Simulations of Atrial and Ventricular Arrhythmias: Using a combination of computer simulations and inverse numerical reconstruction techniques, embodiments of the present invention may be used to show that it is possible to compute electrophysiological wave phenomena, such as action potential or calcium waves, from the 3D deformations of a simulated heart. In this purely computational aspect, the feasibility of the approach may be explored first i) in simplified ventricular geometries, which deform due to focal and reentrant ventricular arrhythmias (VT) created with phenomenological models, next ii) in more realistic ventricular and whole heart geometries and models with more complex arrhythmias (e.g. polymorphic VT or VF) and pathophysiology created with more detailed models, and finally iii) in simulated atria and atrial arrhythmias, such as atrial flutter or AF. Embodiments of the present invention may be utilized to apply two different inverse numerical re-construction techniques to the data, one AI- or deep learning-based and one physics-based data assimilation technique, and may be used in connection with advancing and further refining these techniques or developing new hybrid versions. Embodiments of the present invention may be used to provide a proof-of-concept in silico for ventricular and subsequently for atrial arrhythmias to compute the electrophysiological activation dynamics and 3D arrhythmia morphologies, as well as locate arrhythmia origins such as scars or isthmus locations, from tissue motion in a wide range of scenarios accurately, robustly and in real-time.

Using the AI-based approach, embodiments of the present invention may be used to demonstrate that reconstructions can be performed in or close to real-time (data rate and voxel numbers in computer simulations are comparable to ultrasound imaging) in bulk-shaped tissues using state-of-the-art GPU (graphics processing unit) hardware. Embodiments of the present invention may involve the generation of synthetic simulated training data for the training of the AI-based approach or a deep neural network (e.g. encoder-decoder-like 3D-CNN), which is consequently specialized in computing 3D electrical activation dynamics from the heart's 3D/4D tissue motion and deformation. It is possible to generate sufficiently large training data sets (2-4 TB, >10,000 samples) of ventricular arrhythmias with phenomenological computational models within 3-5 days of computing time. The simulations may initially exclude heterogeneity and other abnormalities to focus solely on the mapping between mechanics and electrics given a particular arrhythmia morphology in an otherwise idealized situation. The simulations may include more intricate scenarios (e.g. ischemia, scar tissue, isthmuses or macro-reentrant circuits, fibrosis, heterogeneous active tension, dissociation phenomena between voltage and calcium, electrical abnormalities, etc.) and the simulations may be refined to match experimental data and to create synthetic 4D ultrasound data. Lastly, the relationship between substrate or arrhythmia origins and mechanical activation dynamics during sinus rhythm will be explored.

High-Resolution Ex Vivo Training Data Generation and First Proof-of-Principle in Intact Excised Porcine Hearts: Embodiments of the present invention may be used to produce high-resolution fluorescence imaging data of arrhythmic hearts in a unique ex vivo imaging environment as training or development data. Intact, isolated porcine hearts (N=50) may be imaged in a unique Langendorff-perfusion and/or working heart setup at speeds of 500 frames per second using a custom 3D panoramic fluorescence imaging system with 16 or 32 high-speed CMOS cameras, measuring the transmembrane potential and/or calcium across the entire 360° contracting heart surface. Simultaneously, in embodiments, the hearts may be imaged at speeds of up to 180 volumes per second using high-speed 4D ultrasound (Siemens Acuson SC2000 system, also certified for clinical use). The two imaging data types will be cross-registered, such that the 3D voltage- and/or calcium-sensitive fluorescent maps of the electrophysiological phenomena on the heart surface can be compared with the volumetric ultrasound data showing the heart's 3D wall motion within the same coordinate system. Using the panoramic fluorescence and ultrasound imaging system, embodiments may generate 4D high-resolution electromechanical data of the arrhythmic heart ex vivo. The data may be processed in such a way that it can i) be used to create and be assimilated into biophysical computer models, ii) be used to create data-driven computer models and, ultimately, be iii) used to train or develop an AI or physics-based mechano-electrical reconstruction technique, respectively. Ultimately, embodiments may comprise training train an AI either i) solely with in silico data or ii) with a mix of in silico and ex vivo data and apply the AI to unseen ex vivo 4D ultrasound data to predict electrical activation patterns from the mechanical deformations with the same precision and at the same high resolutions that are attainable with fluorescence imaging. The high-resolution fluorescence measurements will be used as the ‘ground truth’ or validation data to cross-validate the estimated reconstruction results. A secondary goal will be to extrapolate missing electrical measurement data within the volume of the heart from the surface observations, for instance, during polymorphic VT, VF or AF with epi-endocardial dissociation.

### First Application of AI-Assisted 4D Ultrasound-Based Mapping in Human

Embodiments of the present invention may be used in connection with gathering in human training and development data during ablation procedures of arrhythmias in patients (N=100). The data may be acquired using transthoracic or transesophageal 4D ultrasound (Siemens Acuson SC2000 system used for ex vivo imaging is also available in the clinic) as well as electro-anatomical high-density catheter-mapping during VT or AF ablation procedures. As with the ex vivo data, the in human data will be cross-registered with electrical mapping data to be able to compare electrical and mechanical arrhythmia morphologies and to use the data for the training or development of an AI- or physics-based reconstruction approach. Embodiments of the present invention may be used in connection with the following: i) to explore the feasibility of the approach in a clinical setting, ii) to determine whether the in silico, ex vivo and in human data can be pooled in a general training and development dataset and iii) to provide a proof-of-principle in humans for both ventricular and atrial arrhythmias. The data may be used to guide and refine the computer simulations and numerical reconstruction techniques. Embodiments may be used to show that it is possible to reconstruct, for instance, arbitrary VT morphologies from 4D ultrasound data in patients with structural or ischemic heart disease (e.g. ICM). Embodiments may be used to determine whether and to what extent AI that was trained either solely on in silico data or a mixture of in silico, ex vivo and/or in human data can make accurate predictions about the underlying arrhythmia circuit. Embodiments may further be used in connection with establishing 4D ultrasound-based imaging of heart rhythm disorders such as VT or AF as a routinely, clinically used imaging technique. The imaging may produce visualizations of short arrhythmic episodes (5-10 sec.) and locate arrhythmia origins, such as intramural scars, isthmuses or fibrotic regions, either in real-time or at least within less than 2-3 minutes using GPU hardware.

Development of Tracking and Segmentation Algorithms and Generation of Synthetic 4D Ultrasound Data: To be able to analyze the ultrasound data and measure wall motion accurately, embodiments may comprise developing numerical tools and algorithms for 3D motion tracking, deformation quantification, segmentation and visualization. Embodiments of the present invention may comprise a numerical computer vision framework, with which the applicant and his team will be able to extract, quantify and analyze kinematic information about the 3D motion of the heart, even with poor ultrasound image quality.

In some cases, in embodiments: 1) An AI-based approach may not “generalize”, or, in other words, might fail to make accurate predictions reliably, for instance, i) across a larger or ii) in a distinctly different cohort of patients (in silico vs. ex vivo vs. in vivo, different disease, etc.). In embodiments, this issue, and the issue of eventually having too few clinical samples, may be addressed by iterative refinement of the computer simulations and addition of synthetic and eventually ex vivo training data (e.g. obtained with explanted human hearts from heart transplants). Alternatively, a hybrid or a purely physics-based reconstruction technique may be employed in some embodiments. 2) In some patients, poor image quality during transthoracic ultrasound imaging might prevent numerical analysis. In this case, skipping numerical motion tracking and performing training and reconstruction directly in an image-based fashion might circumvent these issues in embodiments. Lastly, the approach could alternatively be pursued with MRI. 3) In some cases, with severe disease progression (e.g. severe scarring, severely reduced ejection fraction, late-stage heart failure) the inverse mechano-electrical problem may not be able to be solved in some embodiments. 4) Dissociation of the excitation-contraction coupling mechanism may, in some patients, compromise the imaging approach.

Currently, heart rhythm disorders are diagnosed by measuring and analyzing the heart's electrical activity during catheter-based contact mapping procedures. Embodiments of the present invention comprise a radically different approach to diagnose heart rhythm disorders by imaging the heart's deformations using high-resolution 4D ultrasound and predicting the underlying electrical activity and arrhythmia origins from the deformations with precisions and at unprecedented resolutions, which are otherwise only attainable in direct electrical measurements in a cutting-edge research laboratory ex vivo. The central hypothesis of embodiments of the invention is that the motion and deformation of the heart contains a wealth of information, which can serve like a fingerprint of an arrhythmia, and which-if captured in a 4D measurement and analyzed properly—is unique enough to be able to compute the 4D morphology of the electrical dynamics causing the arrhythmia. Subsequently, training an AI to learn the complex relationship between cardiac electrics and tissue mechanics from a well-curated 4D imaging dataset obtained under laboratory conditions and undergirded by clinical and synthetic data, may enable the AI to predict any electrical activation pattern from any deformation in any heart. Just as AI can recognize objects, faces, speech or handwritten text and any other complex high-dimensional feature in data, embodiments of the present invention comprising a specially trained AI will also be able to recognize any arrhythmia morphology solely by observing 4D ultrasound data, although arrhythmias are chaotic in nature and will be different and unique in each patient. A self-supervised deep learning algorithm in silico may be provided in support of this hypothesis. The central assumption that a solution for the ‘inverse mechano-electrical problem’ or, in other words, that a unique mapping between cardiac mechanics and electrophysiology exists and is robust enough that it can be exploited by an AI universally in various conditions is highly innovative and extends far beyond the current frontiers in arrhythmia research. In embodiments, the proposed data-driven approach rejects all conventions to describe cardiac deformation mechanics using human-defined characteristics, such as strain or anatomical length changes, but instead processes spatio-temporal patterns of cardiac motion.

SYSTEMS & COMPUTER READABLE MEDIA

Aspects of the present disclosure further include systems, such as computer-controlled systems, for practicing embodiments of the above methods. In some instances the systems further include one or more computers for complete automation or partial automation of the methods described herein. In some embodiments, systems include a computer having a computer readable storage medium with a computer program stored thereon.

In embodiments, the system includes an input module, a processing module and an output module. The subject systems may include both hardware and software components, where the hardware components may take the form of one or more platforms, e.g., in the form of servers, such that the functional elements, i.e., those elements of the system that carry out specific tasks (such as managing input and output of information, processing information, etc.) of the system may be carried out by the execution of software applications on and across the one or more computer platforms represented of the system.

Systems may include a display and operator input device. Operator input devices may, for example, be a keyboard, mouse, or the like. The processing module includes a processor which has access to a memory having instructions stored thereon for performing the steps of the subject methods. The processing module may include an operating system, a graphical user interface (GUI) controller, a system memory, memory storage devices, and input-output controllers, cache memory, a data backup unit, and many other devices. The processor may be a commercially available processor or it may be one of other processors that are or will become available. The processor executes the operating system and the operating system interfaces with firmware and hardware in a well-known manner, and facilitates the processor in coordinating and executing the functions of various computer programs that may be written in a variety of programming languages, such as Java, Perl, C++, other high level or low level languages, as well as combinations thereof, as is known in the art. The operating system, typically in cooperation with the processor, coordinates and executes functions of the other components of the computer. The operating system also provides scheduling, input-output control, file and data management, memory management, and communication control and related services, all in accordance with known techniques. The processor may be any suitable analog or digital system. In some embodiments, processors include analog electronics which allows the user to manually align a light source with the flow stream based on the first and second light signals. In some embodiments, the processor includes analog electronics which provide feedback control, such as for example negative feedback control.

The system memory may be any of a variety of known or future memory storage devices. Examples include any commonly available random access memory (RAM), magnetic medium such as a resident hard disk or tape, an optical medium such as a read and write compact disc, flash memory devices, or other memory storage device. The memory storage device may be any of a variety of known or future devices, including a compact disk drive, a tape drive, a removable hard disk drive, or a diskette drive. Such types of memory storage devices typically read from, and/or write to, a program storage medium (not shown) such as, respectively, a compact disk, magnetic tape, removable hard disk, or floppy diskette. Any of these program storage media, or others now in use or that may later be developed, may be considered a computer program product. As will be appreciated, these program storage media typically store a computer software program and/or data. Computer software programs, also called computer control logic, typically are stored in system memory and/or the program storage device used in conjunction with the memory storage device.

In some embodiments, a computer program product is described comprising a computer usable medium having control logic (computer software program, including program code) stored therein. The control logic, when executed by the processor the computer, causes the processor to perform functions described herein. In other embodiments, some functions are implemented primarily in hardware using, for example, a hardware state machine. Implementation of the hardware state machine so as to perform the functions described herein will be apparent to those skilled in the relevant arts.

Memory may be any suitable device in which the processor can store and retrieve data, such as magnetic, optical, or solid-state storage devices (including magnetic or optical disks or tape or RAM, or any other suitable device, either fixed or portable). The processor may include a general-purpose digital microprocessor suitably programmed from a computer readable medium carrying necessary program code. Programming can be provided remotely to processor through a communication channel, or previously saved in a computer program product such as memory or some other portable or fixed computer readable storage medium using any of those devices in connection with memory. For example, a magnetic or optical disk may carry the programming, and can be read by a disk writer/reader. Systems of the invention also include programming, e.g., in the form of computer program products, algorithms for use in practicing the methods as described above. Programming according to the present invention can be recorded on computer readable media, e.g., any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; portable flash drive; and hybrids of these categories such as magnetic/optical storage media.

The processor may also have access to a communication channel to communicate with a user at a remote location. By remote location is meant the user is not directly in contact with the system and relays input information to an input manager from an external device, such as a computer connected to a Wide Area Network (“WAN”), telephone network, satellite network, or any other suitable communication channel, including a mobile telephone (i.e., smartphone).

In some embodiments, systems according to the present disclosure may be configured to include a communication interface. In some embodiments, the communication interface includes a receiver and/or transmitter for communicating with a network and/or another device. The communication interface can be configured for wired or wireless communication, including, but not limited to, radio frequency (RF) communication (e.g., Radio-Frequency Identification (RFID), Zigbee communication protocols, WiFi, infrared, wireless Universal Serial Bus (USB), Ultra Wide Band (UWB), Bluetooth® communication protocols, and cellular communication, such as code division multiple access (CDMA) or Global System for Mobile communications (GSM).

In one embodiment, the communication interface is configured to include one or more communication ports, e.g., physical ports or interfaces such as a USB port, an RS-232 port, or any other suitable electrical connection port to allow data communication between the subject systems and other external devices such as a computer terminal (for example, at a physician's office or in hospital environment) that is configured for similar complementary data communication.

In one embodiment, the communication interface is configured for infrared communication, Bluetooth® communication, or any other suitable wireless communication protocol to enable the subject systems to communicate with other devices such as computer terminals and/or networks, communication enabled mobile telephones, personal digital assistants, or any other communication devices which the user may use in conjunction.

In one embodiment, the communication interface is configured to provide a connection for data transfer utilizing Internet Protocol (IP) through a cell phone network, Short Message Service (SMS), wireless connection to a personal computer (PC) on a Local Area Network (LAN) which is connected to the internet, or WiFi connection to the internet at a WiFi hotspot.

In one embodiment, the subject systems are configured to wirelessly communicate with a server device via the communication interface, e.g., using a common standard such as 802.11 or Bluetooth® RF protocol, or an IrDA infrared protocol. The server device may be another portable device, such as a smart phone, Personal Digital Assistant (PDA) or notebook computer; or a larger device such as a desktop computer, appliance, etc. In some embodiments, the server device has a display, such as a liquid crystal display (LCD), as well as an input device, such as buttons, a keyboard, mouse or touch-screen.

In some embodiments, the communication interface is configured to automatically or semi-automatically communicate data stored in the subject systems, e.g., in an optional data storage unit, with a network or server device using one or more of the communication protocols and/or mechanisms described above.

Output controllers may include controllers for any of a variety of known display devices for presenting information to a user, whether a human or a machine, whether local or remote. If one of the display devices provides visual information, this information typically may be logically and/or physically organized as an array of picture elements. A graphical user interface (GUI) controller may include any of a variety of known or future software programs for providing graphical input and output interfaces between the system and a user, and for processing user inputs. The functional elements of the computer may communicate with each other via system bus. Some of these communications may be accomplished in alternative embodiments using network or other types of remote communications. The output manager may also provide information generated by the processing module to a user at a remote location, e.g., over the Internet, phone or satellite network, in accordance with known techniques. The presentation of data by the output manager may be implemented in accordance with a variety of known techniques. As some examples, data may include SQL, HTML or XML documents, email or other files, or data in other forms. The data may include Internet URL addresses so that a user may retrieve additional SQL, HTML, XML, or other documents or data from remote sources. The one or more platforms present in the subject systems may be any type of known computer platform or a type to be developed in the future, although they typically will be of a class of computer commonly referred to as servers. However, they may also be a main-frame computer, a workstation, or other computer type. They may be connected via any known or future type of cabling or other communication system including wireless systems, either networked or otherwise. They may be co-located, or they may be physically separated. Various operating systems may be employed on any of the computer platforms, possibly depending on the type and/or make of computer platform chosen. Appropriate operating systems include Windows, iOS, Oracle Solaris, Linux, IBM i, Unix, and others.

Aspects of the present disclosure further include non-transitory computer readable storage mediums having instructions for practicing the subject methods. Computer readable storage mediums may be employed on one or more computers for complete automation or partial automation of a system for practicing methods described herein. In certain embodiments, instructions in accordance with the method described herein can be coded onto a computer-readable medium in the form of “programming”, where the term “computer readable medium” as used herein refers to any non-transitory storage medium that participates in providing instructions and data to a computer for execution and processing. Examples of suitable non-transitory storage media include a floppy disk, hard disk, optical disk, magneto-optical disk, CD-ROM, CD-R, magnetic tape, non-volatile memory card, ROM, DVD-ROM, Blue-ray disk, solid state disk, and network attached storage (NAS), whether or not such devices are internal or external to the computer. A file containing information can be “stored” on computer readable medium, where “storing” means recording information such that it is accessible and retrievable at a later date by a computer. The computer-implemented method described herein can be executed using programming that can be written in one or more of any number of computer programming languages. Such languages include, for example, Python, Java, Java Script, C, C#, C++, Go, R, Swift, PHP, as well as any many others. The non-transitory computer readable storage medium may be employed on one or more computer systems having a display and operator input device. Operator input devices may, for example, be a keyboard, mouse, or the like. The processing module includes a processor which has access to a memory having instructions stored thereon for performing the steps of the subject methods. The processing module may include an operating system, a graphical user interface (GUI) controller, a system memory, memory storage devices, input-output controllers, cache memory, a data backup unit, and many other devices. The processor may be a commercially available processor, or it may be one of other processors that are or will become available. The processor executes the operating system and the operating system interfaces with firmware and hardware in a well-known manner, and facilitates the processor in coordinating and executing the functions of various computer programs that may be written in a variety of programming languages, such as those mentioned above, other high level or low-level languages, as well as combinations thereof, as is known in the art. The operating system, typically in cooperation with the processor, coordinates and executes functions of the other components of the computer. The operating system also provides scheduling, input-output control, file and data management, memory management, and communication control and related services, all in accordance with known techniques.

### Utility

The methods and systems of the invention, e.g., as described above, find use in a variety of applications where it is desirable to understand the origins and underlying mechanisms of heart disease in general, including, for example, heart rhythm disorders such as, e.g., atrial fibrillation (AF) or ventricular tachycardia (VT) in particular. In some embodiments, the methods and systems described herein find use when it is desirable to improve the diagnosis and treatment of heart rhythm disorders in order to improve patient outcomes. In some embodiments, the subject methods and systems find use in diagnosing a heart rhythm disorder and/or diagnosing other cardiac abnormalities. In some embodiments, the subject methods and systems find use in treating a heart rhythm disorder, e.g., by guiding or directing cardiac ablation. In some embodiments, the methods and systems described herein find use in drug discovery, e.g., by performing or enabling drug discovery simulations.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-232 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

1. A method of configuring a model to reflect electrical and mechanical behavior of a tissue, the method comprising:
collecting initial data comprising corresponding electrophysiological activation patterns of a tissue and mechanical movement patterns of the tissue; and
configuring a model of the tissue based on the corresponding electrophysiological activation patterns of the tissue and mechanical movement patterns of the tissue.
   2. The method of aspect 1, wherein the initial data comprises dynamic structural imaging data.
   3. The method of aspects 1 or 2, wherein the initial data comprises model training data.
   4. The method of any one of one the previous aspects, wherein the initial data comprises model configuration data.
   5. The method of any one of the previous aspects, wherein the initial data reflects an initial state of tissue electrophysiology and mechanical movement.
   6. The method of any one of the previous aspects, wherein the electrophysiological activation patterns comprise one or more of: electrical, optical, acoustical, and/or photo-acoustical measurements of electrical excitation of the tissue and/or structure of the tissue.
   7. The method of any one of the previous aspects, wherein the initial data is obtained from a subject.
   8. The method of aspect 7, wherein the initial data comprises subject-specific data.
   9. The method of aspect 8, wherein the subject-specific data comprises one or more of: demographic data, genetic information, patient history data, structural imaging data, and/or quantitative medical data.
   10. The method of aspects 8 or 9, wherein the method further comprises configuring the model of the tissue based on the subject information.
   11. The method of aspect 10, wherein the model of the tissue is configured based on the subject information after the model is configured based on the electrophysiological activation patterns and mechanical movement patterns of the tissue.
   12. The method of any one of the previous aspects, wherein the dynamic structural imaging data comprises ultrasound imaging data, magnetic resonance imaging (MRI) data, and/or computed tomography (CT) imaging data.
   13. The method of aspect 12, wherein the ultrasound imaging data reflects mechanical movement patterns.
   14. The method of any one of the previous aspects, wherein the dynamic structural imaging data comprises optical data.
   15. The method of aspect 14, wherein the optical data reflects electrophysical activation patterns.
   16. The method of any one of the previous aspects, wherein the dynamic structural imaging data comprises fused imaging and optical data.
   17. The method of any one of the previous aspects, wherein the dynamic structural imaging data comprises imaging data over time.
   18. The method of any one of the previous aspects, wherein the initial data comprises synthetic data.
   19. The method of any one of the previous aspects, wherein the initial data comprises data generated using a numerical model configured to represent intra- and/or extra-cellular processes of the tissue.
   20. The method of any one of the previous aspects, wherein the initial data comprises data generated using a computational technique.
   21. The method of aspect 20, wherein the computational technique comprises one or more of: finite-difference methods (FDM), finite element method (FEM), finite volume method (FVM), smoothed-particle hydrodynamics (SPH), and boundary-element method (BEM) and/or a differentiable version of such technique, optionally comprising a differential SPH simulation fitted to data.
   22. The method of any one of the previous aspects, wherein the model is trained using training data comprising electrophysiological data and mechanical deformation data or fitted to such data using gradient descent techniques.
   23. The method of any one of the previous aspects, wherein electrophysiological activation patterns comprise one or more of: action potential (AP) waves or wave fronts, calcium waves or wave fronts, AP activation maps, calcium activation maps, calcium concentrations or changes in calcium concentrations, oxygenation or changes in oxygenation, and/or pH or changes in pH.
   24. The method of any one of the previous aspects, wherein the electrophysiological activation patterns comprise intramural electrical activity of the tissue.
   25. The method of any one of the previous aspects, wherein the electrophysiological activation patterns comprise electrical activity on the tissue surface.
   26. The method of any one of the previous aspects, wherein the electrophysiological activation patterns comprise a sinus wave pattern, a focal wave pattern, and/or a reentrant wave pattern.
   27. The method of any one of the previous aspects, wherein the electrophysiological activation patterns comprise a three-dimensional wave pattern.
   28. The method of any one of the previous aspects, wherein the mechanical movement patterns comprise tissue deformation.
   29. The method of any one of the previous aspects, wherein the mechanical movement patterns comprise tissue deformation triggered by the electrical activation patterns.
   30. The method of any one of the previous aspects, wherein the method further comprises applying the model to estimate an electrophysiological activation pattern of a tissue of a subject by:
inputting experimental data to the model, wherein the experimental data comprises one or more mechanical movement patterns; and
receiving estimated electrophysiological activation patterns output from the model.
   31. The method of any one of the previous aspects, wherein the model infers electrical activity of the tissue from mechanical deformation of the tissue.
   32. The method of any one of the previous aspects, wherein the model infers internal electrical activity of the tissue from mechanical deformation of the tissue.
   33. The method of any one of the previous aspects, wherein the method further comprises applying the model to estimate a mechanical movement pattern of a tissue of a subject by:
inputting experimental data to the model, wherein the experimental data comprises one or more electrophysiological activation patterns of the tissue; and
receiving estimated mechanical movement patterns output from the model.
   34. The method of any one of the previous aspects, wherein the method further comprises applying the model to estimate electrophysiological activation patterns of a tissue of a subject by:
inputting data to the model, wherein the input data comprises one or more electrophysiological activation patterns of the tissue; and
receiving estimated subsequent electrophysiological activation patterns output from the model.
   35. The method of aspect 34, wherein the electrophysiological activation pattern input to the model comprises experimental data.
   36. The method of aspects 34 or 35, wherein the electrophysiological activation pattern input to the model comprises electrode-recordings.
   37. The method of any one of the previous aspects, wherein the electrophysiological activation pattern input to the model comprises an incomplete measurement of electrical activity of the tissue.
   38. The method of any one of the previous aspects, wherein the electrophysiological activation pattern input to the model comprises sparse measurement data or data with low spatial resolution.
   39. The method of any one of the previous aspects, wherein the electrophysiological activation pattern input to the model comprises simulated data.
   40. The method of any one of the previous aspects, wherein the estimated subsequent electrophysiological activation patterns comprise chaotic wave phenomena.
   41. The method of any one of the previous aspects, wherein the method further comprises applying the model to estimate a mechanical movement pattern of a tissue of a subject by:
inputting experimental data to the model, wherein the experimental data comprises one or more mechanical movement patterns of the tissue; and
receiving estimated subsequent mechanical movement patterns output from the model.
   42. The method of any one of the previous aspects, wherein the method further comprises applying the model to estimate electrophysiological activation patterns and mechanical movement patterns of a tissue of a subject by:
inputting experimental data to the model, wherein the experimental data comprises one or more electrophysiological activation patterns of the tissue or mechanical movement patterns of the tissue; and
receiving estimated electrophysiological activation patterns and mechanical movement patterns output from the model.
   43. The method of any one of the previous aspects, wherein the method further comprises applying the model to estimate the presence of abnormal electrophysiological activation patterns of a tissue of a subject by:
inputting experimental data to the model, wherein the experimental data comprises one or more electrophysiological activation patterns of the tissue or mechanical movement patterns of the tissue; and
receiving an estimate of a presence of abnormal electrophysiological activation patterns of the tissue output from the model.
   44. The method of aspect 43, wherein the abnormal electrophysiological activation patterns comprise electrophysiological activation patterns associated with an arrythmia, tachycardia, and/or the presence of scar tissue.
   45. The method of any one of the previous aspects, wherein the method further comprises:
obtaining ultrasound imaging data of the tissue;
applying the model to the obtained ultrasound imagining data; and
receiving an estimate from the model of the presence of an abnormal physiological symptom.
   46. The method of aspect 45, wherein the abnormal physiological symptom comprises one or more of: an abnormal electrical circuit, an abnormal tissue characteristic, scar tissue, diseased tissue, tissue comprising a discontinuity, and/or fibrotic tissue.
   47. The method of any one of the previous aspects, wherein the method further comprises applying the model to estimate a location of and/or a particular morphology associated with an electrical circuit causing an abnormal electrophysiological activation pattern.
   48. The method of any one of the previous aspects, wherein the method further comprises applying the model to identify tissue associated with an abnormal electrophysiological activation pattern.
   49. The method of any one of the previous aspects, wherein the method further comprises applying the model to identify a location for tissue ablation or other tissue modification to remove an abnormal electrophysiological activation pattern.
   50. The method of any one of the previous aspects, wherein the model is further configured based on subject information comprising one or more of: demographic data, genetic information, patient history data, structural imaging data, and/or quantitative medical data.
   51. The method of any one of the previous aspects, wherein data input to and/or data output from the model comprises spatio-temporal data.
   52. The method of any one of the previous aspects, wherein data input to and/or data output from the model comprises a sequence of images.
   53. The method of any one of the previous aspects, wherein data output from the model comprises a single image.
   54. The method of any one of the previous aspects, wherein data input to the model and data output from the model each comprise three-dimensional representations of the tissue over time.
   55. The method of any one of the previous aspects, wherein the initial data comprises the experimental data.
   56. The method of any one of the previous aspects, wherein the experimental data is distinct from the initial data.
   57. The method of any one of the previous aspects, wherein the model is first configured using the initial data before being subsequently trained using experimental data.
   58. The method of any one of the previous aspects, wherein the experimental data comprises dynamic structural imaging data.
   59. The method of aspect 58, wherein the dynamic structural imaging data comprises ultrasound imaging data.
   60. The method of aspect 58 or 59, wherein the dynamic structural imaging data comprises optical data.
   61. The method of any one of aspects 58-60, wherein the dynamic structural imaging data comprises fused imaging and optical data.
   62. The method of any one of aspects 58-61, wherein the dynamic structural imaging data comprises imaging data over time.
   63. The method of any one of the previous aspects, wherein the experimental data comprises one or more of: ultrasound, MRI, and/or CT scan data.
   64. The method of any one of the previous aspects, wherein the method further comprises generating synthetic data for use configuring or training the model.
   65. The method of any one of the previous aspects, wherein the initial data comprises synthetic data.
   66. The method of any one of the previous aspects, wherein the model is trained using synthetic data.
   67. The method of any one of aspects 64-66, wherein the synthetic data comprises data generated based on a mechanical model of the tissue or an electrophysiological model of the tissue.
   68. The method of any one of aspects 64-67, wherein generating the synthetic data comprises:
generating variations of tissue models in silico; and
applying such variations of tissue models to generate synthetic data.
   69. The method of any one of aspects 64-68, wherein the synthetic data comprises data generated by applying a mass-spring model (MSM), a finite-element model (FEM), and/or a smoothed-particle hydrodynamics (SPH) framework, and/or a differentiable simulation version thereof, optionally comprising a differentiable simulation version of MSM, SPH, and/or FEM.
   70. The method of any one of aspects 64-69, wherein the synthetic data comprises data generated by applying an electromechanically coupled simulation.
   71. The method of any one of the previous aspects, wherein the method further comprises:
generating a plurality of models of structures of the tissue, wherein each of the structures varies in at least one aspect; and
applying the plurality of models to generate synthetic data comprising electrophysiological activation patterns of the tissue and/or mechanical movement patterns of the tissue.
   72. The method of aspect 71, wherein each of the structures varies in one or more of: tissue shape, tissue thickness, tissue stiffness, contractile force, fiber orientations, interconnections between regions of tissues, velocities of electrical activation patterns, durations of electrical activation waves, and/or spatial heterogeneity.
   73. The method of any one of the previous aspects, wherein the method further comprises using numerical simulations of tissue behavior to generate synthetic data. 74. The method of any one of the previous aspects, wherein the synthetic data is configured to mimic experimental data.
   75. The method of any one of the previous aspects, wherein data used to configure or train the model is augmented by one or more of: adding random noise to the data, randomly rotating or flipping the data, removing a random section of the data, randomly scaling the data, blurring the data, and/or adding artifacts to the data.
   76. The method of any one of the previous aspects, wherein the synthetic data comprises electrophysiological activation patterns exhibiting a sinus wave, a focal wave, and/or a reentrant wave.
   77. The method of any one of the previous aspects, wherein data used to configure or train the model comprises electrophysiological activation patterns exhibiting a sinus wave, a focal wave, and/or a reentrant wave.
   78. The method of any one of the previous aspects, wherein the method further comprises:
generating ground-truth electrical activation patterns of the tissue;
using the ground-truth electrical activation patterns to estimate mechanical movement patterns of the tissue;
applying the model to the estimated mechanical movement patterns to estimate the electrical activation patterns.
   79. The method of any one of the previous aspects, wherein the model is trained using training data generated from a plurality of electrophysiological models and mechanical deformation models.
   80. The method of any one of the previous aspects, wherein the data generated by the plurality of electrophysiological models is applied to the plurality of mechanical deformation models to generate data comprising corresponding electrical activation and mechanical movement patterns.
   81. The method of any one of the previous aspects, wherein the model comprises a model of electrophysiological activation patterns of the tissue.
   82. The method of any one of the previous aspects, wherein the model comprises a model of calcium concentrations, oxygenation, and/or pH of the tissue or changes in one or more of calcium concentrations, oxygenation, and/or pH of the tissue.
   83. The method of any one of the previous aspects, wherein the model comprises a model of mechanical movement of the tissue.
   84. The method of any one of the previous aspects, wherein the model comprises a model of three-dimensional images or three-dimensional motion vectors of the tissue.
   85. The method of any one of the previous aspects, wherein mechanical movement of the tissue is represented based on three-dimensional images or three-dimensional motion vectors.
   86. The method of any one of the previous aspects, wherein the model is trained to learn mechanical movement of the tissue based on three-dimensional images or three-dimensional motion vectors.
   87. The method of any one of the previous aspects, wherein the method comprises estimating electrical activity of the tissue based on three-dimensional images or three-dimensional motion vectors representing tissue movement using the model.
   88. The method of any one of the previous aspects, wherein the model comprises a deep learning model.
   89. The method of any one of the previous aspects, wherein the model comprises one or more of: a convolutional neural network, a U-Net, a sparse Res U-Net, a denoising diffusion neural network, a sparse denoising diffusion neural network, a transformer, a graph neural network, and/or a large language model.
   90. The method of aspect 89, wherein the model comprises convolutional or attention mechanisms.
   91. The method of any one of the previous aspects, wherein the model comprises a sparse encoding-decoding convolutional neural network.
   92. The method of any one of the previous aspects, wherein the model comprises a virtual vector model.
   93. The method of any one of the previous aspects, wherein the model comprises a numerical simulation.
   94. The method of any one of the previous aspects, wherein the model comprises a mass-spring model (MSM), a smoothed-particle hydrodynamics (SPH) model, or a finite element method (FEM) model.
   95. The method of any one of the previous aspects, wherein the model comprises a differentiable electrical, mechanical, or electromechanical tissue model.
   96. The method of any one of the previous aspects, wherein the model comprises a simulation core component.
   97. The method of any one of the previous aspects, wherein the initial data comprises tissue motion data, tissue geometry data, and/or other subject-specific data.
   98. The method of any one of the previous aspects, wherein the model is trained using gradient descent optimization.
   99. The method of any one of the previous aspects, wherein the model comprises a numerical model configured to represent intra- and extra-cellular processes of the tissue.
   100. The method of any one of the previous aspects, wherein the model comprises a computational technique comprising one or more of: finite-difference methods (FDM), finite element method (FEM), finite volume method (FVM), smoothed-particle hydrodynamics (SPH), and/or boundary-element method (BEM).
   101. The method of any one of the previous aspects, wherein the model comprises an idealized model of the tissue.
   102. The method of any one of the previous aspects, wherein the model comprises a subject-specific model of the tissue.
   103. The method of any one of the previous aspects, wherein the model comprises a model of the tissue comprising one or more of: a fiber-architecture model of the tissue, scar placement within the tissue, and/or fibrosis placement within the tissue.
   104. The method of any one of the previous aspects, wherein the method further comprises applying the model for drug screening.
   105. The method of any one of the previous aspects, wherein the method further comprises of applying the model for treatment of a subject.
   106. The method of any one of the previous aspects, wherein the model comprises a digital twin of a subject.
   107. The method of any one of the previous aspects, wherein the method further comprises generating a virtual simulation of the tissue using the model.
   108. The method of any one of the previous aspects, wherein the configuring comprises simulating a tissue function as the model learns the contractile motion and electrophysiological wave pattern associated with the tissue.
   109. The method of any one of the previous aspects, wherein the configuring comprises simulating a pumping heart as the model learns the contractile motion and electrophysiological wave pattern associated with the tissue.
   110. The method of any one of the previous aspects, wherein the method further comprises using the model to evolve the behavior of a simulated tissue over time.
   111. The method of any one of the previous aspects, wherein the electrical behavior of the tissue is integrated with the mechanical behavior of the tissue in the model.
   112. The method of any one of the previous aspects, wherein the method further comprises applying the model to estimate a sequence in which different tissue locations are electrically activated.
   113. The method of any one of the previous aspects, wherein the method further comprises applying the model to estimate an electrical activity activation map of the tissue.
   114. The method of any one of the previous aspects, wherein the method further comprises applying the model to estimate a location of scar tissue on the tissue.
   115. The method of any one of the previous aspects, wherein the method further comprises applying the model to estimate a location of tissue that exhibits an abnormal electrical property.
   116. The method of aspect 115, wherein the abnormal electrical activity comprises one or more of abnormal conductivity and/or abnormal conduction speed for the tissue.
   117. The method of any one of the previous aspects, wherein the method further comprises applying the model to estimate a location of tissue that exhibits an abnormal mechanical property.
   118. The method of aspect 117, wherein the abnormal mechanical property comprises abnormal contractility for the tissue.
   119. The method of any one of the previous aspects, wherein the method further comprises validating the model based on clinical treatment of a subject.
   120. The method of any one of the previous aspects, wherein the method further comprises validating the model, wherein validating the model comprises:
using the model to estimate an electrical activation pattern of the tissue based on an observed movement pattern of the tissue; and
confirming the electrical activation pattern is associated with the observed movement pattern of the tissue.
   121. The method of any one of the previous aspects, wherein the method further comprises validating the model, wherein validating the model comprises:
obtaining an electrical activation pattern of a tissue or a mechanical activation pattern of a tissue;
estimating an electrical activation pattern or a mechanical activation pattern by applying the model to the electrical activation pattern or a mechanical activation pattern;
using a second model to estimate an electrical activation pattern or a mechanical activation pattern by applying the second model to the electrical activation pattern or a mechanical activation pattern, wherein the second model comprises a physics-based model; and
assessing the accuracy of the model by comparing the estimates from the model against the estimates from the second model.
   122. The method of any one of the previous aspects, wherein the method further comprises collecting ultrasound imaging data of an in vivo tissue of a subject;
applying the collected imaging data to the model to estimate electrical activation patterns within the tissue of the subject; and
identifying one or more aspects of an abnormal electrophysiological activation pattern of the tissue of the subject.
   123. The method of any one of the previous aspects, wherein the method is a method of non-invasively analyzing electrophysiological processes of the tissue.
   124. The method of any one of the previous aspects, wherein the method is a method of non-invasively mapping a heart's electrical activity.
   125. A method of observing mechanical movement patterns and corresponding electrophysiological activation patterns of a tissue, the method comprising:
introducing a tissue into an imaging apparatus configured to image electrophysiological activation patterns and mechanical movement patterns of the tissue, wherein the imaging apparatus comprises:
   an enclosed interior volume into which the tissue is introduced;
   a substrate on which the tissue is mounted;
   an electrode for electrically stimulating the tissue;
   a perfusion subsystem configured to support physiological conditions of the tissue;
   a plurality of light sources configured for optically illuminating the tissue;
   an imaging system comprising an optical imaging subsystem configured for obtaining optical images of the tissue; and
   a plurality of mounts configured for mounting the light sources and aspects of the imaging system such that the tissue is evenly illuminated, and the imaging system obtains optical images of the tissue from a plurality of different perspectives;
electrically stimulating the tissue with the electrode;
imaging of the tissue by applying the imaging system in response to a stimulation of the tissue, wherein tissue stimulation comprises one or more of electrical, optical, and/or pharmacological stimulation of the tissue;
imaging electrophysiological activation patterns of the tissue by applying the imaging system to obtain voltage- or calcium-sensitive optical mapping images in response to the stimulation of the tissue; and
combining the obtained images of mechanical movement patterns and electrophysiological activation patterns to reconstruct movement of the tissue in response to the stimulation.
   126. The method of aspect 125, wherein the imaging system of the imaging apparatus further comprises an ultrasound imaging subsystem configured for obtaining ultrasound images of the tissue.
   127. The method of aspect 126, wherein the mechanical movement patterns of the tissue are imaged by applying the ultrasound imaging subsystem in response to a stimulation of the tissue.
   128. The method of aspect 125 to 127, wherein the imaging apparatus further comprises a plurality of electrodes configured to detect electrophysiological activation patterns of the tissue.
   129. The method of aspect 128, wherein the plurality of detection electrodes are configured to measure electrocardiograms from the tissue.
   130. The method of aspect 125 to 129, wherein the imaging apparatus further comprises a power source configured to supply power to the imaging system and/or the plurality of light sources.
   131. The method of aspects 125 to 130, wherein the plurality of mounts are configured for mounting aspects of the imaging system such that every point of the surface of the tissue is imaged from two or more perspectives.
   132. The method of aspects 125 to 131, wherein the method further comprises optically stimulating the tissue using the plurality of light-sources.
   133. The method of any one of aspects 125 to 132, wherein the method further comprises pharmacologically stimulating the tissue using pharmacological compounds or drugs.
   134. The method of any one of aspects 125 to 133, wherein the method further comprises pharmacologically immobilizing aspects of the tissue using pharmacological compounds or drugs.
   135. The method of any one of aspects 125 to 134, wherein the imaging apparatus comprises an imaging chamber or an imaging rig.
   136. The method of aspect 135, wherein the imaging chamber or rig comprises a plurality of windows configured for illuminating the tissue and obtaining images of the tissue from a plurality of different perspectives.
   137. The method of any one of aspects 125 to 136, wherein the optical imaging subsystem comprises a multi-camera high-speed imaging system.
   138. The method of any one of aspects 125 to 137, wherein the method comprises obtaining panoramic images of the tissue.
   139. The method of aspect 138, wherein every point on the surface of the tissue is imaged.
   140. The method of any one of aspects 125 to 139, wherein the method further comprises:
repeatedly obtaining representations of corresponding electrophysiological activation patterns of the tissue and mechanical movement patterns of the tissue; and
combining such representations into a data set for configuring a model according to aspects 1 to 124.
   141. The method of aspect 140, wherein the method further comprises:
collecting electrical activation patterns and associated mechanical movement patterns for the tissue present in the imaging chamber; and
applying the electrical activation patterns and associated mechanical movement patterns as training data for the model.
   142. The method of any one of aspects 125 to 141, wherein the method further comprises applying a numerical motion compensation technique to images of the tissue.
   143. The method of any one of aspects 125 to 142, wherein the method further comprises applying an optical mapping technique combined with a numerical motion tracking technique to measure an electrical activation pattern in the tissue moving in the imaging apparatus.
   144. The method of any one of aspects 125 to 143, wherein every point of the surface of the tissue is imaged from two or more perspectives and the method further comprises applying a stereoscopic motion tracking technique to measure an electrical activation pattern and/or a mechanical movement pattern in the tissue moving in the imaging apparatus.
   145. The method of any one of aspects 125 to 144, wherein the method comprises applying a GPU-based motion tracking algorithm to process imaging data of the tissue.
   146. The method of any one of aspects 125 to 145, wherein the method further comprises:
collecting a plurality of raw images of the tissue;
applying an optical flow estimation technique or differentiable motion estimation technique to the raw images to compensate for tissue movement; and
generating a plurality of compensated images of the tissue to estimate tissue deformation.
   147. The method of aspect 146, wherein every point of the surface of the tissue is imaged from two or more perspectives and the optical flow estimation technique is a stereoscopic optical flow estimation technique or a multi-view differentiable rendering technique used for tracking three-dimensional motion.
   148. The method of any one of aspects 125 to 147, wherein the method further comprises applying one or more optical flow estimation techniques, differentiable motion estimation techniques, or motion compensation techniques to images of the tissue moving to mitigate motion artifacts.
   149. The method of any one of aspects 125 to 148, wherein the method comprises imaging the tissue without applying a pharmacological excitation-contraction uncoupling agent to the tissue.
   150. The method of any one of aspects 125 to 149, wherein the method comprises imaging electrophysiological activation patterns of the tissue or mechanical movement patterns of the tissue without a fiducial marker.
   151. The method of any one of aspects 125 to 150, wherein the method comprises imaging electrical activation patterns across a tissue surface as the tissue deforms.
   152. The method of any one of aspects 125 to 151, wherein the method comprises imaging electrical activation patterns across a surface of the tissue (optionally comprising an atrial or a ventricular surface) as the tissue contracts.
   153. A method comprising validating a model according to any one of aspects 1 to 124 based on imaging obtained using the imaging apparatus according to any one of aspects 125 to 152.
   154. The method of any one of the previous aspects further comprising:
validating the model, wherein validating the model comprises:
using the model to estimate an electrical activation pattern of the tissue based on an observed movement pattern of the tissue; and
confirming the electrical activation pattern is associated with the observed movement pattern of the tissue.
   155. The method of any one of the previous aspects further comprising validating the model, wherein validating the model comprises:
obtaining an electrical activation pattern of a tissue or a mechanical activation pattern of a tissue;
estimating the electrical activation pattern or the mechanical activation pattern by applying the model to the electrical activation pattern or a mechanical activation pattern;
using a second model to estimate the electrical activation pattern or the mechanical activation pattern by applying the second model to the electrical activation pattern or a mechanical activation pattern, wherein the second model comprises a physics-based model; and
assessing the accuracy of the model by comparing the estimates from the model against the estimates from the second model.
   156. The method of any one of the previous aspects further comprising:
collecting ultrasound imaging data of an in vivo tissue of a subject;
applying the collected imaging data to the model to estimate electrical activation patterns within the tissue of the subject; and identifying one or more aspects of an abnormal electrophysiological activation pattern of the tissue of the subject.
   157. The method of any one of the previous aspects, wherein the method is a method of non-invasively analyzing electrophysiological processes of the tissue.
   158. The method of any one of the previous aspects, wherein the method is a method of non-invasively mapping a heart's electrical activity.
   159. The method of any one of the previous aspects, wherein the tissue comprises electrically excitable tissue.
   160. The method of any one of the previous aspects, wherein the tissue comprises anisotropic tissue.
   161. The method of any one of the previous aspects, wherein the tissue comprises cardiomyocytes.
   162. The method of any one of the previous aspects, wherein the tissue is cardiac tissue, lung tissue, intestinal tissue, or uterine tissue.
   163. The method of aspect 162, wherein the tissue is cardiac tissue.
   164. The method of any one of the previous aspects, wherein the tissue comprises one or more of: left ventricle tissue, right ventricle tissue, left atrium tissue, and/or right atrium tissue.
   165. The method of aspect 164, wherein the tissue comprises left and right ventricles.
   166. The method of any one of the previous aspects, wherein the tissue comprises a biventricular heart geometry.
   167. The method of any one of the previous aspects, wherein the tissue is human tissue, rabbit tissue, pig tissue, sheep tissue, dog tissue, mouse tissue, rat tissue, or guinea pig tissue.
   168. The method of aspect 167, wherein the tissue is human tissue or pig tissue.
   169. The method of any one of the previous aspects, wherein the tissue comprises a discontinuity.
   170. The method of aspect 169, wherein the discontinuity is scar tissue or fibrotic tissue.
   171. The method of any one of the previous aspects, wherein the tissue comprises an isolated organ.
   172. The method of any one of the previous aspects, wherein the tissue comprises cultured tissue.
   173. The method of any one of the previous aspects, wherein the tissue comprises elastic excitable media.
   174. The method of any one of the previous aspects, wherein the tissue exhibits muscle fiber anisotropy.
   175. A method of assessing a model, the method comprising:
generating ground-truth electrical activation patterns of a tissue;
using the ground-truth electrical activation patterns to estimate mechanical movement patterns of the tissue;
applying a model of the tissue to the estimated mechanical movement patterns of the tissue to estimate associated electrical activation patterns;
comparing the estimated associated electrical activation patterns with the ground-truth electrical activation patterns; and
assessing the accuracy of the model's predictions based on results of comparing the estimated and ground-truth electrical activation patterns.
   176. A method of training a model to estimate electrophysiological activation patterns based on mechanical movement patterns, to estimate mechanical movement based on electrophysiological activation patterns, and/or to estimate a future electromechanical state based on electrophysiological activation patterns or mechanical movement patterns, the method comprising:
collecting a plurality of training data comprising corresponding electrophysiological activation patterns of a tissue and mechanical movement patterns of the tissue;
representing the electrophysiological activation patterns of the tissue as voltages, calcium concentrations, oxygenation, pH, and/or a temporal change thereof over a volume representing the tissue;
representing the mechanical movement of the tissue as three-dimensional images and/or motion vectors over the volume representing the tissue;
training a model using the electrophysiological activation patterns and corresponding mechanical movement.
   177. The method of aspect 176, wherein the model is trained to estimate electrophysiological activation patterns, given a representation of mechanical movement of the tissue.
   178. The method of any one of aspects 176 to 177, wherein the model is trained to estimate electrophysiological activation patterns, including abnormal patterns and locations, over a time period.
   179. The method of any one of aspects 176 to 178, wherein the training data comprises a sequence of images of corresponding electrophysiological activation patterns of the tissue and mechanical movement patterns of the tissue at consecutive times.
   180. The method of any one of aspects 176 to 179, wherein the training data comprises simulation data.
   181. The method of aspect 180, wherein the simulation data comprises results of electromechanical simulations.
   182. The method of any one of any of aspects 176 to 181, further comprising augmenting the training data with simulation data.
   183. The method of aspect 182, wherein augmenting the training data with simulation data comprises:
generating a plurality of computer-based models of structures of the tissue, wherein each of the structures varies in at least one aspect; and
using the plurality of tissue models to generate simulated results of corresponding electrical activation patterns of the modeled tissue and mechanical movement of the modeled tissue.
   184. The method of aspect 183, wherein each of the structures varies in one or more of: tissue shape, tissue thickness, tissue stiffness, contractile force, fiber orientations, interconnections between regions of tissues, velocities of electrical activation patterns, durations of electrical activation waves, and/or spatial heterogeneity.
   185. The method of aspect 184, wherein each of the structures varies in spatial heterogeneity, the spatial heterogeneity varied comprising one or more of: tissue shape, tissue thickness, tissue stiffness, contractile force, fiber orientations, interconnections between regions of tissues, velocities of electrical activation patterns, and/or durations of electrical activation waves.
   186. The method of any one of aspects 183 to 185, wherein the plurality of computer-based models of structures of the tissue comprise numerical simulations comprising one or more of: finite-difference methods (FDM), finite element method (FEM), finite volume method (FVM), smoothed-particle hydrodynamics (SPH), and/or boundary-element method (BEM).
   187. The method of any one of aspects 183 to 186, wherein augmenting the training data with simulation data further comprises:
simulating the introduction of electrophysiological activation patterns, action potential waves, or calcium waves into the modeled tissue.
   188. The method of aspect 187, wherein the electrophysiological activation patterns comprise one or more of a sinus wave, focal wave, and/or reentrant wave.
   189. The method of any one of aspects 176 to 188, wherein the volume representing the tissue comprises an idealized or patient-specific computer-based model of the tissue, optionally comprising one or more of a fiber-architecture model of the tissue, scar or fibrosis placement within the tissue of the tissue.
   190. An imaging apparatus for imaging electrophysiological activation patterns and mechanical movement patterns of a tissue, wherein the imaging apparatus comprises:
an enclosed interior volume configured to receive biological tissue;
a perfusion subsystem configured to support physiological conditions of the tissue;
a substrate on which the tissue is mounted;
a plurality of light sources configured for illuminating the tissue;
an imaging system comprising an optical imaging subsystem configured for obtaining optical images of the tissue; and
a plurality of mounts configured for mounting the light sources and aspects of the imaging system such that the tissue is evenly illuminated, and the imaging system obtains optical images of the tissue from a plurality of different perspectives.
   191. The imaging apparatus of aspect 190, wherein the imaging system further comprises an ultrasound imaging subsystem configured for obtaining ultrasound images of the tissue.
   192. The imaging apparatus of aspect 190 or 191, wherein the imaging apparatus comprises a plurality of windows configured for illuminating the tissue and obtaining images of the tissue from a plurality of different perspectives.
   193. The imaging apparatus of any one of aspect 190 or 192, wherein the imaging apparatus further comprises a plurality of electrodes configured to detect electrophysiological activation patterns of the tissue.
   194. The imaging apparatus of aspect 193, wherein the plurality of detection electrodes are configured to measure electrocardiograms from the tissue.
   195. The imaging apparatus of any one of aspects 190 to 194, wherein the imaging apparatus further comprises a power source configured to supply power to the imaging system and/or the plurality of light sources.
   196. The imaging apparatus of any one of aspects 190 to 195, wherein the imaging apparatus comprises a truncated icosahedron shape.
   197. The imaging apparatus of any one of aspects 190 to 196, wherein the imaging apparatus comprises a soccer ball geometry.
   198. The imaging apparatus of any one of aspects 190 to 197, wherein the imaging apparatus comprises an open top and an open bottom.
   199. The imaging apparatus of any one of aspects 190 to 198, wherein the open top and open bottom comprise fluidic pathways.
   200. The imaging apparatus of any one of aspects 190 to 199, wherein the imaging apparatus comprises 24 penta- or hexagonal surfaces.
   201. The imaging apparatus of any one of aspects 190 to 200, wherein the imaging apparatus comprises a shape with angles of optical axes between two adjacent surfaces of 37.4° or 41.8°.
   202. The imaging apparatus of any one of aspects 190 to 201, wherein the surfaces comprise windows configured for mounting cameras.
   203. The imaging apparatus of any one of aspects 190 to 202, wherein the imaging apparatus comprises a plurality of windows present at vertices between surfaces of the imaging apparatus and configured to receive LEDs to illuminate the interior of the imaging apparatus.
   204. The imaging apparatus of any one of aspects 190 to 203, wherein the interior of the imaging apparatus is configured to be uniformly illuminated.
   205. The imaging apparatus of any one of aspects 190 to 204, wherein the optical imaging subsystem comprises six or more cameras.
   206. The imaging apparatus of any one of aspects 190 to 205, wherein the optical imaging subsystem comprises 12 or more cameras.
   207. The imaging apparatus of any one of aspects 190 to 206, wherein an angle between neighboring cameras is between 35 and 45°.
   208. The imaging apparatus of any one of aspects 190 to 207, wherein the optical imaging subsystem comprises a plurality of CCD or CMOS cameras.
   209. The imaging apparatus of any one of aspects 190 to 208, wherein the optical imaging subsystem is configured for measuring changes in fluorescence of aspects of the tissue.
   210. The imaging apparatus of any one of aspects 190 to 209, wherein the optical imaging subsystem is configured for measuring changes in fluorescence of aspects of the tissue in response to a physiological change of the tissue.
   211. The imaging apparatus of aspect 210, wherein the physiological change comprises a change in transmembrane potential of the tissue.
   212. The imaging apparatus of any one of aspects 190 to 211, wherein the optical imaging subsystem is configured to image from the plurality of cameras simultaneously.
   213. The imaging apparatus of any one of aspects 190 to 212, wherein the optical imaging subsystem comprises a plurality of filters configured to block excitation light originating from the plurality of light sources.
   214. The imaging apparatus of any one of aspects 190 to 213, wherein the perfusion subsystem comprises a constant-pressure Langendorff-perfusion subsystem.
   215. The imaging apparatus of any one of aspects 190 to 214, wherein the volume of the imaging apparatus is approximately 5 L.
   216. The imaging apparatus of any one of aspects 190 to 215, wherein the imaging apparatus further comprises an electrode configured for electrically stimulating the tissue.
   217. The imaging apparatus of aspect 216, wherein the stimulation electrode is configured to introduce regular pacing of electrical stimulation to the tissue.
   218. The imaging apparatus of aspect 216 or 217, wherein the stimulation electrode is configured to introduce burst pacing of electrical stimulation to the tissue.
   219. The imaging apparatus of any one of aspects 190 to 218, wherein the imaging apparatus is configured to obtain 360° imaging of the tissue.
   220. The imaging apparatus of any one of aspects 190 to 219, wherein the imaging apparatus is configured to simultaneously obtain images of every point on the surface of the tissue from two or more perspectives.
   221. The imaging apparatus of any one of aspects 190 to 220, wherein the imaging apparatus is configured to simultaneously obtain images of every point on the surface of the tissue from four or more perspectives.
   222. The imaging apparatus of any one of aspects 190 to 221, wherein the imaging apparatus is configured to obtain images of the tissue while the tissue moves.
   223. The imaging apparatus of any one of aspects 190 to 222, wherein the imaging apparatus is configured to obtain images of the tissue while the tissue deforms.
   224. The imaging apparatus of any one of aspects 190 to 223, wherein the imaging apparatus comprises a 3D-printed substrate.
   225. The imaging apparatus of any one of aspects 190 to 224, wherein the imaging apparatus further comprises a temperature-regulation subsystem.
   226. The imaging apparatus of any one of aspects 190 to 225, wherein the imaging apparatus a processor comprising memory operably coupled to the processor, wherein the memory comprises instructions stored thereon, which, when executed by the processor, cause the processor to control the imaging apparatus.
   227. A system for utilizing a model of a tissue, the system comprising:
a processor comprising memory operably coupled to the processor, wherein the memory comprises instructions stored thereon, which, when executed by the processor, cause the processor to implement the steps of a method of any one of aspects 1 to 189.
   228. The system of aspect 227, wherein the system further comprises an imaging apparatus according to any of aspects 190 to 226, wherein the imaging apparatus is operably connected to the processor and memory.
   229. The system of aspect 227 or 228, wherein the memory further comprises instructions store thereon, which, when executed by the processor cause the processor to:
control the imaging apparatus to collect images; and
apply a numerical motion tracking algorithm to mitigate motion artifacts present in the images.
   230. A non-transitory computer readable storage medium comprising instructions stored thereon, the instructions comprising an algorithm configured to implement the steps of a method of any one of aspects 1 to 189.
   231. A method for identifying a subject as having a heart rhythm disorder, the method comprising:
imaging the heart of the subject using 4D ultrasound in order to generate spatio-temporal mechanical deformation data of the subject's heart during one or more contractions; and
inputting the mechanical deformation data into a model according to any one of claims 1 to 124, wherein the output of the model is used to determine if the subject has a heart rhythm disorder.
   232. A method for performing catheter ablation on the heart of a subject having a heart rhythm disorder, the method comprising:
imaging the heart of the subject using 4D ultrasound in order to generate spatio-temporal mechanical deformation data of the subject's heart during one or more contractions;
inputting the mechanical deformation data into a model according to any one of claims 1 to 124, wherein the output of the model is used to locate tissue associated with an abnormal electrical wave, an abnormal electrical pathway, or scar tissue; and
ablating tissue associated with the abnormal electrical wave, the abnormal electrical pathway, or the scar tissue in order to disrupt formation or propagation of the abnormal electrical wave.
   The following is offered by way of illustration and not by way of limitation:

### Experimental

Embodiments of methods of the present invention were applied in connection with obtaining experimental results described below. The experimental results described below relate to observing, visualizing, configuring, training, and implementing aspects of cardiac tissue models. As demonstrated in the above disclosure, the present invention has a wide variety of applications. The following is put forth so as to provide those of ordinary skill in the art with a complete disclosure and description of how to make and use the present invention and are not intended to limit the scope of what the inventors regard as their invention nor are they intended to represent that the experiments below are all or the only experiments performed. Those of skill in the art will readily recognize a variety of noncritical parameters that could be changed or modified to yield essentially similar results. Efforts have been made to ensure accuracy with respect to numbers used but some experimental errors and deviations should be accounted for.

### Example 1: Reconstruction of Three-Dimensional Scroll Waves in Excitable Media from Two-Dimensional Observations Using Deep Neural Networks

### 1.0. Overview

Scroll wave dynamics are thought to underly life-threatening ventricular fibrillation. However, direct observations of three-dimensional electrical scroll waves remain elusive, as there is no direct way to measure action potential wave patterns transmurally throughout the thick ventricular heart muscle. Here whether it is possible to reconstruct simulated scroll waves and scroll wave chaos using deep learning is studied. Encoding-decoding convolutional neural networks were trained to predict three-dimensional scroll wave dynamics inside bulk-shaped excitable media from two-dimensional observations of the wave dynamics on the bulk's surface. Whether observations from one or two opposing surfaces would be sufficient and whether transparency or measurements of surface deformations enhances the reconstruction was tested. Further, the approach's robustness against noise was evaluated and the feasibility of predicting the bulk's thickness was tested. Isotropic and anisotropic, as well as opaque and transparent, excitable media were distinguished as models for cardiac tissue and the Belousov-Zhabotinsky chemical reaction, respectively. While it was demonstrated that it is possible to reconstruct three-dimensional scroll wave dynamics, it was also shown that it is challenging to reconstruct complicated scroll wave chaos and that prediction outcomes depend on various factors such as transparency, anisotropy, and ultimately the thickness of the medium compared to the size of the scroll waves. In particular, it was found that anisotropy provides crucial information for neural networks to decode depth, which facilitates the reconstructions. This work indicates deep neural networks could be used to visualize intramural action potential wave patterns from epi- or endocardial measurements.

### 1.1. Introduction

Scroll wave dynamics occur in excitable reaction diffusion systems, termed ‘excitable media’. They are conjectured to underly life-threatening heart rhythm disorders, such as ventricular fibrillation. In the heart, nonlinear waves of electrical excitation propagate through the cardiac muscle and initiate its contractions. The electrical waves are conjectured to degenerate into electrical scroll wave chaos via a cascade of wavebreaks during the onset of ventricular fibrillation. However, direct evidence for the existence of scroll waves in the heart is lacking. While the dynamics of scroll waves have been studied extensively in computer simulations [1-3], the direct visualization of scroll waves throughout the depths of the heart muscle remains a challenge.

Spiral wave-like action potential waves can be imaged on the heart surface during ventricular tachycardia or fibrillation using voltage-sensitive optical mapping [4-8], and the surface observations are in agreement with simulated three-dimensional scroll wave dynamics [3]. Otherwise, only few, and indirect experimental evidence of scroll waves in the heart exists. Voltage-sensitive transillumination imaging was used to measure projections of scroll waves on the surface of the isolated right ventricle of porcine and sheep hearts [9-11]. The right ventricles are thinner than the left and can therefore be penetrated (~0.5 cm) by near-infrared light, making them semi-transparent. Consequently, it was possible to locate focal wave sources inside the volume of the right ventricle using transillumination imaging [12, 13]. More recently, it was shown that ultrasound imaging can reveal mechanical vortices in the ventricles of whole isolated porcine hearts, which co-exist with electrical vortices on the epicardial surface, suggesting that the heart's mechanical dynamics reflect electrical scroll wave dynamics [7, 14]. However, it remains difficult to extrapolate the measured projections or surface observations of the electrical dynamics into the depths of the cardiac muscle, and eventually correlate them with mechanical measurements. Fully three-dimensional reconstructions of scroll waves were obtained using optical tomography in the transparent Belousov-Zhabotinsky chemical reaction [15-18], which is an excitable medium that shares very similar wave dynamics with cardiac tissue. In contrast, three-dimensional action potential waves have been directly measured in small rat and zebrafish hearts using laminar optical tomography or light-sheet microscopy [20]. However, attempts to obtain three-dimensional visualizations of scroll waves inside the optically dense cardiac muscle of large mammalian hearts have not yet attained a similar quality, and better measurement and reconstruction techniques are needed.

Multiple numerical approaches for the reconstruction of scroll waves from surface observations have previously been proposed: Berg et al. attempted to recover simulated scroll wave chaos from single-surface observations using a synchronization-based data-assimilation approach. However, while the approach was successful at recovering scroll wave chaos from sparse measurements within the medium, (e.g., [22]), it was not suited to extrapolate scroll wave dynamics into the three-dimensional bulk-shaped medium from surface observations. Hoffman et al. [23, 24] analyzed dual-surface observations (comparable to measuring both the epi- and endocardium) using a different data-assimilation approach, the local ensemble transform Kalman filter [25], to successfully reconstruct simulated scroll waves. The question remains if it is possible to reconstruct truly complex three-dimensional scroll wave chaos from single- or dual-surface observations. More recently, neural networks were used to predict cardiac dynamics from sparse or partial observations with promising results [26-28]. However, the task of predicting scroll wave dynamics from surface observations using neural networks has not yet been established.

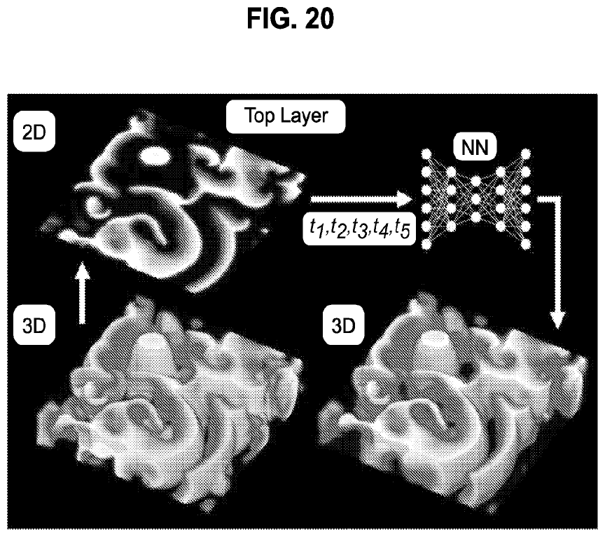

Here, a numerical proof-of-principle that deep encoding-decoding convolutional neural networks, under certain conditions, can be used to reconstruct three-dimensional scroll wave dynamics, including complicated scroll wave chaos, from two-dimensional observations of the dynamics is provided (FIG. 20). It is shown that scroll waves can be recovered fully when the size of the waves is in the order of the thickness of the medium, or when analyzing projections of dynamics with more and smaller waves in transparent anisotropic excitable media. Several deep convolutional neural network architectures were tested, and their reconstruction performance was analyzed depending on opacity, thickness, and anisotropy of simulated excitable media.

FIG. 20: Deep learning-based reconstruction of scroll wave chaos inside a three-dimensional volume from partial observations of the dynamics on its surface. Scroll wave chaos is a model for the electrophysiological dynamics underlying ventricular fibrillation. Computer simulations were performed in isotropic and anisotropic bulk-shaped excitable media. A neural network (NN) predicts scroll wave dynamics underneath the bulk's surface from a short temporal sequence (t1, . . . , t5) of two-dimensional observations (here shown for top layer).

### 1.2. Methods

Simulations of three-dimensional electrical and electromechanical scroll wave dynamics were performed in bulk-shaped isotropic and anisotropic (elastic) excitable media, respectively, and neural networks were used to predict the three-dimensional wave patterns from a short sequence of two-dimensional observations of the dynamics on the bulk's surface. ‘Laminar’ scroll wave dynamics, consisting of 1-3 meandering scroll waves, and ‘turbulent’ scroll wave chaos was distinguished.

### 1.2.1. Scroll Wave Dynamics in Elastic Excitable Media

Electrical scroll wave dynamics were simulated in bulk-shaped excitable media of size 128×128×d_{z} voxels with varying thicknesses or depths d_{z} ∈{8, . . . , 40}. The phenomenological Aliev-Panfilov model was used to simulate nonlinear waves of electrical excitation:

$$\frac{\partial u}{\partial t} = ∇ \cdot ( D ∇ u ) - ku ( u - a ) ( u - 1 ) - ur \qquad ( 1 )$$

$$\frac{\partial r}{\partial t} = ϵ ( u , r ) ( ku ( a + 1 - u ) - r ) \qquad ( 2 )$$

The dynamic variables u and r represent the local electrical excitation (voltage) and refractory state, respectively, and are dimensionless, normalized units. Together with the term (u, r)=ε₀+μ₁r/(u+μ₂), the partial differential equations describe the local excitable kinetics and diffusive dynamics. The parameters k, a, ε₀, u₁ and μ₂, listed in Table 1, influence the properties of the excitation waves. The partial differential equations were integrated using the forward Euler method in a finite differences numerical integration scheme and Neumann (zero-flux) boundary conditions were used.

$$D = {D}_{iso} I \qquad ( 3 )$$

Both isotropic and anisotropic excitable media were simulated with locally varying fiber direction with diffusion coefficients for the parallel D_{∥} fiber direction and perpendicular D_{⊥1}, D_{⊥2} to it [1]:

$$D = ( \begin{matrix}{D}_{11} & {D}_{12} & 0 \\ {D}_{21} & {D}_{22} & 0 \\ 0 & 0 & {D}_{33}\end{matrix} ) \qquad ( 4 )$$

$${D}_{11} = {D}_{‖} {cos}^{2} ( θ ( z ) ) + {D}_{⊥ 1} {sin}^{2} ( θ ( z ) )$$

$${D}_{22} = {D}_{‖} {sin}^{2} ( θ ( z ) ) + {D}_{⊥ 1} {cos}^{2} ( θ ( z ) )$$

$${D}_{12} = {D}_{21} = ( {D}_{‖} - {D}_{⊥ 1} ) cos ( θ ( z ) ) sin ( θ ( z ) )$$

$${D}_{33} = {D}_{⊥ 2}$$

Here, the fiber organization represents ventricular muscle tissue with muscle fibers aligned in sheets in the x-y plane and the sheet-fiber orientation rotating throughout the thickness of the bulk. D_{⊥1} is the diffusivity perpendicular to the fiber axis in the x-y plane and D_{⊥2} transmurally, for simplicity D_{⊥}=D_{⊥1}=D_{⊥2} was set. A varying fiber angle θ(z) ranging from 0° to 90° was used between the top and bottom layer of the bulk for the simulation depth d_{z}=24 voxels:

$$θ ( z ) = z \cdot Δθ \qquad ( 5 )$$

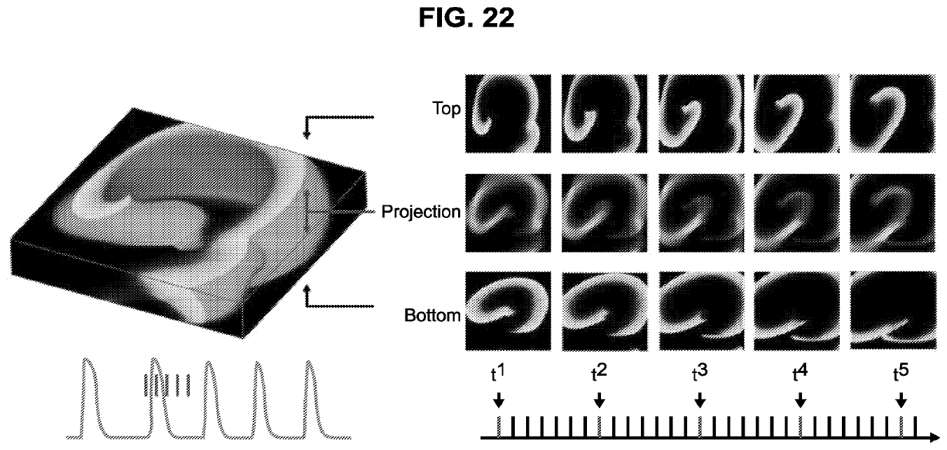

For the other depths, d_{z} ∈{8, 12, 16, 20, 28, 32, 40}, the same Δθ was used as in the d_{z}=24 case. The ratio between the parallel D_{∥} and perpendicular D_{⊥} diffusion coefficients was set to 4:1. D_{⊥}=Dᵢₛₒ=0.05 was chosen, and Δt was adapted for the Euler integration such that 500 simulation time steps correspond to about 0.5-1.0 scroll wave rotations. The simulation time steps required for one scroll wave rotation fluctuates and depends on the parameter values as well as an/-isotropy. To save disk space, only every 80th simulation time step was stored as one ‘snapshot’, such that 5 snapshots covered about a half to one scroll wave rotation, see also FIG. 22.

In addition to the purely electric simulations, electromechanical scroll wave dynamics in deforming excitable media were also simulated, as described in [22]. In short, a three-dimensional mass-spring damper system with hexahedral cells and tunable fiber anisotropy was coupled to the electric simulation. Each cell in the mechanical part of the simulation corresponded to one cell or voxel in the electrical part of the simulation. Active tension generation in each cell was modelled using an active stress variable Tₐ that is directly dependent on the excitation variable u, as described in [31]:

$$\frac{\partial {T}_{a}}{\partial t} = ∈ ( u ) \cdot ( {k}_{T} u - {T}_{a} ) \qquad ( 6 )$$

$$∈ ( u ) = \{ \begin{matrix}10 & if u < 0.05 \\ 1 & if u \geq 0.05\end{matrix}$$

The axis along which each cell exerts active contractile force could be pointed into an arbitrary direction, and, throughout the bulk, the axis alignment matched the rotating orthotropic fiber alignment already defined in the electrical part of the simulation. The mechanical parameter k_{T} and other parameters shown in Table 1 influence the magnitude of contraction and the properties of the elasticity of the mass-spring damper system. The elastic medium's boundaries were non-rigid and confined by elastic springs acting on the medium's boundary [22]. In general, the electromechanical simulation produces three-dimensional deformation patterns that are highly correlated with the electrical scroll wave chaos.

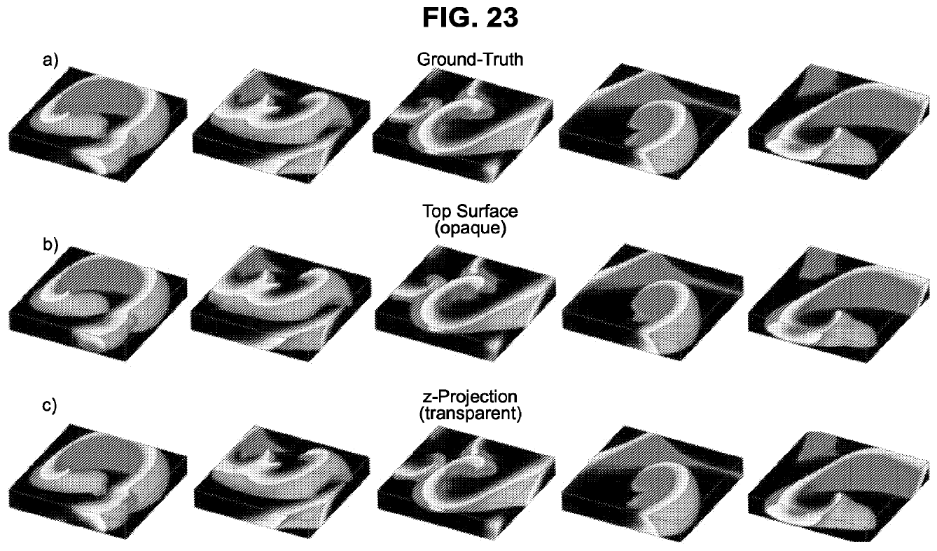

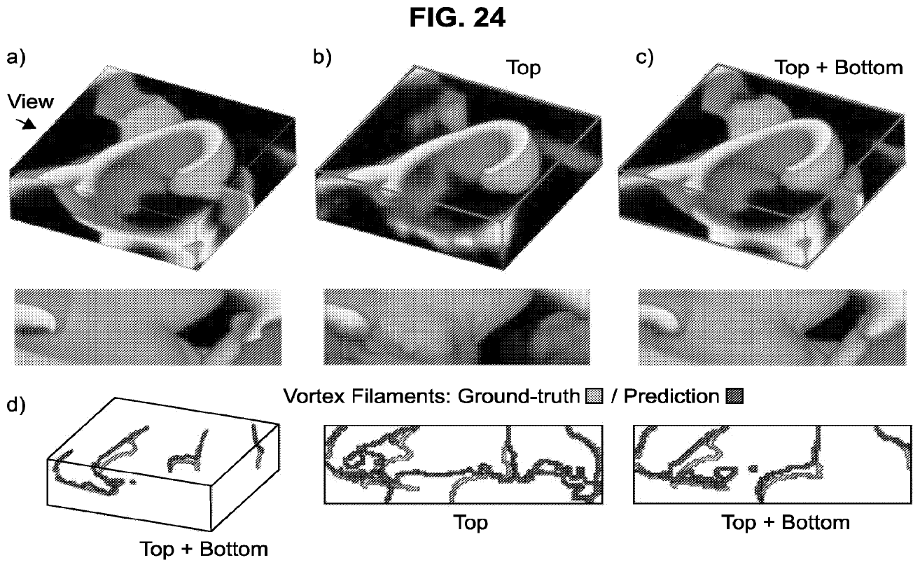

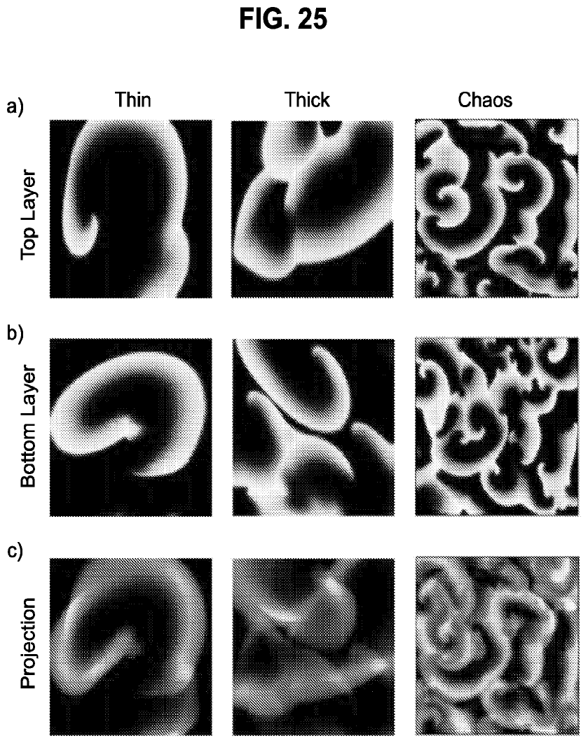

TABLE 1
Electrical (top) and mechanical (bottom) parameters
used to simulate two different regimes of scroll wave
dynamics: a ‘laminar’ regime (see FIGS. 23, 24)
and a fully ‘turbulent’ scroll wave chaos regime
(see FIGS. 25, 26 and 28). Electro-mechanical simulations
were only performed with the ‘turbulent’ parameter set.

| Parameter | ‘Laminar’ Set | ‘Turbulent’ Set |
|---|---|---|
| k | 8 | 8 |
| a | 0.05 | 0.05 |
| ε₀ | 0.002 | 0.002 |
| μ₁ | 0.8 | 0.2 |
| μ₂ | 0.3 | 0.3 |
| k_{T} | — | 3 |
| kᵢⱼ | — | 5 |
| kⱼ | — | 0.5 |
| k_{f} | — | 4 |
| c_{f} | — | 10 |

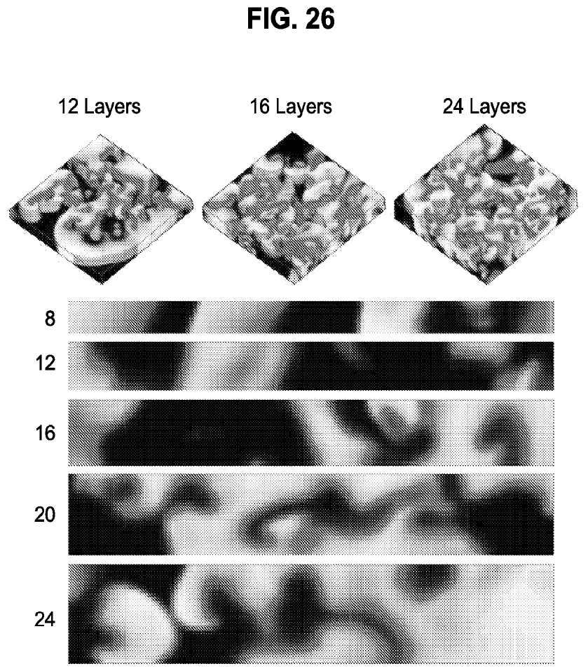

Two different regimes of scroll wave dynamics were simulated: 1) a ‘laminar’ regime with 1-3 meandering scroll waves with wavebreaks as shown in FIGS. 23 and 24 and 2) a fully ‘turbulent’ scroll wave chaos regime as shown in FIGS. 26-28 (see Table 1 for the respective parameter values). For both parameter regimes, 125 isotropic and anisotropic simulations of electrical scroll wave chaos were performed for different bulk depths-d_{z} ∈{16, 24, 32, 40} voxels for the ‘laminar’ regime and d_{z} ∈{8, 12, 16, 20, 24, 28, 32} for the ‘turbulent’ regime. 100 simulations were used during generation of the training dataset and 25 simulations were exclusively used for evaluation. Furthermore, 125 simulations of electromechanical scroll wave chaos were performed for a thickness of d_{z}=24, with the same split between training and evaluated dataset. The electrical and mechanical parameters were identical in each simulation. However, the initial conditions ut=0 (x, y, z), rₜ=0 (x, y, z) were randomized and therefore different in each simulation. Cross-field stimulation was used to set ut=0, rₜ=0 such that two scroll waves are induced at random positions (x₁, y₁), (x₂, y₂). Additionally a small amount of Gaussian noise (standard deviation σ=0.1) was added to the initial conditions uₜ=0, rₜ=0. With both parameter sets, the dynamics quickly diverged. The first 75 snapshots of each simulation were discarded (approximately 15 scroll wave rotation periods) and the remaining 500 snapshots were used (approximately 100 scroll wave rotation periods) for generating the training or evaluation data, respectively. If the excitation in a simulation decayed (uₜ≈0 V (x, y, z)), the simulation was restarted with new initial conditions uₜ=0, rₜ=0. The training dataset consists of 20,000 randomly selected samples from the 100 training simulations, and the evaluation dataset uses 5,000 randomly chosen samples from the 25 evaluation simulations. Consequently, training and evaluation datasets were completely separate datasets. The numerical simulation was implemented in C++, and the source code for the simulations is available in [22].

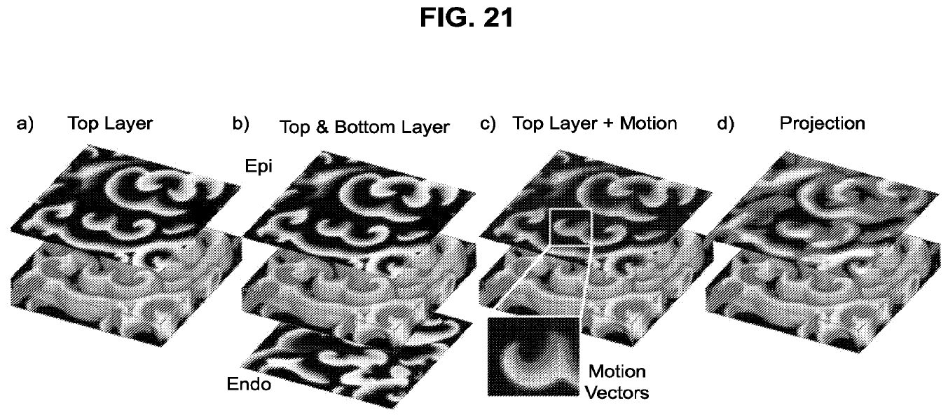

FIGS. 21A-21D: surface observations and projections of three-dimensional scroll wave chaos (‘turbulent’ parameter regime) in a bulk medium. A) Observation of the top surface of the bulk (layer 1) in single-surface mode. B) Observation of the top and bottom surfaces of the bulk (layers 1 and 24) in dual-surface mode. C) Observation of the electrical activity and mechanical motion on the top surface of the bulk (layer 1) in single-surface mode (red: motion vectors). In a-c), the medium is opaque and does not allow observations of the dynamics inside the medium below the top layer. D) Observation of the projection (transillumination) of the three-dimensional dynamics along the depth of the bulk. The projection is calculated by summing the values in all 24 layers along the z-axis for a specific (x, y)-coordinate and dividing the sum by the number of layers. All layers 1-24 are cross-sections in the x-y plane.

FIG. 22: Observations of three-dimensional scroll wave (‘laminar’ parameter set) on the top and bottom surfaces of an opaque medium, and in the projection of the full dynamics in a transparent medium in the bulk's z-direction (depth). In each case, the neural network analyzes a short sequence of 5 snapshots, which are sampled at discrete times (red) over the period of the scroll wave from the simulation data, see section II B for details. In the simulations, one rotational period corresponds to about 500 simulation time steps.

### 1.2.2. Deep Learning-based Reconstruction of Scroll Wave Dynamics

Deep neural networks were implemented and tested, which each analyze a short temporal sequence of 5 subsequent two-dimensional snapshots of electrical wave patterns ûₜ (x, y) to reconstruct a single fully three-dimensional snapshot uₜ (x, y, z) of scroll wave dynamics:

$$( {\tilde{u}}_{1} ( x , y ) , … , {\tilde{u}}_{5} ( x , y ) ) \to {u}_{1} ( x , y , z ) \qquad ( 7 )$$

It was empirically found that 5 snapshots provide sufficient information about the dynamics (see, e.g., [26] or [32]). Reconstructing the dynamics with a series of 10 snapshots was also tested and no improvement in performance was observed compared to 5 snapshots. Consequently, 5 snapshots was used by default. Further, it was found that the reconstruction accuracy does not depend on whether the network analyzes the current snapshots plus 4 snapshots sampled in the past or in the future with respect to the current snapshot, or whether 2 are sampled in the past and 2 in the future, respectively. The two-dimensional snapshots (see FIGS. 21, 22 and 25) are either i) the top surface layer (single-surface mode, FIG. 21A):

$${\tilde{u}}_{t} ( x , y ) = {u}_{t} ( x , y , 1 ) \qquad ( 8 )$$

ii) both the top and bottom surface layer (dual-surface mode, FIG. 21B):

$${\tilde{u}}_{t} ( x , y ) = ( {u}_{t} ( x , y , 1 ) , {u}_{t} ( x , y , dz ) ) \qquad ( 9 )$$

iii) both the electrical wave dynamics and the mechanical displacements \vec{d}=(dx, dy) which occur in corresponding electromechanical simulations from the top surface layer (single-surface mode), see FIG. 21C):

$${\tilde{u}}_{t} ( x , y ) = ( {u}_{t} ( x , y , 1 ) , dx ( x , y , 1 ) , dy ( x , y , 1 ) ) \qquad ( 10 )$$

or iv) a projection of all u-values along the z-direction (depth) of the bulk (FIG. 21D):

$${\tilde{u}}_{t} ( x , y ) = \frac{1}{{d}_{z}} {\sum }_{i = 1}^{{d}_{z}} {u}_{t} ( x , y , i ) \qquad ( 11 )$$

For the top+bottom case (iii) and the mechanical displacement case (iv), the snapshots were interleaved. The neural network architectures chosen require three-dimensional input samples ((128, 128, 5) for case (i) and (ii)), whereas in cases (iii) and (iv) the sample shape is (128, 128, 5, 3) and (128, 128, 5, 2) respectively. Their shape was changed to be three-dimensional by stacking the components: e.g., in case (iv) the resulting shape is (128, 128, 10) using the top layers for the even indices and bottom frames for the odd indices (u₁ (x, y, 1), u₁(x, y, d_{z}), u₂(x, y, 1) . . . u₅(x, y, d_{z}).

Four different neural network architectures were evaluated with basic and more intricate designs for the three-dimensional bulk prediction task (Eq. 7). While a U-Net [33] architecture was primarily use, it was validated against a simple Encoder-Decoder architecture, TransUNet [34] and MIRNet [35]. The Encoder-Decoder convolutional neural network (CNN) is similar to the architecture used in [26, 32]. It consists of an encoder stage where the spatial resolution is progressively decreased, a latent space, and a decoder stage where the spatial resolution is progressively increased back to the original resolution. The encoding and decoding steps consist of three steps, in each two padded two-dimensional convolutional layers (2D-CNN) with filter size 3×3 and rectified linear unit (ReLU) activation are applied, followed by batch normalization and maxpooling (encoder) or upscaling (decoder), respectively. The number of filters in 2D-CNN layers in order are 128, 128, 256, 256, 512, 512, 256, 256, 128 and 128. The U-Net architecture is identical to the Encoder-Decoder CNN architecture, except that skip connections are added between the encoder and decoder stages (see [33]). The TransUNet combines the U-Net architecture with self-attention mechanisms of Transformers in its latent space. The MIRNet architecture is different from the other evaluated architectures, as it contains parallel multi-resolution branches with information exchange, as well as spatial and channel attention mechanisms [35]. It aims at maintaining spatially precise high-resolution representations through the entire network, while simultaneously receiving strong contextual information from the low-resolution representations. For all neural network architectures the generalized Charbonnier loss function was use [39, 40]:

$$l ( u , \hat{u} ) = \sqrt{{( \hat{u} - u )}^{2} + {∈}^{2}} \qquad ( 12 )$$

where u is the three-dimensional ground truth, û the prediction and ∈=0.001 was chosen. The Charbonnier loss function behaves like L2 loss (mean squared error) when u≈û and like L1 loss (mean absolute error) otherwise. The accuracy of the predictions was evaluated on the evaluation datasets with the root mean squared error (RMSE) on each z-axis layer:

$$RMSE ( z ) = \sqrt{\frac{1}{N} {\sum }_{x , y , t} {( \hat{u} ( x , y , z , t ) - u ( x , y , z , t ) )}^{2}} \qquad ( 13 )$$

To validate the findings, it was studied if a neural network can accomplish a simpler task than the three-dimensional prediction: estimate the depth d_{z} of the simulation bulk from 5 two-dimensional observations. Both a depth regression and a depth classification neural network were tested, which each predict the depth d_{z} of the simulation bulk:

$$( {\tilde{u}}_{1} ( x , y ) , … , {\tilde{u}}_{5} ( x , y ) ) \to {d}_{z} \qquad ( 14 )$$

The depth regression network predicts the depth d_{z} as a continuous value, while the depth classification network predicts the depth d_{z} as one of {8, 12, 16, 20, 24, 28, 32}. For this task the encoder part from the Encoder-Decoder architecture was used, followed by a global average pooling layer, two dense layers with 1024 filters with batch normalization and ReLU activation, and ultimately an output dense layer with one filter (for regression) or seven filters (for classification). For the depth classification neural network a categorical cross-entropy loss function was used with a softmax activation function for the last layer, and for the depth regression network mean squared error as loss function and ReLU as activation function. The datasets for the depth estimation was generated from the bulk prediction task datasets. 4,000 random samples were used for each depth for the training dataset and 500 samples for the evaluation dataset (in total 28,000 training samples and 3,000 evaluation samples).

The networks were trained using the Adam [41] optimizer with a learning rate of 10⁻³ for the bulk prediction tasks and 10⁻⁵ for the depth regression and classification task for 20 epochs. A batch size of 32 was used for the Encoder-Decoder and U-Net architectures and a batch size of 4 was used for TransUNet and MIRNet. All neural network models were implemented in Tensorflow using [42] Keras [43]. Training and reconstructions were performed on NVIDIA RTX 24000 graphics processing units (GPUs).

TABLE 2
Different neural network architectures used in this study
and their respective number of trainable parameters and
training times for 20 epochs. Training was
performed on a single NVIDIA RTX 24000 GPU.

| Model | Parameters | Training Time |
|---|---|---|
| Encoder-Decoder | 6,829,309 | 44 min |
| U-Net | 8,278,168 | 55 min |
| TransUNet | 406,899,608 | 19 hours |
| MIRNet | 145,358,026 | 17 hours |
| d_{z} Regression | 426,593 | 5 min |
| d_{z} Classification | 432,743 | 5 min |

FIGS. 23A-23C: Predictions of three-dimensional ‘laminar’ scroll wave dynamics from two-dimensional observations using deep convolutional encoding-decoding neural network (U-Net) in an anisotropic excitable medium (128×128×24 voxels). A) Ground-truth scroll wave dynamics (5 random representative snapshots). The simulations exhibit scroll waves with meandering and curved vortex cores (see also FIG. 24D), wavebreak, and dissociation between the top and bottom surface dynamics (see FIG. 25). B) Predictions from two-dimensional wave pattern visible only on the top surface of the bulk when the medium is completely opaque. The reconstruction accuracy decreases slightly with increasing depth (wave pattern becomes fuzzy towards the bottom). C) Predictions from two-dimensional projection of the whole three-dimensional dynamics along the z-axis in a transparent medium. The prediction accuracy is slightly better than in B), particularly towards the bottom layers of the bulk. Overall, the predictions and the ground-truth are visually difficult to distinguish from each other. The data was not seen by the network during training.

FIGS. 24A-24D: Predictions of three-dimensional scroll wave dynamics (‘laminar’ parameter set) and their vortex filaments from either single- or dual-surface observations in a thick, opaque, anisotropic excitable medium (128×128×40 voxels). A) Ground-truth scroll wave dynamics. B) Prediction from the top surface only. C) Prediction from both the top and bottom surfaces. In dual-surface mode, the network is able to recover the dynamics sufficiently well. Arrow indicates direction of cross-sectional view. D) Ground-truth vortex filaments (gray) and vortex filaments calculated from predicted scroll wave dynamics (red). In single-surface mode, predictions become unreliable (wave pattern becomes fuzzy/vortex filaments do not match) towards the bottom of the bulk. Reconstructions performed with U-Net.

FIGS. 25A-25C: Different electrical wave patterns as seen in the A) top layer, B) bottom layer and C) projection of all layers of a three-dimensional bulk. Bulk sizes are 128×128×24 voxels (left and right) and 128×128×40 voxels (center), respectively. With increasing bulk thickness or smaller scroll waves, the top and bottom layers are dissociated because the dynamics become increasingly three-dimensional. The projection is calculated for a given (x, y)-coordinate by averaging the u-values (u ∈[0, 1]) along the depth (z-direction) of the bulk. Data (from left to right) shown in FIGS. 23, 24 and 28, respectively. The left and center snapshots are from the ‘laminar’ parameter regime, while the ones on the right are from the ‘turbulent’ chaotic parameter regime.

### 1.3. Results

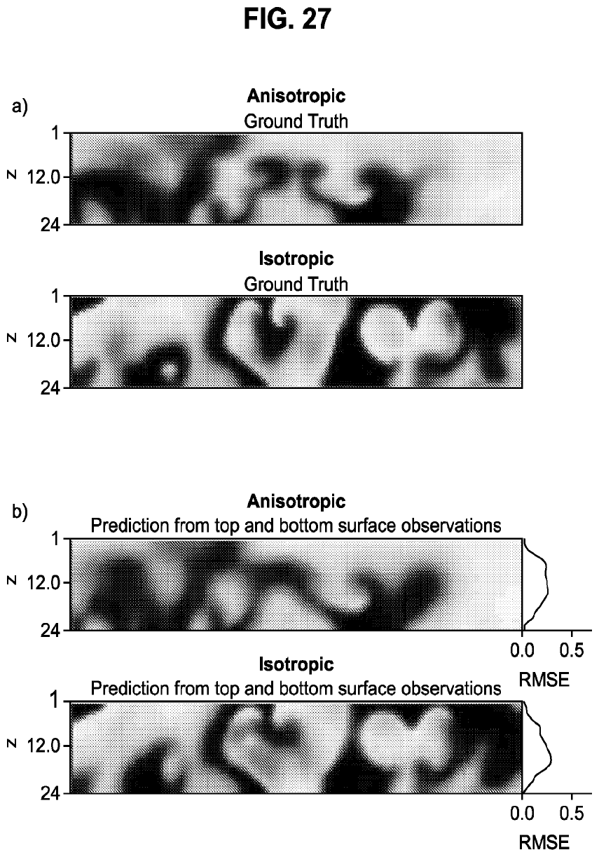

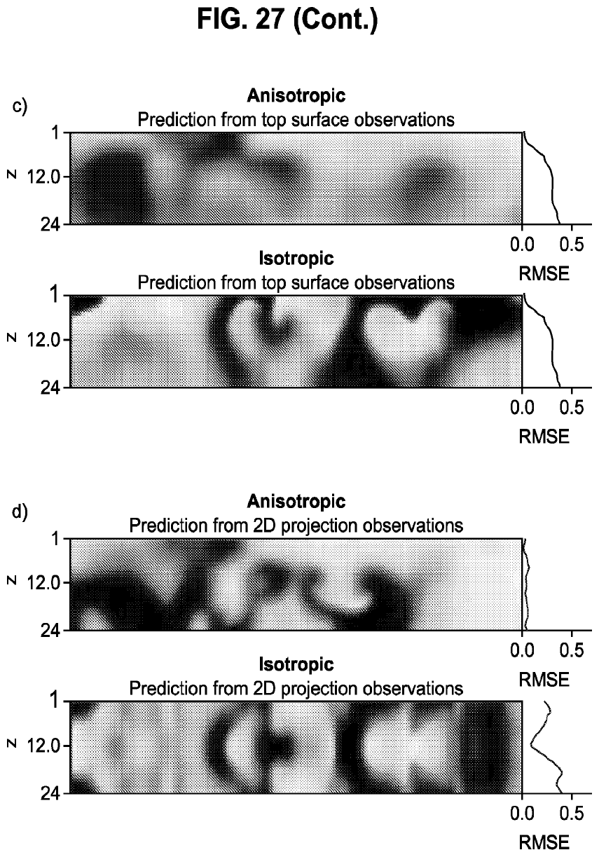

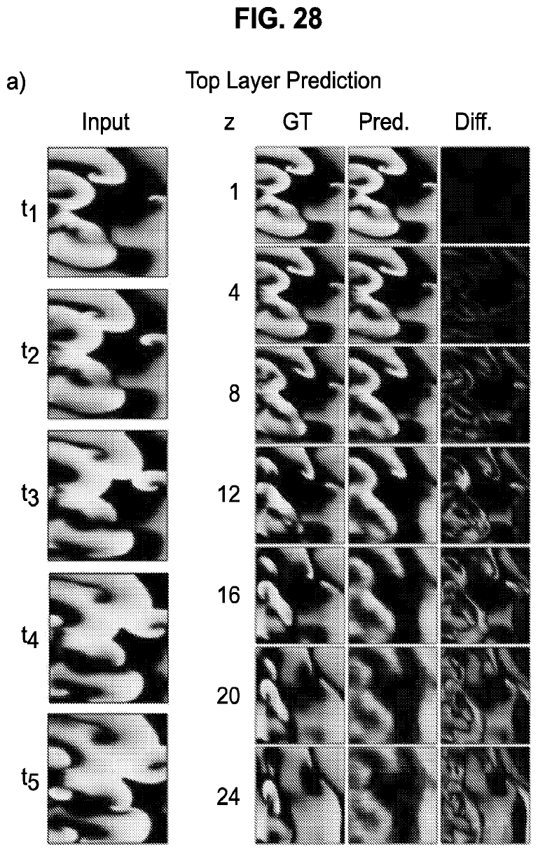

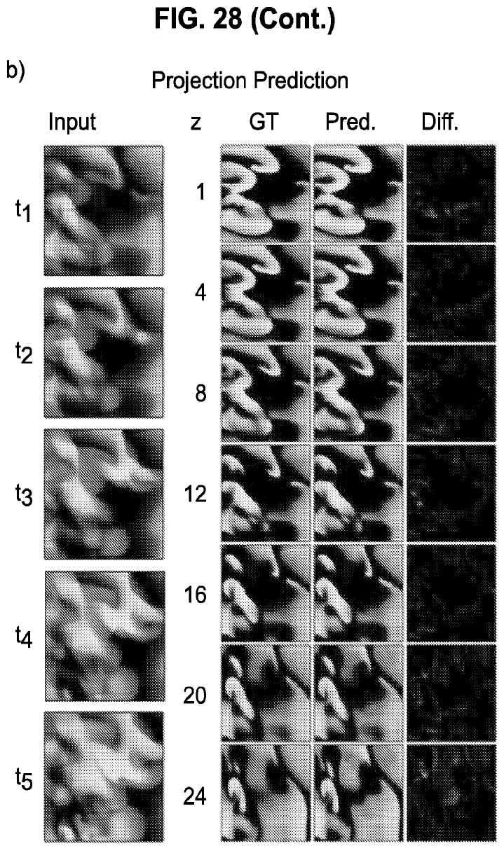

Using deep convolutional neural networks, it is possible to reconstruct three-dimensional scroll wave dynamics inside an excitable medium when the medium's thickness is not much thicker than the scroll wave (see, e.g., FIG. 23 and section 1.3.1 below). Reconstructions become increasingly difficult in thicker excitable media or with smaller scroll waves and more complicated dynamics (see, e.g., FIGS. 24, 28A and sections 1.3.1-1.3.3 below). However, complicated scroll wave chaos can be reconstructed in transparent anisotropic excitable media (see FIGS. 27D, 28B, 29C and 29F), or with dual-surface observations in thinner opaque excitable media (see FIGS. 24C, 27B and section 1.3.2 below).

1.3.1. Medium Thickness vs. Scroll Wave Size

The thickness of the excitable medium and the size of the scroll waves with respect to the thickness of the medium determine to what extent and with which accuracy scroll wave dynamics can be reconstructed. To test the reconstruction performance with different size ratios, ‘laminar’ scroll waves (see FIGS. 23 and 24) and ‘turbulent’ scroll wave chaos (see FIGS. 26, 27C and 28A) were simulated in bulks with varying thicknesses. While the reconstructions from single surface observations are very accurate for the ‘laminar’ scroll wave dynamics in the thinner bulk (with thickness d_{z}=24) shown in FIGS. 23A-23B, reconstructions become increasingly inaccurate with increasing thickness and prediction depth. The reconstructions of the ‘laminar’ scroll wave dynamics in the thicker bulk (with thickness d_{z}=40), shown in FIG. 24A-24B, exhibit artifacts towards the bottom half of the bulk. Accordingly, the vortex filaments computed from the predicted scroll wave dynamics (red) exhibit substantial mismatches compared to the ground-truth vortex filaments (gray) in the lower half of the bulk, see FIG. 24D. With increasing bulk thickness, the three-dimensional character and complexity of the wave dynamics increases, which is reflected by the dissociation of the top and bottom layers (see FIGS. 25 and 26) and also by the various orientations of the vortex filaments in FIG. 24D. The degree of dissociation and the average scroll wave size relative to the medium's thickness ultimately determine the prediction accuracy at deeper layers. The ‘horizon’ up to which the predictions are successful appears to be approximately one scroll wavelength, also compare the bottom half of the thick bulk with ‘laminar’ scroll wave dynamics in FIG. 24B to the bottom half of the thinner bulk with ‘turbulent’ scroll wave chaos in FIGS. 27B and 27A. In general, the reconstructions become increasingly difficult the deeper one aims to predict in opaque excitable media, and they do not succeed beyond the first layer of scroll waves.

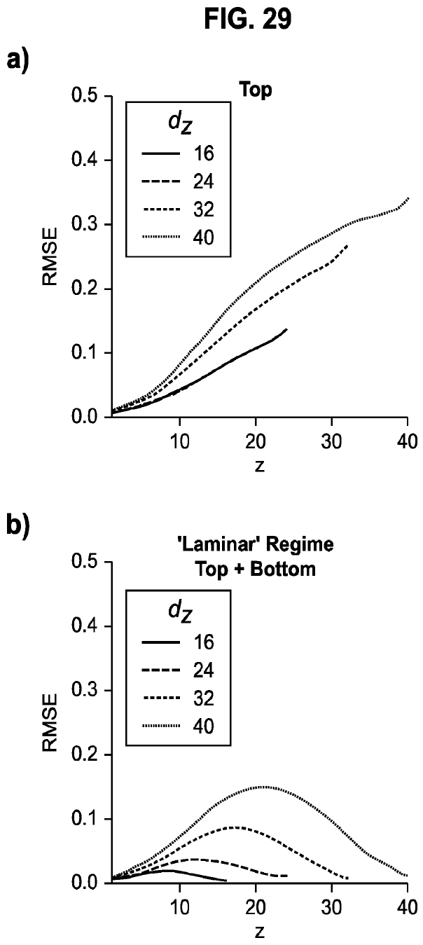

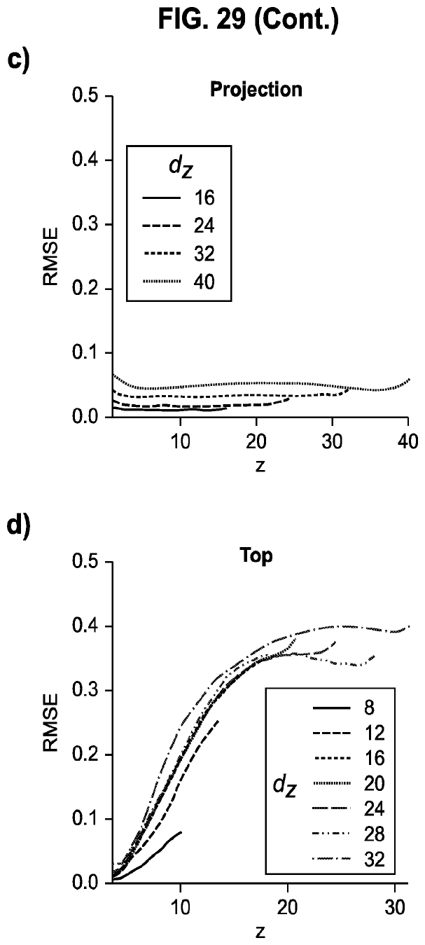

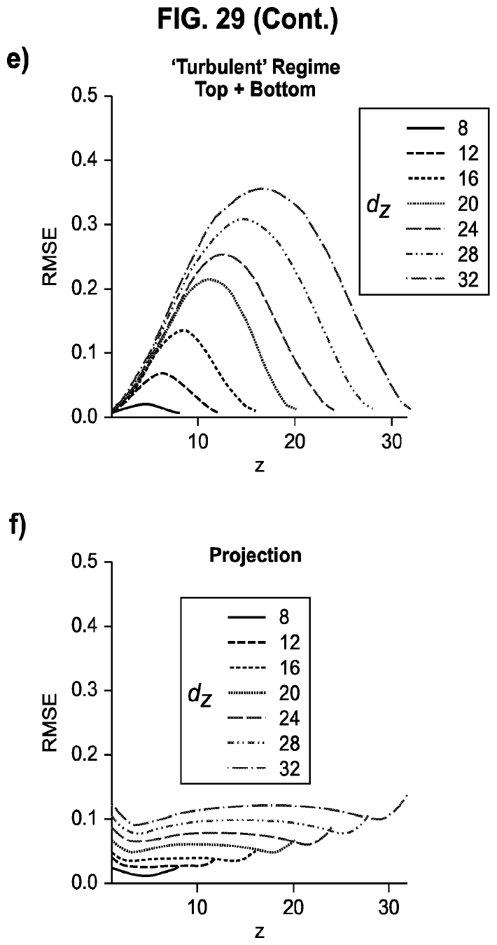

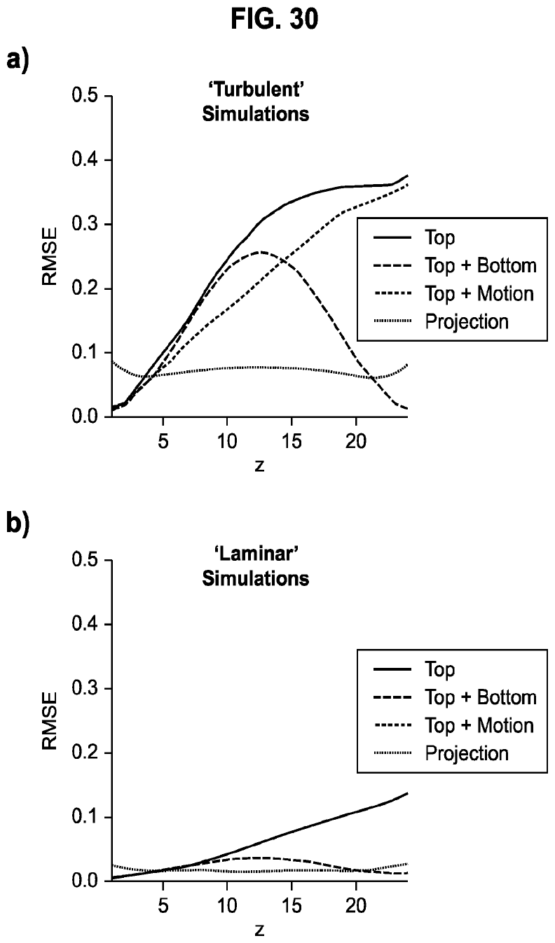

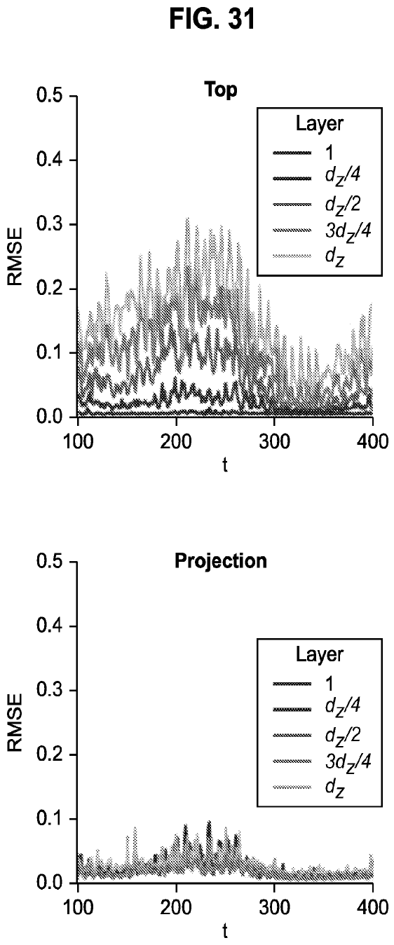

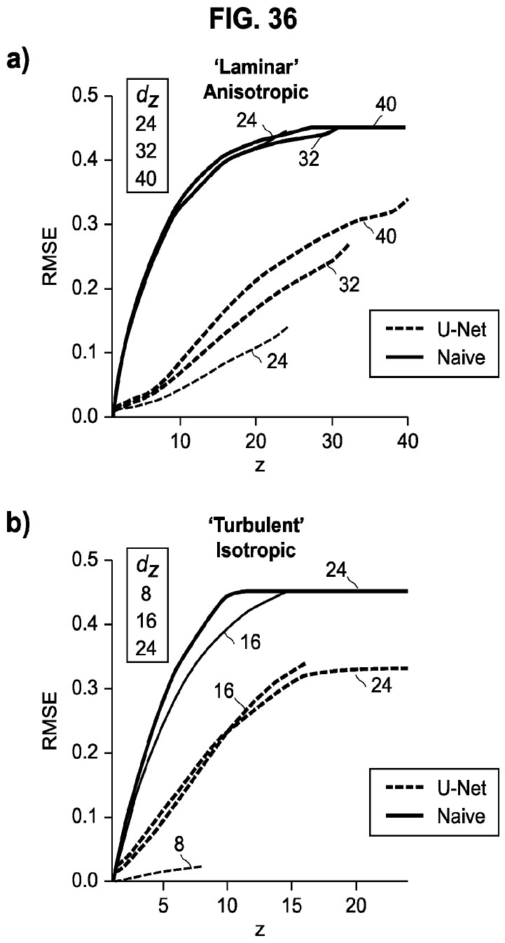

Correspondingly, the plots in FIG. 29A-29D show how the prediction error increases in opaque excitable media with increasing depth z (in bulks with different depths d_{z}=8, 12, . . . , 40) with ‘laminar’ and ‘turbulent’ scroll wave dynamics, respectively. The error increases approximately linearly with increasing depth and increases faster with thicker bulks and faster with ‘turbulent’ than with ‘laminar’ scroll wave dynamics. The error profiles obtained with the deep learning-based reconstruction were compared with a naive reconstruction in which the top layer is simply repeated in each following layer in FIG. 36. The naive reconstruction produces significantly steeper error curves with both ‘laminar and ‘turbulent’ scroll wave dynamics. The curves in FIGS. 29A and 29D indicate that the average reconstruction accuracies in FIG. 23B and FIG. 24B are approximately 0.05 and 0.15 (RMSE) at midwall, respectively, while the deepest layers shown in FIG. 24B yield reconstruction errors in the order of 0.2-0.5 (RMSE). By comparison, the worst average reconstruction error at the maximal depth of the ‘laminar’ scroll wave shown in FIG. 25B is about 0.1 (RMSE) (see blue curve in FIG. 30B). Note that a root mean squared error of 0.1 (RMSE) corresponds to about 0.05 mean absolute error (MAE). The average single-surface reconstruction error for the ‘laminar’ scroll wave in FIG. 25B is better than 95%. Overall, the reconstruction error fluctuates moderately over time, remains small at smaller depths, and increases as the reconstruction error increases with larger depths (see FIG. 31). Videos demonstrating the reconstructions for ‘laminar’ and ‘turbulent’ scroll wave dynamics and giving an impression of the temporal stability of the reconstructions were produced.

FIG. 26: Bulk thickness and transmurality of scroll wave dynamics. ‘Turbulent’ scroll wave chaos with different bulk thicknesses d_{z}={8, 12, 16, 20, 24}, also shown in corresponding cross-sections. The dynamics are quasi two-dimensional with d_{z}=8. Dissociation between top and bottom layers starts to emerge at d_{z}=12 as the dynamics become increasingly three-dimensional. At d_{z}>12 the dynamics are fully three-dimensional.

FIGS. 27A-27D: Predictions of (‘turbulent’) electrical scroll waves in subsurface layers of anisotropic (left) and isotropic (right) bulk tissue with dimension 128×128×24 voxels. A) Ground-truth scroll wave dynamics (representative snapshots). B) Prediction in dual-surface mode analyzing the top and bottom layers of an opaque bulk tissue. C) Prediction in single-surface mode analyzing the top surface layer of an opaque bulk. D) Prediction analyzing the z-projection of the dynamics along its depth (or z-axis) in a transparent bulk. The depth-profile of the prediction error (RMSE: root mean squared error) along the z-axis is shown to the right of each prediction. The reconstruction is successful in anisotropic transparent media, but fails in isotropic transparent media, as shown in D). In opaque media, the reconstruction performs sufficiently well in dual-surface mode with larger errors emerging at midwall of the bulk, as shown in B). In transparent isotropic media, the subsurface prediction fails because the network is unable to infer the depth of the layers. Cross-sections intersect the bulk at its center. All reconstruction with U-Net.

FIG. 31: Average reconstruction error per layer (depth) over time in an anisotropic excitable medium with thickness d_{z}=24 shown for 5 different depths. Left: In opaque media and single-surface mode, the prediction error increases and fluctuates more with increasing depth. Right: In transparent media, the prediction error stays small throughout the different depths. Both plots derived for the ‘laminar’ scroll wave dynamics as shown in FIG. 23.

1.3.2. Single-Surface vs. Dual-Surface Observations

Low prediction depths in opaque excitable media can be overcome by analyzing both the top and bottom surface layers in dual-surface mode rather than single-surface mode, respectively. FIGS. 24C and 27B demonstrate how the reconstruction improves in a thick bulk with the ‘laminar’ scroll wave and in a thin bulk with ‘turbulent’ scroll wave chaos, respectively. The vortex filaments (red) in the thick bulk in FIG. 24C match the ground-truth vortex filaments (gray) much better in dual than in single-surface mode. The plots in FIGS. 29B, 29E and 30 show how the profile of the reconstruction error changes in dual-surface mode. Surprisingly, the network does not appear to benefit from the additional information from both surfaces of the bulk in dual-surface mode with scroll wave chaos: the steep linear increase in the error persists on both sides and the network is not able to significantly reduce the error at midwall (see FIG. 29B). Nevertheless, it is possible to slightly reduce the error at midwall when reconstructing larger scroll waves (see FIGS. 24C, 29B and 30B). The data suggests that it could be possible to reconstruct scroll waves in the heart using epi- and endocardial optical mapping recordings if the scroll wavelength is not much shorter than the thickness of the ventricular wall.

1.3.3. Transparent vs. Opaque Excitable Media

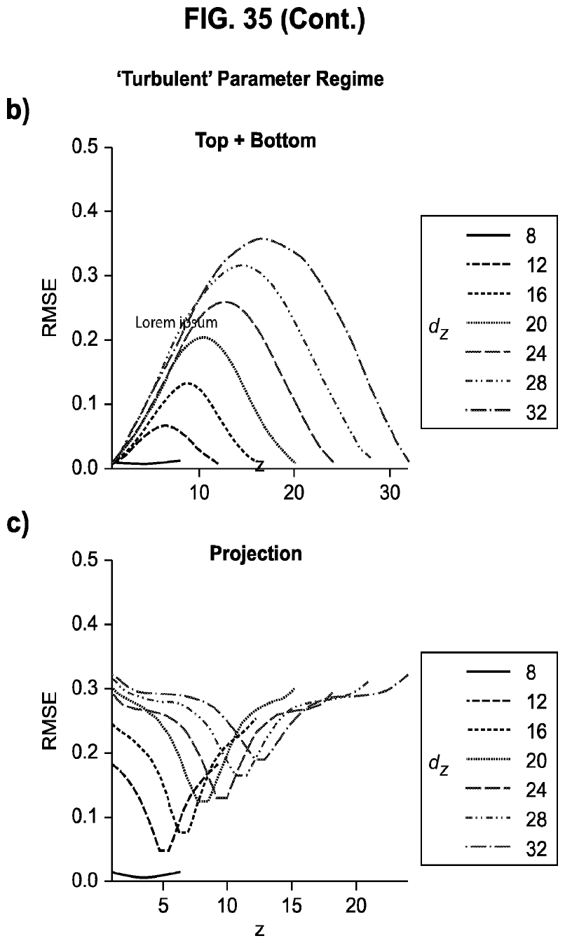

While it is challenging to reconstruct scroll wave dynamics or scroll wave chaos in thicker opaque excitable media, the reconstructions succeed in transparent excitable media of any thickness (of the thicknesses tested). However, the reconstructions only succeed under the condition that the excitable media are anisotropic. FIGS. 27 and 28 show a comparison of reconstructions obtained in an opaque and transparent excitable medium, respectively. FIG. 27 shows cross-sections along the depth of the bulk (z-direction) whereas FIG. 28 shows layers parallel to the surface in the x-y plane of the bulk. Comparing FIGS. 27C and 27D and FIGS. 28A and 28B, it becomes immediately apparent that it is possible to obtain highly accurate reconstructions of scroll wave chaos in transparent, anisotropic excitable media, while it would not be possible to obtain similar reconstructions in opaque media (see also plots in FIG. 35 with isotropy). FIG. 29C shows that the reconstruction error stays small transmurally throughout the entire bulk if the excitable medium is transparent and anisotropic. The prediction error is <0.1 (RMSE) with various bulk depths (d_{z}=8-32) and with the ‘laminar’ and ‘turbulent’ scroll wave dynamics. Importantly, as can be seen in the right panel in FIG. 27D, the reconstruction completely fails in isotropic transparent excitable media (see also FIG. 35). The effect of anisotropy on the prediction is discussed in more detail below.

FIGS. 28A-28B: Predictions of electrical scroll waves within subsurface layers of a bulk-shaped ‘turbulent’ anisotropic excitable medium from observing either A) the top layer of the bulk or B) the projection of the three-dimensional wave pattern in a transparent bulk along its depth (or z-axis). The bulk's dimensions are 128×128×24 voxels and predictions were performed with the U-Net architecture. Predictions are shown for the 24 layers along the z-axis of the bulk, where the first layer is the top layer and the 24th layer is the bottom layer. First row: The five two-dimensional frames (t1, . . . , t₅) which are the input for the neural network prediction. Second row: Ground truth (GT) electrical excitation wave pattern within cross-sectional layers (1-24), of which layers 2-24 cannot be observed. Note that the pattern changes from layer to layer throughout the bulk. Third row: Prediction of the current cross-sectional layer (1-24) by the neural network. Fourth row: Absolute difference per voxel between prediction and ground-truth.

FIGS. 29A-29F: Average reconstruction error over bulk depth (RMSE: root mean squared error along z-axis) in opaque (A, B, D, E) or transparent (C, F) excitable media with anisotropy (with varying bulk depths of d_{z} ∈{8, 12, . . . , 40}). All reconstructions were performed with U-Net. A-C) ‘Laminar’ scroll wave dynamics as shown in FIGS. 23 and 24: A) Single-surface mode (see also FIGS. 25B and 24B). B) Dual-surface mode. C) Projection (see also FIGS. 25C and 24C). ‘Turbulent’ scroll wave chaos as shown in FIG. 26: D) Single-surface mode (see also FIGS. 27C and 28A). E) Dual-surface mode (see also FIGS. 21B and 27B). F) Projection (see also FIGS. 27D and 28B). An error of 0.1 (RMSE) corresponds to a mean absolute error (MAE) of about 5%. In opaque excitable media, the reconstruction error increases approximately linearly with depth. In transparent media with anisotropy the error remains flat and below 0.1. Separate U-Net neural networks were trained for each combination. See also FIG. 35 for a comparison with isotropic excitable media and FIG. 36 for a comparison of the deep learning-based reconstruction with a naive reconstruction in which the top layer is simply repeated in each following layer.

### 1.3.4. Anisotropy

While ventricular muscle tissue is highly anisotropic (orthotropic muscle fiber organization), the Belousov-Zhabotinsky chemical reaction is isotropic. Both systems exhibit scroll waves, but the scroll wave morphology can be very different in anisotropic versus isotropic excitable media. In anisotropic media, scroll waves are elongated in fiber direction as they propagate faster along the fiber direction. This phenomenon can often be observed in optical mapping recordings. In the simulated anisotropic bulk, the waves are elongated differently at different depths, which is presumably why the reconstructions succeed in transparent excitable media as shown in FIGS. 29C and 29F. By contrast, in isotropic excitable media the scroll waves are similarly shaped throughout the bulk, and therefore the network cannot distinguish scroll waves closer to the surface from scroll waves deeper in the bulk (see also FIG. 36). Anisotropy does not affect the reconstructions in opaque excitable media, as the reconstruction does not rely on depth information.

### 1.3.5. Analyzing Surface Deformation

If the network analyzes the mechanical deformation of the surface in addition to the excitable wave patterns visible on the same surface, the reconstruction improves slightly (see plot ‘Top+Motion’ in FIG. 30A). This behavior was tested with ‘turbulent’ scroll wave chaos (the U-Net architecture and the single-surface configuration shown in FIG. 21C) and it was found that the reconstruction improves slightly, but not substantially. This is a surprising finding, because deformation on the surface may also occur due to contractile activity within the bulk. The reconstruction error does not increase as steeply with increasing depth as when analyzing electrics in single-surface mode alone. The reconstruction accuracy improves by roughly 25% at midwall (with a bulk thickness of d_{z}=24 layers). It was found that there was no significant difference between analyzing only two-dimensional in-plane displacements \vec{u}=(uₓ, u_{y}) with x- and y-components versus three-dimensional displacements with also a z-component (see also eq. (10) in section 1.2.2). That the latter finding is specific to the methodology of this example and the mechanical boundary conditions that were used in the simulations cannot be excluded.

FIGS. 30A-30B: Comparison of reconstruction errors obtained with different imaging configurations in opaque anisotropic excitable medium (with thickness d_{z}=24 layers) with A) ‘turbulent’ scroll wave chaos and B) ‘laminar’ scroll wave dynamics. In the different configurations, the network (U-Net) analyzes i) in single-surface mode the electrics on the top layer (blue), ii) in single-surface mode the electrics and motion on the top layer (green), iii) in dual-surface mode the electrics on both top and bottom layers (orange), and iv) the z-projection of the three-dimensional electrics (red). All reconstruction errors were calculated per depth as root mean squared error (RMSE).

### 1.3.6. Noise

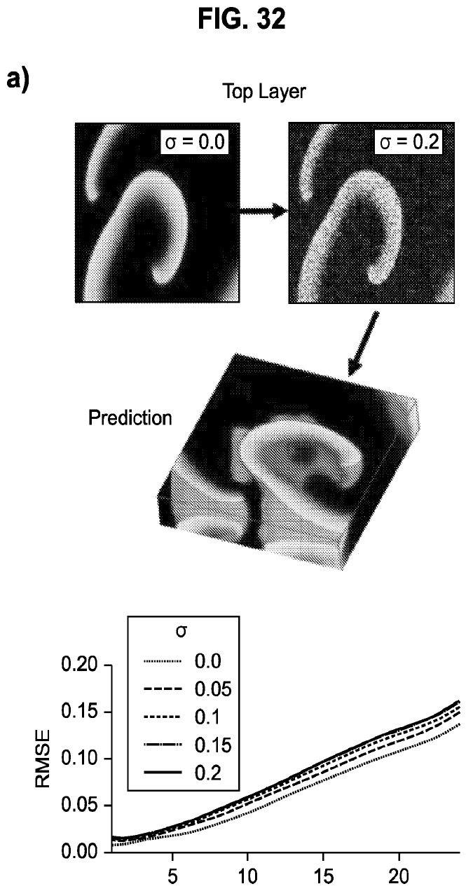

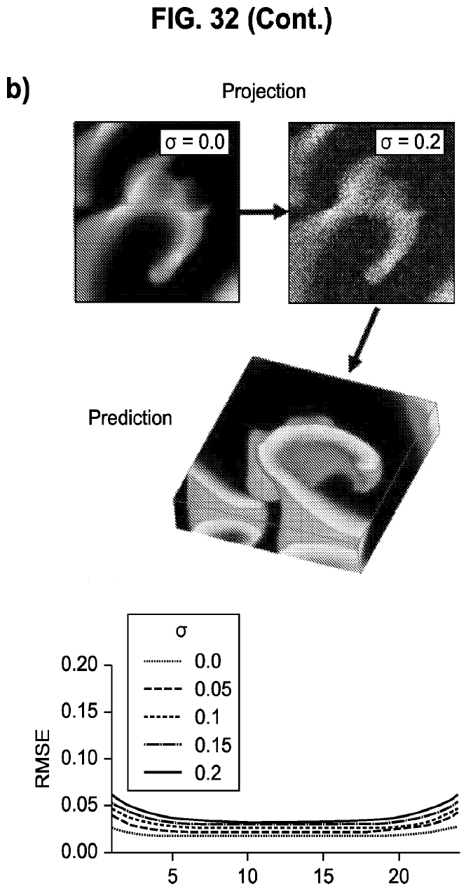

Reconstructions can be performed with noise (see, e.g., FIG. 32) if the network was previously trained with noise, similarly as described in [26] and [32]. The noise can be present in either the surface observations or projections. FIGS. 32A and 32B show reconstructions of scroll wave dynamics with noise in an opaque and a transparent anisotropic excitable medium (thickness: d_{z}=24 layers), respectively. In the opaque bulk in single-surface mode, the slope of the reconstruction error is slightly steeper with noise than without. In the transparent bulk, the error profile remains flat and stays below 0.1 (RMSE) with noise. This behavior was tested with Gaussian noise and noise levels of up to σ=0.2, shown in FIG. 32 (top right in each panel).

FIGS. 32A-32B: Noise does not pose a limitation for the deep learning-based reconstructions (performed with U-Net on ‘laminar’ scroll wave dynamics). Reconstructions succeed in the presence of various noise levels (σ=0.05, . . . , 0.2) in both A) opaque and B) transparent anisotropic excitable media. Top: Gaussian noise with standard deviation σ was added onto the input images. Bottom: Error profiles along depth increase only slightly (light pink curve: σ=0.0, black curve: σ=0.2).

### 1.3.7. Network Types

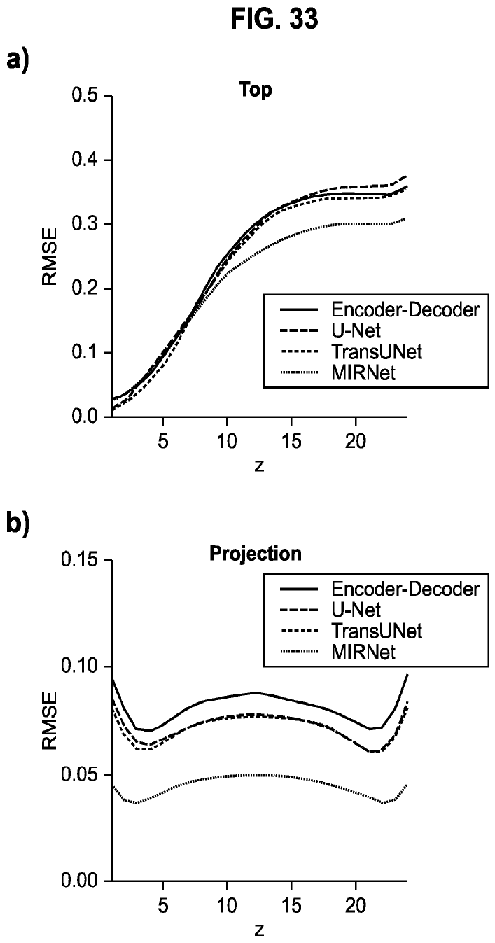

Several deep neural network architectures were tested (basic Encoder-Decoder, U-Net, TransUNet and MIRNet, see also section 1.2.2) on the ‘turbulent’ scroll wave chaos prediction task and it was found that the prediction behavior is very similar across the different architectures (see FIG. 33). It was observed that the MIRNet architecture produces the lowest reconstruction error, while Encoder-Decoder, U-Net, and TransUNet all have similar but slightly higher reconstruction errors. In opaque excitable media, the prediction error (RMSE: mean root squared error) rises linearly and steeply with deeper layers equally with all networks, as seen for the single-surface reconstructions shown in FIG. 33A for anisotropic scroll wave chaos in a bulk with thickness d_{z}=24. The prediction error saturates equally with all networks at depths d_{z}>10 where they produce maximal prediction errors of about 0.3 (RMSE). MIRNet provides a slightly lower maximal error than the other networks. All networks achieve small prediction errors of <0.1 (RMSE) in transparent excitable media with anisotropy, as seen for the projection reconstructions shown in FIG. 33B for anisotropic scroll wave chaos in a bulk with thickness d_{z}=24. MIRNet provides the lowest error of less than 0.05 (RMSE), whereas the other networks produce errors ranging between 0.06-0.1 (RMSE). All networks produce the same characteristic error profile. Note that an RMSE of 0.1 corresponds to a mean absolute error (MAE) of about 5%. Therefore, all networks achieve reconstruction accuracies of greater than 95% in transparent excitable media. MIRNet even achieves reconstruction accuracies in the order of 97%-98%.

As described in section 1.2.2, U-Net differs from the Encoder-Decoder architecture in the inclusion of long skip connections, while TransUNet is a U-Net with a Transformer as the latent space. MIRNet is different in that it has multi-resolution branches with information exchange as well as self-attention mechanisms. The training times for 20 epochs and the number of trainable parameters for each network architecture are listed in Table 2. Given that TransUNet and MIRNet provided either no or incremental improvements in prediction accuracy, while requiring significantly longer time to train, U-Net was primarily used in this study. U-Net required about an hour to train, while being competitive with the accuracy of MIRNet, which required almost a full day to train. All other results in FIGS. 23-28 were obtained with the U-Net architecture, if not stated otherwise. Several U-Net sizes were tested: a small model with 0.5M parameters, a medium network with 2M parameters and a large model with 8M parameters, it was determined that larger models perform significantly better, and subsequently the largest model was used. In some circumstances U-Net and TransUNet exhibited a significantly better reconstruction performance than the Encoder-Decoder network, but significant differences in accuracy between U-Net and TransUNet was not observed.

FIGS. 33A-33B: Reconstruction errors obtained with different neural network architectures for ‘turbulent’ scroll wave chaos in a bulk with thickness of d_{z}=24. A) Steep increase of reconstruction error with all networks (Encoder-Decoder, U-Net, TransUNet, MIRNet) in opaque excitable media from a single surface (top). B) Low and relatively flat reconstruction errors (below 10%) with all networks in transparent anisotropic excitable media (projection). MIRNet performs slightly better than the other networks. All reconstruction errors stated as root mean squared error (RMSE).

### 1.3.8. Depth Estimation

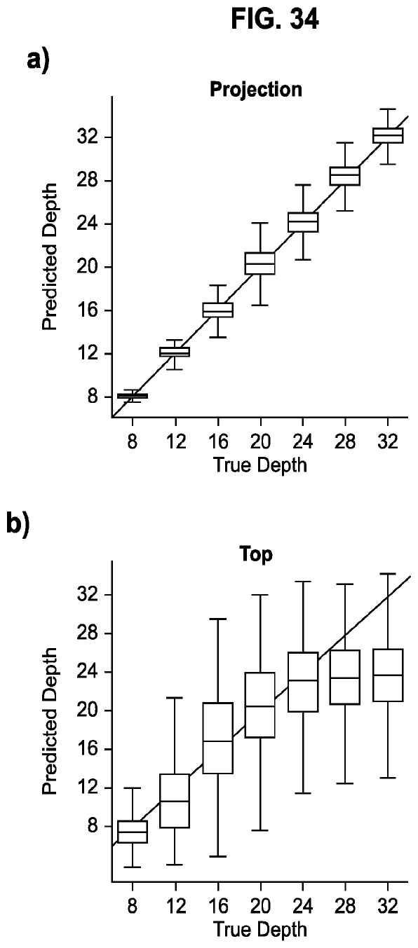

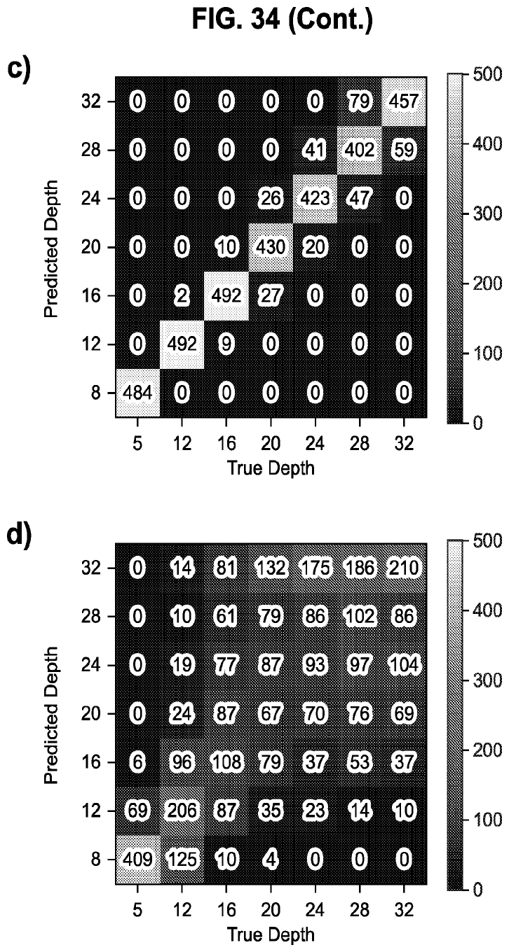

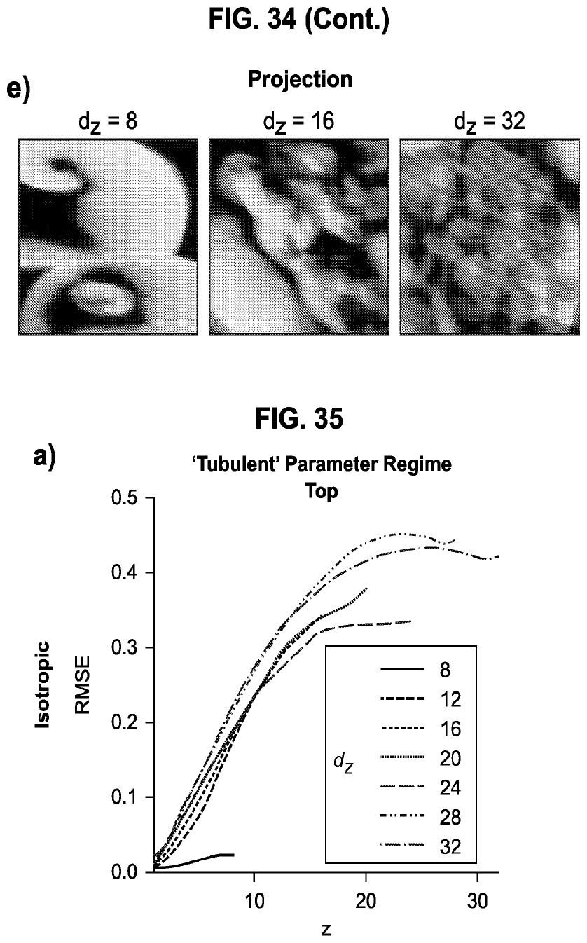

It is possible to estimate the thickness or depth d_{z} of transparent bulks from projections of the corresponding scroll wave dynamics using either a regression or classification neural network. By contrast, it is not possible to reliably predict the thickness of opaque bulks using either approach. FIG. 34A shows predictions obtained with a regression neural network in transparent media, which estimates the depth accurately with floating point precision (with a certain degree of uncertainty). FIG. 34C shows a confusion matrix with depth predictions obtained with a classification neural network also in transparent media, which performs better than the regression. Out of ~500 predictions per thickness, only few attempts falsely classify the thickness (off-diagonal values). For both panels 34A and 34C, predictions were made from two-dimensional observations as shown in FIGS. 25C and 34E. In particular, FIG. 34E shows how the contrast of the waves decreases with increasing bulk thickness as more and more waves are superimposed and the signal is averaged along the z-axis. The neural network presumably associates the bulk's thickness with the contrast.

Both the regression and classification neural networks are unable to predict the depth of opaque media correctly, as shown in FIGS. 34B and 34D. The classification neural network predicts random thicknesses and fails completely at correctly classifying the bulk's actual thickness. For both panels 34B and 34D, predictions were made from two-dimensional observations as shown in FIG. 25A or 25B. The data demonstrates that predicting the extend of scroll wave chaos is challenging with opacity, at least with the methodology used as discussed in this example. While scroll wave dynamics can vary qualitatively with different bulk thicknesses, in particular with thinner bulks, as shown in FIGS. 25-27, there is a critical thickness beyond which the dynamics are dominated by the intrinsic excitable kinetics and are less influenced by the bulk's geometry and its boundaries, thus making depth predictions from surface observation challenging.

FIGS. 34A-34E: Prediction of bulk thickness from surface observations of scroll wave chaos in A,C) transparent bulk medium with projection observations and B,D) opaque bulk medium with top surface observations. The bulk thickness was predicted using either A,B) a regression (black line shows ideal prediction d_{prediction}=dₜᵣᵤₑ) or C,D) a classification neural network, respectively (all in anisotropic media). In transparent media, the thickness can be predicted from observations as shown in FIG. 25C, whereas in opaque media neither the regression nor classification neural networks predict the thickness correctly. E) Exemplary projection images for bulk thicknesses or depths d_{z}=8, d_{z}=16, and d_{z}=32. Due to the averaging, the contrast of the waves decreases with increasing depths.

### 1.4. Analysis and Conclusions

It was demonstrated that deep neural networks can be used to reconstruct three-dimensional scroll wave dynamics from two-dimensional observations of the dynamics on the surface of excitable media. Reconstructions succeed throughout fully opaque excitable media when the scroll wave size is not much smaller than the medium's thickness. In that case, scroll waves can take on complex shapes with their vortex filaments being arbitrarily oriented in space, producing significant dissociation between the surface dynamics on two opposing surfaces of the medium. However, multiple layers of scroll waves are challenging to reconstruct using encoding-decoding convolutional neural networks in opaque media, even when the dynamics are analyzed from two opposing surfaces of the medium. Reconstructions can be performed particularly well in transparent anisotropic excitable media, in which it is possible to reconstruct complicated scroll wave chaos far into the medium.

That encoding-decoding convolutional neural networks can reconstruct three-dimensional dynamics from two-dimensional observations is facilitated by their training on tens of thousands of similar examples. Further generalization can be achieved by diversifying the training dataset, e.g., by adding simulations with a broad range of parameters to the training data or by performing data augmentation. The amount of information that encoding-decoding convolutional neural networks can extract from the short sequence of snapshots to perform the 2D-to-3D prediction task is remarkable. However, it is also revealing that the dual-surface reconstruction does not provide any benefit or synergistic effects over the single-surface reconstruction. It is as if the network performs two separate reconstructions from either side. This highlights fundamental limitations of convolutional encoding-decoding neural networks in this particular application.

One interesting detail that was found is that in transparent excitable media the reconstruction outcomes are very good with anisotropy, but poor with isotropy. Scroll wave chaos cannot be reconstructed at all in transparent isotropic excitable media, and reconstructions of simpler scroll wave dynamics exhibit artifacts. These findings show that anisotropy is crucial because it implicitly encodes depth. The neural network learns to associate the alignment of the waves with the underlying fiber alignment which varies with depth. Moreover, it is able to decode this encoding even when multiple waves are superimposed in the projections (see FIGS. 25C, 28B and 34E). Accordingly, the reconstructions exhibit artifacts or fail entirely in isotropic transparent excitable media as the network lacks depth information. Presumably, it would equally fail in anisotropic excitable media with uniform linearly transverse anisotropy. Unfortunately, this means that this feature cannot be exploited and is neither directly applicable to ventricular fibrillation, because the ventricular muscle is opaque, nor to the Belousov-Zhabotinsky reaction, which is transparent but isotropic.

Nevertheless, the methodology of this example discussed above could still be used to reconstruct intramural action potential waves including scroll waves inside cardiac tissue: 1) Transillumination imaging [9-13], near-infrared optical mapping [44], or other optical techniques [19, 20], which allow imaging of action potential waves deeper inside cardiac tissue, could be used in the transilluminated right ventricle, in the atria or in small animal hearts such as mouse or zebrafish hearts. The muscle fiber architecture in the projections of the transilluminated translucent tissues could enable the depth encoding. 2) Dual-surface imaging with superficial electrode mapping or fluorescent dyes lacking the penetration depth (such as Di-4-ANEPPS) could be used to reconstruct ‘laminar’ episodes of ventricular tachycardia or atrial fibrillation. The wavelengths of single scroll waves or macro-reentries during ventricular arrhythmias are larger than the thickness of the right and left ventricular walls. The atria, which exhibit epi- and endocardial dissociation during atrial fibrillation [45-49], are presumably thin enough for dual-surface reconstructions to succeed. However, the training data would have to account for the complex anatomy of the atria as well as the particular wave dynamics. Whether it will be possible to create ground-truth data or to train a neural network on simulated data and subsequently apply it to experimental data needs to be determined in future research. It was found that the latter approach is in principle feasible. Lastly, the deep learning-based reconstructions can be performed very efficiently within milliseconds on a graphics processing unit, and they do not require the collection of long time-series data.

Similar encoding-decoding convolutional neural networks were used for the prediction of electrical scroll wave chaos from three-dimensional mechanical deformation [32], as well as for the prediction of phase maps and phase singularities from two-dimensional electrical spiral wave chaos [26]. While the networks performed very well in these applications, some of the results presented in this study, particularly the results for scroll wave chaos, are more sobering. This study is another example of the more general notion that cardiac dynamics, and chaotic dynamics more generally, are challenging to predict [27, 51-55]. It is well known that classical deep learning approaches excel at interpolating, but do not perform well at extrapolating, which is what this study aimed to do. Therefore, the complete reconstruction of complicated fine-scaled scroll wave dynamics from surface observations in opaque excitable media will require more sophisticated techniques than encoding-decoding convolutional neural networks.

It was demonstrated that it is possible to reconstruct three-dimensional scroll wave dynamics from two-dimensional observations using deep encoding-decoding convolutional neural networks. Reconstructions succeed under two conditions: either i) the medium is transparent and anisotropic with spatially varying anisotropy or ii) the medium is opaque, and the dynamics are observed on two opposing surface layers while the scroll wavelength is not much shorter than the medium's thickness. In the future, the above discussed methodology of this example could be used to reconstruct transmural action potential wave dynamics from epicardial or endocardial measurements.

### 1.5. References

The numbering related to the following references apply with respect to the experimental results presented in Example 1:
[1] F. Fenton and A. Karma, Vortex dynamics in three-dimensional continuous myocardium with fiber rotation: Filament instability and fibrillation, Chaos: An Interdisciplinary Journal of Nonlinear Science 8, 20 (1998).
[2] R. H. Clayton, Vortex filament dynamics in computational models of ventricular fibrillation in the heart, Chaos: An Interdisciplinary Journal of Nonlinear Science 18, 043127 (2008).
[3] P. Pathmanathan and R. A. Gray, Filament dynamics during simulated ventricular fibrillation in a high-resolution rabbit heart, BioMed Research International 2015, 10.1155/2015/720575 (2015).
[4] J. M. Davidenko, A. V. Pertsov, R. Salomonsz, W. Baxter, and J. Jalife, Stationary and drifting spiral waves of excitation in isolated cardiac muscle, Nature 355, 349 (1992).
[5] A. M. Pertsov, R. Davidenko, J. M. Salomonsz, W. T. Baxter, and J. Jalife, Spiral waves of excitation underlie reentrant activity in isolated cardiac muscle, Circulation Research 72, 631 (1993).
[6] A. Winfree, Electrical turbulence in three-dimensional heart muscle, Science 266, 1003 (1994).
[7] J. Christoph, M. Chebbok, C. Richter, J. Schroder-Schetelig, P. Bittihn, S. Stein, I. Uzelac, F. H. Fenton, G. Hasenfuss, R. J. Gilmour, and S. Luther, Electromechanical vortex filaments during cardiac fibrillation, Nature 555, 667 (2018).
[8] I. Uzelac, S. Iravanian, N. K. Bhatia, and F. H. Fenton, Spiral wave breakup: Optical mapping in an explanted human heart shows the transition from ventricular tachycardia to ventricular fibrillation and self-termination, Heart Rhythm (2022).
[9] W. T. Baxter, S. F. Mironov, A. V. Zaitsev, J. Jalife, and A. M. Pertsov, Visualizing excitation waves inside cardiac muscle using transillumination, Biophysical Journal 80, 516 (2001).
[10] O. Bernus, K. S. Mukund, and A. M. Pertsov, Detection of intramyocardial scroll waves using absorptive trans-illumination imaging, Journal of Biomedical Optics 12, 014035 (2007).
[11] B. G. Mitrea, M. Wellner, and A. M. Pertsov, Monitoring intramyocardial reentry using alternating trans-illumination, in 2009 Annual International Conference of the IEEE Engineering in Medicine and Biology Society (2009) pp. 4194-4197.
[12] V. D. Khait, O. Bernus, S. F. Mironov, and A. M. Pertsov, Method for the three-dimensional localization of intramyocardial excitation centers using optical imaging, Journal of Biomedical Optics 11, 34007 (2006).
[13] B. J. Caldwell, M. L. Trew, and A. M. Pertsov, Cardiac response to low-energy field pacing challenges the standard theory of defibrillation, Circulation: Arrhythmia and Electrophysiology 8, 685 (2015).
[14] A. Molavi Tabrizi, A. Mesgarnejad, M. Bazzi, S. Luther, J. Christoph, and A. Karma, Spatiotemporal organization of electromechanical phase singularities during high-frequency cardiac arrhythmias, Phys. Rev. X 12, 021052 (2022).
[15] B. J. Welsh, J. Gomatam, and A. E. Burgess, Three-dimensional chemical waves in the Belousov-Zhabotinskii reaction, Nature 304, 611 (1983).
[16] T. Bansagi and O. Steinbock, Three-dimensional spiral waves in an excitable reaction system: Initiation and dynamics of scroll rings and scroll ring pairs, Chaos: An Interdisciplinary Journal of Nonlinear Science 18, 026102 (2008).
[17] P. Dahmlow, S. Alonso, M. Bar, and M. J. B. Hauser, Twists of opposite handedness on a scroll wave, Phys. Rev. Lett. 110, 234102 (2013).
[18] C. Bruns and M. J. B. Hauser, Dynamics of scroll waves in a cylinder jacket geometry, Phys. Rev. E 96, 012203 (2017).
[19] E. M. C. Hillman, O. Bernus, E. Pease, M. B. Bouchard, and A. Pertsov, Depth-resolved optical imaging of transmural electrical propagation in perfused heart, Opt. Express 15, 17827 (2007).
[20] L. Sacconi, L. Silvestri, E. C. Rodriguez, G. A. Armstrong, F. S. Pavone, A. Shrier, and G. Bub, KHz-rate volumetric voltage imaging of the whole zebrafish heart, Biophysical Reports 2, 100046 (2022).
[21] S. Berg, S. Luther, and U. Parlitz, Synchronization based system identification of an extended excitable system, Chaos: An Interdisciplinary Journal of Nonlinear Science 21, 10.1063/1.3613921 (2011).
[22] J. Lebert and J. Christoph, Synchronization-based reconstruction of electromechanical wave dynamics in elastic excitable media, Chaos: An Interdisciplinary Journal of Nonlinear Science 29, 10.1063/1.5101041 (2019).
[23] M. J. Hoffman, N. S. LaVigne, S. T. Scorse, F. H. Fenton, and E. M. Cherry, Reconstructing three-dimensional reentrant cardiac electrical wave dynamics using data assimilation, Chaos: An Interdisciplinary Journal of Non-linear Science 26, 013107 (2016).
[24] M. J. Hoffman and E. M. Cherry, Sensitivity of a data-assimilation system for reconstructing three-dimensional cardiac electrical dynamics, Philosophical Transactions of the Royal Society A: Mathematical, Physical and Engineering Sciences 378, 20190388 (2020).
[25] B. R. Hunt, E. J. Kostelich, and I. Szunyogh, Efficient data assimilation for spatiotemporal chaos: A local ensemble transform kalman filter, Physica D: Nonlinear Phenomena 230, 112 (2007), data Assimilation.
[26] J. Lebert, N. Ravi, F. H. Fenton, and J. Christoph, Rotor localization and phase mapping of cardiac excitation waves using deep neural networks, Frontiers in Physiology 12, 10.3389/fphys.2021.782176 (2021).
[27] S. Herzog, R. S. Zimmermann, J. Abele, S. Luther, and U. Parlitz, Reconstructing complex cardiac excitation waves from incomplete data using echo state networks and convolutional autoencoders, Frontiers in Applied Mathematics and Statistics 6, 10.3389/fams.2020.616584 (2021).
[28] C. H. Martin, A. Oved, R. A. Chowdhury, E. Ullmann, N. S. Peters, A. A. Bharath, and M. Varela, EP-PINNs: Cardiac electrophysiology characterisation using physics-informed neural networks, Frontiers in Cardiovascular Medicine 8, 10.3389/fcvm.2021.768419 (2022).
[29] R. R. Aliev and A. V. Panfilov, A simple two-variable model of cardiac excitation, Chaos, Solitons & Fractals 7, 293 (1996).
[30] D. Bourguignon and M. Cani, Controlling anisotropy in mass-spring systems, Computer Animation and Simulation, Springer, 113 (2000).
[31] M. Nash and A. Panfilov, Electromechanical model of excitable tissue to study reentrant cardiac arrhythmias, Progress in Biophysics and Molecular Biology 85, 501 (2004).
[32] J. Christoph and J. Lebert, Inverse mechano-electrical reconstruction of cardiac excitation wave patterns from mechanical deformation using deep learning, Chaos: An Interdisciplinary Journal of Nonlinear Science 30, 123134 (2020).
[33] O. Ronneberger, P. Fischer, and T. Brox, U-net: Convolutional networks for biomedical image segmentation, in Lecture Notes in Computer Science (Springer International Publishing, 2015) pp. 234-241.
[34] J. Chen, Y. Lu, Q. Yu, X. Luo, E. Adeli, Y. Wang, L. Lu, A. L. Yuille, and Y. Zhou, Transunet: Transformers make strong encoders for medical image segmentation, CoRR abs/2102.04306 (2021), 2102.04306.
[35] S. W. Zamir, A. Arora, S. Khan, M. Hayat, F. S. Khan, M.-H. Yang, and L. Shao, Learning Enriched Features for Real Image Restoration and Enhancement, in Computer Vision-ECCV 2020, Lecture Notes in Computer Science, edited by A. Vedaldi, H. Bischof, T. Brox, and J.-M. Frahm (Springer International Publishing, 2020) pp. 492-511.
[36] V. Nair and G. E. Hinton, Rectified linear units improve restricted boltzmann machines (Omnipress, Madison, WI, USA, 2010) p. 807-814.
[37] S. loffe and C. Szegedy, Batch normalization: Accelerating deep network training by reducing internal covariate shift, in Proceedings of the 32nd International Conference on Machine Learning, Proceedings of Machine Learning Research, Vol. 37, edited by F. Bach and D. Blei (PMLR, Lille, France, 2015) pp. 448-456.
[38] A. Vaswani, N. Shazeer, N. Parmar, J. Uszkoreit, L. Jones, A. N. Gomez, L. Kaiser, and I. Polosukhin, Attention is all you need, in Advances in Neural Information Processing Systems 30: Annual Conference on Neural Information Processing Systems 2017 Dec. 4-9, 2017, Long Beach, CA, USA, edited by I. Guyon, U. von Luxburg, S. Bengio, H. M. Wallach, R. Fergus, S. V. N. Vishwanathan, and R. Garnett (2017) pp. 5998-6008.
[39] A. Bruhn, J. Weickert, and C. Schnorr, Lucas/Kanade meets Horn/Schunck: Combining local and global optic flow methods, Int. J. Comput. Vis. 61, 211 (2005).
[40] J. T. Barron, A General and Adaptive Robust Loss Function, in 2019 IEEE/CVF Conference on Computer Vision and Pattern Recognition (CVPR) (2019) pp. 4326-4334.
[41] D. P. Kingma and J. Ba, Adam: A method for stochastic optimization, in 3rd International Conference on Learning Representations, ICLR 2015, San Diego, CA, USA, May 7-9, 2015, Conference Track Proceedings, edited by Y. Bengio and Y. LeCun (2015).
[42] M. Abadi, A. Agarwal, P. Barham, E. Brevdo, Z. Chen, C. Citro, G. S. Corrado, A. Davis, J. Dean, M. Devin, S. Ghemawat, I. Goodfellow, A. Harp, G. Irving, M. Isard, Y. Jia, R. Jozefowicz, L. Kaiser, M. Kudlur, J. Levenberg, D. Man′e, R. Monga, S. Moore, D. Murray, C. Olah, M. Schuster, J. Shlens, B. Steiner, I. Sutskever, K. Talwar, P. Tucker, V. Vanhoucke, V. Vasudevan, F. Viegas, O. Vinyals, P. Warden, M. Wattenberg, M. Wicke, Y. Yu, and X. Zheng, TensorFlow: Largescale machine learning on heterogeneous systems, https://www.tensorflow.org (2015).
[43] F. Chollet et al., Keras, https://keras.io (2015).
[44] B. J. Hansen, N. Li, K. M. Helfrich, S. H. Abudulwahed, E. J. Artiga, M. E. Joseph, P. J. Mohler, J. D. Hummel, and V. V. Fedorov, First in vivo use of high-resolution near-infrared optical mapping to assess atrial activation during sinus rhythm and atrial fibrillation in a large animal model, Circulation: Arrhythmia and Electrophysiology 11, e006870 (2018).
[45] R. B. Schuessler, T. Kawamoto, D. E. Hand, M. Mitsuno, B. I. Bromberg, J. L. Cox, and J. P. Boineau, Simultaneous epicardial and endocardial activation sequence mapping in the isolated canine right atrium., Circulation 88, 250 (1993).
[46] J. Eckstein, B. Maesen, D. Linz, S. Zeemering, A. van Hunnik, S. Verheule, M. Allessie, and U. Schotten, Time course and mechanisms of endo-epicardial electrical dissociation during atrial fibrillation in the goat, Cardiovascular Research 89, 816 (2010).
[47] J. Eckstein, S. Zeemering, D. Linz, B. Maesen, S. Verheule, A. van Hunnik, H. Crijns, M. A. Allessie, and U. Schotten, Transmural conduction is the predominant mechanism of breakthrough during atrial fibrillation, Circulation: Arrhythmia and Electrophysiology 6, 334 (2013).
[48] N. de Groot, L. van der Does, A. Yaksh, E. Lanters, C. Teuwen, P. Knops, P. van de Woestijne, J. Bekkers, C. Kik, A. Bogers, and M. Allessie, Direct proof of endo-epicardial asynchrony of the atrial wall during atrial fibrillation in humans, Circulation: Arrhythmia and Electrophysiology 9, e003648 (2016).
[49] T. E. Walters, G. Lee, A. Lee, R. Sievers, J. M. Kalman, and E. P. Gerstenfeld, Site-specific epicardium-to-endocardium dissociation of electrical activation in a swine model of atrial fibrillation, JACC: Clinical Electrophysiology 6, 830 (2020).
[50] J. Zhao, B. J. Hansen, T. A. Csepe, P. Lim, Y. Wang, M. Williams, P. J. Mohler, P. M. Janssen, R. Weiss, J. D. Hummel, and V. V. Fedorov, Integration of high-resolution optical mapping and 3-dimensional microcomputed tomographic imaging to resolve the structural basis of atrial conduction in the human heart, Circulation: Arrhythmia and Electrophysiology 8, 1514 (2015).
[51] J. Pathak, B. Hunt, M. Girvan, Z. Lu, and E. Ott, Model-free prediction of large spatiotemporally chaotic systems from data: A reservoir computing approach, Phys. Rev. Lett. 120, 024102 (2018).
[52] J. Pathak, A. Wikner, R. Fussell, S. Chandra, B. R. Hunt, M. Girvan, and E. Ott, Hybrid forecasting of chaotic processes: Using machine learning in conjunction with a knowledge-based model, Chaos: An Interdisciplinary Journal of Nonlinear Science 28, 041101 (2018).
[53] S. Herzog, F. Worgotter, and U. Parlitz, Data-driven modeling and prediction of complex spatio-temporal dynamics in excitable media, Frontiers in Applied Mathematics and Statistics 4, 10.3389/fams.2018.00060 (2018).
[54] S. Shahi, C. D. Marcotte, C. J. Herndon, F. H. Fenton, Y. Shiferaw, and E. M. Cherry, Long-time prediction of arrhythmic cardiac action potentials using recurrent neural networks and reservoir computing, Frontiers in Physiology 12, 10.3389/fphys.2021.734178 (2021).
[55] S. Shahi, F. H. Fenton, and E. M. Cherry, A machine learning approach for long-term prediction of experimental cardiac action potential time series using an autoencoder and echo state networks, Chaos: An Interdisciplinary Journal of Nonlinear Science 32, 063117 (2022).

### Example 2: Dreaming of Electrical Waves: Generative Modeling of Cardiac Excitation Waves using Diffusion Models

### 2.0. Overview

Electrical waves in the heart form rotating spiral or scroll waves during life-threatening arrhythmias such as atrial or ventricular fibrillation. The wave dynamics are typically modeled using coupled partial differential equations, which describe reaction-diffusion dynamics in excitable media. More recently, data-driven generative modeling has emerged as an alternative to generate spatio-temporal patterns in physical and biological systems. Here, denoising diffusion probabilistic models were explore for the generative modeling of electrical wave patterns in cardiac tissue. Diffusion models were trained with simulated electrical wave patterns to be able to generate such wave patterns in unconditional and conditional generation tasks. For instance, inpainting tasks, such as reconstructing three-dimensional wave dynamics from superficial two-dimensional measurements, and evolving and generating parameter-specific dynamics were explored. The diffusion-generated solutions were characterized and compared to solutions obtained with biophysical models and it was found that diffusion models learn to replicate spiral and scroll waves dynamics so well that they could serve as an alternative data-driven approach for the modeling of excitation waves in cardiac tissue. For instance, it was found that it is possible to initiate ventricular fibrillation (VF) dynamics instantaneously without having to apply pacing protocols in order to induce wavebreak. The VF dynamics can be created in arbitrary ventricular geometries and can be evolved over time. However, it was also found that diffusion models ‘hallucinate’ wave patterns when given insufficient constraints. Regardless of these limitations, diffusion models are an interesting and powerful tool with many potential applications in cardiac arrhythmia research and diagnostics.

### 2.1. Introduction

Waves in excitable media exhibit complex spatiotemporal dynamics [1, 2]. In two-dimensional media, they form linear, focal, or rotating spiral-shaped waves or compositions thereof. In three-dimensional media, they manifest as planar or spherical focal waves, or take on more complicated rotational shapes referred to as scroll waves. Spiral and scroll wave dynamics have been studied for many decades, as they are associated with heart rhythm disorders, such as atrial fibrillation, polymorphic ventricular tachycardia, or ventricular fibrillation [2-12]. In the heart, electrical excitation initiates the contraction of the heart muscle, and it is hypothesized that the abnormal rapid and irregular contractions during tachyarrhythmias are caused by spiral- and scroll-shaped waves of electrical excitation.

The electrical waves can be reproduced and studied in computer simulations using biophysical models [13-15]. These models consist of coupled partial differential equations (PDEs), which describe the electrical excitability u and refractoriness r of cardiac muscle cells and the coupling between them (see eqs. (1-2) of this example). The equations model reaction-diffusion dynamics, where the exchange of currents through ion channels between cells are modeled as a diffusive process and the cells as nonlinear oscillators. Integrating these equations in time and over space in a spatially extended system using, for instance, the finite difference or finite element method produces nonlinear waves of electrical excitation mediated via diffusion.

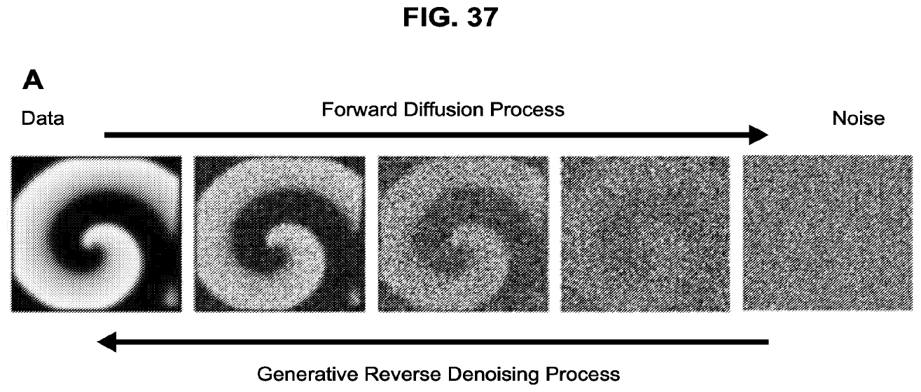

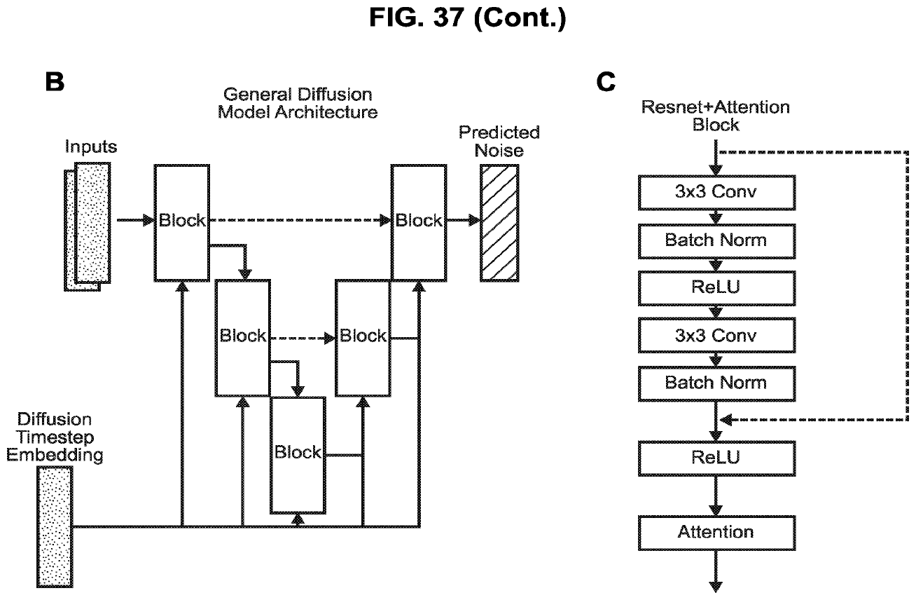

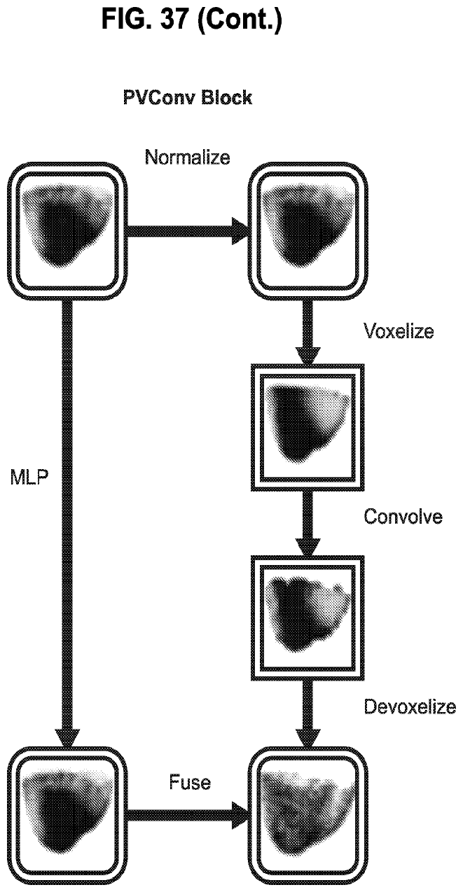

Diffusion, on the other hand, is a term that has recently emerged in the field of artificial intelligence (AI), referring to a class of generative neural networks which employ a diffusive process to generate data [16-18]. During the training procedure, noise is iteratively added to the training data and the neural networks, termed denoising diffusion probabilistic models (DDPMs) or diffusion models, learn to reverse this process, ultimately enabling them to create data from noise, see FIG. 37. Diffusion models are very successful in generating data such as images [19-21], videos [22], and audio [23], and they are increasingly also used for technical applications in physics, engineering, medicine, and biology [24-27]. Diffusion models likely also have many useful applications in cardiology that have yet to be explored. For example, they could be used in electrophysiological studies to generate synthetic action potential wave patterns and arrhythmia morphologies, either to fill in or reconstruct missing measurement data, or to simulate cardiac dynamics in a data-driven fashion. Diffusion-generated solutions could be particularly useful in situations in which measurements can only be obtained partially or indirectly, or when biophysical model equations or parameters are lacking.

Here, diffusion models were explored for their application in cardiac electrophysiology and arrhythmia research. In this numerical study, whether diffusion models can be used to reconstruct or simulate electrical impulse phenomena, such as spiral and scroll waves, in computer simulations of excitable media was investigated. Electrical spiral and scroll waves were simulated in two- and three-dimensional square-, bulk- and heart-shaped tissues with isotropic and anisotropic diffusive spread of the excitation, and trained different diffusion models to i) inpaint spiral wave dynamics (see section 2.3.2), ii) reconstruct three-dimensional scroll wave dynamics from two-dimensional observations (see section 2.3.1), iii) generate parameter-specific wave dynamics (see section 2.3.7), and iv) predict the evolution of wave dynamics over time in analogy to integrating the dynamics (see section 2.3.5). It was determined how reliable diffusion models are when generating such spatio-temporal physiological dynamics. Generative neural networks, such as diffusion models, generative adversarial networks (GANs), or large language models (LLMs) are known to be capable of producing a continuum of output including false or undesired output, which is often referred to as ‘hallucination’. It was shown that diffusion models can generate electrical waves in many different ways: out of the blue in an unconstrained generative process or when the generative process is guided or constrained by parameters or boundary conditions such as partial data, or a recent dynamical state of the system. In particular, the latter generative mode corresponds to diffusion-based data-driven modeling of cardiac dynamics. It was found that hallucination occurs when the generation task is insufficiently constrained, which raises concerns over the reliability of diffusion models in diagnostic applications.

FIGS. 37A-37D: Diffusion-based generative modeling of electrical wave dynamics in cardiac tissue. A) Forward diffusion process and generative reverse denoising process. The training data consists of spiral and scroll wave dynamics in excitable media. B) General diffusion model architecture for processing image data with underlying U-Net architecture. C) ResNet Attention block. D) Diffusion model for generating scroll waves in heart-shaped geometries represented as pointclouds with corresponding scalar-valued data (Point-Voxel Diffusion [28]).

### 2.2. Methods 2.2.1. Simulations of Electrical Wave Dynamics in Heart Muscle Tissue

Nonlinear waves of electrical excitation were simulated in i) two-dimensional rectangular-shaped, ii) three-dimensional bulk-shaped, and iii) three-dimensional heart-shaped geometries, respectively. In all three cases, the phenomenological Aliev-Panfilov model was used to simulate nonlinear waves of electrical excitation:

$$\frac{\partial u}{\partial t} = ∇ \cdot ( D ∇ u ) - ku ( u - a ) ( u - 1 ) - ur \qquad ( 1 )$$

$$\frac{\partial r}{\partial t} = ( {ϵ}_{0} + \frac{{µ}_{1} r}{u + {µ}_{2}} ) ( ku ( a + 1 - u ) - r ) \qquad ( 2 )$$

The dynamic variables u and r represent the local electrical excitation and refractoriness in dimensionless, normalized units, respectively. The parameters k, a, ϵ₀, μ₁ and μ₂ determine the properties of the waves (e.g. excitability, wavelength, conduction speed, number of waves, etc.). The parameters k and co were varied to change the properties of the excitation waves and produce different training data for different tasks (Task 1-6), (see Table 3 and sections 2.2.1-2.2.6). The simulations in the simplified (rectangular, bulk) and heart-shaped geometries were performed as described in [29] and [31], respectively. Correspondingly, the system of equations (1-2) was integrated using the forward Euler method and the smoothed particle hydrodynamics method [32, 33], respectively. The two-dimensional simulations were isotropic, whereas the three-dimensional simulations were anisotropic with a locally varying fiber direction and faster wave propagation along the fiber direction. The fiber architectures were created as described in [29, 31]. In particular, the bi-ventricular heart geometries and underlying rule-based fiber architectures were randomly initialized as described in [31].

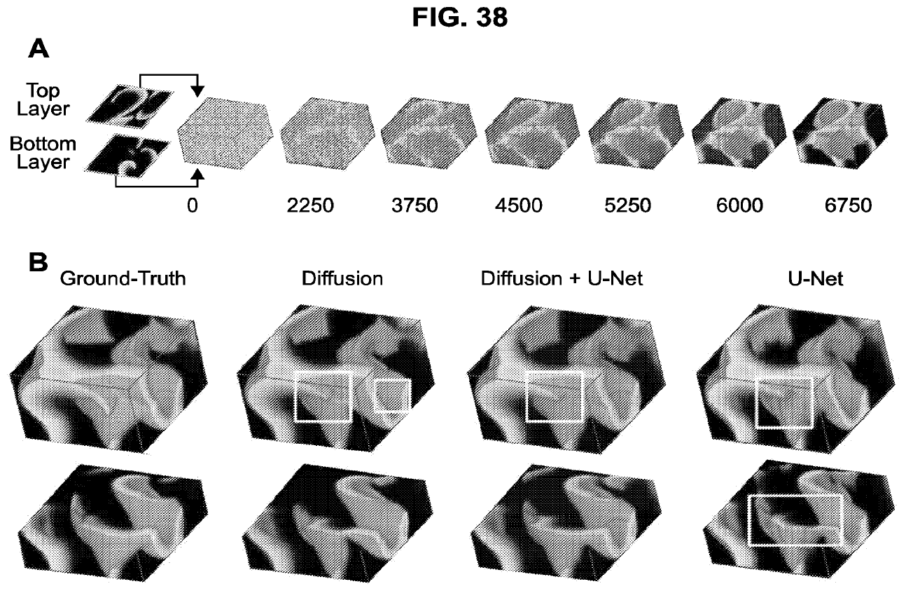

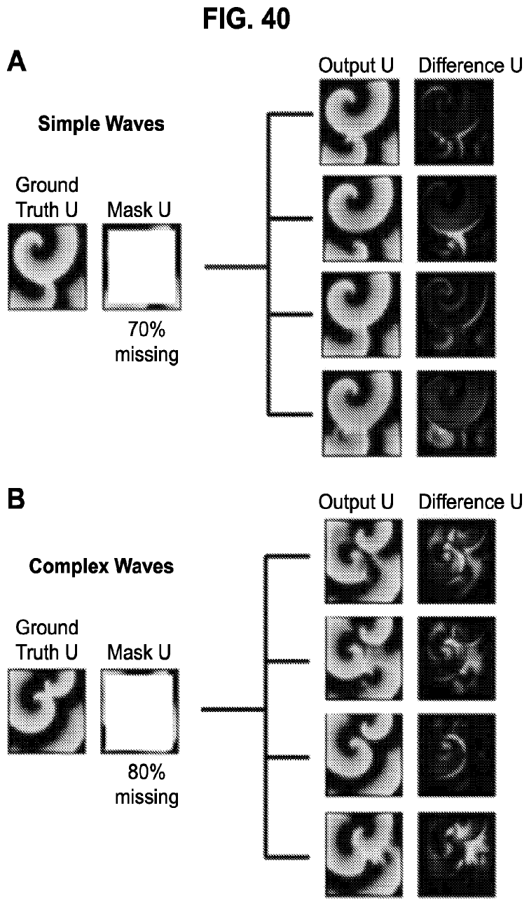

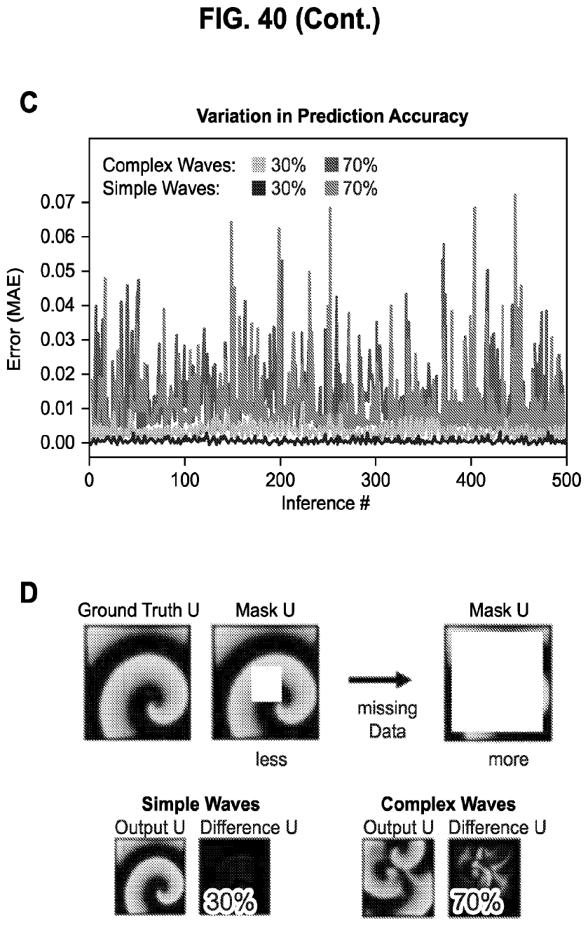

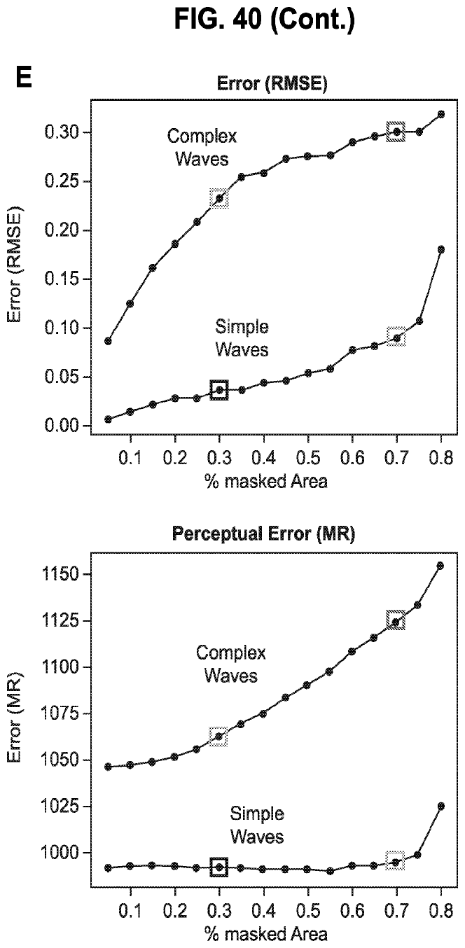

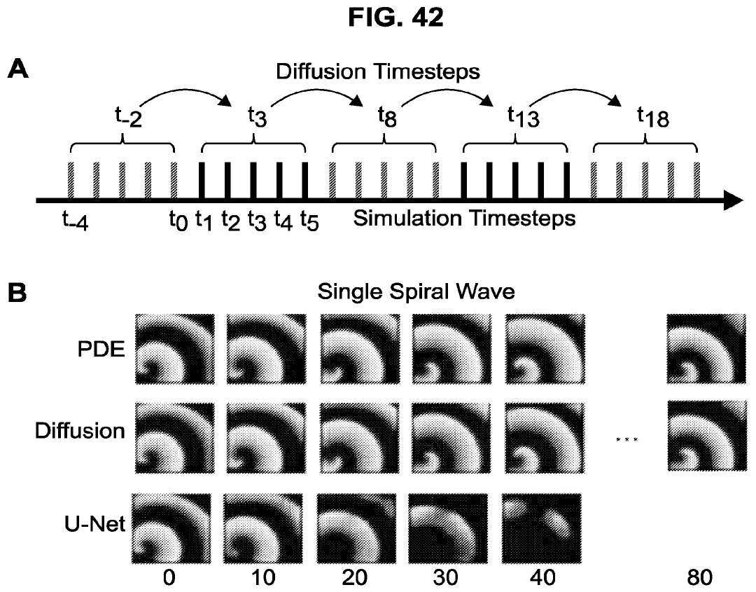

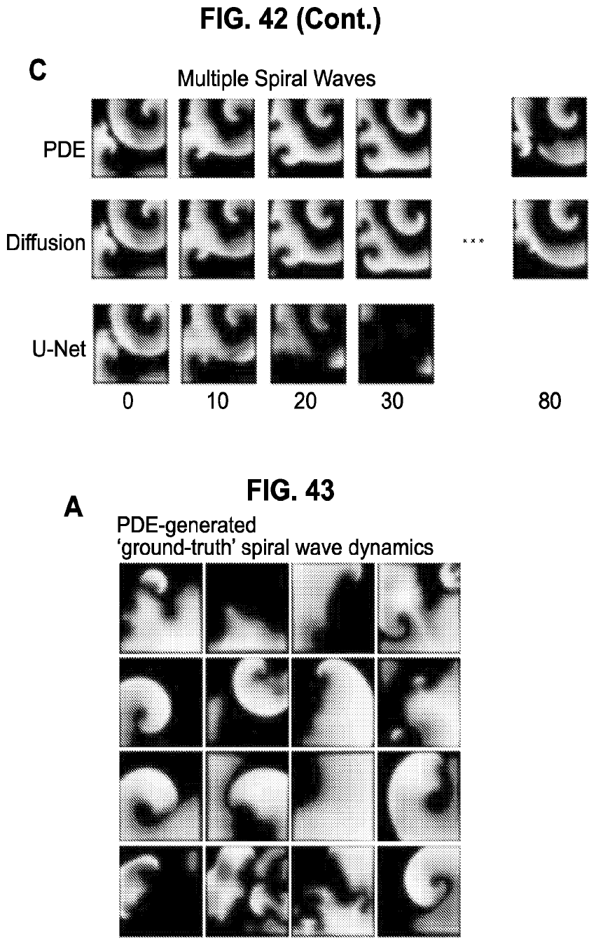

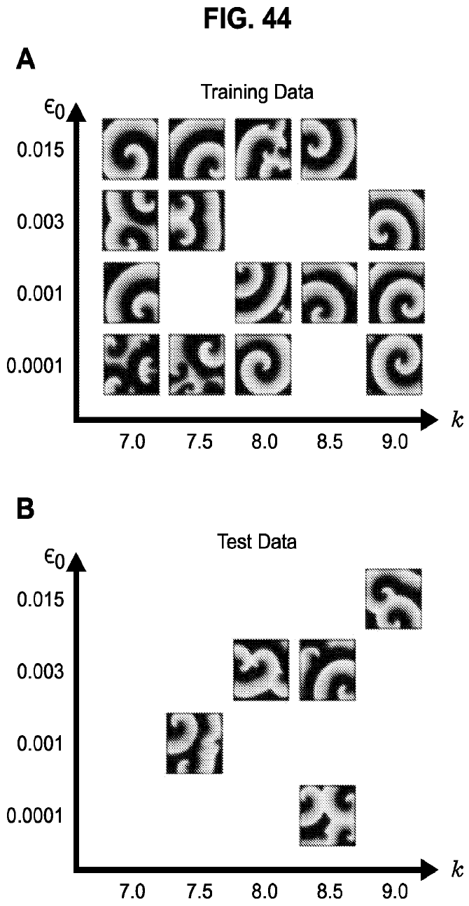

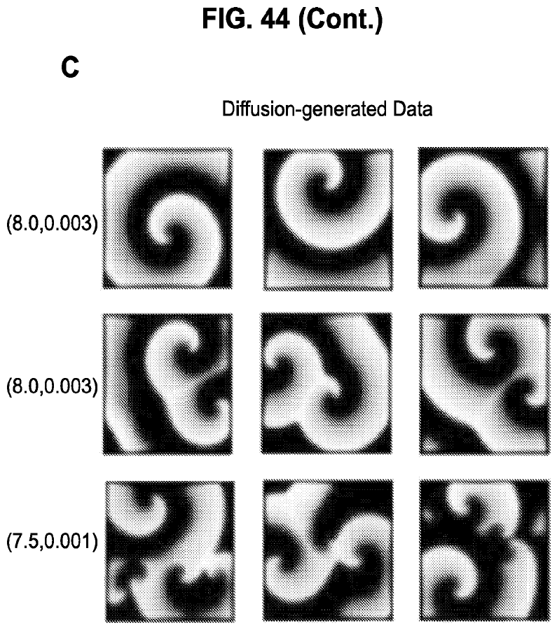

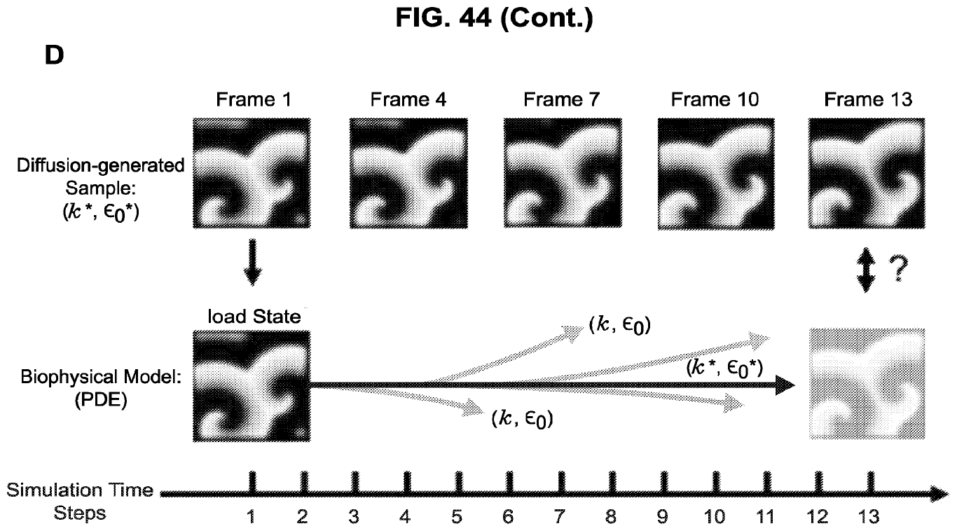

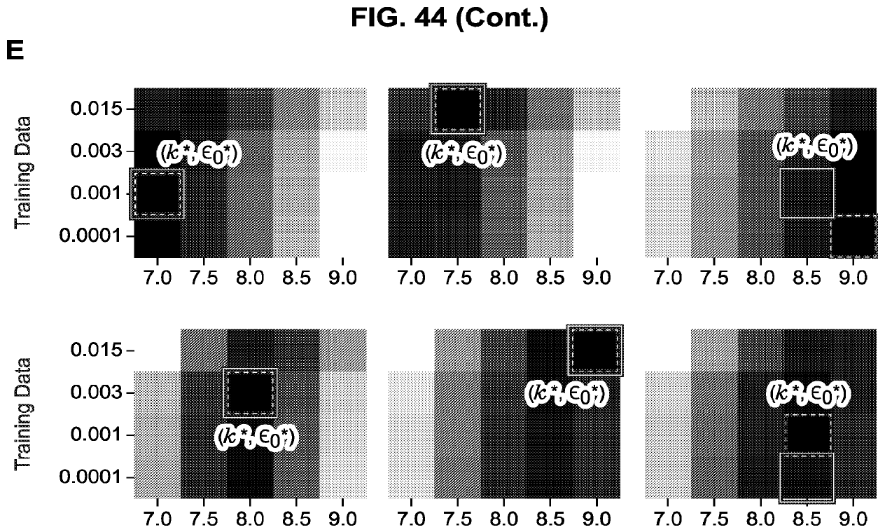

The model parameters were chosen specifically for each task (see Table 3). Scroll wave dynamics were simulated in a bulk with 128×128×40 voxels as shown in FIG. 38 using a fixed set of parameters (Task 1). Two different regimes of spiral wave dynamics were simulated, as shown in FIGS. 40 and 42, using two different parameter sets: one with few (Task 2a, 5a) and one with more spiral waves (Tasks 2b, 5b). A range of parameter-specific spiral wave dynamics were simulated (Task 6), as seen in FIGS. 44A and 44B, by varying the parameters k and ε₀. For each task, hundreds of simulations were performed to generate sufficient training data. For example, for Task 1, 125 simulations were performed, where 100 simulations were used for training and 25 for evaluation, as described in [29]. The initial conditions u₀, r₀ were randomized and therefore different in each simulation, see also [31]. If the spiral or scroll wave dynamics self-terminated prematurely, the simulation was restarted.

Using the simulation data, different training datasets were generated for each task, see Tasks 1-6 in section 2.2.2 for details. Each training dataset consisted of thousands of samples randomly chosen from the different training simulations. Correspondingly, each evaluation dataset consisted of thousands of samples randomly chosen from the evaluation simulations. Training and evaluation datasets were completely separate datasets.

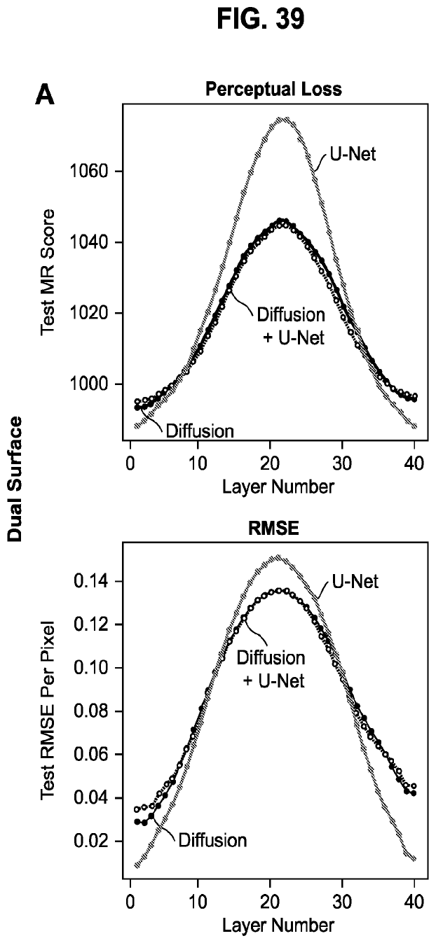

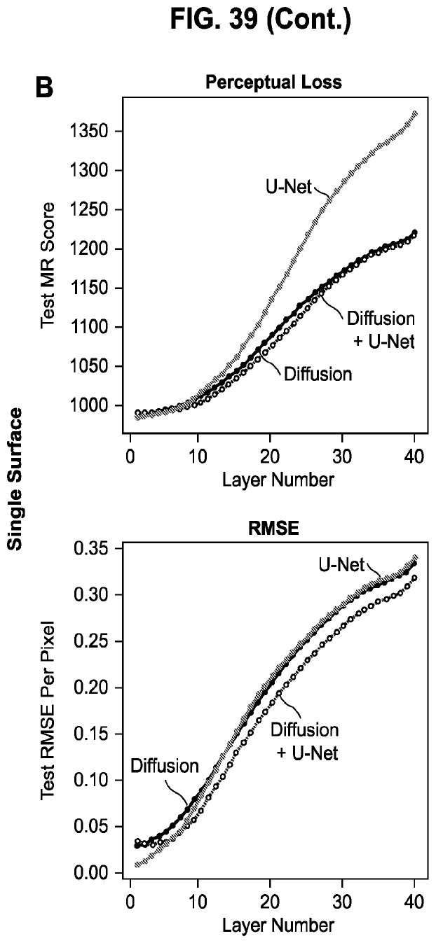

FIGS. 38A-38B: Diffusion-based reconstruction of scroll wave dynamics inside a three-dimensional bulk from two-dimensional observations of the dynamics on the bulk's top and bottom surface. The bulk is fully opaque, and measurements can only be obtained from its surface. A) Illustration of diffusion process over denoising iterations. B) Scroll wave dynamics (left: ground-truth) and reconstructed scroll waves with diffusion (center left), U-Net (right) and U-Net refined with diffusion (center right). While diffusion produces smoother wave patterns than U-Net, particularly at deeper layers, the overall reconstruction accuracies are not significantly different across the three approaches (see also FIG. 39). White squares highlight slight differences between reconstructions and ground-truth. The bulk is 128×128×40 voxels (aspect ratio was altered to emphasize transmural wave morphology), see also [29]. The simulation parameters are shown in Table 3 (Task 1).

### 2.2.2. Denoising Diffusion Model

A denoising diffusion probabilistic modeling neural network architecture was used, which is referred to as diffusion model in this study for simplicity. Diffusion models consist of a forward diffusion process and a reverse diffusion process (see FIG. 37). During the forward diffusion process, gaussian noise is added incrementally to an input image until it is indistinguishable from random noise. This produces a sequence of samples (x₀, . . . , x_{T}) with increasing noise, starting from the data point x₀ from the real data distribution q(x) and ending with what is indistinguishable from an isotropic Gaussian distribution.

$$q ( {x}_{t} \vert {x}_{t - 1} ) = N ( {x}_{t} ; \sqrt{1 - {β}_{t}} {x}_{t - 1} , {β}_{t} I ) \qquad ( 3 )$$

The step sizes are controlled by a variance schedule βₜ.

$$q ( {x}_{1 : T} \vert {x}_{0} ) = {\prod }_{t = 1}^{T} q ( {x}_{t} \vert {x}_{t - 1} ) \qquad ( 4 )$$

When sampling new data from the data distribution q(x), a model p_{θ} is learned to estimate q(xₜ₋₁|xₜ), which is also approximated by a gaussian distribution. This is referred to as the reverse diffusion process.

$${p}_{θ} ( {x}_{0 : T} ) = p ( {x}_{T} ) {\prod }_{t = 1}^{T} {p}_{θ} ( {x}_{t} \vert {x}_{t - 1} ) \qquad ( 5 )$$

$${p}_{θ} ( {x}_{t} \vert {x}_{t - 1} ) = N ( {x}_{t - 1} ; {µ}_{θ} ( {x}_{t} , t ) , {Σ}_{θ} ( {x}_{t} , t ) ) ) \qquad ( 6 )$$

This allows the model p_{θ} to only have to estimate the two parameters u and o of the estimated denoising step. Commonly, σ_{θ} is fixed to a constant variance schedule and is not learnable. This means that in order to estimate pe, a model needs to learn μ_{θ}(xₜ, t). Electrical wave dynamics can be treated as image-like data and the U-Net architecture from Dhariwal and Nichol [34] is used to estimate the noise at each step of the reverse diffusion process. The model is trained using pairs taken from the forward diffusion process xₜ, xₜ₋₁ and taking the mean squared error (MSE) between the noise estimated by the model and the true noise at that step. Different versions of diffusion models for different tasks were implemented, as described in the following sections. The conditioned diffusion models for sections 2.2.2, 2.2.5, and 2.2.6 were implemented following Saharia et al. [20] using an implementation by L. Jiang and Y. Belousov [35]. The unconditioned diffusion model for section 2.2.3 was implemented following Ho et al. [18] using the Diffusers library [36]. The diffusion model for section 2.2.4 was implemented following Zhou et al. [28] using the official codebase. All diffusion models include a U-Net [37] architecture and were implemented in PyTorch [38].

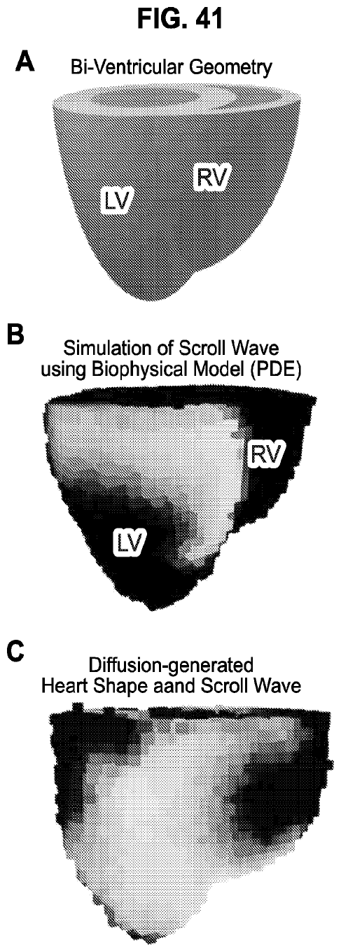

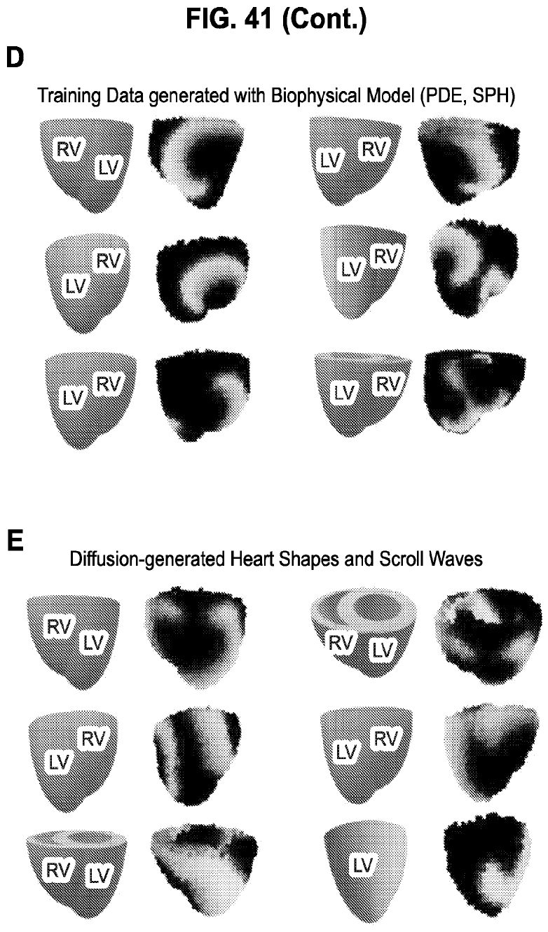

TABLE 3
Parameters of biophysical model [15] used to simulate
electrical wave patterns.
Task 1: Scroll wave dynamics in anisotropic 3D bulk shown in
FIG. 38. Tasks 2, 3, 5: Spiral wave dynamics in 2D isotropic
medium (see FIGS. 40, 42, 43). Task 4: Scroll wave dynamics
in (bi-ventricular) heart-shaped medium (see FIG. 41). Task
6: Parameter-specific generation of spiral waves (see
FIG. 44). Dₐ is an anisotropic diffusion
tensor (see also [29, 31]).

| Param. | Task 1 | Task 2a/5a | Task 2b/5b | Task 3 | Task 4 | Task 6 |
|---|---|---|---|---|---|---|
| D | Dₐ | 1 | 1 | 1 | Dₐ | 1 |
| k | 8 | 8.5 | 7.5 | 8 | 8 | k′ |
| a | 0.05 | 0.1 | 0.1 | 0.05 | 0.2 | 0.1 |
| ε₀ | 0.002 | 0.003 | 0.001 | 0.002 | 0.002 | ε′₀ |
| μ₁ | 0.8 | 0.16 | 0.16 | 0.2 | 0.2 | 0.16 |
| μ₂ | 0.3 | 0.3 | 0.3 | 0.3 | 0.3 | 0.3 |

### Task 1: Reconstruction of 3D Scroll Wave Dynamics

A diffusion model was trained to predict three-dimensional scroll wave dynamics inside a bulk from two-dimensional observations of the dynamics on the surface of the bulk (Task 1), as described in and shown in FIG. 38. The model was trained to predict a single three-dimensional snapshot of the excitatory variable uₜ (x, y, z) at a given time t at every voxel in a bulk with 128×128×40 voxels from 5 subsequent two-dimensional snapshots of the dynamics on the bulk's surface:

$$( {u}_{1} ( x , y ) , … , {u}_{5} ( x , y ) ) \to {\tilde{u}}_{5} ( x , y , z ) \qquad ( 7 )$$

where ũ is a prediction of the true dynamics u (ground-truth). The snapshots were measured either i) on the top surface only (single-surface mode) resulting in a spatio-temporal measurement consisting of 5 snapshots (u₁ (x, y, 1), . . . , u₅(x, y, 1)) or ii) on the top and bottom surface (dual-surface mode) resulting in 2. 5 snapshots (u₁ (x, y, 1), u₁ (x, y, 40), u₂(x, y, 1), . . . , u₅(x, y, 40)), as described in [29]. The 5 snapshots were sampled at equidistant times t₁, t₂, t₃, t₄, t₅=t_{-4T}, t_{-3T}, t_{-2T}, t_{-T}, t with uᵢ=u(tᵢ) over about one rotational period T of the scroll wave dynamics (T=t_{-T}≈T/5), which were found to provide sufficient information to reconstruct the dynamics, as described in, e.g., [29]. Accordingly, the diffusion model was conditioned by concatenating these sequences as additional channels in the U-Net inputs (interleaved in dual-surface mode, odd indices for the top layer and even indices for the bottom layer). To explore an alternative extension of the reconstruction approach, the diffusion model was conditioned using the output of a generic U-Net model, which was trained and applied as described in [29], to create a combined model that potentially can take advantage of the strengths of both the U-Net and diffusion models (see also FIG. 39). Accordingly, the combined model was conditioned with the sequences of 5 (or 2·5) two-dimensional snapshots and 1 three-dimensional prediction u (x, y, z) of the U-Net model, which analyzed in turn also 5 snapshots as input. The two- and three-dimensional inputs were concatenated to obtain (128×128×45) or (128×128×50) samples in single-vs. dual-surface mode as conditions, respectively. This leads to a total of 4 conditioning modes that were tested (single-vs. dual-surface, diffusion vs. combination of diffusion+U-Net). Generally, the different model versions required three-dimensional input samples, e.g. (128×128×5) or (128×128×45). To denoise a 128×128×40 volume image with 5·128×128 snapshots as conditioning, the model corresponds to an R(128×128×45)→R (128×128×40) function. However, internally, because the denoising diffusion process works on the intermediate noisy bulk data, the overall data consists of the conditioning data concatenated to the noisy data which then results in an array size of, for example, 128×128×80. Training was performed with 20,000 training samples, which were generated in 100 simulations (see also section 2.2.1), and the model was evaluated on 5,000 separate samples. The same simulation data and parameters were used as in [29] (see also Table 3).

### Task 2: Inpainting of 2D Spiral Wave Dynamics

A diffusion model was trained to inpaint missing data of two-dimensional spiral wave dynamics (Task 2), as shown in FIG. 40. Data from a square region at the center of the 128×128-pixel simulation domain was masked or left out and the network was trained with corresponding pairs of masked uₘ(x, y) and ground-truth data u(x, y) to reconstruct the missing parts of the spiral wave pattern:

$$( {u}_{m , 1} ( x , y ) , … , {u}_{m , 5} ( x , y ) ) \to {\tilde{u}}_{5} ( x , y ) \qquad ( 8 )$$

where ũ is a prediction of the ground-truth dynamics u and the model reads a short spatio-temporal sequence of 5 two-dimensional snapshots as above. Masked pixels were replaced by zeros. How the reconstruction accuracy of the network changes with different mask sizes m ∈[0.05, . . . , 0.8] (percentage masked area vs. total area from 5% to 80% in increments of 5%) and with less and more complex wave patterns (see section 2.3.2) was tested. Two spiral wave regimes were simulated, and two separate models were trained: i) one with largely only one spiral wave (Task 2a) and ii) one with multiple, more chaotic spiral waves (Task 2b), see Table 3 for the corresponding simulation parameters. Both models were trained equally with a range of masks with uniform distribution (of the percentage of masked area vs. total area). Training and evaluation was performed with 27,500 and 6,000 samples, respectively.

### Task 3: Unconditional Generation of Spiral Wave Patterns

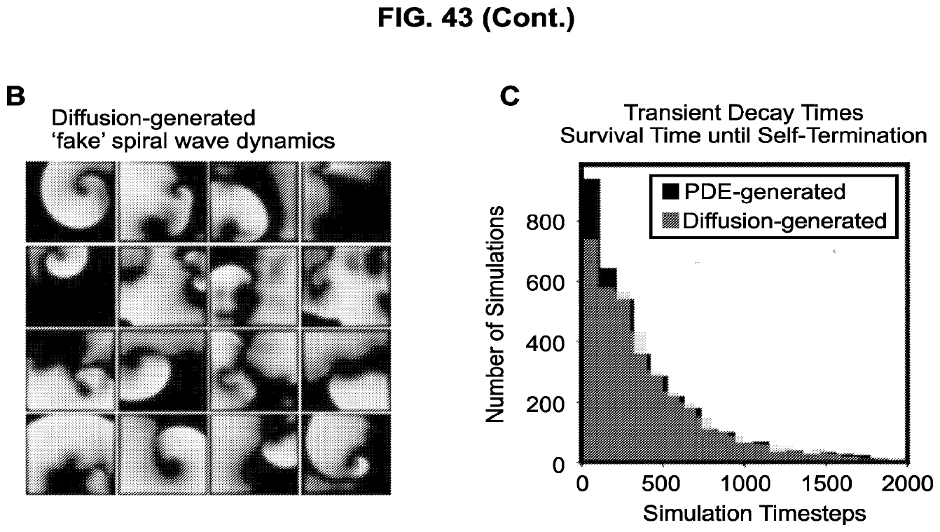

a diffusion model was trained to generate two-dimensional spiral wave patterns in an unconditional fashion (Task 3), as also shown in FIG. 43. This means that the model can dream up any spiral wave pattern it can come up with (depending on what data it was trained on) starting from random noise ξ:

$$ξ ( x , y ) \to ( \tilde{u} , \tilde{r} ) ( x , y ) \qquad ( 9 )$$

The model is completely unrestricted in that it is not trained to perform certain tasks, such as inpainting, nor conditioned by certain boundary conditions, such as top or bottom layers, or parameters which guide the generative process, as in Task 6 (below). The model was trained with 50,000 training samples generated from spiral wave patterns simulated in an isotropic excitable medium with a fixed parameter set from (see also Table 3). Each training sample consisted of a single spiral wave pattern defined by its dynamical variables (u, r)ₜ (x, y) in a 128×128-pixel simulation domain. The training samples were sampled from the last 300 timesteps of 1,000 simulations which ran for a fixed simulation time and were randomly initiated following a random pulse protocol (similarly as used in [42]). After training, the diffusion model outputs a pair of two-dimensional (ũ, \tilde{r})ₜ(x, y)-fields or a snapshot of the system's dynamical state. The output together with the parameters and boundary conditions (no-flux) defines a complete current dynamical state of the spiral wave dynamics. The diffusion model outputs were then used as initial states for the biophysical model defined in eqs. (1-2) and 5,000 simulations were initiated with these diffusion-generated initial conditions with identical model parameters as during training data generation and measured the time until each simulation self-terminated over the ensemble of simulations (see section 2.3.6).

### Task 4: Generation of Scroll Waves in Heart-Shaped Geometries

A diffusion model was trained to generate scroll waves in bi-ventricular-shaped geometries (Task 4), similarly as described in the above Task 3 and as shown in FIG. 40. Both the shape and the wave pattern were generated by the diffusion model simultaneously. This means that the diffusion model outputs an arbitrary bi-ventricular geometry as well as a corresponding anisotropic scroll wave pattern, the anisotropy reflecting the underlying fiber architecture. The generation was unconditional as described in the previous Task 3. The simulated training data (see Task 1) consists of pointclouds of (~32,000) vertices p(\vec{x})ᵢ located within bi-ventricular heart shapes and scalars representing the excitatory variable uᵢ per vertex with index i (see FIG. 41). The data was down-sampled to 16,000 points and Point-Voxel Diffusion was used trained on 5,000 training samples obtained from the simulations. The model was trained to output 16,384 points (with a latent dimension of 512).

Task 5: Generation of Spiral Wave Dynamics and Integration of their Spatio-Temporal Evolution

A diffusion model was trained to calculate an immediate future time step of a given spatio-temporal excitation wave pattern (Task 5):

$$( u , r ) ( \vec{x} , t ) \to ( \tilde{u} , \tilde{r} ) ( \vec{x} , t + τ ) \qquad ( 10 )$$

where (u, r) are the dynamic variables from eqs. (1-2) and Tis an infinitesimal temporal increment or the integration time step. In other words, a diffusion model was trained to be able to evolve cardiac excitation wave dynamics. More precisely, the model was trained to predict the next 5 timesteps from the previous 5 timesteps of the dynamics, resulting in a temporal integration scheme that updates a brief spatio-temporal pattern instead of a static spatial pattern. This approach was found to be more stable than auto-regressively integrating the dynamics, e.g., updating the dynamics one time step at a time and predicting the next time step from the previous 5 timesteps. With this spatio-temporal integration eq. (10) corresponds to:

$$( u , r ) ( \vec{x} , {t}_{- 4} , … , {t}_{0} ) \to ( \tilde{u} , \tilde{r} ) ( \vec{x} , {t}_{1} , … , {t}_{5} ) \qquad ( 11 )$$

where t₋₄, . . . , to are the 4 previous and the current time steps and t₁, . . . , t₅ are the next 5 future timesteps (see FIG. 42A). The model was trained and evaluated with 15,000 and 5,000 samples, respectively, showing two-dimensional spiral wave dynamics with the simple and complex parameter sets from Task 2 (see FIGS. 42B and 42C).

### Task 6: Parameter-Specific Generation of Spiral Waves

A diffusion model was trained to generate parameter-specific spiral wave dynamics (Task 6), as shown in FIG. 44:

$$( ξ ( x , y ) , k , {ϵ}_{0} ) \to ( \tilde{u} , \tilde{r} ) ( x , y , {t}_{1} , {t}_{4} , {t}_{7} , {t}_{10} , {t}_{13} ) \qquad ( 12 )$$

where k and co are parameters of the biophysical model in eqs. (1-2) of this example and (ũ, \tilde{r}) is a short sequence of 5 predictions of the dynamical state of the spiral wave patterns at future times t₁, t₄, t₇, t₁₀, t₁₃ given in simulation time steps (see detailed explanation below). The diffusion model generates spiral wave patterns from the initial noise ξ(x, y) and this generation process was refer as ‘conditioned by the parameters k and ϵ₀′. The model generates a sequence of 5 subsequent spiral wave patterns for validation purposes (see section 2.3.7).

A training dataset was generated in which the parameters k and co were varied systematically and spiral wave dynamics were initiated with these parameters for 20 different parameter pairs (k, ϵ₀) (see FIGS. 44A and 44B). 200 simulations were run over the 20 parameter pairs on the grid k=[7, 7.5, 8, 8.5, 9] and ϵ0=[0.0001, 0.001, 0.003, 0.015] with 10 simulations per parameter combination, and data was selected from 15 of the 20 parameter sets to train the model and the remaining 5 for testing, as shown in FIGS. 44A and 44B. The diffusion model was then conditioned by concatenating the parameters to the initial noisy distribution § (x, y), adding two channels to the input of the underlying U-Net (2×128×128 pixels). Aside from the parameter conditioning, the generation was unconditioned allowing the diffusion model to dream up any spiral wave pattern, as described in Task 3 above.

To test the specificity of the parameter-specific generations, the training and prediction process were set up as follows: for each simulation, 150 frames were extracted and every 15 frames were grouped into one training sample {(u, r)₁, . . . , (u, r)₁₅}. Of these 15 frames, only 5 frames were used per training sample, subsampling the data in time by a factor of 3: {(u, r)₁, (u, r)₄, (u, r)₇, (u, r)₁₀, (u, r)₁₃}. Using this data as the target during training, the diffusion model was trained to predict a short spatio-temporal sequence of spiral wave dynamics consisting of 5 frames with an offset of 3 frames (see eq. (12) of this example). Whether the diffusion model can generate different parameter-specific dynamics was then evaluated by loading the first predicted dynamical state (ũ, ñ)₁ created with a specific parameter pair (k*, ϵ₀*) and evolving it using the biophysical model (integrating the PDEs) for 15 simulation timesteps using either the same parameter pair (k*, ϵ₀*) or a different one (k, ϵ₀). This process was repeated with all 20 parameter pair combinations and the 5-th diffusion-generated frame (\tilde{e}, \tilde{r})₁₃ (x, y) was compared to the output of the simulation at time step 13 by calculating the error (RMSE) between the two frames (see FIG. 44D).

### 2.2.3. Training Details

The networks were trained using the Adam optimizer with a learning rate of 10⁻⁴ for the bulk prediction tasks and 10⁻³ for all other tasks. A batch size of 8 was used for the bulk prediction tasks and a batch size of 32 was used for all the other tasks. All neural network models were implemented in PyTorch [38]. Training and reconstructions were performed on a NVIDIA RTX 24000 graphics processing unit (GPU).

TABLE 4
Different model sizes (trainable parameters) and training
times used in this study. Training was performed
on a single NVIDIA RTX 24000 GPU.

| Model | Trainable Parameters | Training Time |
|---|---|---|
| Task 1 | 965,266,792 | 9 days |
| Task 1 | 62,640,193 | 1.5 days |
| Task 1 | 113,673,219 | 1.5 days |
| Task 1 | 31,092,676 | 1 days |
| Task 1 | 62,644,805 | 1.5 days |
| Task 1 | 250,430,474 | 4 days |
| Classification | 11,689,512 | 5 min |

### 2.2.4. Evaluation

The diffusion model's accuracies were evaluated using the root mean squared error (RMSE), the mean absolute error (MAE), or the multi-resolution perceptual error (MR) [41] depending on the model and task. The errors were computed per frame, averaging over all frames of a separate evaluation dataset that was not part of the training dataset. While RMSE and MAE correspond to a measure of the average difference per pixel, the perceptual error is a measure for the similarity of two patterns as it calculates the difference on the embedding in the network [41] (see also FIG. 40 and section 2.3.2).

### 2.3. Results

2.3.1. Reconstruction of Three-Dimensional Scroll Wave Dynamics from Surface Observations

Measuring electrophysiological wave phenomena beneath the heart surface is a challenge in cardiovascular research and diagnostics. Catheter electrodes or optical mapping provide only data from the surface of the heart and intramural measurements from within the heart muscle using, for instance, plunge needle electrodes provide only sparse information. To address this challenge, various numerical methods were introduced which aim at reconstructing transmural wave patterns from observations of the dynamics on the tissue's surface [29, 30, 44, 45]. The numerical reconstructions are particularly relevant in the context of tachyarrhythmias, such as ventricular or atrial fibrillation, as they may provide a better understanding of the underlying three-dimensional spatio-temporal organization of the electrical waves within the heart muscle. Recently, Lebert et al. [29] and Stenger at al. [30] demonstrated that convolutional encoding-decoding neural networks (different U-Net-types) can be used to reconstruct three-dimensional scroll wave dynamics inside a thick bulk-shaped excitable medium from two-dimensional observations of the dynamics on the top and/or bottom surfaces (representing the epi- and endocardium). At the same time, Stenger et al. demonstrated this briefly also with a diffusion model [30]. However, several aspects of the deep learning-based reconstructions remain underexplored, in particular with the diffusion-based approach. The deep learning-based reconstructions assume scroll waves inside the tissue as the neural networks are trained with thousands of corresponding pairs of three- and two-dimensional data of scroll waves and observations thereof. Therefore, the training data implicitly restricts the approach to a particular distribution of data and its characteristics (specific electrophysiological model that produces waves with a particular shape, isotropic vs. anisotropic wave patterns, wavelength relative to medium thickness), and the approach is task-specific (single-vs. dual-surface observations).

Here, a diffusion model was compared to a U-Net model for predicting three-dimensional scroll waves inside an anisotropic bulk-shaped excitable medium (see FIGS. 38-39). Both models predict a three-dimensional scroll wave pattern from a short sequence (N=5) of two-dimensional observations on either only the top (single-surface mode) or ii) both the top and bottom surface layers (dual surface mode) of the bulk, as in [29] (see also FIG. 39). The bulk is completely opaque and thick enough to sustain three-dimensional scroll wave dynamics (128×128×40 voxels, 1-2 wavelengths) (see FIG. 38. FIG. 38A shows the generative denoising process of the diffusion model. Interestingly, the rough shape of the scroll wave pattern is already captured early in the denoising process, while later stages enhance finer structures. It was found that both diffusion and U-Net produce sufficiently accurate reconstructions, the diffusion model slightly outperforming U-Net (see FIG. 39). In it was found that diffusion performs substantially better than U-Net with long observations of the surface (32 snapshots). Here, fewer observations (only 5 snapshots) were used which likely causes this discrepancy. Another methodological difference to is that the diffusion model of this example (above) predicts the bulk at once and not layer by layer. FIG. 38B shows a comparison of the ground-truth scroll wave pattern with predictions obtained with i) diffusion, ii) U-Net, and iii) diffusion refining the U-Net output (see Task 1 for details). While U-Net reconstructions become fuzzier with increasing depth, diffusion maintains the shape and smoothness or overall look of the scroll waves throughout the bulk. This is also reflected by the perceptual error, see also section 2.3.2. Unlike in [30], the model of this example (above) produces scroll wave patterns without residual noise. However, even though the visual impression suggests otherwise, on average no dramatic improvement of the reconstruction accuracy (RMSE) was find with diffusion over U-Net (see FIG. 39). Upon closer inspection, one notices that diffusion produces minor mismatches at deeper layers (white boxes in FIG. 38B), suggesting that its output looks better but is not necessarily more accurate than with U-Net (see also section 2.3.2). Whether guiding the diffusion model with the output from the U-Net model could mitigate these issues was tested. However, refining the U-Net output with diffusion increases the reconstruction accuracy only marginally (see FIG. 39).

FIGS. 39A-39B: Transmural reconstruction error per layer number or depth with diffusion (blue), U-Net (green) or diffusion+U-Net (orange), see also FIG. 38. The reconstruction error was quantified using either the perceptual error (left) or absolute difference (RMSE, right). A) Dual-surface reconstruction analyzing top and bottom layers: U-Net performs worse at midwall, but slightly better closer to the surfaces than diffusion. Perceptual error and RMSE produce very different error profiles. B) Single-surface prediction: the perceptual error increases more steeply with U-Net than with diffusion, while all models perform similar with RMSE.

### 2.3.2. Hallucination During Inpainting of 2D Spiral Wave Dynamics

Generative models are known to hallucinate, which means that they may generate output that looks, sounds, or reads convincing but is inaccurate. For example, recent large language models (LLMs) are known to confidently present made-up knowledge as if it was factual. In computer vision, diffusion models may generate unexpected scenes which are abstract and not part of human day-to-day experience.

While it is easy to identify hallucination in diffusion-generated visual scenes, it is not necessarily obvious with spiral or scroll wave patterns when they include hallucinations, see also section 2.3.4. In FIG. 38, the diffusion-generated reconstructed scroll wave patterns at midwall look convincing and can be misinterpreted as accurate solutions, but they are just as inaccurate as the output from the U-Net model.

This hallucinating behavior was explored further in a two-dimensional inpainting task of spiral wave dynamics, see FIG. 40, and can confirm that hallucination occurs, particularly when the task is insufficiently constrained. The size of a square region at the center of the medium was varied, within which the diffusion model was tasked to interpolate the missing spiral wave pattern from the surrounding data (see section Task 2 above for details). Hallucination is minimal with a small square, which is reflected by the error (RMSE) on the left sides of the graphs in FIG. 40E. However, it was observed that the diffusion model comes up with many different spiral wave patterns for the same task when the square region is large (see FIG. 40A-40B). The graph in FIG. 40C shows the variation of the diffusion model output when the same task is repeated over and over (500 times with simple vs. complex waves and 30% vs. 70% missing data respectively). The variation in the output as quantified by the error (MAE) between the ground-truth and the individual predicted spiral wave pattern is particularly large with 70% missing data. Hallucination becomes also stronger when the wave dynamics are more complicated (two parameter sets were tested, see Table 3) (compare panels 40A and 40B and the upper and lower curves in 40E) (average error calculated over 500 unique samples per data point). In FIG. 40E, the root mean squared error (RMSE), which reflects the pixel-wise congruency of the ground-truth and the predicted pattern, was compared with a perceptual error (MR), which reflects similarities or differences in the patterns independently from spatial mismatches (as it is calculated on the embedding of the pattern). The perceptual error indicates that with simple waves the variations in the output of the diffusion model are small regardless of the size of the masked area. In other words, differences in the waves only correspond to slight spatial mismatches (which cause high RMSE), while the wave shapes are very similar qualitatively. The spikes in FIG. 40C (with 70%), on the other hand, correspond to large qualitative changes of the wave pattern which occur occasionally with both complex and simple waves. The data suggests that the diffusion model hallucinates if it has the freedom to generate many possible fitting solutions to a problem, that hallucination can be associated with qualitative changes in the topology of the wave pattern, and that hallucination can be mitigated by sufficiently constraining the task that the diffusion model is supposed to perform.

FIG. 40A-40E: Hallucination of diffusion model during inpainting of electrical spiral wave dynamics. A) The diffusion model predicts missing data inside a square region (white) at the center of the medium. With most of the data missing (70%), the diffusion model dreams up wave patterns which look convincingly like spiral wave patterns but deviate substantially from the ground-truth: 4 repetitive predictions for the same interpolation task. B) The effect becomes more severe with more complex waves and fewer data (80% missing) (see also panel D). C) The model generates significantly different output when the same prediction task is performed repeatedly (500 times, black: simple waves with 30%, gray: simple waves with 30%, orange: complex waves with 70%, pink: complex waves with 70% missing data, respectively). D) Combinations of less vs. more missing data (30% vs. 70%) and simple and complex waves. E)

Average prediction error (RMSE vs. perceptual MR) with increasing percentage of missing data. Hallucination is minimal with simpler wave patterns and 10⁻³⁰% missing data and increases steeply with complex wave patterns. The perceptual error indicates a higher agreement between inpainted and ground-truth patterns which is not reflected by RMSE (see also FIG. 39).

### 2.3.3. Generation of Reentrant Scroll Waves in Heart-Shaped Geometries

FIGS. 41A-41D show three-dimensional scroll waves in biventricular heart shapes that were generated using the diffusion model detailed in Task 4 above. The generative process was completely unconstrained, and the model generates any scroll wave pattern it can come up with. Exemplary solutions of the biophysical model and the diffusion model, which are shown in panels 41C and 41D respectively, are visually indistinguishable from each other (see also FIG. 43 and section 2.3.4). The diffusion model generates randomly shaped bi-ventricular heart geometries and corresponding electrical scroll wave patterns simultaneously. The scroll wave patterns are anisotropic because the training data was generated in an anisotropic bi-ventricular fiber architecture which implicitly defines anisotropy during the generations. The data was generated in a pointcloud-based fashion, see Task 4 above, and was voxelized for visualization purposes. The biventricular template geometry in panel 41B was deformed randomly during the training process generating a variety of shapes and the diffusion model comes up with similar shapes during the generative process (see also [31]). Even though the diffusion model generates only a single scroll wave pattern u(\vec{x}, t), it is easy to imagine how this pattern could also be evolved over time u (x, t₁, t₂, . . . ) as described in section 2.3.5 and shown in FIG. 42.

FIGS. 41A-41E: Diffusion-based modeling of reentrant electrical waves in heart-shaped bi-ventricular geometries. A) Bi-ventricular template geometry, which was deformed to create 1,000 unique geometries for the training data generation, as described in [29]. B) Example of three-dimensional electrical scroll wave in bi-ventricular heart model simulated using a biophysical model (Aliev-Panfilov), see eqs. (1)-(2) of this example, integrated using SPH-method [32, 33], see section 2.2.1. The data was voxelized and volume rendered. C) Diffusion-generated electrical scroll wave in bi-ventricular heart shape with muscle fiber anisotropy. Both the geometry and the wave pattern were generated simultaneously. D) The training data consists of 5,000 training samples showing scroll wave patterns. Each simulation consists of 32,000 particles, the data was subsampled to 16,000 particles for training. E) Further examples of diffusion-generated electrical scroll waves in bi-ventricular heart shapes. The diffusion model generates a bi-ventricular shape (each shape different but only template geometry shown) as well as a full state with both dynamic variables (u, r) (x). The scroll wave patterns are anisotropic following the underlying ventricular muscle fiber organization.

FIGS. 42A-42C: Data-driven modeling of spiral wave dynamics using diffusion models. A) Spatio-temporal prediction (Task 5) of 5 frames from previous 5 frames (see Task 5 above). B,C) Comparison of ground-truth data (GT) simulated with biophysical model (finite differences) and data-driven methods (Diffusion vs. U-Net) to evolve the wave pattern. With a single spiral wave, the output of the diffusion model is visually indistinguishable from the ground truth for many rotations, while U-Net quickly fails to sustain the wave pattern. With more complicated wave patterns, the diffusion models begins to deviate from the biophysical model after 2-3 spiral rotations (80 timesteps).

2.3.4. Visual Similarity of Diffusion-vs. PDE-generated Spiral Waves

Diffusion-generated ‘fake’ spiral waves are visually indistinguishable from real spiral wave patterns simulated with the biophysical model and partial differential equations (PDEs) (see FIGS. 43A and 43B). Each panel shows 4×4 randomly chosen, representative examples of spiral wave patterns simulated with PDEs or diffusion, respectively. The diffusion-generated spiral wave patterns in 43B were generated using the diffusion model described in section 2.2.2, which was trained on spiral wave patterns obtained with a fixed parameter set, shown in 43A. It was found to be impossible to distinguish the two patterns visually (this was tested systematically with

different persons). Despite the visual similarity, a ResNet18 classifier fine-tuned on the two classes of images in panels 43A and 43B) is able to distinguish the two groups of spiral wave patterns with an accuracy of 99.7% (separate training and validation/test datasets). This may be due to invisible artifacts from the denoising process or the capability of CNNs to learn minute differences between classes. This is well explored, and was in part the motivation behind the joint generator-discriminator training process of GANs. Each specific diffusion-generated spiral wave image was generated from a random distribution of noise & (x, y), which was the only information that guided the generation process.

FIGS. 43A-43C: Comparison of spiral wave dynamics generated in computer simulations using a biophysical model (ground-truth) and ‘fake’ diffusion-generated spiral wave dynamics. A,B) The two datasets are visually indistinguishable from each other. C) Distribution of self-termination times measured for an ensemble of 5,000 randomly initiated spiral wave dynamics and 5,000 diffusion-generated spiral wave dynamics. Most spiral wave dynamics self-terminated after 1,000 simulation time steps, regardless of whether they were simulated in a conventional fashion using partial differential equations or initiated by diffusion.

### 2.3.5. Generative Diffusion-based Simulation of Electrical Wave Dynamics

Electrical impulse propagation in the heart is usually simulated by integrating or evolving a biophysical model consisting of partial differential equations in space and over time, e.g., by using the finite difference or finite element methods to solve a set of partial differential equations. Here, it was shown that, alternatively, diffusion-based data-driven modeling can be employed to simulate excitation waves in cardiac tissue. The simulated spiral wave data (from Task 2 and FIG. 40) was used to train a diffusion model in such a way that it can calculate the next dynamical state given a current state of wave dynamics χₜ: (u, r) (x, y, t) enabling it to evolve the dynamics χₜ→χₜ+1 (see Task 5 above). FIGS. 42B and 42C show the ground-truth (GT) spiral wave patterns for up to 80 simulation timesteps and the corresponding evolved spiral wave patterns predicted either with the diffusion model or a correspondingly trained U-Net model. While the dynamics quickly degenerate with U-Net, the diffusion model successfully sustains and evolves the dynamics over a very long time. The ability to sustain the wave pattern is likely related to diffusion models being able to learn and mimic shapes. The diffusion model produces spiral waves with either stable or meandering cores which exhibit breakup and (self-) interactions. With the single spiral wave in FIG. 42B, the diffusion model's output matches the biophysical model's output for many rotations. Eventually, the original (ground-truth) dynamics diverge from the diffusion generated dynamics. However, this divergence is to some extent to be expected as the dynamics would also diverge with, for instance, two different integration methods (forward Euler vs. third-order methods) or integration time steps even with the same biophysical model. With the more complex spiral wave dynamics in FIG. 42C, the diffusion output diverges rapidly within less than 2 rotations of the spiral wave pattern. Interestingly, the diffusion model appears to produce more stable wave dynamics (less wavebreak), which could be associated with a bias in the training data towards stable waves. It was found that the diffusion-based integration works best when updating multiple time steps at a time (e.g., t₋₄, . . . , 0→t₁, . . . , 5) (see also FIG. 42A), instead of updating the dynamics one time step at a time t₁→t₂ as, for instance, with the forward Euler integration method. The multi time step integration also works better than auto-regressively predicting the next frame while including previous predictions. Updating the dynamics in a 128×128-pixel simulation domain takes 1.1 ms on a NVIDIA 24000 GPU. Together with the findings in 2.3.3, the data suggests that the generative diffusion-based integration of spatio-temporal dynamics could also be used to evolve scroll wave dynamics in three-dimensional heart shaped geometries.

### 2.3.6. Transient Behavior of Diffusion-generated Spiral Waves

Spiral wave dynamics are known to eventually self-terminate over time with an exponential decay rate [42]. FIG. 43C shows that diffusion-generated spiral wave patterns also self-terminate following the same law with approximately the same decay rate. The decay rates of the original simulated spiral wave dynamics (black) and the diffusion-generated spiral wave dynamics (gray), which were initiated using the (u, r) (x, y) outputs from the diffusion model and evolved with the biophysical model, are close to identical, see section 2.2.2 (Task 3) for details. The two distributions of survival times calculated from the two ensembles of dynamics indicate that most spiral wave dynamics self-terminate before 500 simulation time steps and that only few survive longer than 1,500 simulation time steps. The slight discrepancy between the two distributions towards short survival times may result from the way the measurement was performed: the diffusion model was trained with data sampled from a particular sampling time step including the previous 300 timesteps, while the PDE-generated distribution was calculated from an ensemble of simulations measured only at exactly that particular sampling time step. The data suggests that the diffusion-generated spiral wave patterns stem from the same part of the chaotic attractor of the dynamics. Training samples were created from that part of the attractor, the diffusion model was trained with this particular distribution of samples and the diffusion model generates new spiral wave patterns and reproduces the same distribution of spiral wave patterns, which then, when used as initial conditions and evolved further using the biophysical model, collectively exhibit the same self-termination rates. The diffusion model appears to capture the underlying parameter-specific characteristics of the dynamics.

### 2.3.7. Parameter-Specific Generation of Spiral Wave Dynamics

The diffusion model described in section 2.2.2 (Task 6) can generate parameter-specific spiral wave dynamics when given a set of parameters as input (see FIG. 44). This means that the generative process can be guided by parameters to produce spiral wave dynamics with specific properties which would similarly arise with a biophysical model with the same parameters. Spiral wave dynamics can be very different depending on the chosen model parameters. For instance, spiral wave cores can drift or be stationary depending on the parameters [47]. Here, the Aliev-Panfilov model produces regimes with fewer or more spiral waves when varying the parameters k and ε₀ in eqs. (1)-(2) of this example (see FIGS. 44A and 44B). The diffusion model reproduces these different parameter-specific regimes when conditioned with the respective parameters during the generation process (see FIGS. 44C and 44E). Importantly, the diffusion model can generate parameter-specific spiral wave dynamics even if it was not trained with the specific parameter combination. The simulation data in parameter space was split into training and test data, as shown in FIGS. 44A and 44B, and some of the generations in 44C were obtained with parameter combinations not included during the training process. It was verified that these diffusion-based generations correspond to parameter-specific dynamics as follows: 1) the diffusion model was let to generate a short sequence of 5 frames {(ũ, ñ)₁, (ũ, \tilde{r})₄, (ũ, \tilde{r})₇, (ũ, \tilde{r})₁₀, (ũ, ñ)₁₃} (x, y) with a parameter combination (k*, ϵ₀*) as conditioning (see FIG. 44D). The frames correspond to every third frame in a sequence of 15 frames, as described in section 2.2.2 (Task 6). 2) Then the first predicted frame (ũ, \tilde{r})₁ (x, y) was used to initialize the biophysical model together with either i) the same parameter combination (k*, ϵ₀*) or ii) a mismatching parameter combination (k, ϵ₀) from k ∈[7.5, . . . , 9.5] and ϵ₀ ∈[0.0001, . . . , 0.015]. 3) Then this state was evolved by integrating the PDEs of the biophysical model in space and over the next 15 timesteps. Lastly, the 5-th frame generated with the diffusion model was compared to the 13-th timesteps simulated with the biophysical model and the error (RMSE) between them was calculated. This way whether the diffusion model and the biophysical model co-evolve, and whether the diffusion model generates a dynamical state that, when evolved over time using the biophysical model, evolves as expected with that parameter combination and matches the diffusion model's output at a later time could be checked. A short simulation interval of 15 timesteps was chosen to minimize the influence of the model's intrinsic dynamics. This procedure was repeated for all 20 combinations of (k, ϵ₀) to check whether the biophysical model co-evolves best with the right parameter combination (k*, ϵ₀*) and it was found that the error is minimal when the parameters are close or match (k, ϵ₀)=(k*, ϵ₀*) (see FIG. 44E). This was seen as an indication that the parameter-specific output of the diffusion model is representative of the simulation data at that parameter combination. In other words, this means that the diffusion model generates spiral wave patterns that the biophysical model also produces with these parameters. The minimum was expected to vary and not be located at (k, ϵ₀)=(k*, ϵ₀*) if the output of the diffusion model was more arbitrary and unspecific. The fact that the minimum does not always strictly occur at (k, ϵ₀)=(k*, ϵ₀*) is likely related to very similar dynamics for close parameter combinations (see also FIG. 44C). Overall, the diffusion model appears to be able to interpolate in parameter space and generate valid parameter-specific dynamics in between parameter combinations it was trained on. This suggests that diffusion models do not need to be trained meticulously on all possible parameter combinations and can yet generate wave dynamics for many more parameter combinations than just the ones they were trained on. Note that all tested parameter combinations except (k=9.0, ϵ₀=0.015) lie in between the training parameter combinations.

FIGS. 44A-44E: Parameter-specific generation of spiral wave dynamics using diffusion generative modeling. A) The diffusion model was trained on spiral wave data simulated with different parameters, varying the parameters k and ε₀ in eqs. (1)-(2) of this example. Some parameter combinations produce more spiral waves and some produce less. B) Test data which was left out of the training data. C) Examples of diffusion-generated spiral wave dynamics with parameter combinations (k, ϵ₀)={(7. 5, 0.001), (8, 0.003)}. With the same parameter combination, the waves can look very different. Vice versa, with different parameter combinations, the waves can look very similar. D) Scheme to test whether diffusion-based generations are parameter-specific. Top: Sequence of 5 diffusion-generated frames. Overloading of first frame into biophysical model as initial state for testing whether state co-evolves, see section 2.3.7 for details. The biophysical model co-evolves the closest (black arrow) when parameters of the simulation match the parameters (k*, ϵ₀*) with which the diffusion generation was conditioned. E) Confirmation that generations are parameter-specific: the error (RMSE) between simulated and diffusion-generated output (frame 13/5) is the lowest for matching or close parameter combinations (k, ϵ₀)≈(k*, ϵ₀*). Green box: parameter combination (k*, ϵ₀*) of diffusion-generated data, see top in panel 44D. Pink box: combination producing minimum mismatch (RMSE, black: low, white: high) between simulation and diffusion-generated sequence (k and co combinations used for simulation).

### 2.4. Analysis and Conclusions

Generative modeling provides many promising applications in the biological and biomedical sciences. Here, it was demonstrated that denoising diffusion probabilistic models can be used to model waves of electrical excitation in cardiac tissue. Diffusion models can be used to reconstruct or create parameter-specific wave patterns, and, most importantly, simulate electrical wave propagation in a data-driven manner. In other words, diffusion models can learn to evolve cardiac wave dynamics from previously seen data without knowledge about the underlying physics. Therefore, they could potentially be used to create a data-driven model of the heart's electrophysiological system from measurement data. It was found that diffusion models not only generate electrical wave dynamics that look like and are visually indistinguishable from simulated wave dynamics, but the diffusion-generated dynamics also preserve some of the inherent characteristics of the original dynamics. For instance, it was found that diffusion-generated spiral wave dynamics exhibit the same self-termination statistics as their counterparts in excitable media (see section 2.3.6).

While there is some confidence that the diffusion-generated waves are indeed legitimate solutions, there also remains to be caution and further research is needed to confirm whether diffusion models provide a valid and robust alternative modeling approach to conventional biophysical modeling. At this point, it cannot be ruled out that diffusion models merely emulate rather than simulate spiral wave dynamics. This concern is particularly critical when the dynamics are chaotic and sensitive to slight physical perturbations or differences in the numerical integration. Both simpler and more complicated spiral wave dynamics were evolved, and while the diffusion model generated plausible looking simpler spiral wave dynamics for very long times (in contrast to U-Net) which co-evolved over a reasonable period of time with the ground-truth dynamics (keep in mind that even different solvers would lead to diverging results), the more complicated spiral wave dynamics diverged very quickly from and exhibited less wavebreak than their ground-truth counterpart. The latter observation could be an indication that bias in the training data influences the behavior of the dynamics in a non-deterministic way.

A major concern with diffusion models is their ability to hallucinate. Hallucination is an inherent property of generative modeling and a feature and bug at the same time. Diffusion models can generate a continuum of outputs of which some are made up and false. The main issue is that the false output is hard to identify as diffusion models excel at learning the data distribution and generating realistically looking data points from this distribution. This raises concerns over the applicability of diffusion models in healthcare, where they could produce misleading output which could lead to an incorrect diagnosis or treatment. Here, it was found that the extent to which diffusion models hallucinate is related to how much the task that the network is supposed to perform is constrained. If the problem is more constrained, then the space of possible solutions becomes smaller and there is less potential for hallucination (e.g. when evolving dynamics). Therefore, sufficiently constraining diffusion models as well as developing methods to quantify and mitigate hallucination is essential. Nevertheless, the perceived weaknesses with regard to hallucination can also be a major advantage in other situations: diffusion models excel when tasked to generate a starting point in under-constrained tasks, and therefore they could serve as a powerful prior for difficult cardiac modeling or reconstruction tasks.

Overall, despite the potential drawbacks, diffusion models are a promising tool with many potential applications in cardiac research and diagnostics. Diffusion models can in principle generate parameter- or even model-specific (scroll) wave dynamics in three-dimensional heart-shaped geometries, suggesting that they could be used to simulate atrial or ventricular fibrillation in an individualized segmentation of a patient's heart while also integrating measurement data (e.g. catheter mapping data) and generating patient- or disease-specific wave dynamics. In particular, diffusion models offer the possibility to learn different integration time scales, perform simulations at arbitrary resolutions, and skip the tedious part of finding the right initial conditions for simulating such dynamics as they can readily generate spiral and scroll wave patterns instantaneously.

It was demonstrated that denoising diffusion probabilistic models (DDPMs) can be used for generating electrophysiological wave patterns in cardiac tissue. More specifically, they can be used for recovering missing data, evolving spatio-temporal dynamics, or generating specific parameter-dependent dynamics, among other tasks. The diffusion-generated waves are visually indistinguishable from, and behave as, waves simulated with biophysical models. However, diffusion models tend to hallucinate with insufficient constraining and require significant computational resources during training. In the future, diffusion models could be used for data-driven modeling of various physiological phenomena in the heart.

### 2.5. References

The numbering related to the following references apply with respect to the experimental results presented in Example 2:
[1] V. I. Krinsky and H. L. Swinney, Waves and Patterns in Chemical and Biological Media (The MIT Press, 1991).
[2] A. Winfree, Electrical turbulence in three-dimensional heart muscle, Science 266, 1003 (1994).
[3] S. Alonso, M. Bar, and B. Echebarria, Nonlinear physics of electrical wave propagation in the heart: a review, Reports on Progress in Physics 79, 096601 (2016).
[4] W. J. Rappel, The physics of heart rhythm disorders, Physics Reports 978, 1 (2022), the physics of heart rhythm disorders.
[5] A. M. Pertsov, R. Davidenko, J. M. Salomonsz, W. T. Baxter, and J. Jalife, Spiral waves of excitation underlie reentrant activity in isolated cardiac muscle, Circulation Research 72, 631 (1993).
[6] R. A. Gray, J. Jalife, A. Panfilov, W. T. Baxter, C. Cabo, J. M. Davidenko, and A. M. Pertsov, Nonstationary vortexlike reentrant activity as a mechanism of polymorphic ventricular tachycardia in the isolated rabbit heart, Circulation 91, 2454 (1995).
[7] P. Pathmanathan and R. A. Gray, Filament dynamics during simulated ventricular fibrillation in a high-resolution rabbit heart, BioMed Research International 2015, 10.1155/2015/720575 (2015).
[8] J. Christoph, M. Chebbok, C. Richter, J. Schroder-Schetelig, P. Bittihn, S. Stein, I. Uzelac, F. H. Fenton, G. Hasenfuss, R. J. Gilmour, and S. Luther, Electromechanical vortex filaments during cardiac fibrillation, Nature 555, 667 (2018).
[9] I. Uzelac, S. Iravanian, N. K. Bhatia, and F. H. Fenton, Spiral wave breakup: Optical mapping in an explanted human heart shows the transition from ventricular tachycardia to ventricular fibrillation and selftermination, Heart Rhythm (2022).
[10] F. Fenton and A. Karma, Vortex dynamics in three-dimensional continuous myocardium with fiber rotation: Filament instability and fibrillation, Chaos: An Interdisciplinary Journal of Nonlinear Science 8, 20 (1998).
[11] O. Berenfeld and A. M. Pertsov, Dynamics of intramural scroll waves in three-dimensional continuous myocardium with rotational anisotropy, Journal of Theoretical Biology 199, 383 (1999).
[12] Z. Qu, J. Kil, F. Xie, A. Garfinkel, and J. N. Weiss, Scroll wave dynamics in a three-dimensional cardiac tissue model: Roles of restitution, thickness, and fiber rotation, Biophysical Journal 78, 2761 (2000).
[13] R. FitzHugh, Impulses and physiological states in theoretical models of nerve membrane, Biophysical Journal 1, 445 (1961).
[14] J. Nagumo, S. Arimoto, and S. Yoshizawa, An active pulse transmission line simulating nerve axon, Proceedings of the IRE 50, 2061 (1962).
[15] R. R. Aliev and A. V. Panfilov, A simple two-variable model of cardiac excitation, Chaos, Solitons & Fractals 7, 293 (1996).
[16] J. Sohl-Dickstein, E. A. Weiss, N. Maheswaranathan, and S. Ganguli, Deep unsupervised learning using nonequilibrium thermodynamics, CoRR abs/1503.03585 (2015), 1503.03585.
[17] Y. Song and S. Ermon, Generative modeling by estimating gradients of the data distribution, CoRR abs/1907.05600 (2019), 1907.05600.
[18] J. Ho, A. Jain, and P. Abbeel, Denoising diffusion probabilistic models (2020), arXiv: 2006.11239 [cs.LG].
[19] R. Rombach, A. Blattmann, D. Lorenz, P. Esser, and B. Ommer, High-resolution image synthesis with latent diffusion models, CoRR abs/2112.10752 (2021), 2112.10752.
[20] C. Saharia, W. Chan, H. Chang, C. A. Lee, J. Ho, T. Salimans, D. J. Fleet, and M. Norouzi, Palette: Image-toimage diffusion models, CoRR abs/2111.05826 (2021), 2111.05826.
[21] C. Saharia, W. Chan, S. Saxena, L. Li, J. Whang, E. Denton, S. K. S. Ghasemipour, B. K. Ayan, S. S. Mahdavi, R. G. Lopes, T. Salimans, J. Ho, D. J. Fleet, and M. Norouzi, Photorealistic text-to-image diffusion models with deep language understanding (2022), arXiv: 2205.11487 [cs.CV].
[22] U. Singer, A. Polyak, T. Hayes, X. Yin, J. An, S. Zhang, Q. Hu, H. Yang, O. Ashual, O. Gafni, D. Parikh, S. Gupta, and Y. Taigman, Make-a-video: Text-to-video generation without text-video data (2022), arXiv: 2209.14792 [cs.CV].
[23] Z. Kong, W. Ping, J. Huang, K. Zhao, and B. Catanzaro, Diffwave: A versatile diffusion model for audio synthesis (2021), arXiv: 2009.09761 [eess.AS].
[24] W. H. L. Pinaya, P.-D. Tudosiu, J. Dafflon, P. F. da Costa, V. Fernandez, P. Nachev, S. Ourselin, and M. J. Cardoso, Brain imaging generation with latent diffusion models (2022), arXiv: 2209.07162 [eess.IV].
[25] J. L. Watson, D. Juergens, N. R. Bennett, B. L. Trippe, J. Yim, H. E. Eisenach, W. Ahern, A. J. Borst, R. J. Ragotte, L. F. Milles, B. I. M. Wicky, N. Hanikel, S. J. Pellock, A. Courbet, W. Sheffler, J. Wang, P. Venkatesh, I. Sappington, S. V. Torres, A. Lauko, V. D. Bortoli, E. Mathieu, R. Barzilay, T. S. Jaakkola, F. DiMaio, M. Baek, and D. Baker, Broadly applicable and accurate protein design by integrating structure prediction networks and diffusion generative models, bioRxiv 10.1101/2022.12.09.519842 (2022).
[26] Z. Guo, J. Liu, Y. Wang, M. Chen, D. Wang, D. Xu, and J. Cheng, Diffusion models in bioinformatics and computational biology, Nature Reviews Bioengineering 10.1038/s44222-023-00114-9 (2023).
[27] A. Kazerouni, E. K. Aghdam, M. Heidari, R. Azad, M. Fayyaz, I. Hacihaliloglu, and D. Merhof, Diffusion models in medical imaging: A comprehensive survey, Medical Image Analysis 88, 102846 (2023).
[28] L. Zhou, Y. Du, and J. Wu, 3d shape generation and completion through point-voxel diffusion, CoRR abs/2104.03670 (2021), 2104.03670.
[29] Example 1: Reconstruction of Three-Dimensional Scroll Waves in Excitable Media from Two-Dimensional Observations Using Deep Neural Networks (provided above).
[30] R. Stenger, S. Herzog, I. Kottlarz, B. Ruchardt, S. Luther, F. Worgotter, and U. Parlitz, Reconstructing in-depth activity for chaotic 3d spatiotemporal excitable media models based on surface data, Chaos: An Interdisciplinary Journal of Nonlinear Science 33, 013134 (2023), https://doi.org/10.1063/5.0126824.
[31] Example 3: Deep Learning-based Prediction of Electrical Arrhythmia Circuits from Cardiac Motion: An In-Silico Study (provided below).
[32] C. Zhang, M. Rezavand, Y. Zhu, Y. Yu, D. Wu, W. Zhang, J. Wang, and X. Hu, Sphinxsys: An open-source multi-physics and multi-resolution library based on smoothed particle hydrodynamics, Computer Physics Communications 267, 108066 (2021).
[33] C. Zhang, J. Wang, M. Rezavand, D. Wu, and X. Hu, An integrative smoothed particle hydrodynamics method for modeling cardiac function, Computer Methods in Applied Mechanics and Engineering 381, 113847 (2021).
[34] P. Dhariwal and A. Nichol, Diffusion models beat gans on image synthesis, CoRR abs/2105.05233 (2021), 2105.05233.
[35] L. Jiang and Y. Belousov, Unofficial implementation of Palette: Image-to-image diffusion models, https://github.com/Janspiry/Palette-Image-to-Image-Diffusion-Models (2022).
[37] P. von Platen, S. Patil, A. Lozhkov, P. Cuenca, N. Lambert, K. Rasul, M. Davaadorj, and T. Wolf, Diffusers: State-of-the-art diffusion models.
[37] O. Ronneberger, P. Fischer, and T. Brox, U-net: Convolutional networks for biomedical image segmentation, in Lecture Notes in Computer Science (Springer International Publishing, 2015) pp. 234-241.
[38] A. Paszke, S. Gross, F. Massa, A. Lerer, J. Bradbury, G. Chanan, T. Killeen, Z. Lin, N. Gimelshein, L. Antiga, A. Desmaison, A. Kopf, E. Yang, Z. DeVito, M. Raison, A. Tejani, S. Chilamkurthy, B. Steiner, L. Fang, J. Bai, and S. Chintala, Py Torch: An Imperative Style, High-Performance Deep Learning Library, in Advances in Neural Information Processing Systems 32, edited by H. Wallach, H. Larochelle, A. Beygelzimer, F. d'Alch'e Buc, E. Fox, and R. Garnett (Curran Associates, Inc., 2019) pp. 8024-8035.
[39] J. Christoph and J. Lebert, Inverse mechano-electrical reconstruction of cardiac excitation wave patterns from mechanical deformation using deep learning, Chaos: An Interdisciplinary Journal of Nonlinear Science 30, 123134 (2020).
[40] J. Lebert, N. Ravi, F. H. Fenton, and J. Christoph, Rotor localization and phase mapping of cardiac excitation waves using deep neural networks, Frontiers in Physiology 12, 10.3389/fphys.2021.782176 (2021).
[41] R. Kazmierczak, G. Franchi, N. Belkhir, A. Manzanera, and D. Filliat, A study of deep perceptual metrics for image quality assessment (2022), arXiv: 2202.08692 [cs.CV].
[42] T. Lilienkamp, J. Christoph, and U. Parlitz, Features of chaotic transients in excitable media governed by spiral and scroll waves, Phys. Rev. Lett. 119, 054101 (2017).
[43] D. P. Kingma and J. Ba, Adam: A method for stochastic optimization, in 3rd International Conference on Learning Representations, ICLR 2015, San Diego, CA, USA, May 7-9, 2015, Conference Track Proceedings, edited by Y. Bengio and Y. LeCun (2015).
[44] M. J. Hoffman, N. S. LaVigne, S. T. Scorse, F. H. Fenton, and E. M. Cherry, Reconstructing three-dimensional reentrant cardiac electrical wave dynamics using data assimilation, Chaos: An Interdisciplinary Journal of Non-linear Science 26, 013107 (2016).
[45] M. J. Hoffman and E. M. Cherry, Sensitivity of a data-assimilation system for reconstructing three-dimensional cardiac electrical dynamics, Philosophical Transactions of the Royal Society A: Mathematical, Physical and Engineering Sciences 378, 20190388 (2020).
[46] K. He, X. Zhang, S. Ren, and J. Sun, Deep residual learning for image recognition, CoRR abs/1512.03385 (2015), 1512.03385.
[47] Z. Qu, F. Xie, A. Garfinkel, and J. N. Weiss, Origins of spiral wave meander and breakup in a two-dimensional cardiac tissue model, Annals of Biomedical Engineering 28, 755-771 (2000).

### Example 3: Deep Learning-Based Prediction of Electrical Arrhythmia Circuits from Cardiac Motion: An in-Silico Study

### 3.1. Introduction

Ventricular arrhythmias, such as monomorphic or polymorphic ventricular tachycardia (VT) or Torsade de Pointes (TdP), are caused by abnormal three-dimensional electrical wave phenomena which propagate through the thick ventricular heart muscle and initiate irregular, asynchronous contractions. Cardiac electrophysiologists perform catheter-based mapping of the electrical waves and their interactions with the underlying tissue substrate to determine the origin of heart rhythm disorders. However, current mapping techniques only provide point-by-point measurements across the heart's surface, while the intramural substrate and electrical wave dynamics remain hidden. Intramural measurements can be performed with needle catheters, but their use is invasive, provides only localized information, and the highly specialized diagnostics are only performed by a few laboratories. Therefore, alternative measurement and visualization methods of intramural electrical activation dynamics are highly needed.

It has been proposed to calculate the electrical wave phenomena from the heart muscle deformation that occurs in response to the electrical excitation. More precisely, it has been proposed to calculate regions of active stress as an approximation for the electrical waves, because electrical excitation triggers intracellular calcium release, which binds to actin-myosin filaments and promotes their contraction and therefore generates active contractile stress and deformation. It was subsequently shown for a planar active stress wave in a simulated cube of myocardial tissue that this inverse problem can be solved using a biophysical model for heart muscle tissue and inverting this model to solve for active stress. These findings were interpreted as early indications that the deformation of the heart wall could reveal electrical wave patterns. However, cardiac arrhythmia circuits often involve more complex electrical wave morphologies, such as reentrant waves. Subsequently, Lebert et al. [2] and Beam et al. [3] showed that also more complicated rotating electrical wave patterns, such as spiral waves and even three-dimensional scroll waves, can be reconstructed from deformation in simulated slabs of myocardial tissue using a data assimilation and a Kalman filter approach, respectively. With both techniques, the electrical waves were reconstructed directly, skipping active stress, by making generalized assumptions about the coupling between electrics and mechanics. However, these assumptions involved biophysical formulations for the electrical dynamics, coupling between electrics and mechanics, the tissue's contractility and elastic behavior, and information such as the muscle fiber organization, which is not known per se when imaging the heart.

More recently, it was shown that a purely data-driven deep learning-based approach can be used to solve the inverse mechano-electrical problem, even with very complicated three-dimensional electrical scroll wave dynamics, noise and low spatial resolutions [4], the approach outperforming the previous physics-based approaches [2, 3]. The deep learning-based predictions of the electrical wave dynamics were visually indistinguishable from the ground-truth even though the scroll wave patterns were fine-scaled, rapidly changing and chaotic, and the tissue retained an orthotropic muscle fiber organization which was not known to the algorithm. Next to its computational efficiency, the data-driven approach has also the advantage that no biophysical laws or model equations need to be known as in [1-3]. Instead, a convolutional encoding-decoding neural network was trained with pairs of electrical and mechanical data, which subsequently learned to predict electrical wave patterns directly from mechanics, skipping the biophysics of the excitation-contraction coupling machinery entirely.

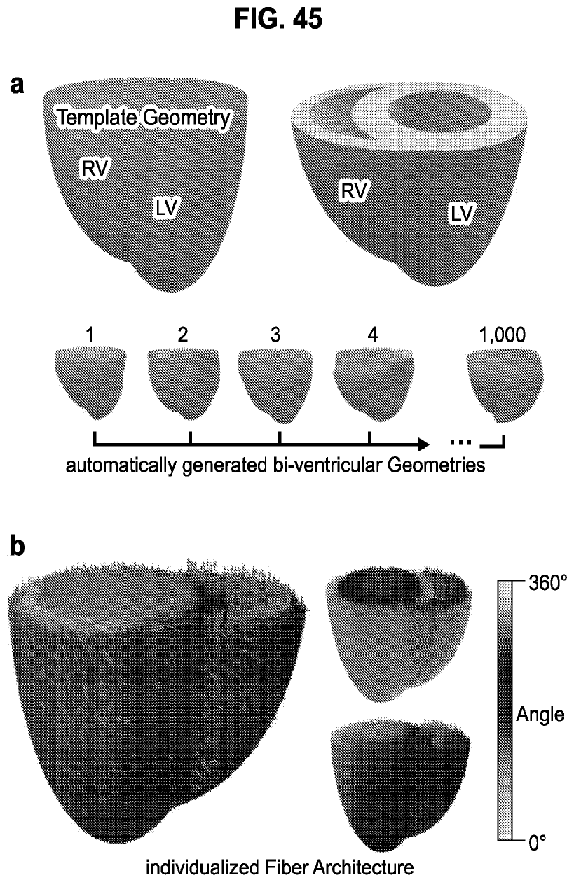

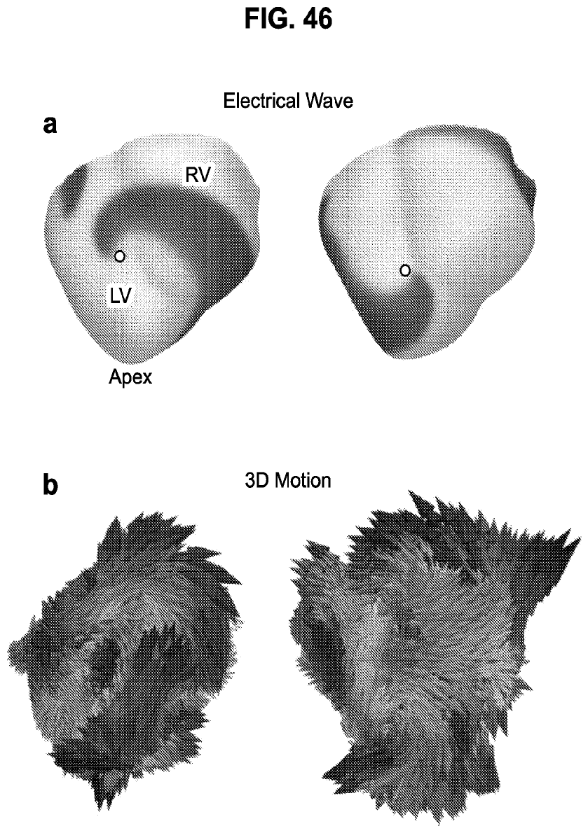

Based on these preliminary studies, an aim of the experimental results presented here was to investigate whether deep learning could also be applied to solve the inverse mechano-electrical problem in more realistic and arbitrarily shaped tissue geometries, such as different ventricular geometries that could stem from different patients, and in different tissues with significantly different underlying electrophysiology and mechanics. An embodiment was therefore configured that performed thousands of electro-mechanical computer simulations with bi-ventricular heart geometries, as shown in FIG. 45 and FIG. 46, with and without scars. To obtain a rich and diverse training dataset of ventricular arrhythmias, different electrophysiological and mechanical models were randomly chosen with varying model parameters influencing the wavelength and propagation speed of the electrical waves, for instance, as well as the tissue's contractile and elastic behavior. A neural network was then trained with the data and it was explored whether it can learn the relationship between three-dimensional electrical waves and ventricular deformation, and subsequently devise a unique, generalizable and robust mapping between electrics and mechanics, despite the complexity of the problem.

FIGS. 45A-B show generation of 1,000 different bi-ventricular heart geometries for generation of electromechanical training dataset consisting of tens of thousands of training samples of ventricular arrhythmias, according to an embodiment. FIG. 45A shows 10 different manually created template geometries were deformed using automatic remeshing and free-form deformation algorithms to create 1,000 individual bi-ventricular geometries, according to an embodiment. FIG. 45B shows each bi-ventricular shape comprises its own, unique muscle fiber architecture generated using a rule-based method, according to an embodiment. Shading/coloring: Fiber angle in the y-z-(left), x-y- (top right) and x-z-plane (bottom right).

FIGS. 46A-B show computer simulation of ventricular deformation during reentrant arrhythmia, according to an embodiment. FIG. 46A shows electrical wave (dark: depolarized tissue, gray: resting tissue) rotating around a core (white dot) or ‘reentering’ the ventricles. FIG. 46B shows corresponding ventricular deformation caused by the electrical wave. Motion displayed as three-dimensional vectors (darker/red), indicating instantaneous motion with respect to a previous deformed state. In connection with obtaining the experimental results presented here, embodiments of the invention were deployed to ask the question: does the mechanical motion that was caused by the electrical excitation contain sufficient information to be able to reconstruct the electrical wave pattern.

Experimental work has provided evidence for a strong correlation between electrical and mechanical spatiotemporal patterns in the heart [5-8]. For example, it was shown during ventricular arrhythmias that focal or rotational electrical wave phenomena visible on the heart surface cause focal or rotational mechanical deformations within the heart wall [7, 9]. These observations motivate aspects of embodiments of the present invention and suggest that embodiments of the present invention are feasible in practice.

### 3.2. Methods

A sparse encoding-decoding convolutional neural network was developed and trained using electromechanical computer simulations to predict electrical excitation waves from ventricular tissue deformation.

### 3.2.1. Computer Simulations of Bi-Ventricular Electro-Mechanics

Embodiments of the invention were deployed in connection with performing computer simulations of electromechanical wave dynamics in idealized bi-ventricular geometries with muscle fiber anisotropy, such as those shown in FIGS. 45A-B. The simulations reproduced two fundamental types of electromechanical activation dynamics in the ventricles which were assumed to underlie ventricular tachyarrhythmias: either i) focal or ii) reentrant wave dynamics. Both focal or reentrant wave patterns can underlie either monomorphic VT, polymorphic VT or Torsade de Pointes (TdP). Monomorphic VT (mVT) was simulated as being caused by either a single focal wave originating from a point source located in a random position in the ventricles or a single stable macro-reentrant wave or scroll wave, respectively. Polymorphic VT (pVT) was simulated as a single meandering scroll wave or multiple scroll waves, respectively. The latter dynamical state is similar to TdP or ventricular fibrillation (VF). Reentrant waves were induced using an S1-S2 stimulation protocol with a first S1 stimulus in a random location and a second S2 stimulus timed to occur in the vulnerable window to cause wavebreak. This protocol was repeated to induce multiple scroll waves. Throughout the study performed to generate the experimental results presented here, electrical models and mechanical models, according to embodiments, are referred to. The electrical models are phenomenological numerical models for action potential waves in cardiac tissue. For simplicity, mechanical models are referred to when it is meant to actually refer to different mechanical material properties. Only forward electromechanical coupling using phenomenological laws is employed, coupling the mechanics to the electrics, omitting feedback effects of the mechanics onto the electrics.

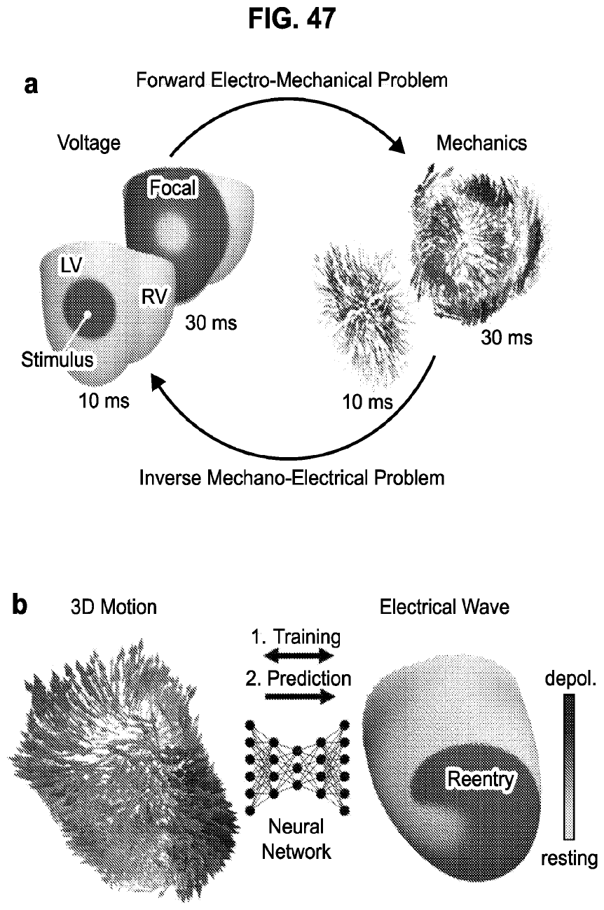

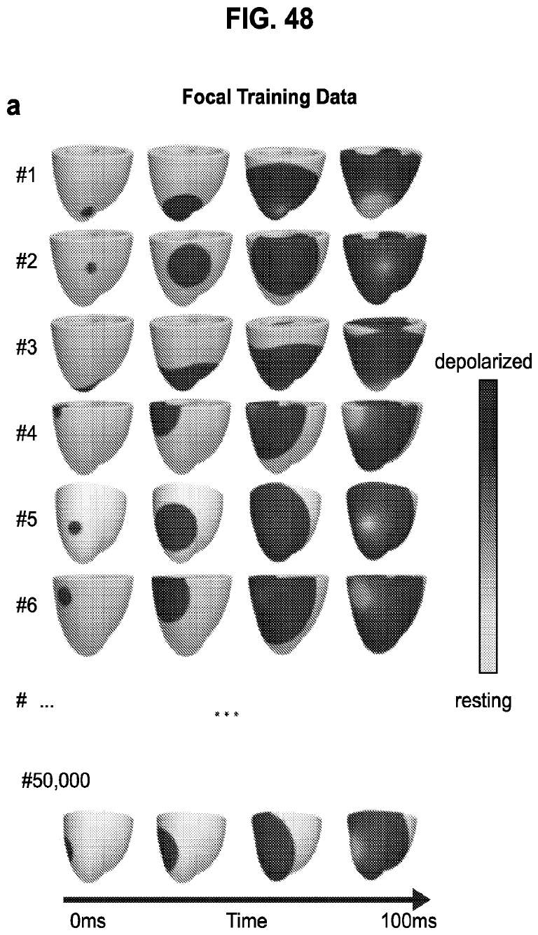

FIGS. 47A-B show deep learning-based prediction of electrical arrhythmia circuits from mechanical deformation in simulations of the heart's ventricles, according to embodiments. The possibility to compute action potential waves from cardiac tissue motion could help improve diagnosing heart rhythm disorders and other heart disease. FIG. 47A shows forward and inverse problem: In the forward electro-mechanical problem, computer simulations are used to compute electrical waves which propagate through the heart muscle and initiate contractile motion. The example shows a focal wave that originates from a point, propagates outwards and induces focal-like contractions (tissue motion is indicated by red three-dimensional displacement vectors). In the inverse mechano-electrical problem, it is an aim to compute electrical waves from tissue motion. FIG. 47B shows a neural network is used to solve the inverse mechano-electrical problem, according to embodiments. After training with many pairs of mechanical input and electrical target data it can perform predictions with previously unseen data.

### 1. Smoothed Particle Hydrodynamics Simulations

To be able to generate large quantities of training data with tens of thousands of training samples, an efficient numerical electromechanical model was used based on the smoothed particle hydrodynamics (SPH) method implemented in the general-purpose open-source SPHinXsys framework developed by Zhang et al. [10, 11]. The SPH method is a meshfree Lagrangian numerical method that discretizes the simulation domain using particles and offers the ability to handle complex tissue geometries. This simulation framework was modified and extended for use in connection with generating the experimental results presented here. In total, 1,000 simulations were performed with 1,000 different, unique bi-ventricular geometries with unique muscle fiber anisotropy, which were created automatically as described herein and illustrated in FIGS. 45A-B.

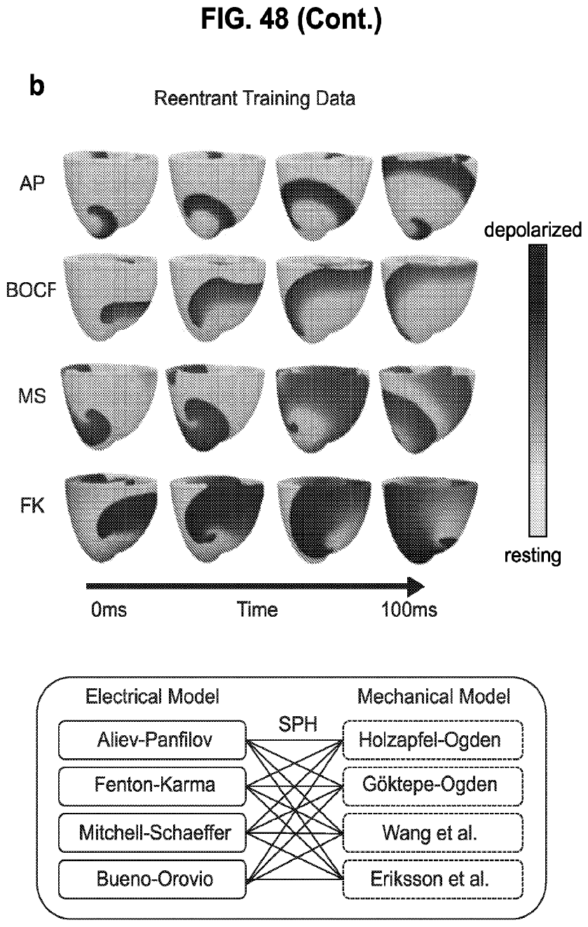

Electrical action potential wave patterns were modelled using 4 different phenomenological electrophysiological models (Aliev-Panfilov [12], Fenton-Karma [13], Mitchell-Schaeffer [14], Bueno-Orovio-Cherry-Fenton [15]), which were selected randomly before each simulation. Each model produces distinctly different wave patterns, as illustrated in FIG. 48B. However, qualitatively, all models produce electrical wave patterns typically observed in cardiac muscle tissue: focal and reentrant scroll waves or compositions thereof, as illustrated in FIG. 48A-B. FIG. 4B illustrates the quantitative differences between the electrophysiological models for scroll wave patterns. Note that the scroll wave dynamics are a snapshot of the model dynamics for a given parameter set and that each model can exhibit different and more diverse dynamics. After one of the 4 electrophysiological models was randomly selected, the electrical model parameters were set to random values varying uniformly within a defined interval around a default value. The default values for the 4 electrophysiological models are shown in Table 5. The electrical parameters controlling properties of the wave dynamics, such as action potential duration and wave speed, were chosen from a range of potential values.

TABLE 5
Default parameter values for the 4 electrophysiological models
(AP, FK, MS, BOCF) used for training data generation.
Aliev-Panfilov (AP) model

| | | | | | | | | | | | |
|---|---|---|---|---|---|---|---|---|---|---|---|
| a | 0.2 | b | 0.2 | k | 8 | μ₁ | 0.2 | μ₁ | 0.3 | ∈ | 0.002 |

Fenton-Karma (FK) model

| | | | | | | | | | | | |
|---|---|---|---|---|---|---|---|---|---|---|---|
| τᵥ⁺ | 10 | τᵥ₁⁻ | 18.2 | τᵥ₂⁻ | 18.2 | τ_{w}⁺ | 1020 | τ_{w}⁻ | 80 | τ_{d} | 1/5.8 |
| τ₀ | 12.5 | τᵣ | 130 | τₛᵢ | 127 | k | 10 | u_{c} | 0.13 | u_{c}^{si} | 0.85 |
| uᵥ | — | | | | | | | | | | |

Mitchell-Schaeffer (MS) model

| | | | | | | | | | |
|---|---|---|---|---|---|---|---|---|---|
| τᵢₙ | 0.3 ms | τₒᵤₜ | 6 ms | τₒₚₑₙ | 120 ms | τ_{close} | 150 ms | u_{gate} | 0.13 |

Bueno-Orovio-Cherry-Fenton (BOCF) model

| | | | | | | | | | | | |
|---|---|---|---|---|---|---|---|---|---|---|---|
| u₀ | 0 | uᵤ | 1.58 | θᵥ | 0.3 | θ_{w} | 0.015 | θᵥ⁻ | 0.015 | θₒ | 0.006 |
| τᵥ₁⁻ | 60 | τᵥ₂⁻ | 1150 | τᵥ⁺ | 1.4506 | τ_{w1}⁻ | 70 | τ_{w2}⁻ | 20 | u_{w}⁻ | 0.03 |
| τ_{w}⁺ | 280 | τ_{fi} | 0.11 | τₒ₁ | 6 | τₒ₂ | 6 | τₛₒ₁ | 43 | τₛₒ₂ | 0.2 |
| kₛₒ | 2 | uₛₒ | 0.65 | τₛ₁ | 2.7342 | τₛ₂ | 3 | kₛ | 2.0994 | uₛ | 0.9087 |
| τₛᵢ | 2.8723 | τ_{w∞} | 0.07 | w*_{∞} | 0.94 | | | | | | |

FIGS. 48A-C show simulated training data of focal and reentrant ventricular arrhythmias generated with electromechanical computer simulations, according to embodiments. FIG. 48A show focal training data was generated using a single S1 stimulation pulse in a random location in the ventricles, according to embodiments. Focal waves originate from a single point source. Up to 1,000 different simulations were performed with a single randomly placed stimulation pulse, randomly selected electrophysiological and mechanical models, and randomly chosen model parameters varying properties such as excitability, wave propagation speed, action potential duration, tissue stiffness and contractile strength, among others. FIG. 48B shows reentrant training data generated using a S1-S2 stimulation protocol in which a S1 stimulation pulse was first applied in a random location and then a S2 stimulation pulse was applied in the vulnerable phase of the wave causing wavebreak and reentry, according to embodiments. Reentrant waves rotate around a core or an anatomical site (such as a scar or an occlusion). Different reentrant scroll wave dynamics with the Aliev-Panfilov [12] (AP), Bueno-Orovio-Cherry-Fenton [15] (BOCF), Mitchell-Schaeffer [14] (MS) and Fenton-Karma [13] (FK) models. FIG. 48C shows combinations of electrophysiological and mechanical models used for the simulations in FIGS. 48A-B, according to embodiments.

The ventricular mechanics were modeled following the approach of Nash and Panfilov [16] by decomposing the Piola-Kirchhoff stress tensor P as the sum of passive (Pₚ) and active (Pₐ) stress components. The passive stress is derived using a strain-energy function W, which accounts for the anisotropic and nonlinear mechanical properties of the myocardium:

$$W = U + {W}_{iso} + {W}_{aniso} \qquad ( 1 )$$

where U represents a volumetric function, and Wᵢₛₒ and Wₐₙᵢₛₒ denote the isotropic and anisotropic volume preserving functions, respectively. The volumetric function U was chosen as U(J)=μₖ In(J)²/2, where J=detF is the volume ratio defined as the determinant of the deformation gradient tensor F, and μₖ is the bulk modulus that acts as a penalty parameter to enforce incompressibility. The isotropic and anisotropic strain energy functions, Wᵢₛₒ and Wₐₙᵢₛₒ, are defined using the constitutive law proposed by Holzapfel and Ogden [17]:

$${W}_{iso} = \frac{a}{2 b} {e}^{b ( {I}_{1} - 3 )} \qquad ( 2 )$$

$${W}_{aniso} = {\sum }_{i = f , s} ( \frac{{a}_{i}}{2 {b}_{i}} ( {e}^{{{b}_{i} ( {I}_{4 i} - 1 )}^{2}} - 1 ) ) + \frac{{a}_{fs}}{2 {b}_{fs}} ( {e}^{{b}_{fs} {I}_{8 fs}} - 1 ) \qquad ( 3 )$$

Here, a and b are parameters related to the matrix response, a_{f} and b_{f} account for the myocardial fiber contributions, as and bs denote the parameters for the fiber sheet contributions, and a_{fs} and b_{fs} characterize the shear effects in the sheet plane. For definitions of the invariants I₁, I_{4f}, I₄ₛ, and I_{8fs}, as well as the formulation of the passive stress tensor Pp see Zhang et al. [11]. The active stress component modeling the internal active contraction stress was defined as Pₐ=Tₐ(u)Ff₀⊗f₀, where Tₐ(u) represents the active fiber tension in dependence of the transmembrane potential u and f₀ is the local fiber direction. The evolution of Tₐ(u) governed by the following ODE [16]:

$${\partial }_{t} {T}_{a} = {ϵ}_{T} ( u ) ( {k}_{T} u - {T}_{a} ) \qquad ( 4 )$$

where ϵ_{T}(u) is a delay function controlling the rate of activation and relaxation. The smoothly varying form of ϵ_{T}(u) was employed proposed by Goktepe and Kuhl [18]:

$${ϵ}_{T} ( u ) = {ϵ}_{0} + ( {ϵ}_{∞} - {ϵ}_{0} ) exp ( - exp ( - {ξ}_{T} ( u - {u}_{0} ) ) ) \qquad ( 5 )$$

where ε₀ and ϵ∞ represent the lower and upper bounds of ϵ_{T}(u), respectively, while ξ_{T} controls the rate of transition between these values around u₀. The corrected relation ϵ∞<ϵ₀ was adopted, as noted by Eriksson et al. [19], and ϵ∞==0.1, ϵ₀=1.0, ξ_{T}=100, and u₀=0.05 were chosen. To generate a dataset of diverse mechanical responses and mechano-electrical correspondences the following parameters were varied for each simulation: (1) the bulk modulus μₖ, (2) the material parameters of the Holzapfel-Ogden constitutive law (a, b, a_{f}, b_{f}, aₛ, bₛ, a_{fs}, and b_{fs}), and (3) the maximum active force k_{⊥}. First, the bulk modulus μₖ was set as a function of the Young's modulus E using μₖ=E/(3 (1-2v)) with the Poisson's ratio v=0.4995. E was chosen from a uniform distribution between 5 kPa to 15 kPa for each simulation. Second, the passive material parameters a, b, a_{f}, b_{f}, aₛ, bₛ, a_{fs}, and b_{fs} were chosen in two ways: a base value parameter set from four different sources, and a scaling factor kₐ which scales a, a_{f}, aₛ, a_{fs}. Table 5 lists the base value parameter sets from Holzapfel and Ogden [17], Eriksson et al. [19], Goktepe et al. [20], and Wang et al. [21], which stem from different fits on shear test data of porcine ventricular myocardium [22]. For each simulation, one parameter set was randomly selected and the scaling factor kₐ was chosen randomly from the range 0.8 to 1.2.

TABLE 5
Material parameter choices for the Holzapfel-Ogden constitutive model.

| Source | a [kPa] | b | a_{f} [kPa] | b_{f} | aₛ [kPa] | bₛ | a_{fs} [kPa] | b_{fs} |
|---|---|---|---|---|---|---|---|---|
| Holzapfel [17] | 0.059 | 8.023 | 18.472 | 16.026 | 2.481 | 11.120 | 0.216 | 11.436 |
| Goktepe [20] | 0.496 | 7.209 | 15.196 | 20.417 | 3.283 | 11.176 | 0.662 | 9.466 |
| Wang [21] | 0.2362 | 10.810 | 20.037 | 14.154 | 3.7245 | 5.1645 | 0.4108 | 11.300 |
| Eriksson [19] | 0.333 | 9.242 | 18.535 | 15.972 | 2.564 | 10.446 | 0.417 | 11.602 |

### 2. Finite Element Method Simulations

Additional electromechanical simulations were carried out using a finite element method (FEM). Simulations were carried out in bi-ventricular geometries that were distinct from the bi-ventricular geometries used with the SPH method described herein. The geometries were also generated as described herein using a template geometry distinct from the ones used with the SPH method. The FEM methodology was not known to individuals developing the deep learning methodology.

3. Automatic Generation of Unique Ventricular Geometries with Muscle Fiber Architecture

Unique idealized bi-ventricular geometries were created automatically for each individual simulation by selecting 1 out of 10 different template geometries and randomly deforming this geometry into a unique new geometry. The 10 template bi-ventricular geometries were created manually with computer-aided design software (Onshape). Each template had different wall thicknesses, diameter, height, offsets between left and right ventricles, etc. First, the template geometry mesh was remeshed to achieve a uniform distribution of vertices using a Voronoi clustering technique [24] implemented in the pyacvd library [24]. The geometry was then deformed using a free-form deformation (FFD) algorithm [25] to smoothly transform the remeshed template geometry into a new, distinctive shape using a random deformation field. FFD utilizes a lattice-based control structure to manipulate the shape of the underlying object in a smooth and continuous manner by operating on the vertices of the mesh. The deformation process is parameterized by a lattice control structure of dimensions N_{deform}×N_{deform}×N_{deform}, where N_{deform} represents the number of control points in each direction. A random deformation was created by modifying the lattice control point positions, perturbing them in all directions using Gaussian noise with mean 0 and standard deviation σ_{deform}. The values of N_{deform} and σ_{deform} are both randomly chosen. Table 6 presents a summary of parameter ranges used. The FFD was performed using the PyGeM library [26], and the resulting geometry was refined using the MeshFix algorithm [27] to ensure a mesh without self-intersections.

TABLE 6
Summary of the parameter ranges used to generate
the simulation dataset. For all numerical ranges a uniform
distribution was used to choose a value.
Geometry Generation

| Base Geometry | N_{deform} | σ_{deform} | θₑₚᵢ | θ_{endo}-θₑₚᵢ |
|---|---|---|---|---|
| {#1, . . . , #13} | 8 to 20 | 0.05 to 0.3 | −90° to −60° | 80° to 120° |

Electrophysiology

| Model | Stimulation | D_{iSO} | Dᵣₐₜᵢₒ | ξₕ | ρ_{scar} |
|---|---|---|---|---|---|
| {AP, FK, MS, BOCF} | {S1, S1-S2} | 0.2 to 0.4 (S1) | 2.0 to 4.0 | 0 to 0.3 | 0% to 20% |
| | | 0.1 (S1-S2) | | | |

Mechanics

| Passive Parameters | kₐ | k_{T} | E |
|---|---|---|---|
| {#1, . . . , #4} | 0.8 to 1.2 | 80 to 120 kPa | 5 to 15 kPa |

System
Δtₛₐᵥₑ
13 ms to 32 ms (corresponds to 31 Hz to 77 Hz)

The corresponding ventricular muscle fiber architecture was created for each individual ventricular geometry using a rule-based method developed by Quarteroni et al. [28]. Fiber and sheet directions were assigned to each particle or element as part of the initialization process of each simulation. The algorithm ensures the rotation of the fiber and sheet directions through the ventricular wall by defining a rotation angle θ with

$$θ = ( {θ}_{epi} - {θ}_{endo} ) ϕ + {θ}_{endo} \qquad ( 6 )$$

where the transmural position φ varies between 0 and 1, with φ=0 corresponding to the endocardial surface and φ=1 corresponding to the epicardial surface. This approach allows for a smooth and continuous change in fiber orientation throughout the myocardial wall. A value for θₑₚᵢ was randomly chosen from the range −90 degrees to −60 degrees and for θ_{endo}-θₑₚᵢ from the range 80 degrees to 120 degrees from a uniform distribution for each simulation.

### 4. Spatial Heterogeneity Generation

Cardiac tissue frequently exhibits non-homogeneous dynamical properties that vary spatially throughout the heart. To account for these variations and to generate a broader range of electrical dynamics, three-dimensional Perlin noise [29, 30] was employed in the SPH simulations with the goal to create smooth, spatially varying noise patterns. These patterns were then used to perturb the electrical dynamics of the tissue, simulating the inherent heterogeneity of the heart. Perlin noise was used for three different purposes: i) to perturb the diffusion process dynamics, ii) to alter the dynamics of the slow/gating variables of the electrophysiological models, and iii) to procedurally generate scar tissue areas that mimic an infarct.

A spatially varying conductivity coefficient c was generated to perturb the diffusion process dynamics. The conductivity coefficient scales the local diffusion rates, introducing spatial heterogeneity in the propagation of action potential waves. The 3×3 diffusion matrix Dᵢ for a particle i is defined as

$${D}_{i} = {c}_{i} ( {D}_{iso} I + {D}_{aniso} {f}_{0} ⊗ {f}_{0} ) \qquad ( 7 )$$

where I is the identity matrix, f₀ is the local fiber direction, ⊗ is the outer product, and Dᵢₛₒ and Dₐₙᵢₛₒ represent the isotropic and anisotropic components of the diffusion coefficient, respectively. The scalar value ci is determined at simulation initialization by the spatially varying Perlin noise:

$${c}_{i} = 1 + {ξ}_{h} {ξ}_{i} \qquad ( 8 )$$

where ξₙ is a global scaling factor and ξᵢ=ξ_{Perlin}(X_{i,t=0}) is the Perlin noise value for particle i with initial position x_{i,t=0}. In the simulation, the Dᵢₛₒ and Dₐₙᵢₛₒ diffusion coefficients were varied. For this purpose it was defined

$${D}_{ratio} = ( {D}_{iso} - {D}_{aniso} ) / {D}_{iso} \qquad ( 9 )$$

Dᵣₐₜᵢₒ was randomly chosen from the range 2.0 to 4.0 with a uniform distribution. For Dᵢₛₒ, a value of 0.1 mm²/ms was chosen for reentrant simulations and randomly from a range 0.2 to 0.4 mm²/ms for the focal simulations.

The dynamics were spatially altered:

$${\partial }_{t} v \to ( 1 + {ξ}_{h} {ξ}_{i} ) {\partial }_{t} v \qquad ( 10 )$$

### 5. Scar Tissue Generation

Scar or infarcted tissue was modelled in the SPH simulations by procedurally generating areas of non-conducting and non-contracting areas using Perlin noise [29, 30]

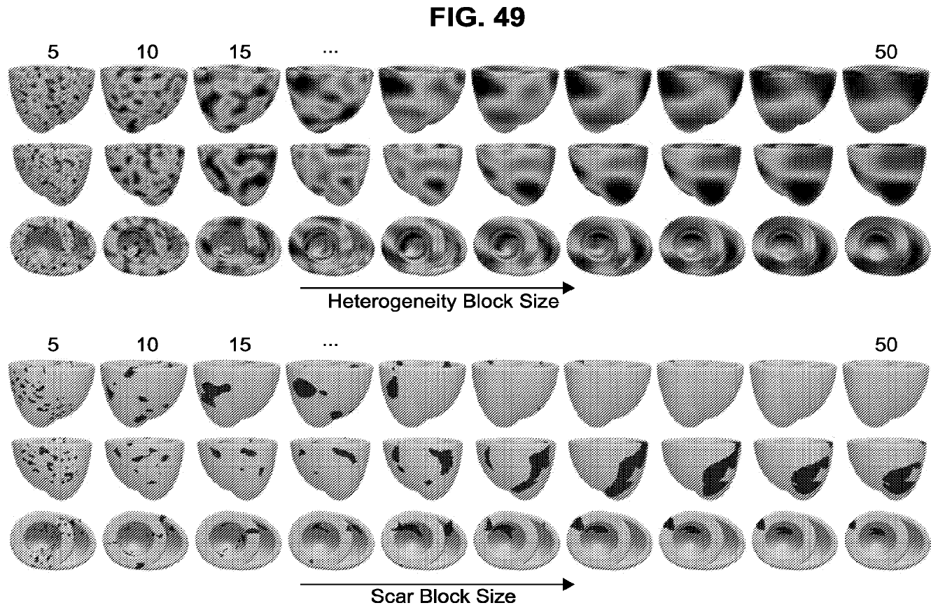

FIGS. 49A-B show spatial heterogeneity and scar tissue in the ventricles automatically generated for each simulation. FIG. 49A shows varying degrees of tissue heterogeneity. FIG. 49B shows non-conducting and non-contracting tissue simulating scarred ventricular tissue.

### 3.2.2. Training Data Generation

40,000 training samples of pairs of electrical activation sequences and mechanical deformation were generated during focal and reentrant arrhythmias using the data generated with the smoothed particle hydrodynamics (SPH) method, as described herein. The data generated with the finite element method (FEM), as described herein, was excluded from the training data. A dataset consisting of 1,000 SPH simulations was generated, each with a distinct set of parameters. For each of the 4 electrophysiological models (AP, FK, MS, BOCF), 125 simulations for both focal and reentrant dynamics were produced, in total 2. 4. 125 simulations. The focal and reentrant simulations were run for 2.6 s and 3.9 s (simulation time), respectively. If the excitation of a reentrant simulation ceased completely, the simulation was restarted. To mimick various imaging speeds, the simulation state was saved every Atsave seconds (simulation time). In each simulation, Atsave was randomly chosen from the range 13 ms to 32 ms. This would correspond to imaging acquisition rates of 31 Hz to 77 Hz during 4D ultrasound imaging. Several training samples were sampled from one of the SPH simulations without overlap between samples. One training sample consists of 5 three-dimensional displacement vector fields describing the deformation of the ventricles and one corresponding three-dimensional set of points describing the cellular transmembrane voltage or three-dimensional electrical wave pattern. Data augmentation was used to increase this initial training dataset size. Data augmentation consists of rotating, translating, scaling, cropping and subsampling the data or lowering the resolution of the data. The augmentations were equally applied to the vector-valued input data and the scalar-valued target data.

### 3.2.3. Neural Network Architecture

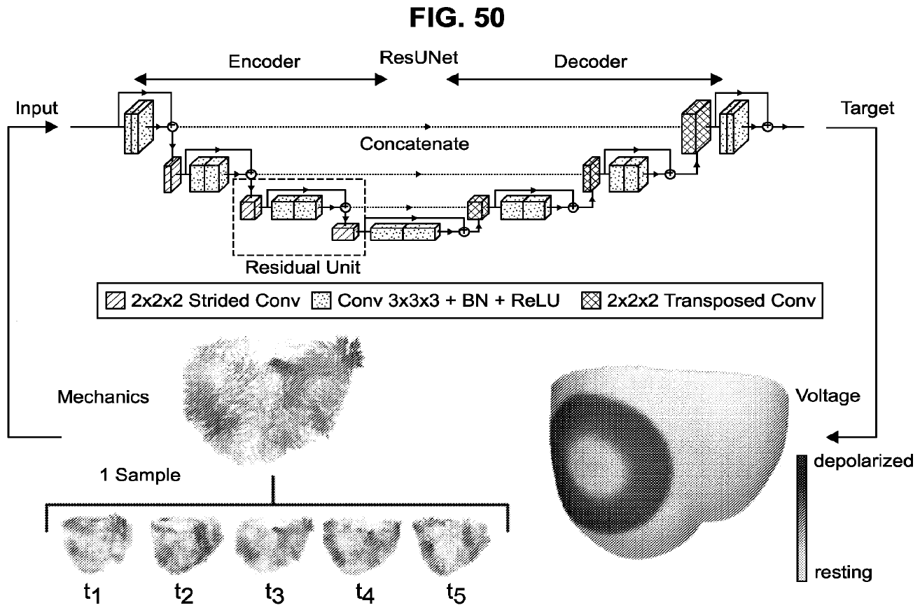

A sparse fully convolutional encoder decoder neural network was developed to predict a scalar-valued point cloud representing cellular transmembrane voltage from a sequence of vector-valued point clouds representing a short spatio-temporal sequence of tissue motion, as illustrated in FIG. 50. To improve the regularization and performance of the network, U-Net-like long skip connections [31] were included and ResNet-like short skip connections [32], also known as residual blocks were included. To reduce memory and computational requirements, the neural network operates on sparse tensors instead of dense tensors utilizing the Minkowski Engine library [33] implemented in PyTorch [34]. The Minkowski Engine voxelizes the pointcloud input data into a sparse tensor and performs sparse convolutional operations.

FIG. 50 shows Neural network architecture and training samples for inverse mechano-electrical prediction of electrical arrhythmia circuits from ventricular deformation. The network architecture is a sparse fully convolutional ResUNet based on the U-Net architecture with encoding and decoding stage and skip connections as well as residual skip connections. The input to the network (left) is a short spatio-temporal sequence of displacement vector fields (here shown for 5 snapshots). Each vector indicates the motion of a tissue particle in three-dimensional space. The target or output of the network (right) during training or inference, respectively, is a three-dimensional scalar-valued field with normalized values for the transmembrane voltage for each tissue particle.

In more detail, in the embodiment, the neural network comprises an encoding part and a decoding part with a latent space in between. The input to the neural network is a sparse tensor with N channels generated by the Minkowski Engine using a quantization of 1. The main building blocks of each part are residual blocks consisting of two 3D padded convolutional layers with a kernel size of 3×3×3, rectified linear unit (ReLU) activation [35] and batch normalization [36]. The input of a residual block is passed through a convolutional layer with kernel size 1×1×1 and is added up element-wise to the output of the second 3D convolutional layer to form the residual connection. This short skip connection can improve performance and help to alleviate vanishing gradient problems [32]. Additionally, long skip connections between encoding and decoding residual blocks are added by concatenating the output of the encoding block to the input of the decoding block. The encoding part consists of three residual blocks with 64, 128, and 256 filters for each convolutional layer. In the decoding part, three residual blocks were used with the same number of filters in reverse order, 256, 128, and 64. In the encoding part, a strided convolutional layer was used with kernel size 2×2×2, a stride of 2×2×2 after each block to downsample the input by a factor of two. Conversely, decoding residual blocks are processed by a transposed convolutional layer with the same parameters as the downsampling layers for upsampling. The downsampling and upsampling layers use the same number of filters as their respective residual block. The final layer of the decoding part is a convolutional layer with kernel size 1×1×1 with sigmoid activation and a single filter to map the output to a single scalar value. In this study, N=5 input channels were used as the network was intended to analyze 5 subsequent deformation states of the tissue. Accordingly, because the input is 5 three-dimensional vector-valued pointclouds, per point, the network receives 5. 3=15 values as input (x-, y- and z-components) and maps these values to a single scalar. It was found empirically that 5 temporal snapshots provide sufficient information about the dynamics, see also [4, 37, 38]. In total, the neural network has 27 million trainable parameters.

Training was performed on a NVIDIA RTX 25000 graphics processing unit in PyTorch version 1.12.1 with mean squared error (MSE) as the loss function, the AdamW optimizer [39], a learning rate of 0.001 and a weight decay of 0.01. The training took about 60 hours with 40,000 samples for 64 epochs with a batch size of 32 samples.

### 3.3. Results

It was found that it is possible to predict focal and reentrant electrical wave patterns in simulated bi-ventricular heart geometries from the deformation that occurs in response to the electrical activation. The predictions can be performed in unseen, arbitrarily-shaped ventricular geometries with complex muscle fiber architecture, even with significant tissue heterogeneity and the partial presence of non-conducting and non-contracting scar tissue.

### 3.3.1. Focal Waves

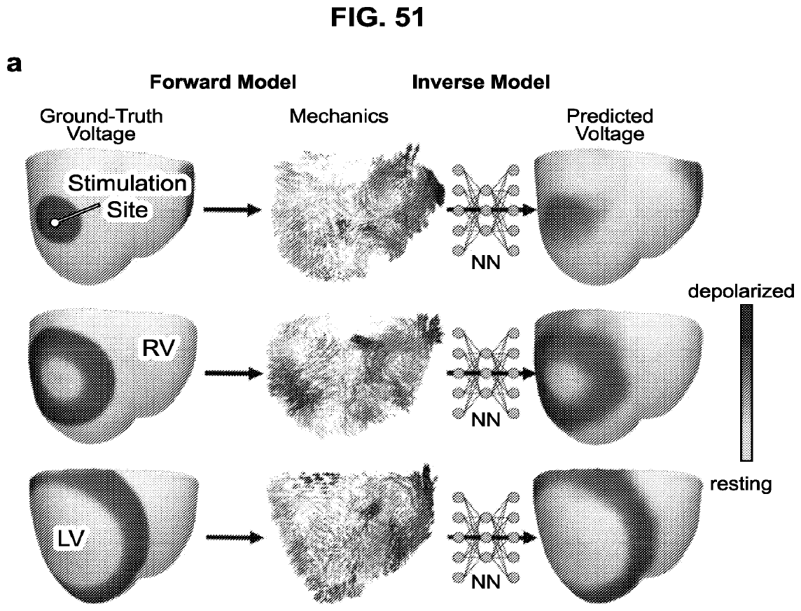

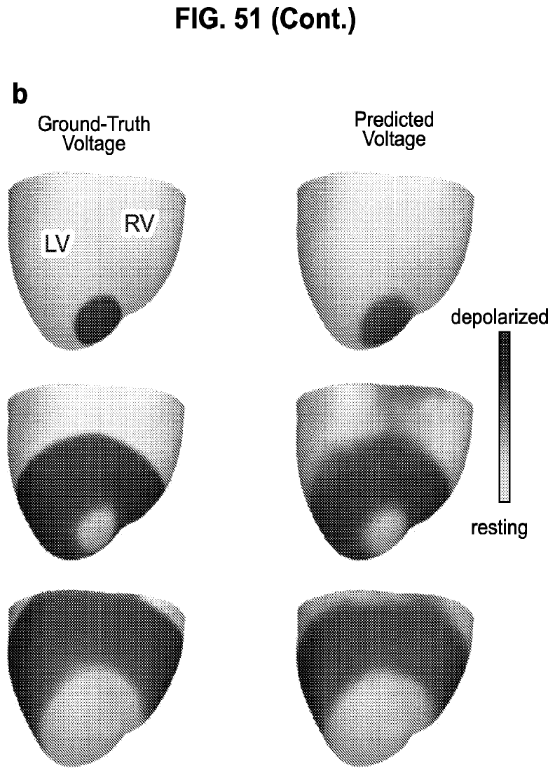

Focal waves can cause premature ventricular contractions (PVCs) and may underlie slower hemodynamically stable monomorphic ventricular tachycardia (VT). PVCs commonly originate from the papillary muscles, but can also originate from other locations in the ventricles. FIGS. 51A-B show that an embodiment of a deep learning algorithm of the invention can identify focal electrical wave patterns from the ventricular deformation that occurs in response to these focal wave patterns. The predictions show focal electrical wave patterns that propagate outwards and away from their focal origin. The algorithm produces a sequence of predictions which is temporally smooth and consistent over time, even though each prediction was performed individually without knowledge about the electrical wave patterns in previous time steps. FIGS. 51A-B show two representative examples with shorter and longer wavelengths, and it was found that the predictions perform equally well across all wave regimes, electrical and mechanical models and model parameters, and the focal waves can originate from any random location in the ventricles. The predicted electrical wave patterns are three-dimensional and transmural. Overall, the prediction accuracies with focal waves are in the order of 90-95% across all combinations of the electrical and mechanical models and parameters.

FIGS. 51A-B show deep learning-based predictions of focal electrical (action potential) waves from ventricular mechanical deformation. FIG. 51A shows representative example of a focal voltage wave (left, ground-truth) originating from the left ventricle causing contractions and subsequent deformation of both ventricles (center, red displacement vectors), which is analyzed by a neural network (NN) to predict the electrical wave pattern (right). Each prediction was performed individually and the sequence of predictions yields a temporally consistent focal wave pattern. FIG. 51B shows second example with ground-truth and predicted electrical focal wave originating from apex region. The data was not seen by the neural network during training (neither the particular bi-ventricular geometry nor the focal wave).

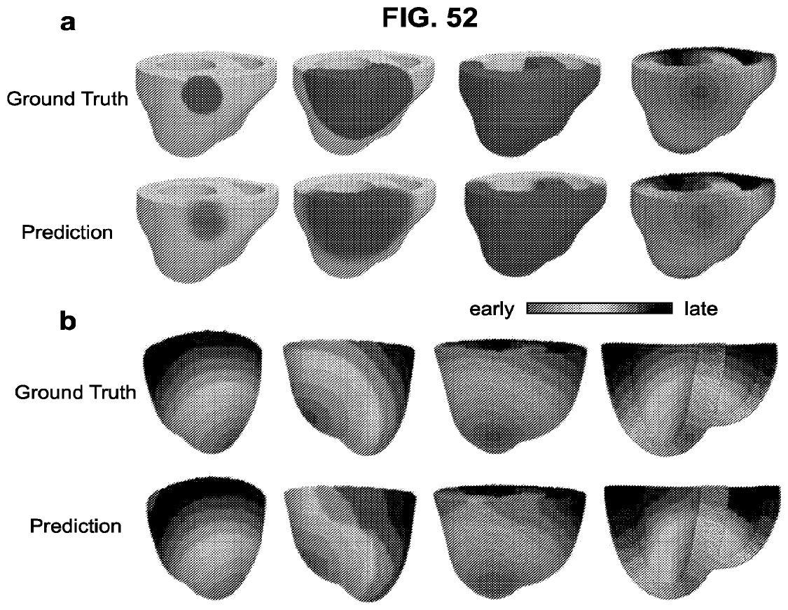

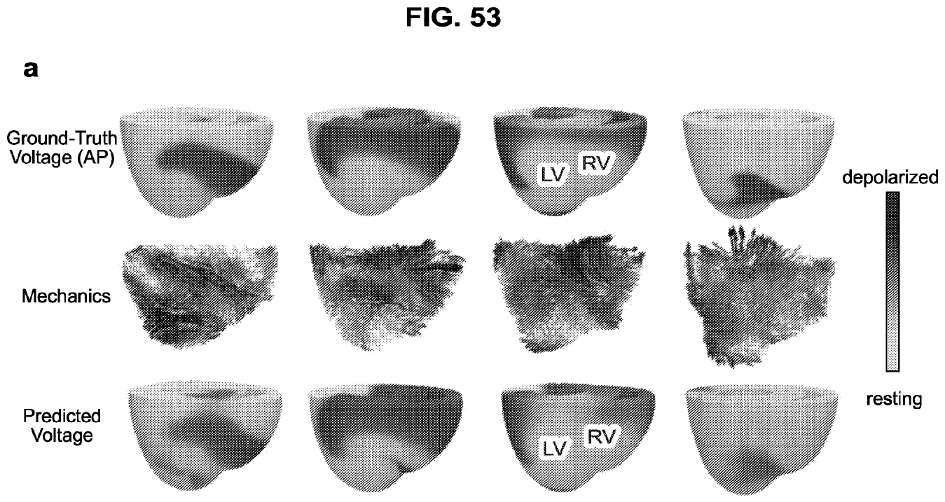

Using the sequence of predictions, it is possible to locate the origin of focal electrical waves within the ventricles. FIGS. 52A-B show electrical activation maps, which were computed from the sequence of predicted electrical wave patterns, and therefore computed from the ventricles motion. FIG. 52A shows a sequence of a ground-truth and predicted focal electrical wave pattern and the two corresponding electrical activation maps, which are visually nearly identical. FIG. 52B shows other representative examples of ground-truth and predicted activation maps, one of which shows an intramural wave source approximately at midwall close to the apex (right). The examples shown in FIGS. 51 and 52 were not part of the training data and were therefore not seen by the neural network during training. In particular, neither the bi-ventricular geometries nor the particular focal waves with their origin, wavelength and conduction velocity were part of the training data. The wave sources can even be located analyzing ventricular deformation data that was generated with an unknown electromechanical computer model. While the examples in FIGS. 51 and 52 do not include any scar tissue, it was found that predictions of focal waves are also possible in the presence of scars with slightly lower accuracies.

### 3.3.2. Reentrant Waves

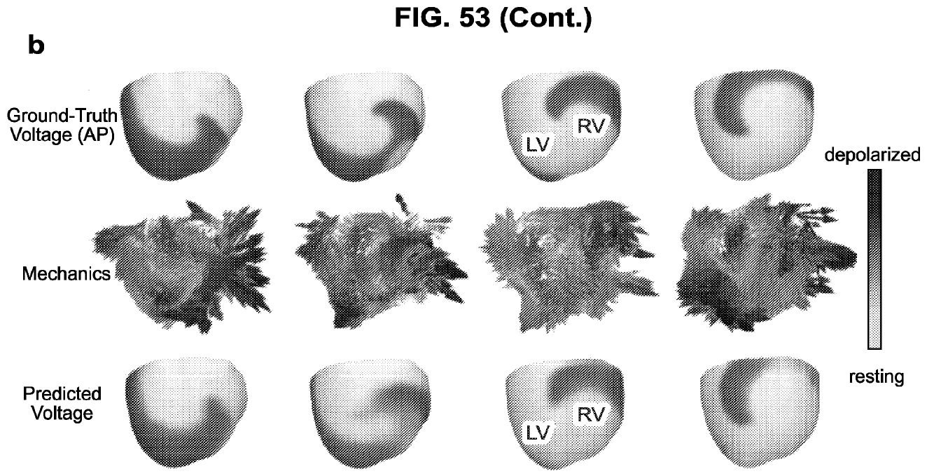

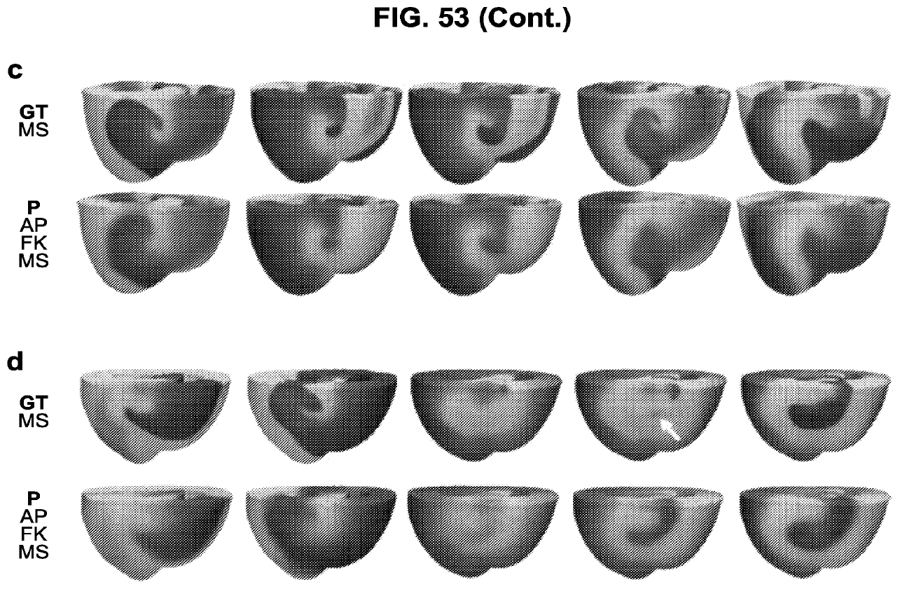

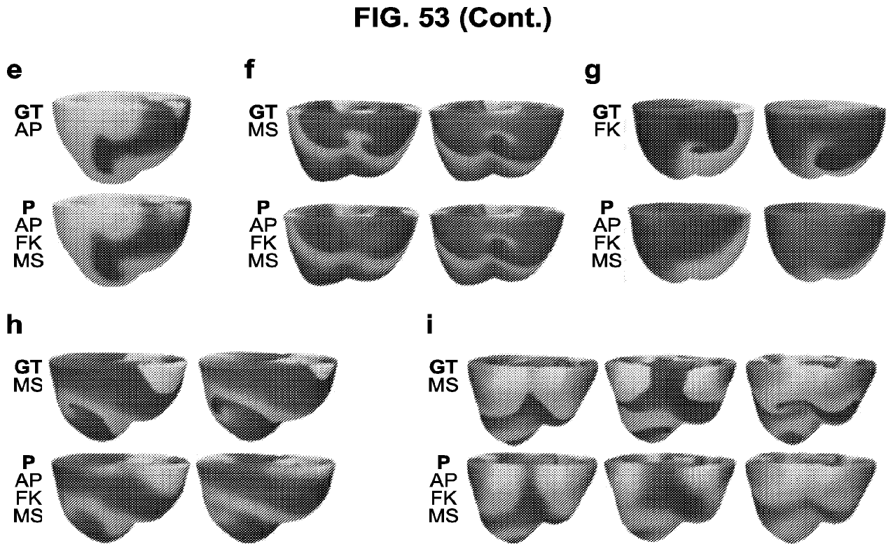

Reentrant waves underlie faster and hemodynamically unstable monomorphic or polymorphic ventricular tachycardia (pVT), Torsade de Pointes (TdP) or ventricular fibrillation (VF). FIGS. 53A-I show that it is possible to reconstruct three-dimensional reentrant electrical wave patterns from the ventricular motion that these patterns caused using a deep learning approach according to an embodiment. FIGS. 53A-B show reentrant electrical scroll waves produced with the Aliev-Panfilov model (AP) [12], their corresponding mechanical ventricular deformations and predictions. The predictions (P) exhibit minor artifacts, but are very similar to the original ground-truth (GT) wave pattern. The sequence of predictions is temporally smooth and consistent over time, even though the predictions were performed independently from each other without knowledge about the electrical waves in previous time steps. The waves in FIGS. 53A-B are idealized for illustration purposes and are relatively ‘well-behaved’. FIG. 53C shows a more chaotic and intricate clock-wise rotating reentrant wave produced with the Mitchell-Schaeffer model (MS) [14]. The wave meanders very strongly and evolves quickly into a completely different wave pattern after a few rotations. The dynamics are more comparable to pVT, TdP or VF. Overall, the deep learning model reconstructs a blurred version of the spiral-like wave pattern that is consistent with the global wave pattern throughout the volume of both ventricles, but lacks to reproduce finer details of the wave pattern. FIG. 53D shows another clock-wise rotating wave generated with the MS model, which breaks through the epicardial surface (white arrow). The prediction is able to recapitulate this breakthrough, indicating that the approach is able to resolve transmurality, but struggles with finer details of the wave pattern. FIGS. 53E-F show successful predictions of double rotors or figure-of-eight patterns with the AP and MS models, respectively. Here the model succeeded with reproducing some of the finer details of the wave pattern. FIG. 53G shows a large clock-wise rotating reentrant wave generated with the Fenton-Karma model (FK) [13]. Overall, the deep learning model performed poorly with reentrant waves generated with the FK model, presumably because it exhibited the most diverse dynamics among the 4 electrophysiology models. The FK model produced both larger macro-reentrant waves as well as very fine-scaled VF-like waves, and the deep learning model might require more training data. FIG. 53H shows another example with the MS model in which the predictions appear like low-pass filtered versions of the ground-truth wave pattern. FIG. 53I shows predictions of ‘fully turbulent’ scroll wave dynamics. Again, the predictions capture the global wave patterns but smear out finer details. The data suggests that with further improvements this approach could be used to reconstruct three-dimensional action potential wave patterns underlying VF.

FIGS. 53A-I show deep learning-based predictions of reentrant electrical (action potential) waves from ventricular motion. The deep learning model was trained on reentry data generated with the Aliev-Panfilov (AP) [12], the Mitchell-Schaeffer (MS) [14] and the Fenton-Karma (FK) [13] electrophysiology models without scars but with significant spatial heterogeneity in electrical and mechanical model parameters. FIGS. 53A-B show counter clock-wise rotating electrical scroll waves, corresponding mechanical deformation of the ventricles and predictions. FIG. 53C shows clock-wise rotating scroll wave (GT: ground-truth) and prediction (P) generated with MS model. FIG. 53D shows epicardial breakthrough generated with MS model. FIGS. 53E-F show double rotors or figure-of-eight patterns. FIG. 53G shows poor prediction outcome with FK model. FIG. 53H shows predictions are low-pass filtered versions of GT wave pattern. FIG. 53I shows predictions of fully turbulent scroll wave dynamics. The (GT) data on which the predictions were performed was not seen by the neural network during training (neither the particular bi-ventricular geometry nor the reentrant waves). The electrical and mechanical tissue properties were different in each simulation.

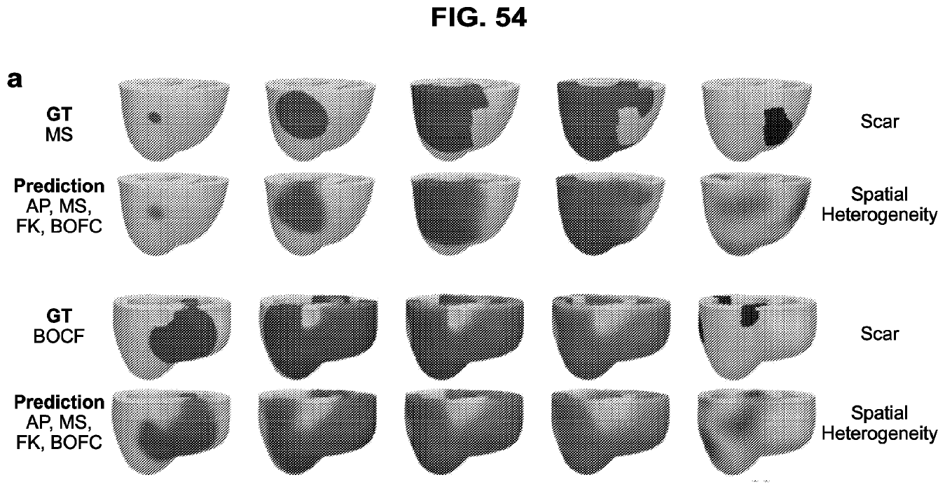

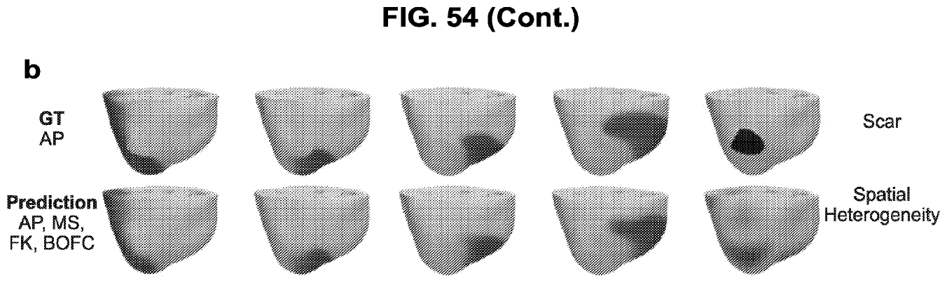

In summary, it is possible to reconstruct even complicated reentrant wave patterns from ventricular motion. The prediction accuracy is in the order of 80-95% depending on whether the wave dynamics are simpler or more complex VF-like and depending on which electrophysiological model was used. The deep learning model used in FIGS. 53A-I was trained with reentrant data generated with the AP, FK and MS models without scars, but with significant spatial heterogeneity in the electrical and mechanical parameters. Likewise, all predictions were performed in tissues without scars, but with significant spatial heterogeneity in the electrical and mechanical parameters. FIGS. 54A-B shows predictions with scars in the ventricles.

FIGS. 54A-B show deep learning-based prediction of electrical (action potential) waves from ventricular mechanical deformation in the presence of scar tissue. Scars were placed in random locations in the ventricles (deep red). Spatial heterogeneity was present in all simulations throughout this study (pink-green). FIG. 54A shows focal waves evolving around scar with MS and BOCF models. In the predictions, the scar location does not become electrically activated. However, there is significant blurring in and around the scar location. FIG. 54B shows reentrant wave generated with AP model rotating around scar. The prediction does not exhibit electrically activated tissue in scar region. The data on which the predictions were performed was not seen by the neural network during training.

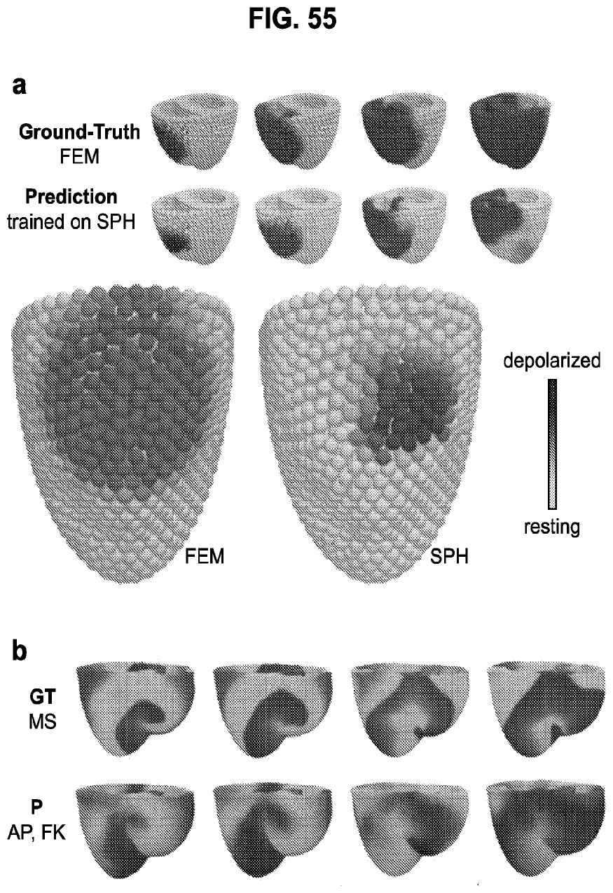

All predictions were performed on unseen data that was not part of the training data. However, the data was generated with electrical and mechanical models that were also used to generate the training data (AP, MS, FK and their combinations with one of the mechanical models). Consequently, the data to which the deep learning model was applied stems from within the training distribution, and the dynamics were in principle already known to the deep learning model from the training data. FIG. 55A-B show predictions on out-of-distribution data.

FIG. 55A-B show training of deep learning model on simulated data generated with the smoothed particle hydrodynamics (SPH) method and application to simulated data generated with the finite element method (FEM).

### 3.3.3. Heterogeneity and Scar Tissue

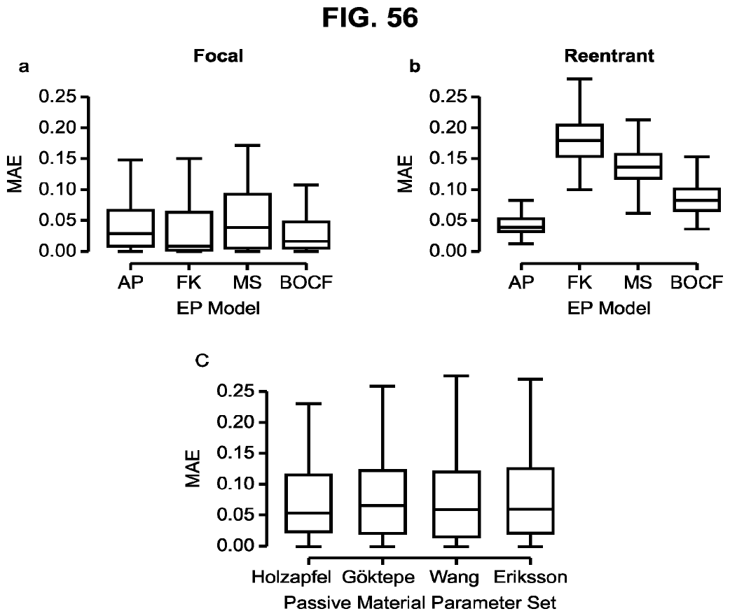

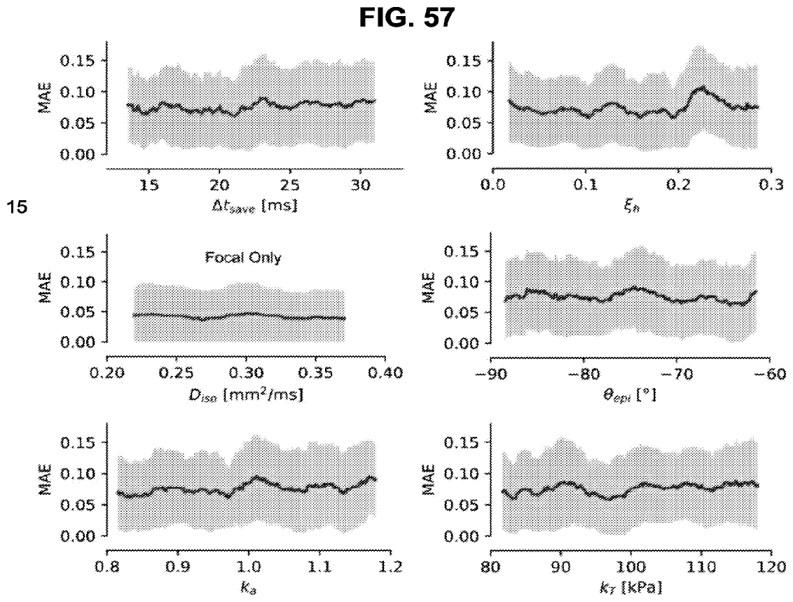

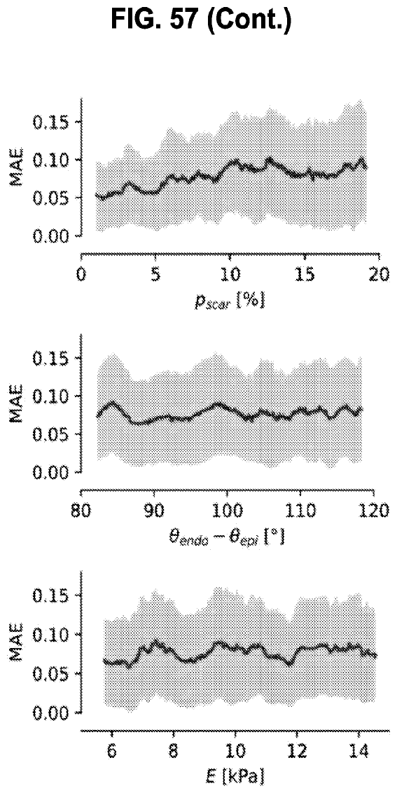

Tissue heterogeneity or scar tissue does not pose a limitation to the inverse mechano-electrical neural network. FIGS. 54A-B demonstrate that electrical waves can be predicted from tissue motion even in the presence of scars and with spatial heterogeneity. Scars were simulated as non-conducting and non-contracting tissue and were placed in random locations in the ventricles (red). FIG. 54A shows focal waves evolving around a scar with the Mitchell-Schaeffer (MS) and the Bueno-Orovio-Cherry-Fenton (BOCF) models, respectively, and FIG. 54B shows a reentrant wave generated with the Aliev-Panfilov (AP) model rotating in counter clock-wise direction around a scar. Spatial heterogeneity of tissue parameters was present in all simulations throughout this study, as shown on the right side (pink-green color-code) in FIGS. 54A-B independently from scars. The predictions do not exhibit electrical activity in the scar location. However, there is significant blurring of the wave pattern in and around the scar. Overall, the prediction accuracies are in the order of 90% with scars. FIG. 56A shows prediction accuracies in the order of 95% with focal waves and scars with all 4 electrophysiology models (AP, FK, MS, BOCF, evenly split training mix). Accordingly, FIG. 56B shows prediction accuracies with reentrant waves in tissues with scars. The prediction accuracy varies from about 95% with the AP model to 80% with the Fenton-Karma model (FK). There is no significant difference with different mechanical models (material properties) on the prediction accuracy (see FIG. 56C) when evaluating on a mix of both focal and reentrant waves. Prediction accuracies decrease with increasing scar size or scar content (scar-to-healthy tissue ratio) (see FIG. 57, top right panel) but become only marginally worse with scars overall. Spatial heterogeneity in tissue parameters was part of all training data and did not have an effect onto the prediction accuracy (see FIG. 57, top center panel). All other parameters in FIG. 57 did not influence the prediction accuracy (MEA: mean absolute difference). The data on which the predictions were performed was not seen by the neural network during training (neither the particular bi-ventricular geometry nor the scars nor the wave patterns). Both training data and data on which predictions were performed in FIGS. 54, 56 and 57 contained varying degrees of scar sizes and spatial heterogeneity. In summary, the deep learning-based algorithm of an embodiment of the invention can predict electrical wave patterns from ventricular motion robustly even in the presence of significant heterogeneity and non-conducting and non-contracting tissue.

FIG. 56A-C show prediction accuracies of deep learning-based inverse reconstruction of electrical waves from ventricular deformation of the heart on synthetic simulated data with different models. Prediction accuracies for focal (FIG. 56A) and reentrant waves (FIG. 56B) with 4 different electrophysiological models (AP: Aliev-Panfilov [12], FK: Fenton-Karma [13], MS: Mitchell-Schaeffer [14], BOCF: Bueno-Orovio-Cherry-Fenton [15]). While the prediction error (MAE: mean absolute error) stays small in the order of 5% for all models with focal waves, reentrant waves produce higher prediction errors with some of the models, particularly the ones that exhibit more complex meandering scroll wave dynamics. Prediction accuracies (FIG. 56C) with 4 different mechanical material properties (Holzapfel and Ogden [17], Goktepe et al. [20], Wang et al. [21], Eriksson et al. [19]). The data suggests that the neural network performs equally well across different electrical and mechanical models, unless the dynamics are more complex as with the Fenton-Karma model and the model has not seen sufficient training data.

FIG. 57 shows the prediction accuracy of the neural network stays the same over a wide parameter range and is on average greater than 90% for all parameters except the scar size (top left panel) and for both focal and reentrant waves (except the Fenton-Karma model). The prediction error increases slightly from 5% to 10⁻¹⁵% with increasing scar size (percentage [%] healthy to scarred tissue). Δtsave: imaging speed, ξh, ka: ?, kT: active contracile strength, E: Young's modulus.

### 3.3.4. Generalization

One of the most critical questions regarding deep learning models is whether they generalize to distinctly different data, or whether they produce reliable predictions when applied to data that is well outside of the training data distribution. A deep learning model should perform sufficiently well on data that was obtained in a different patient population that was not included in the training and is unknown to the deep learning model. To test this behaviour, the deep learning model was trained on data that was distinctly different from the training data. Most importantly, the deep learning model was trained exclusively on data generated with SPH and subsequently applied it to data generated with FEM (see FIG. 55A). The physics of the FEM model was not known during the development of the training dataset, and therefore the SPH simulations were not calibrated to match the behaviour of the FEM simulations and, as a result, the behaviour of the two simulations was quite different. For example, the mechanical boundary conditions of the SPH and FEM implementations were different in that in the SPH simulations the base of the ventricles was attached to an elastic medium that was fixed in space, whereas in the FEM simulations the base was fixed in vertical direction but could rotate in the horizontal plane. In FIG. 55A, it is shown that the neural network was able to predict the evolution of a focal electrical waves analyzing FEM data, even though it was trained solely on SPH data and therefore unfamiliar with the electromechanical dynamics of the FEM model. Despite the differences in the SPH and FEM modelling, the neural network predicted a focal wave source to appear first in the right ventricle and subsequently predicts this wave to propagate outwards and through the ventricles, in agreement with the ground-truth wave pattern. The predictions are less accurate than with SPH data, but capture the essence of the electrical activation pattern. The prediction quality is expected to improve if the SPH simulations are further refined to match the FEM simulations (e.g. matching mechanical boundary conditions). In this case, however, the mismatch between the two methodologies was intentional and serves the purpose to demonstrate the generalization capabilities of the network.

We also trained the deep learning model on data generated solely with the SPH method and with a subset of the 4 electrophysiological models (leaving one of the models out of the training data), and then applied it to data that was generated with the model that was left out from the training. FIG. 55B shows such an ‘out-of-distribution’ prediction with reentrant electrical waves, where the (ground-truth) data, which the deep learning model analyzed to reconstruct the electrical wave, was generated with the Mitchell-Schaeffer (MS) model, and the deep learning model was trained with the AP and FK models. Even though the predicted electrical waves are blurry, the overall shape and evolution of the predicted pattern matches the ground-truth and it is possible to identify a reentrant scroll wave pattern from the predictions. Together, FIG. 55B and associated video data demonstrate that predictions can be performed, even if the deep learning model has never seen the particular electrical dynamics before.

Lastly, the learning model was tested on geometries which are completely different from the biventricular shape. Associated videos shows predictions of electrical waves propagating through a teapot that deforms accordingly (this type of arrhythmia is referred to as “VTea”). While the predictions do not produce they perform sufficiently well to be able to identify the waves. The video suggests that the neural network can perform predictions also on ventricular geometries.

Overall, the findings suggest that the neural network has the capability to generalize and can recognize electrical wave patterns in significantly different mechanical data that lies well outside of the distribution of the training data. This finding indicates that the neural network is very generally able to recognize mechanical patterns and associate them with corresponding electrical wave patterns independently from the particular methodology used to create the data.

### 3.4. Analysis

In this numerical study, it was shown that deep convolutional neural networks are able to reconstruct three-dimensional electrical wave patterns from ventricular deformation. After training a network that is an embodiment of the invention with a diverse electromechanical dataset of simulated ventricular arrhythmias, it is able to devise a unique, generalizable mapping between mechanical deformation and electrical wave patterns in any ventricular geometry, despite the many factors potentially influencing this relationship: i) the infinitely many possible electrical wave patterns, ii) the arbitrarily-shaped ventricular geometries, iii) the long range elastic forces acting throughout the tissue causing non-local interactions between tissue segments (tissue shape affecting mechanics and vice versa), v) the complex fiber architecture, vi) the spatial heterogeneity in electrophysiological and mechanical parameters, vii) the noncontracting heterogeneities in the tissue, etc. Given the variety of electromechanical tissue dynamics and heterogeneity that were simulated, it is remarkable that the neural network was able to produce a universal mapping that translates any given mechanical spatio-temporal pattern into a corresponding electrical spatio-temporal pattern and can identify an underlying electrical arrhythmia circuit from any three-dimensional deformation pattern of the ventricles. The test on completely unknown data generated with a completely different methodology (FEM) suggests that the network has learned the essential features of the dynamics, is able to generalize, and could be applied to any other data as well, even experimental data. At the same time, the network has no explicit knowledge about the underlying physics and parameters. For instance, the fiber orientation does not need to be known to the network. This could be a decisive advantage over physics-based approaches [1-3] when applying the approach of the present invention to experimental data, for which such parameters are often unknown.

A central approach was to use efficient phenomenological modelling to be able to generate a diverse and extensive electromechanical training dataset in hopes that the distribution of training samples would be so broad that it spans across data that the network would later encounter during application ‘in the wild’. On the one hand, the phenomenological modelling was a necessity, because more realistic computer models of the contracting heart during arrhythmias are prohibitively expensive (computationally). On the other hand, phenomenological modelling and idealized biventricular geometries were intentionally used because it is believes that training a neural network with a very broad distribution, even if it includes exaggerated or idealized data and dynamics, will strengthen the network's robustness, promote generalization, and prevent overfitting. It is believed that a diverse phenomenological dataset with idealized dynamics can capture the most essential components of the relationship between electrics and mechanics and provide a foundation for future use. It is anticipated that the mapping that a neural network establishes from such data might be so universally applicable that the model could later be fine-tuned to more realistic data. It is expected that more realistic computer simulations would not alter the findings and implications of the experimental results presented here, and that a deep learning model trained on such data alone could still fail in practice, especially if it was trained on narrowly distributed data. One of the difficulties in pursuing a ‘trawl fishing’ approach, which is quite a typical problem in deep learning, is creating a uniform distribution of training samples in a hyperdimensional parameter space. More than 15 parameters were varied, often sampling each single parameter from a uniform distribution, with the goal to produce a diverse ensemble of dynamics. However, even if the training samples were evenly distributed in hyper-parameter space, the dynamics are likely not evenly represented and some dynamics might be more prevalent than others as certain parameter values may have a stronger influence than others.

Experimental data of ventricular motion is available in the same form as shown in FIGS. 46 and 47 as a time varying sequence of three-dimensional displacement vector fields when tracking motion in a sequence of three-dimensional images. These images could be obtained with ultrasound as in [7] or magnetic resonance imaging, for example. Different imaging speeds were mimicked by providing training samples which cover either a shorter or a longer fraction of the cycle length to the network. Using this training strategy, the network of the embodiment of the invention subsequently learned to perform predictions independently from the imaging speed. The tissue displacements in imaging data can be computed either from one frame to the next or for all frames with respect to one of the frames. It was found in [4] that both frames of reference perform equally well, as long as either approach is used consistently during training and prediction. Noteworthy, the network does not need to know an undeformed relaxed tissue configuration, which means that any arrhythmic episode can be imaged. Whether the network will be able to analyze imaging data needs to be determined in future work, and will depend on how far off the simulated data will be from the imaging data. In a different study [37], it was found that a neural network that was trained solely on simulated data can be applied to detect core regions or phase singularities of reentrant waves in optical mapping data. However, while the simulated mechanical data in the current training dataset contains noise and other features of imaging data, such as partial or cropped views, imaging data may have more convoluted features that are not yet present in the training dataset. For instance, ultrasound data is very noisy, which could produce very specific artifacts in the tracking data. Furthermore, in some embodiments, modelling does not include the atria and does not account for many of the other physiological processes in the human body, such as blood flow, for instance. This lack of realism may in certain situations prevent such model from being able to directly apply a simulation-trained neural network to patient data. However, in other cases training would be performed with in silico, ex vivo and in vivo human data.

In principle, the deep learning approach of embodiments of the invention is not limited to predicting electrical waves from motion and could also be used to predict calcium waves, activation maps, locations of active stress, scars or other predictors for arrhythmias, if the network was trained accordingly, see also [4]. For instance, training data for deep learning-based scar predictions could stem from either late gadolinium-enhanced magnetic resonance imaging, from simulations alone, or from physics-based approaches that estimate active stress or scar tissue, such as in [1, 40]. While it was found that the approach according to embodiments performs well with diverse ventricular geometries and can also be applied to some extent to a random geometry (e.g. the tea pot geometry), it is difficult to anticipate whether it will be applicable to the atria. The atria have different electrophysiological and mechanical properties as well as arrhythmia dynamics from the ventricles. Significant heterogeneity does not pose a limitation to the approach used to generate experimental results presented here, and these findings suggest that neural networks might even be able to establish a mapping between electrical and mechanical activation in patients with significant scarring, e.g. in heart failure patients as described in [8]. Even though significant electrical and contractile heterogeneity was incorporated, it was assumed that calcium waves always follow electrical waves. It is well known that this is not always the case, especially during tachyarrhythmias such as atrial or ventricular fibrillation, during which the coupling between voltage and calcium can become bi-directional or otherwise degenerated [41-44] or calcium can become silenced [45, 46]. Determining the impact of such electromechanical dissociation phenomena onto the approach requires further investigation.

### 3.5. Conclusions

A numerical proof-of-principle that electrical arrhythmia circuits in the heart can be computed from ventricular deformation mechanics using deep learning was provided. It was demonstrated that deep neural networks can learn to associate the deformation of arbitrarily shaped ventricular tissues with the corresponding three-dimensional morphology of an electrical wave pattern that caused the deformation, regardless of whether it is a simpler focal or more complicated reentrant rhythm, even in the presence of heterogeneity and scar tissue. The results suggest that a similar artificial intelligence-based approach could be used in combination with high-speed imaging, such as ultrasound or magnetic resonance imaging, to visualize action potential waves, activation maps and other electrical predictors non-invasively in patients with ventricular arrhythmias.

### 3.6. References

The numbering related to the following references apply with respect to the experimental results presented in Example 3:
[1] N. F. Otani, S. Luther, R. Singh, and R. F. Gilmour, Transmural ultrasound-based visualization of patterns of action potential wave propagation in cardiac tissue, in Annals of Biomedical Engineering (2010) pp. 3112-3123.
[2] J. Lebert and J. Christoph, Synchronization-based reconstruction of electromechanical wave dynamics in elastic excitable media, Chaos: An Interdisciplinary Journal of Nonlinear Science 29, 10.1063/1.5101041 (2019).
[3] C. B. Beam, C. A. Linte, and N. F. Otani, Reconstructing cardiac wave dynamics from myocardial motion data, in Computing in Cardiology, CinC 2020, Rimini, Italy, Sep. 13-16, 2020 (IEEE, 2020) pp. 1-4.
[4] J. Christoph and J. Lebert, Inverse mechano-electrical reconstruction of cardiac excitation wave patterns from mechanical deformation using deep learning, Chaos: An Interdisciplinary Journal of Nonlinear Science 30, 123134 (2020).
[5] B. T. Wyman, W. C. Hunter, F. W. Prinzen, and E. R. Z. Mcveigh, Mapping propagation of mechanical activation in the paced heart with mri tagging, Am J Physiol. 276, H881 (1999).
[6] J. Provost, V. T.-H. Nguyen, D. Legrand, S. Okrasinski, A. Costet, A. Gambhir, H. Garan, and E. E. Konofagou, Electromechanical wave imaging for arrhythmias, Physics in Medicine and Biology 56, L1 (2011).
[7] J. Christoph, M. Chebbok, C. Richter, J. Schroder-Schetelig, P. Bittihn, S. Stein, I. Uzelac, F. H. Fenton, G. Hasenfuss, R. J. Gilmour, and S. Luther, Electromechanical vortex filaments during cardiac fibrillation, Nature 555, 667 (2018).
[8] F. Maffessanti, T. Jadczyk, R. Kurzelowski, F. Regoli, M. L. Caputo, G. Conte, K. S. Golba, J. Biernat, J. Wilczek, M. Dabrowska, S. Pezzuto, T. Moccetti, R. Krause, W. Wojakowski, F. W. Prinzen, and A. Auricchio, The influence of scar on the spatio-temporal relationship between electrical and mechanical activation heart failure patients, EP Europace 22, 777 (2020).
[9] A. Molavi Tabrizi, A. Mesgarnejad, M. Bazzi, S. Luther, J. Christoph, and A. Karma, Spatiotemporal organization of electromechanical phase singularities during high-frequency cardiac arrhythmias, Phys. Rev. X 12, 021052 (2022).
[10] C. Zhang, M. Rezavand, Y. Zhu, Y. Yu, D. Wu, W. Zhang, J. Wang, and X. Hu, Sphinxsys: An open-source multi-physics and multi-resolution library based on smoothed particle hydrodynamics, Computer Physics Communications 267, 108066 (2021).
[11] C. Zhang, J. Wang, M. Rezavand, D. Wu, and X. Hu, An integrative smoothed particle hydrodynamics method for modeling cardiac function, Computer Methods in Applied Mechanics and Engineering 381, 113847 (2021).
[12] R. R. Aliev and A. V. Panfilov, A simple two-variable model of cardiac excitation, Chaos, Solitons & Fractals 7, 293 (1996).
[13] F. Fenton and A. Karma, Vortex dynamics in three-dimensional continuous myocardium with fiber rotation: Filament instability and fibrillation, Chaos: An Interdisciplinary Journal of Nonlinear Science 8, 20 (1998).
[14] C. C. Mitchell and D. G. Schaeffer, A two-current model for the dynamics of cardiac membrane, Bulletin of Mathematical Biology volume 65, 767-793 (2003).
[15] A. Bueno-Orovio, E. M. Cherry, and F. H. Fenton, Minimal model for human ventricular action potentials in tissue, Journal of Theoretical Biology 253, 544 (2008).
[16] M. P. Nash and A. V. Panfilov, Electromechanical model of excitable tissue to study reentrant cardiac arrhythmias, Prog. Biophys. Mol. Biol. 85, 501 (2004).
[17] G. A. Holzapfel and R. W. Ogden, Constitutive modelling of passive myocardium: A structurally based framework for material characterization, Philosophical Transactions of the Royal Society A: Mathematical, Physical and Engineering Sciences 10.1098/rsta.2009.0091 (2009).
[18] S. Goktepe and E. Kuhl, Electromechanics of the heart: A unified approach to the strongly coupled excitation-contraction problem, Computational Mechanics 45, 227 (2010).
[19] T. Eriksson, A. Prassl, G. Plank, and G. Holzapfel, Influence of myocardial fiber/sheet orientations on left ventricular mechanical contraction, Mathematics and Mechanics of Solids 18, 592 (2013).
[20] S. Goktepe, S. N. S. Acharya, J. Wong, and E. Kuhl, Computational modeling of passive myocardium, International Journal for Numerical Methods in Biomedical Engineering 27, 1 (2010).
[21] H. M. Wang, H. Gao, X. Y. Luo, C. Berry, B. E. Griffith, R. W. Ogden, and T. J. Wang, Structure-based finite strain modelling of the human left ventricle in diastole, International Journal for Numerical Methods in Biomedical Engineering 29, 83 (2012).
[22] S. Dokos, B. H. Smaill, A. A. Young, and I. J. LeGrice, Shear properties of passive ventricular myocardium, American Journal of Physiology-Heart and Circulatory Physiology 283, H2650 (2002).
[23] S. Valette, J.-M. Chassery, and R. Prost, Generic remeshing of 3d triangular meshes with metric-dependent discrete voronoi diagrams, IEEE Transactions on Visualization and Computer Graphics 14, 369 (2008).
[24] A. Kaszynski, pyacvd (2022).
[25] T. W. Sederberg and S. R. Parry, Free-form deformation of solid geometric models, ACM SIGGRAPH Computer Graphics 20, 151 (1986).
[26] M. Tezzele, N. Demo, A. Mola, and G. Rozza, PyGeM: Python geometrical morphing, Software Impacts 7, 100047 (2021).
[27] M. Attene, A lightweight approach to repairing digitized polygon meshes, The Visual Computer 26, 1393 (2010).
[28] A. Quarteroni, T. Lassila, S. Rossi, and R. Ruiz-Baier, Integrated Heart-Coupling multiscale and multi-physics models for the simulation of the cardiac function, Computer Methods in Applied Mechanics and Engineering Special Issue on Biological Systems Dedicated to William S. Klug, 314, 345 (2017).
[29] K. Perlin, An image synthesizer, ACM SIGGRAPH Computer Graphics 19, 287 (1985).
[30] K. Perlin, Improving noise, ACM Transactions on Graphics 21, 681 (2002).
[31] O. Ronneberger, P. Fischer, and T. Brox, U-net: Convolutional networks for biomedical image segmentation, in Lecture Notes in Computer Science (Springer International Publishing, 2015) pp. 234-241.
[32] K. He, X. Zhang, S. Ren, and J. Sun, Deep residual learning for image recognition, in 2016 IEEE Conference on Computer Vision and Pattern Recognition (CVPR) (IEEE, 2016).
[33] C. Choy, J. Gwak, and S. Savarese, 4D spatio-temporal ConvNets: Minkowski convolutional neural networks, in 2019 IEEE/CVF Conference on Computer Vision and Pattern Recognition (CVPR) (IEEE, 2019).
[34] A. Paszke, S. Gross, F. Massa, A. Lerer, J. Bradbury, G. Chanan, T. Killeen, Z. Lin, N. Gimelshein, L. Antiga, A. Desmaison, A. Kopf, E. Z. Yang, Z. DeVito, M. Raison, A. Tejani, S. Chilamkurthy, B. Steiner, L. Fang, J. Bai, and S. Chintala, Pytorch: An imperative style, high-performance deep learning library, in Advances in Neural Information Processing Systems 32: Annual Conference on Neural Information Processing Systems 2019 (NeurIPS 2019), edited by H. M. Wallach, H. Larochelle, A. Beygelzimer, F. d'Alche-Buc, E. B. Fox, and R. Garnett (2019) pp. 8024-8035.
[35] V. Nair and G. E. Hinton, Rectified linear units improve restricted boltzmann machines (Omnipress, Madison, WI, USA, 2010) p. 807-814.
[36] S. loffe and C. Szegedy, Batch normalization: Accelerating deep network training by reducing internal covariate shift, in Proceedings of the 32nd International Conference on Machine Learning, Proceedings of Machine Learning Research, Vol. 37, edited by F. Bach and D. Blei (PMLR, Lille, France, 2015) pp. 448-456.
[37] J. Lebert, N. Ravi, F. H. Fenton, and J. Christoph, Rotor localization and phase mapping of cardiac excitation waves using deep neural networks, Frontiers in Physiology 12, 10.3389/fphys.2021.782176 (2021).
[38] Example 1: Reconstruction of Three-Dimensional Scroll Waves in Excitable Media from Two-Dimensional Observations Using Deep Neural Networks (provided above).
[39] I. Loshchilov and F. Hutter, Decoupled weight decay regularization, in 7th International Conference on Learning Representations, ICLR 2019, New Orleans, LA, USA, May 6-9, 2019 (OpenReview.net, 2019).
[40] E. Kovacheva, L. Thamer, T. Fritz, G. Seemann, M. Ochs, O. Dossel, and A. Loewe, Estimating cardiac active tension from wall motion—an inverse problem of cardiac biomechanics, International Journal for Numerical Methods in Biomedical Engineering 37, e3448 (2021).
[41] C. Omichi, S. T. Lamp, S.-F. Lin, J. Yang, A. Baher, S. Zhou, M. Attin, M.-H. Lee, H. S. Karagueuzian, B. Kogan, Z. Qu, A. Garfinkel, P.-S. Chen, and J. N. Weiss, Intracellular ca dynamics in ventricular fibrillation, American Journal of Physiology-Heart and Circulatory Physiology 286, H1836 (2004).
[42] M. Warren, J. F. Huizar, A. G. Shvedko, and A. V. Zaitsev, Spatiotemporal relationship between intracellular calcium dynamics and wave fragmentation during ventricular fibrillation in isolated blood-perfused pig hearts, Circulation Research 101, e90 (2007).
[43] J. Weiss, A. Garfinkel, H. S. Karagueuzian, P. S. Chen, and Z. Qu, Early after depolarizations and cardiac arrhythmias, Heart Rhythm 7, 1891 (2010).
[44] N. Voigt, N. Li, Q. Wang, W. Wang, A. W. Trafford, I. Abu-Taha, Q. Sun, T. Wieland, U. Ravens, S. Nattel, X. H. Wehrens, and D. Dobrev, Enhanced sarcoplasmic reticulum calcim leak and increased Na—Ca exchanger function underlie delayed afterdepolarizations in patients with chronic atrial fibrillation, Circulation 125, 2059 (2012).
[45] M. Greiser, W. J. Lederer, and U. Schotten, Alterations of atrial Ca2+ handling as cause and consequence of atrial fibrillation, Cardiovascular Research 89, 722 (2010).
[46] M. Greiser, B.-G. Kerfant, G. S. Williams, N. Voigt, E. Harks, K. M. Dibb, A. Giese, J. Meszaros, S. Verheule, U. Ravens, M. A. Allessie, J. S. Gammie, J. van der Velden, W. J. Lederer, D. Dobrev, and U. Schotten, Tachycardia-induced silencing of subcellular ca2+ signaling in atrial myocytes, The Journal of Clinical Investigation 124, 4759 (2014).

### Example 4: Real-Time Optical Mapping of Contracting Cardiac Tissues With GPU-Accelerated Numerical Motion Tracking

### 4.1. Introduction

Voltage- or calcium-sensitive optical mapping can resolve electrophysiological wave phenomena in cardiac tissue at high spatial and temporal resolutions (1-6), and can be performed across various spatial scales: in hearts as small as a zebrafish's heart (7-9), in single cardiomyocytes and cell cultures (10-13), and large hearts including human hearts (14). Optical mapping supersedes other imaging techniques in terms of spatial resolution and provides the advantage of non-contact measurement.

However, to date, there are two major technical challenges associated with optical mapping which have so far inhibited exploiting the technique's full potential: First, due to its severe sensitivity to motion, optical mapping is yet widely performed in contraction-inhibited tissues to avoid so-called motion artifacts using pharmacological excitation-contraction uncoupling agents. Motion artifacts arise quickly during optical mapping even with the slightest motion if the measurements are performed in an ordinary fashion: extracting and analyzing the optical signals pixel by pixel without numerical motion tracking (15, 16). As a result, over the past 30 years, the motion has been avoided at all costs in optical mapping studies (17). To overcome this limitation, optical mapping has more recently been combined with numerical motion tracking and stabilization techniques to track and measure fluorescence on the deforming tissue surface in a co-moving reference frame, which effectively inhibits motion artifacts (6, 16, 18-20). Second, the high data rates associated with the fast acquisition rates and high spatial resolutions provided by modern high-speed CCD or CMOS cameras lead to large amounts of data, and the handling, processing, and analysis of the data pose a significant challenge, particularly when numerical motion tracking and stabilization is performed. At present, optical mapping is typically performed at imaging speeds of about 200 to 2,000 frames per second (fps) with video image sizes of 100×100, 128×128, or 256×256 pixels, and in some applications even with video image sizes greater than 1,000×1,000 pixels. Accordingly, a single recording results in tens of thousands to millions of parallel optical measurements with data rates ranging in the order of 10⁻¹⁰⁰ million samples per second. With advances in camera sensor technology, the adoption of low-cost high-resolution CMOS cameras (21, 22), and multi-camera imaging systems (23, 24), the data rates will continue to increase, generating large amounts of imaging data that need to be processed.

Myocardial tissue is an electrically excitable tissue, which contracts and deforms in response to electrical excitation, and it is, therefore, crucial to develop measurement tools, with which it is possible to observe electrical wave phenomena and tissue mechanics simultaneously at high spatial and temporal resolutions. Optical mapping combined with computer vision is ideally suited to perform these measurements. The adaptation of computer vision techniques, such as numerical motion tracking, will expand the list of possible applications for optical mapping and provide novel opportunities in cardiovascular research. However, this development also poses a significant challenge in terms of data processing and data handling, which needs to be addressed before the techniques can find more widespread applications.

In this study, 5 numerical motion tracking algorithms were evaluated, which are implemented on a graphics processing unit (GPU), and these algorithms were applied to numerically inhibit motion artifacts in optical mapping recordings. GPU-based algorithms can provide substantial accelerations in terms of processing speed. The performance and applicability of these algorithms to various optical mapping data were evaluated and it was explored whether their use would allow the numerical tracking and compensation of motion artifacts in real-time. All tested algorithms are open-source and freely available as they are implemented in the open-source computer vision library OpenCV (25). As OpenCV is actively maintained, supported, and available on all major operating systems, it should be possible for other optical mapping practitioners to test and replicate the results of this study or use the findings as guidance for their data analysis.

### 4.1.1. Tracking Motion in Fluorescence Imaging Videos

Tracking motion in optical mapping videos is different from tracking motion in most other applications. The particular properties of optical mapping videos can adversely affect the motion tracking process and add significant error to the tracking results (16). Motion tracking algorithms can be broadly distinguished into two categories: either optical flow or feature tracking techniques. Optical flow algorithms are “dense” motion tracking algorithms in that they track motion in every pixel. Feature tracking algorithms track individual points of interest (landmarks, features, etc.) identifiable in an image. Due to the higher resolution of optical flow algorithms, they are better suited to stabilize motion and inhibit motion artifacts in optical mapping videos, because even the slightest sub-pixel shifts can cause severe motion artifacts. The algorithms tested are all dense optical flow estimation algorithms, which track optical flow automatically pixel-by-pixel in a two-dimensional scene. For a survey of various numerical motion tracking techniques refer to (26-28). The term optical flow, or image motion, is used in computer vision to refer to the apparent motion or deformation between two images. This two-dimensional image motion is the projection of the three-dimensional motion or deformation of objects relative to a camera sensor onto its image plane, refer to FIGS. 58A-C. An optical flow field is a two-dimensional vector field describing the displacements of brightness patterns from one image to another. Optical flow can be caused relative motion as well as the deformation of objects. Relative motion is either movement of objects with respect to a static viewpoint or movement of the viewpoint or both.

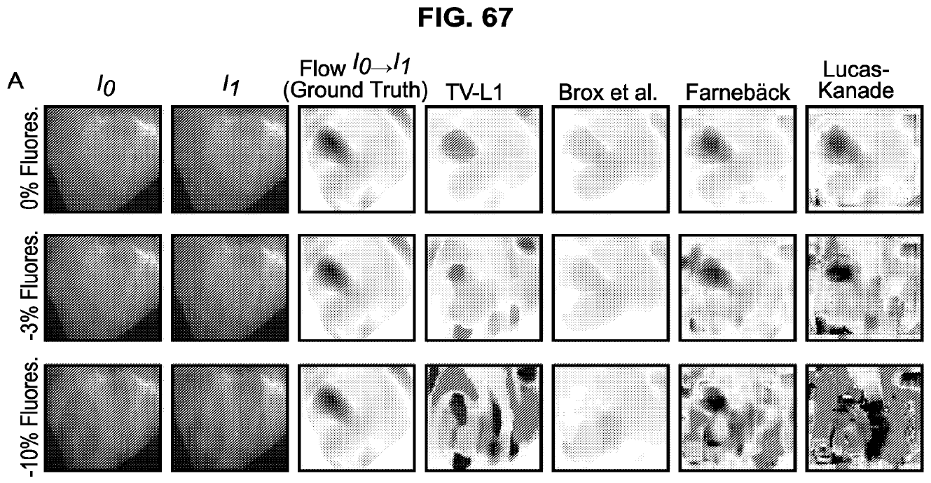

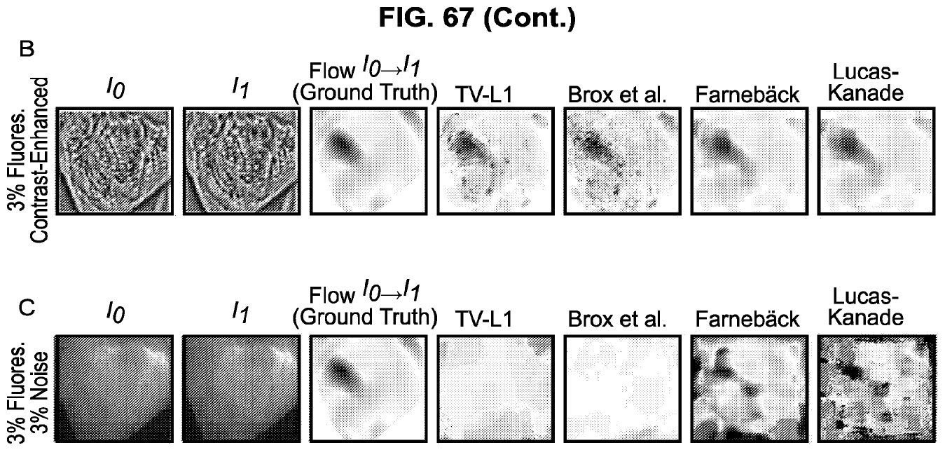

FIGS. 58A-C shows optical mapping combined with numerical motion tracking for measurement of an action potential or calcium waves in moving and deforming cardiac tissues, such as an isolated heart during voltage-sensitive optical mapping. FIG. 58A shows two-dimensional in-plane displacements on the camera sensor depict the motion of the tissue between two video frames from time ti to time tref. FIG. 58B shows optical flow field describing the deformation of the imaged tissue pixel-by-pixel. In FIG. 67, the displacement vector fields are HSV color-coded: the orientation and magnitude of the displacements are depicted by hue (color) and saturation, respectively. FIG. 58C shows numerical motion tracking in a sequence of video images with a calculation of the displacements with respect to a reference frame (first frame or any arbitrary frame).

One key assumption commonly made by optical flow algorithms is brightness constancy, which means that the intensity of objects in a scene does not change over time (29, 30). With optical mapping, however, this assumption is violated because the fluorescent signals, which are caused by (voltage- or calcium-sensitive) fluorescent markers, can change significantly over time. Consequently, the visual appearance of fluorescing tissue can change from video frame to video frame, which makes determining the optical flow and tracking motion between frames challenging. While brightness constancy is almost never perfectly fulfilled in most other real-life scenarios as well, during optical mapping the brightness changes can become particularly severe as they are an intended feature and central part of the measurement: the stronger the brightness changes, the better the measured signal. In previous studies, refer to review by Nesmith et al. (31), the numerical tracking was either facilitated by attaching physical markers to the tissue (18, 32, 33) or performed entirely without markers solely tracking the tissue's natural features (6, 15, 16, 19, 20, 34-36). Particularly in the latter case, when solely the fluorescing tissue is imaged, brightness changes can lead to tracking artifacts (16), and it is, therefore, critical to carefully consider these brightness changes when assessing the performance of numerical motion tracking algorithms. In the experimental results presented here, the GPU algorithm's sensitivity to noise and fluorescence-induced brightness changes was tested.

### 4.2. Methods

Five dense optical flow algorithms were evaluated, which are part of the popular, freely available, open-source computer vision library OpenCV (25): 1) Lucas and Kanade (37) with OpenCV modifications, 2) Farnebäck (38), 3) Brox (39), 4) TV-L1 (40), and 5) the ‘NVIDIA Optical Flow SDK’. All algorithms are executed on a GPU. The algorithms were applied to track and stabilize motion and deformation in voltage- and calcium-sensitive optical mapping videos recorded with 4 different high-speed cameras, as well as in synthetic optical mapping videos which were generated in computer simulations. In particular, the synthetic data provides ground-truth displacement data, which allows a precise evaluation of the results of the tracking algorithms. The performance of the algorithms with regard to processing speed and accuracy on video data with different noise levels and fluorescence intensities were evaluated and compared. The processing speeds of two GPU-based algorithms with their corresponding CPU implementations were also compared. Processing was performed on a workstation with Ryzen Threadripper 3970X 3.7 GHz CPU (AMD Inc., USA) equipped with 32 cores and a Geforce RTX 3070 GPU (NVIDIA Corp. USA). The backbone of numerical motion tracking methods is dense optical flow motion tracking algorithms, which estimate motion or displacements in every pixel of an image, from one image to another, in a fully automatic fashion. All numerical routines were implemented in Python and/or C++.

### 4.2.1. Imaging Data

### 4.2.1.1. Synthetic Data

To evaluate the tracking accuracy of the motion tracking algorithms, synthetic optical mapping videos as described in Christoph and Luther (16) were generated. In short, the synthetic imaging data was created by deforming grayscale video still frames showing a heart during an optical mapping experiment using an electromechanical computer simulation. The video frame was not only deformed but the simulated action potential wave patterns were superimposed as intensity decreases over the parts of the images showing the heart tissue, refer to FIG. 67A. Furthermore, the images were deformed as if the tissue was deforming in response to the action potential wave patterns. The deformed images were subsequently resampled. Because the deformations are simulated, the actual displacements are known in every pixel as ground-truth displacement data. Accordingly, a dataset was created consisting of synthetic image pairs showing the tissue in one and another deformed state at times t1 and t2 and the ground-truth deformation vector field between the two images. The electrical excitation of the simulation was denoted at time t as v (x, y, t) and the deformation with respect to the initial mechanical configuration as d\vec{ }(x,y,t). A synthetic image Ĩ (x, y, t) for a given fluorescence strength f, which denotes the fractional change in fluorescence ΔF/F exhibited by fluorescent probes in optical mapping experiments, is generated as follows:

$$\tilde{I} ( x , y , t ) = ( 1 + f \cdot v ( x , y , t ) ) \cdot {I}_{texture} ( x , y ) \qquad ( 1 )$$

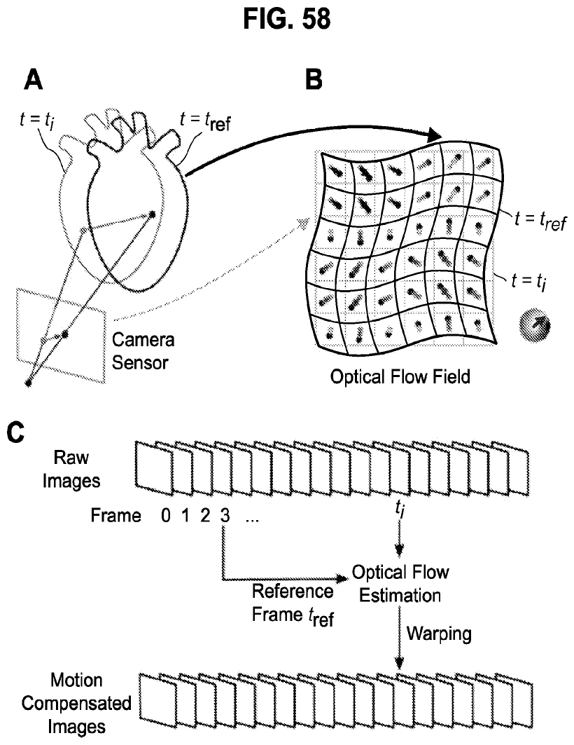

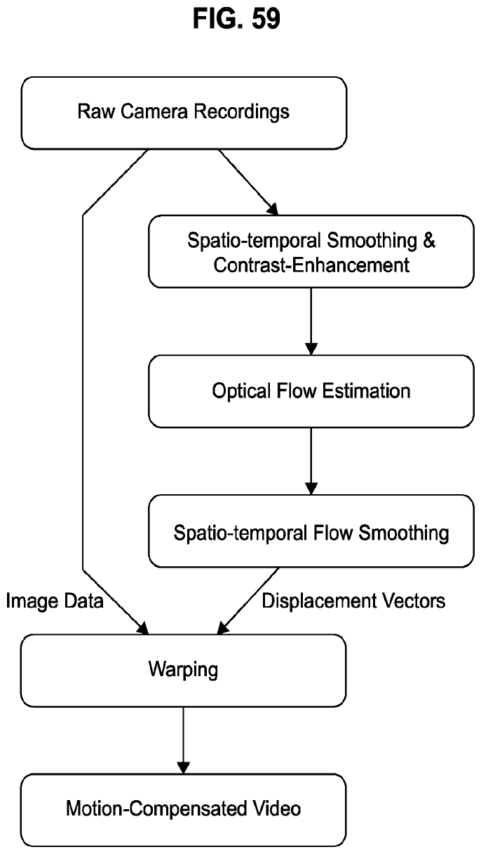

To generate an image pair, the experimental image Iₜₑₓₜᵤᵣₑ (x, y) was chosen, onto which the action potential wave patterns are superimposed, and two simulation time points t₁ and t₂ at random. The first image of an evaluation image pair is Ĩ (x, y, t₁), the second image is the image Ĩ(x, y, t₂) deformed by the displacement vectors u\vec{ }(x,y)=d\vec{ }(x,y,t2)−d\vec{ }(x,y,t1). Here, the displacement vector field u\vec{ }(x,y) is used to warp one image into the other. The central step in the motion-compensation algorithm is the estimation of the deformation vector field u\vec{ }(x,y) between the first and second image using an optical flow algorithm, refer to FIG. 59.

FIG. 59 shows a flow diagram depicting the sequential order of the fully automatic numerical pre- and post-processing and tracking of optical mapping data. Raw videos are smoothed simultaneously in space and time and then contrast-enhanced [also refer to Christoph and Luther (16)] before performing the optical flow estimation. The displacement vectors resulting from the optical flow estimation are smoothed in space and time before being used to warp the original raw video frames to obtain a motion-compensated video.

### 4.2.1.2. Experimental Data

The experimental imaging data processed and analyzed in this study consists of voltage-sensitive optical mapping videos of a mouse and a rabbit heart imaged ex vivo with a Basler acA720-520 μm camera (720×540 pixels full resolution), voltage-sensitive optical mapping videos of a rabbit heart imaged ex vivo with a Brainvision Scimedia MiCAM N256 camera (256×256 pixels full resolution) and an IDS μEye UI-3060CP-M-GL camera (1,936×1,216 pixels full resolution), voltage-sensitive optical mapping videos of a pig heart imaged ex vivo with a Photometrics Teledyne Evolve 128 camera (128×128 pixels full resolution), and calcium- and voltage-sensitive optical mapping videos of a cardiomyocyte culture, differentiated from human induced pluripotent stem cells (iPSCs), imaged in vitro with an IDS μEye UI-3060CP-M-GL camera (1,936×1,216 pixels full resolution). In addition, calcium-sensitive optical mapping data of an isolated stomach strip imaged in vitro with an Oxford Instruments Andor camera (1,280×1,080 pixels full resolution) which were analyzed in another study with methodology was discussed (36). All tissues contract and deform strongly as they were imaged in the absence of pharmacological excitation-contraction uncoupling agents.

### 4.2.2. Optical Flow Estimation Motion Tracking Algorithms

Five different GPU-based dense optical flow estimation algorithms implemented in the popular open-source OpenCV computer vision library (25), version 4.4.0 were tested. The estimation of optical flow between two images is an ill-posed inverse problem, and a solution, a vector field describing the affine transformation of one image into another, can be approximated with optimization techniques. To find the appropriate flow field between two images, optical flow algorithms employ a variety of assumptions on both the data, such as the brightness constancy assumption, as well as the solution, such as smoothness constraints. The choice of assumptions and optimization techniques is an important degree of freedom in designing optical flow algorithms and has led to a wide range of methods over the past several decades (26).

Lucas and Kanade (37) and Horn and Schunck (41) are seminal works in the development of optical flow methods, which broadly represent two different categories of motion tracking algorithms upon which most modern algorithms are based. Lucas and Kanade (37) is a so-called local method: it computes the flow vectors for all pixels separately. It assumes that all pixel intensity changes between the two images are caused by the movement of the underlying objects and that the flow is constant within a local neighborhood of each pixel. Horn and Schunck (41) is a variational method for optical flow estimation: it is a global method that estimates the optical flow for all pixels jointly. It computes the optical flow as the solution to a minimization problem using regularization, relying on the assumption that the brightness of a pixel is constant over time.
Lucas-Kanade: the ‘DensePyrLKOpticalFlow’ implementation in OpenCV was used, which extends the Lucas and Kanade (37) algorithm to give dense optical flow estimates, using an iterative coarse-to-fine scheme (42). This coarse-to-fine approach is a commonly used concept in optical flow estimation to address large displacements: by repeatedly reducing the resolution of the images over several steps, and starting from the smallest image resolution. The Lucas-Kanade algorithm is applied at each resolution and then successively refines the estimated optical flow by applying the Lucas-Kanade algorithm to the higher resolution images.
Farneback: The algorithm introduced in Farneback (38) is a local method similar to Lucas-Kanade. Among other differences, it assumes an affine flow, as opposed to a constant flow, in the local pixel neighborhood.
Brox: Brox et al. (39) proposed a variational method for optical flow estimation, which aims to allow larger displacements compared to Horn and Schunck (41).
TV-L1: There exist several variational algorithms which are based on total variation (TV) regularization using the L1 norm to preserve discontinuities in the optical flow field. Here, the algorithm introduced in Zach et al. (40) was used, whose implementation is described in Sánchez Párez et al. (43).
NVIDIA: In 2019, the NVIDIA Corporation introduced the ‘NVIDIA Optical Flow SDK’, a software library for the estimation of the relative motion of pixels between images. The library relies on dedicated hardware capabilities of NVIDIA Turing and Ampere GPUs for the optical flow computation. The library requires a minimum image size of approximately 160×160 pixels, images sized 128×128 pixels to 256×256 pixels were scaled to overcome this limitation. NVIDIA has not detailed how the optical flow algorithm works but provides the source code upon request.

With all optical flow algorithms, the default parameters in OpenCV were used. The OpenCV implementations of all algorithms except the Brox algorithm require 8-bit images as input. The Brox algorithm also supports 16-bit images. Optical mapping recordings are usually captured using 12-bit (pixel intensity values between 0 and 4,095), 14-bit (pixel intensity values between 0 and 16,383), or 16-bit (pixel intensity values between 0 and 65,535) camera sensors. The synthetic camera images generated were 16-bit images. With the exception of the Brox optical flow algorithm, the generated images were reduced to the 8-bit range (values between 0 and 255) for optical flow estimation. However, there is no loss in image precision for the motion-compensated (warped) videos as the resulting 32-bit floating-point tracking data is then applied to the original data.

### 4.2.3. Tracking Accuracy

The tracking accuracy or tracking error of the algorithms was determined by calculating the average end-to-end point error (EPE), which is the average Euclidean distance between the displacement vectors estimated by the motion tracking algorithms and the ground-truth or reference displacement vectors being the output of simulations or a reference algorithm, respectively. The EPE is averaged over all pixel locations for every displacement vector and then averaged over all vectors in 20,000 video images correspondingly. In this study, the displacement vector fields are shown as color-coded HSV maps instead of vector fields, the hue (color) indicating the orientation and the saturation showing the magnitude of the displacements, respectively, refer to FIG. 58.

### 4.2.4. Pre- and Post-processing

Accurate motion tracking and motion artifact compensation of optical mapping data typically require pre-processing of the imaging data and post-processing of computed displacement vector fields. FIG. 59 depicts a flow diagram of the computational steps for computing a motion-compensated video of a raw optical mapping video. The diagram follows the procedure detailed in Christoph and Luther (16). In short, the pre-processing of experimental data consists of i) smoothing the video data in both space and time (for further details refer to results section) and ii) computing a locally contrast-enhanced version of the original video. The contrast-enhanced video I_{c}(x, y, t) is obtained by renormalizing each pixel's intensity value I(x, y, t) using the maximal and minimal pixel intensities within a small circular region S(x, y, t) around the pixel:

$$( 2 )$$

$${I}_{c} ( x , y , t ) = ( I ( x , y , t ) - min ( S ( x , y , t ) ) ) / ( max ( S ( x , y , t ) ) - min ( S ( x , y , t ) ) )$$

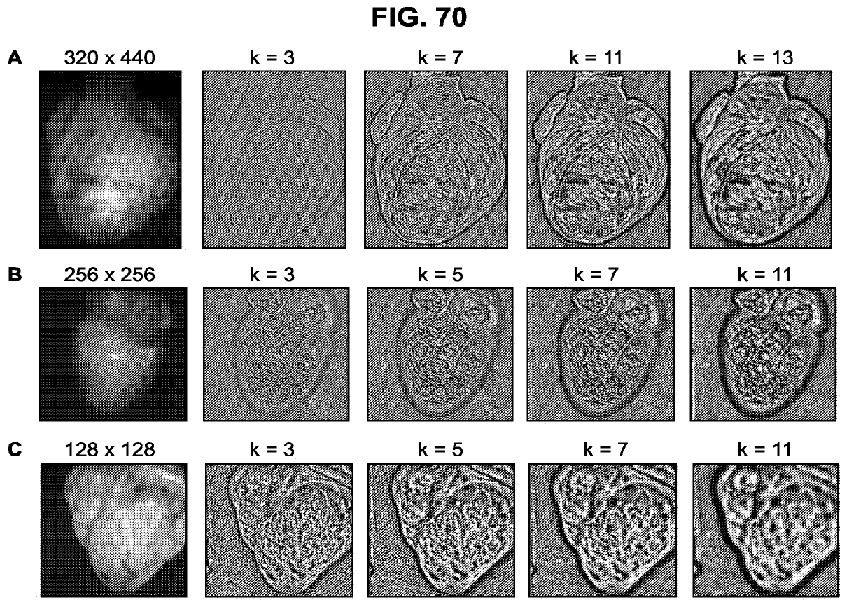

As a result, local grayvalue patterns representing the tissue become amplified yielding videos with maximal spatial contrast, refer to FIG. 70. At the same time, time-varying signals caused by fluorescent reporters are inhibited in contrast-enhanced videos, refer to Christoph and Luther (16). This can reduce tracking artifacts caused by the violation of the brightness constancy assumption in the optical flow estimation, refer to FIG. 67. The necessity of contrast-enhancement and the effect on different optical flow algorithms is analyzed in Section 4.3.2. The optical flow algorithm then estimates the displacement vector field u\vec{ }(x,y,t) of the contrast-enhanced video with respect to a reference frame as shown in FIG. 58. The displacement vector fields u\vec{ } (x,y,t) are then spatio-temporally smoothed (for further details refer to results section) to further reduce tracking artifacts and enforce temporal smoothness. Finally, the motion-compensation video is computed by warping the original raw video with the smoothed displacement vector fields. EPEs were computed on displacement vector fields that were not smoothed.

### 4.3. Results

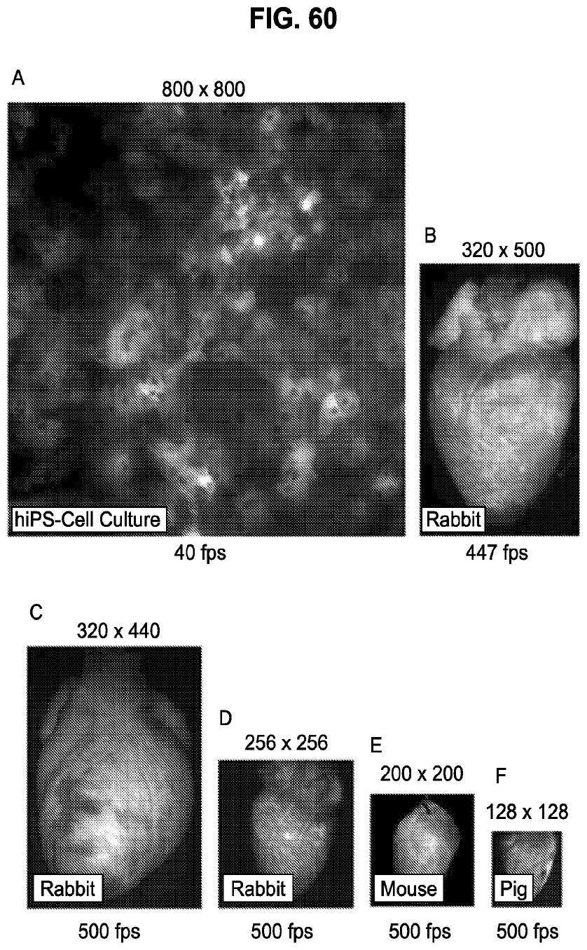

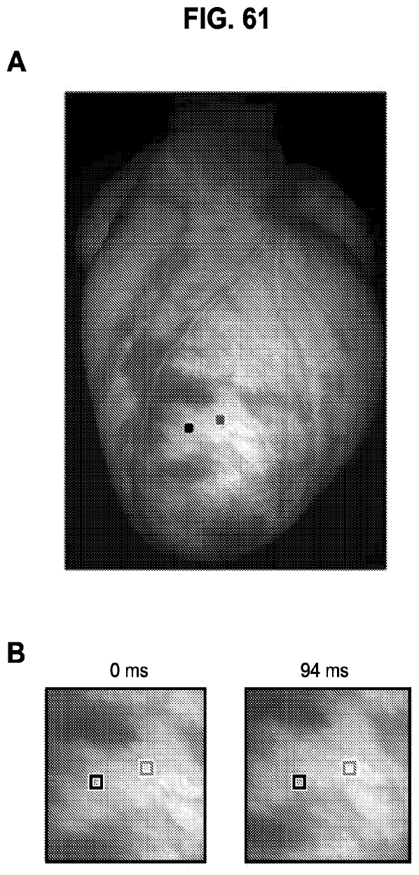

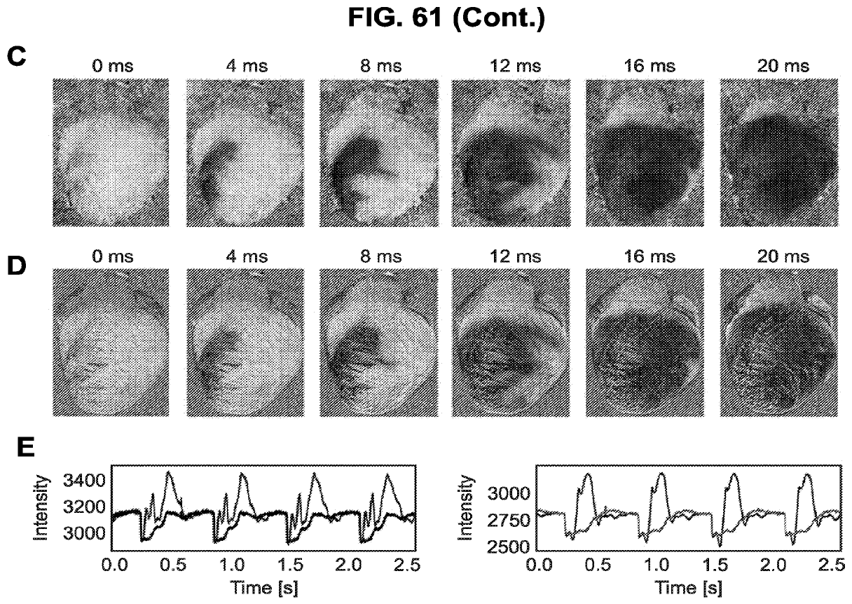

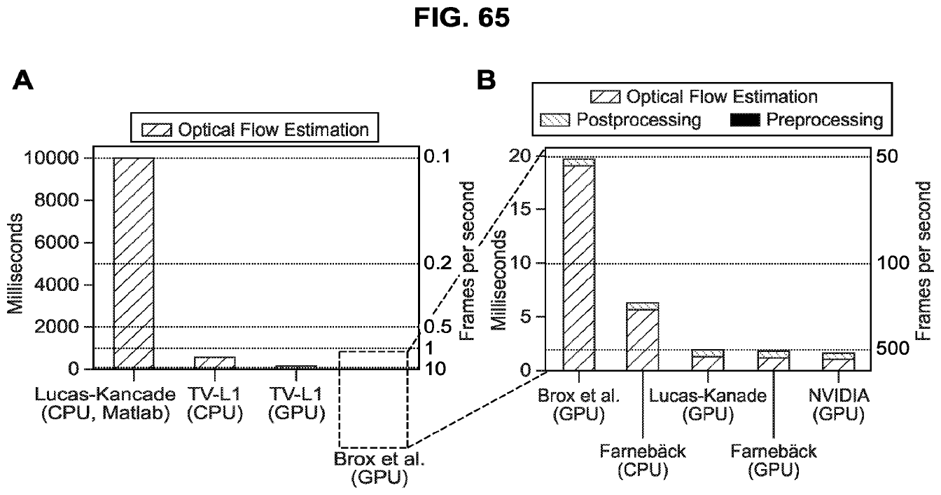

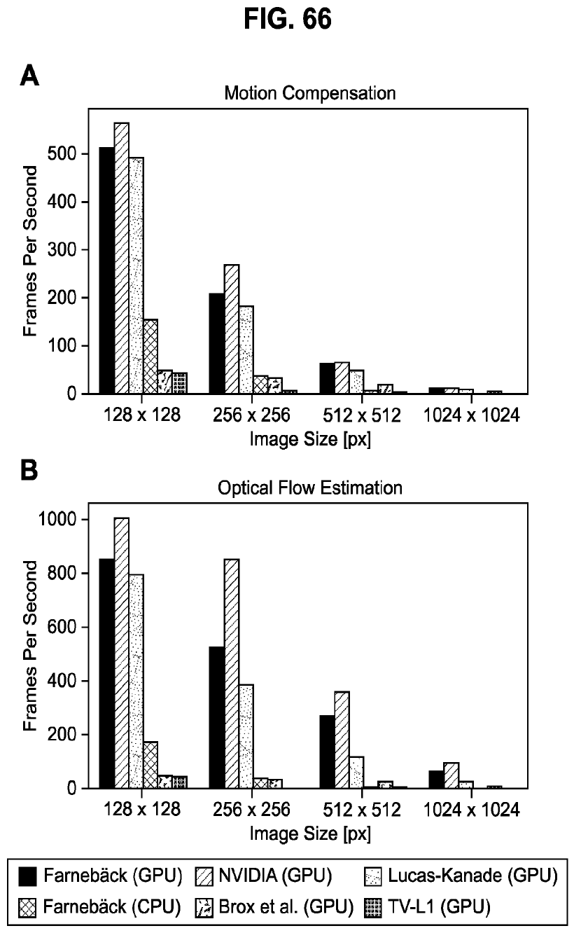

FIGS. 60A-F show typical examples of optical mapping data acquired with 4 different high-speed cameras, the μEye UI-3060CP-M-GL camera by IDS, the acA720-520 μm camera by Basler, the Micam N256 camera by Brainvision Scimedia, and the Evolve 128 camera by Teledyne Photometrics. The 4 cameras, among others, are commonly used in optical mapping studies in basic cardiovascular research. The images show to scale and illustrate the spatial and temporal resolutions that can be achieved with most modern high-speed cameras used in optical mapping studies (although the Micam N256 camera achieves higher imaging speeds than shown). The images are shown in FIG. 60A a human iPSC-derived cardiomyocyte monolayer culture, in FIGS. 60B-D rabbit hearts, in FIG. 60E a mouse heart, and in FIG. 60F a pig heart. All hearts were stained with the voltage-sensitive fluorescent dye Di-4-ANEPPS. All videos were processed using the Farneback motion tracking algorithm, which were determined to be best suited for processing optical mapping data, refer to FIGS. 61-64. With the high processing speeds provided by GPU-based motion tracking algorithms, the video data from low-cost CMOS cameras (the ones by IDS and Basler) was processed, which produce larger video images, at high (near real-time) speeds, refer to FIGS. 61-64. Furthermore, the low-resolution video data (128×128 pixels, acquired at 500 fps) shown in FIG. 60F on the right, as well as the high-resolution video data (800×800 pixels, acquired at 40 fps) shown in FIG. 60A on the left in real-time (using an NVIDIA RTX 3070 GPU, approximately $500) were processed, refer to also Section 4.3.1 and FIGS. 65, 66 for more details on processing speeds. Overall, the GPU-accelerated processing makes the analysis of high-resolution video data and real-time analysis feasible.

FIGS. 60A-F show comparison (to scale) of video data obtained with different cameras in calcium- and voltage-sensitive optical mapping experiments. All hearts were stained with voltage-sensitive fluorescent dye Di-4-ANEPPS. FIG. 60A shows human iPSC-derived cardiomyocyte monolayer expressing the genetically encoded calcium indicator GCaMP-X imaged at 40 fps with 800×800 pixel resolution (0.74 mm field of view) using an IDS μEye UI-3060CP-M-GL camera. FIG. 60B shows rabbit heart imaged at 447 fps with 320×500 pixel resolution using an IDS μEye UI-3060CP-M-GL camera. FIG. 60C shows rabbit heart imaged at 500 fps with 320×440 pixel resolution using a Basler acA720-520 μm camera. FIG. 60D shows rabbit heart imaged at 500 fps with 256×256 pixel resolution using a Brainvision Scimedia MiCAM N256 camera. FIG. 60E shows mouse heart imaged at 500 fps with 200×200 pixel resolution (cropped from original video imaged with 720×320 pixels) using a Basler acA720-520 μm camera. FIG. 60F shows porcine heart imaged at 500 fps with 128×128 pixel resolution using a Teledyne Photometrics Evolve 128 camera. The video data in FIGS. 60A-F can be processed in real-time, faster than the acquisition speeds at 40 and 500 fps, respectively, using GPU-accelerated processing, refer to FIGS. 65, 66.

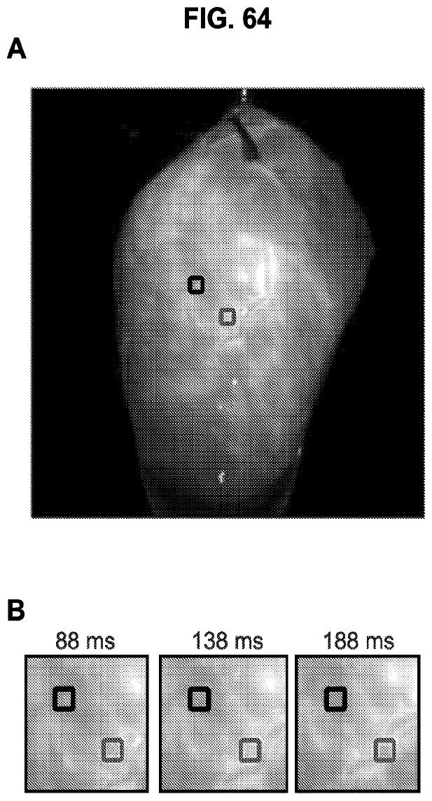

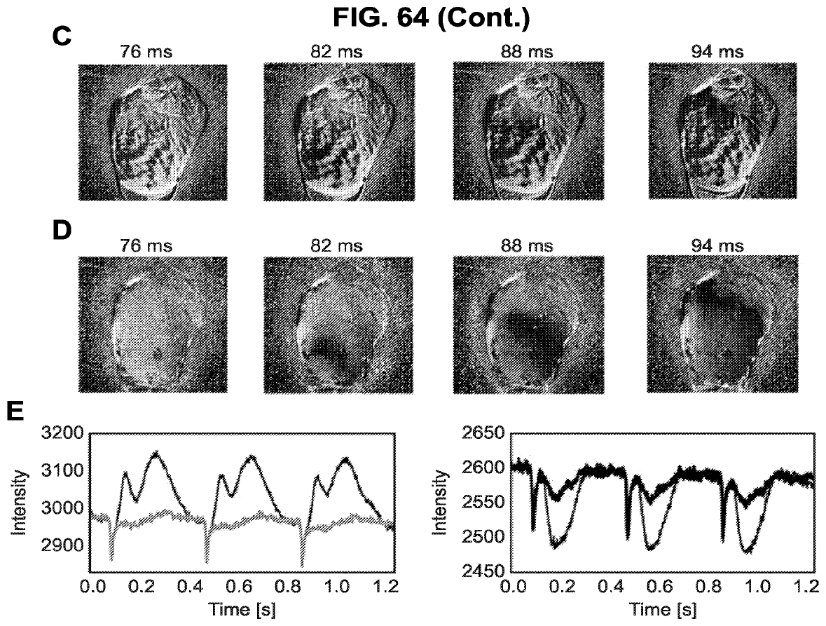

FIGS. 61A-E shows the performance of the Farneback GPU algorithm with sinus rhythm data imaged in an isolated, contracting rabbit heart. The optical maps in FIGS. 61C-D show the effect of the numerical tracking and motion artifact inhibition, while an action potential wave (black: voltage-sensitive staining with Di-4-ANEPPS) propagates across the left ventricle. While motion artifacts are suppressed in FIG. 61C due to the motion tracking, FIG. 61D shows the same but non-stabilized data that was not tracked. The non-stabilized optical maps contain strong motion artifacts. Note that the optical maps were pixel-wise normalized (with a sliding window length of 10 frames or 20 ms), also refer to Section 4.2.4. FIG. 61E shows the optical traces measured in the two locations shown in FIGS. 61A-B before (black) and after (blue and green) numerical motion tracking and motion artifact inhibition. The uncorrected optical traces reflect in large parts motion of the tissue (aside from the rapid downstrokes) and correspond almost solely to motion artifacts, also refer to FIG. 64. The corrected, motion-compensated optical traces exhibit an action potential-like shape. While motion artifacts are significantly reduced in the motion-compensated optical traces, they yet contain residual illumination-related motion artifacts, which cannot be compensated with numerical motion tracking and stabilization alone, also refer to Kappadan et al. (20) and Section 4.4.1. Illumination-related motion artifacts are suppressed in FIG. 61C due to the short sliding window used with the pixel-wise normalization, c.f. FIG. 64 for a comparison with a longer window.

FIGS. 61A-F shows contracting rabbit heart imaged during sinus rhythm using voltage-sensitive optical mapping (staining with Di-4-ANEPPS). Motion artifacts were inhibited using GPU-accelerated numerical motion tracking and motion compensation. FIG. 61A shows video image from original recording (500 fps, 320×440 pixels, Basler acA720-520 μm camera). ROI (green and blue, 6×6 pixels) used to extract optical traces shown in FIG. 61E. FIG. 61B shows motion and deformation of heart surface around ROIs shown in FIG. 61A during diastole and systole (region 100×100 pixels), the movement is about 25 pixels (13% of the total image width). FIG. 61C shows series of pixel-wise normalized (20 ms sliding window) optical maps obtained after numerical motion tracking and motion compensation using the GPU-accelerated Farnebäck algorithm. Action potential wave (black: depolarized tissue) propagating from bottom left to top right across the left ventricle. FIG. 61D shows series of pixel-wise normalized (20 ms sliding window) optical maps without numerical motion tracking and motion compensation. Without motion compensation, the optical maps are distorted by motion artifacts. The action potential wave is yet visible due to the short sliding-window normalization and a relatively strong signal (|ΔF/F|~7%). FIG. 61E shows optical traces averaged from ROIs in FIG. 61A before (black) and after (blue and green) numerical motion compensation using Farneback GPU-based motion tracking algorithm. Motion artifacts become significantly reduced after numerical tracking and stabilization. However, residual illumination-related motion artifacts cannot be overcome with numerical motion tracking alone, refer to Kappadan et al. (20).

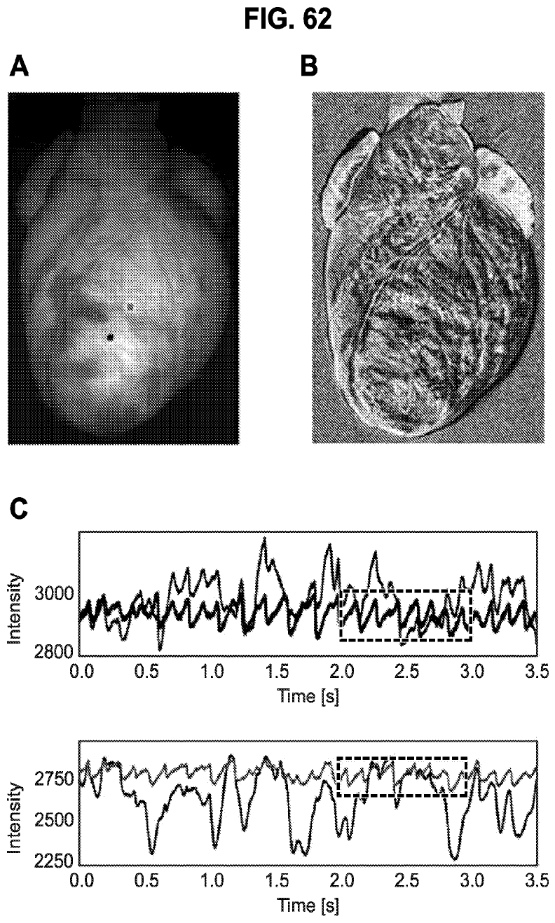

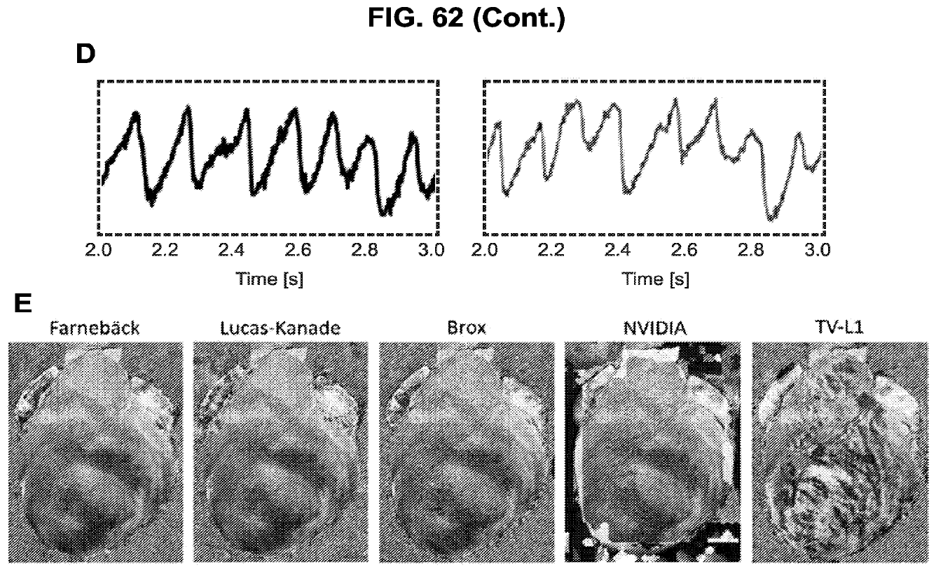

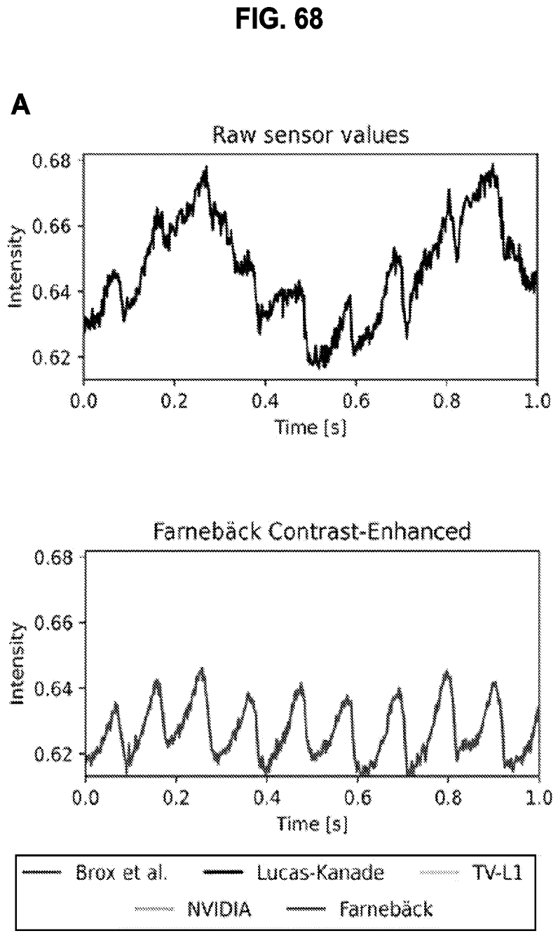

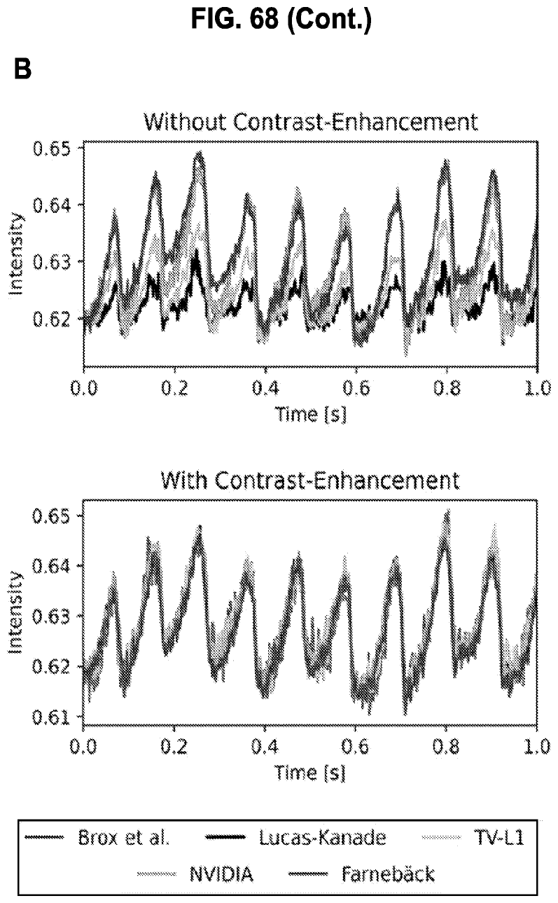

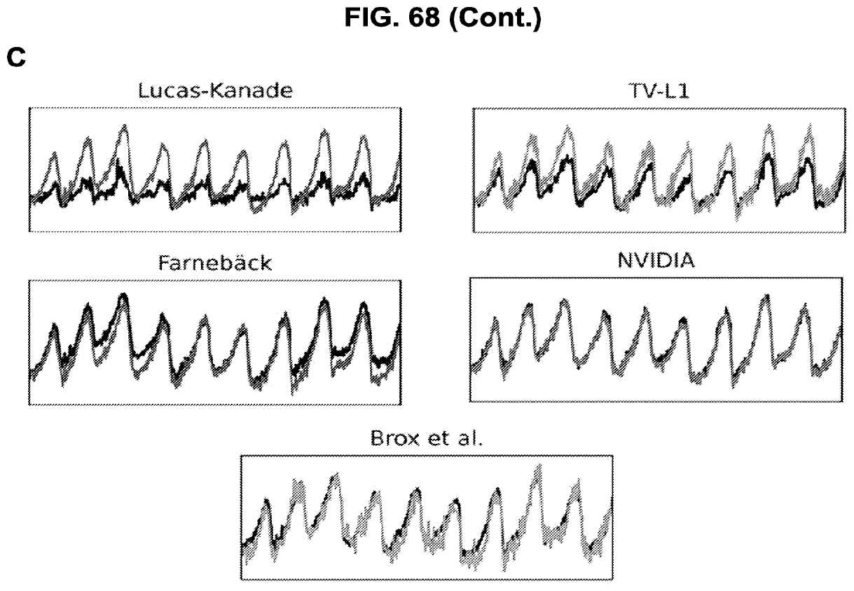

FIGS. 62A-E demonstrate the performance of the numerical motion tracking and motion-stabilization procedure on optical mapping data showing a rabbit heart during ventricular fibrillation. During ventricular fibrillation, the contractile and translational motion of the heart is significantly reduced and illumination-related motion artifacts play less of a role than during sinus rhythm. FIG. 62A shows the original video image. FIG. 62B shows the pixel-wise normalized (0.5 s sliding window) but otherwise unprocessed non-tracked video image. Due to the motion, action potential waves are not visible and the pixel-wise normalized optical maps are dominated by motion artifacts (motion artifacts are also stronger due to the longer sliding window than in FIG. 61). FIGS. 62C-D show the optical traces measured in the two locations in FIG. 62A before (black) and after (blue and green) numerical motion tracking and stabilization. Without numerical motion compensation, the fluctuations of the signal are much larger (approximately 3-5×) and more erratic than with motion compensation. By contrast, with motion compensation, the signals are more regular with more clearly defined action potential upstrokes and a more uniform amplitude across action potentials. FIG. 68A shows shows other examples of optical traces measured during ventricular fibrillation on the contracting heart surface (of a pig heart recorded with the Photometrics camera). Residual baseline drifts in the tracked motion-corrected signals are caused by residual illumination-related motion artifacts and by the relatively long sliding window during the (temporal) pixel-wise normalization. FIG. 62E shows motion-corrected optical maps, which were retrieved with the 5 different GPU motion tracking algorithms: Farnebäck, Lucas-Kanade, Brox, NVIDIA, and TV-L1 (from left to right and sorted by robustness/accuracy). Good results were obtained with the Farneback and the Lucas-Kanade algorithm.

FIGS. 62A-E show contracting rabbit heart imaged during ventricular fibrillation (VF) using voltage-sensitive optical mapping (staining with Di-4-ANEPPS). Motion artifacts were inhibited using GPU-accelerated numerical motion tracking and motion compensation. FIG. 62A shows video image from original recording (500 fps, 320×440 pixels, Basler acA720-520 μm camera). ROI (green and blue, 6×6 pixels) used to extract optical traces shown in FIG. 62C. FIG. 62B shows pixel-wise normalized (0.5 s sliding window) optical map without numerical motion tracking and motion compensation. Without motion compensation, the optical maps are dominated by motion artifacts. Action potential waves are not visible. FIG. 62C shows optical traces measured from two sites shown in FIG. 62A before (black) and after (blue and green) numerical motion compensation using Farnebäck GPU-based motion tracking algorithm. Motion artifacts become significantly reduced. Traces averaged over the two rectangular regions shown in FIG. 62A. FIG. 62D shows Close-up of motion-corrected optical traces in FIG. 62C showing a series of action potentials (downstrokes correspond to action potential upstrokes due to staining). During VF, residual illumination-related motion artifacts are small due to the minimal motion of the heart, refer to Kappadan et al. (20). FIG. 62E shows motion-compensated, pixel-wise normalized optical maps derived with 5 different motion tracking algorithms (Farnebäck, Lucas-Kanade, Brox, NVIDIA, TV-L1; all GPU, sorted from left to right by robustness/accuracy) show vortex-like action potential waves (dark) on the fibrillating ventricular surface.

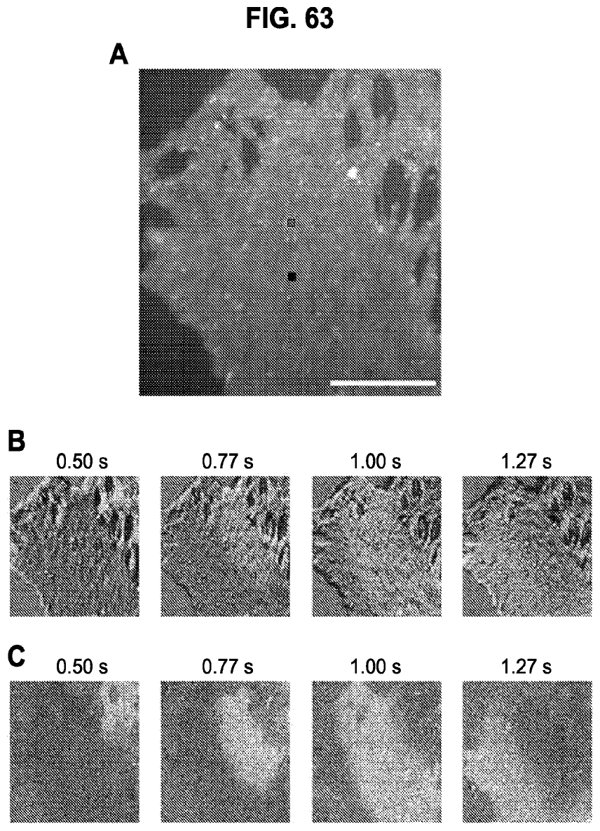

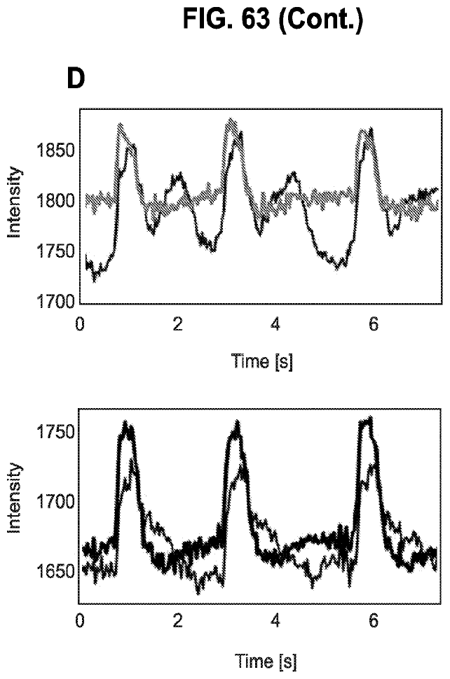

FIGS. 63A-D show the effectiveness of the numerical motion tracking and motion-stabilization procedure using the Farneback motion tracking algorithm with cell culture data. The human iPSC-derived cardiomyocyte culture deforms very strongly. It was stained with the fluorescent dye FluoVolt (resulting in a positive fractional change in fluorescence) and was imaged at 40 fps with 800×800 pixel resolution using the IDS camera. FIG. 63A shows the original video image. FIG. 63B shows the pixel-wise normalized (6 s sliding window) but otherwise unprocessed non-tracked video image. Due to the motion, the pixel-wise normalized optical maps are superimposed by motion artifacts and the action potential wave is barely visible. FIG. 63C shows the tracked and motion-stabilized pixel-wise normalized (6 s sliding window) optical maps without motion artifacts. The action potential is clearly visible as it propagates from the top right to the bottom left corner. FIG. 63D shows the optical traces measured in the two locations in Figure FIG. 63A before (black) and after (blue and green) numerical motion tracking and stabilization.

FIGS. 63A-D show numerical motion tracking and motion artifact compensation performed with Farneback GPU-based motion tracking algorithm with optical mapping video of strongly contracting and deforming human iPSC-derived cardiomyocyte culture stained with voltage sensitive dye (FluoVolt), refer to Supplementary Videos 5, 8. FIG. 63A shows cropped video image (400×400 pixels, scalebar 1 mm) from original recording (40 fps, 800×800 pixels, IDS μEye UI-3060CP-M-GL camera). ROI (green and blue, 10×10 pixels) used to extract optical traces shown in FIG. 63D. FIG. 63B shows pixel-wise normalized (with 4 s sliding window) video without motion compensation with motion artifacts. FIG. 63C shows pixel-wise normalized video after motion compensation without motion artifacts. The action potential wave is visible as a bright wave. FIG. 63D shows optical traces averaged from ROIs in FIG. 63A obtained from videos before (black) and after (red and blue) motion tracking and artifact compensation (y-axis: intensity counts).

FIGS. 64A-E show other examples of sinus rhythm in a contracting mouse and rabbit heart, respectively. As in FIGS. 61-63, the motion was tracked and the effectiveness of the tracking can be evaluated by comparing original and motion-stabilized videos and the respective optical maps. It is important to note that, while numerical motion tracking can effectively inhibit motion, it cannot entirely eliminate motion artifacts with strong motion. The baseline in between two action potentials in the corrected (blue and green) optical traces in FIG. 64E exhibit residual fluctuations. These (often sinusoidally shaped) fluctuations are residual motion artifacts, which are caused by relative motion between the heart and the light sources in the experimental setup. The relative motion persists even after numerical motion tracking and can principally not be removed with numerical motion tracking and stabilization alone, also refer to Kappadan et al. (20) and Section 4.4.1. To overcome these residual motion artifacts, it is either necessary to combine numerical motion tracking with ratiometric imaging, refer to Bachtel et al. (44), Zhang et al. (18), and Kappadan et al. (20), or to estimate and correct for the three-dimensional light-field within which the heart moves, refer to Christoph et al. (19).

FIGS. 64A-E shows numerical motion tracking and motion artifact compensation performed with Farneback GPU-based motion tracking algorithm with optical mapping video of mouse heart during sinus rhythm with voltage sensitive-staining (Di-4-ANEPPS). FIG. 64A shows cropped video image (200×200 pixels) from the original recording (500 fps, 720×320 pixels, Basler acA720-520 μm camera). ROI (green and blue, 6×6 pixels) used to extract optical traces shown in FIG. 64E. FIG. 64B shows motion and deformation of heart surface around ROIs shown in FIG. 64A over time (40×40 pixels). FIG. 64C shows pixel-wise normalized video without motion compensation with motion artifacts. FIG. 64D Pixel-wise normalized video after motion tracking and compensation without motion artifacts. The action potential wave is visible as a dark wave. FIG. 64E shows optical traces averaged from ROIs in FIG. 64A obtained from videos before (black) and after (green and blue) motion tracking and artifact compensation.

Comparing the 5 different motion tracking algorithms, it was found that the Lucas-Kanade algorithm is very sensitive to fluorescence brightness changes, and the TV-L1 and Farneback algorithms are fairly sensitive to these signals. Therefore, it is very likely that these 3 algorithms unintentionally track action potential waves rather than motion with larger fluorescent signal strengths. However, it was confirmed that numerical contrast-enhancement as introduced in Christoph and Luther (16) can alleviate these issues. Contrast-enhancement ensures that the algorithms can be used safely with optical mapping videos in conditions with violated brightness constancy caused by fluorescence. Furthermore, it was found that both the NVIDIA and Lucas-Kanade algorithms exhibit a severe sensitivity to noise, which likely prohibits their use in most fluorescence imaging applications. Overall, it was found that it is possible to perform optical mapping experiments with numerical motion tracking and motion artifact compensation on various optical mapping data, and that GPU-acceleration drastically shortens processing times, which allows real-time or high-throughput processing.

### 4.3.1. Processing Speed

One of the major practical limitations which was faced in previous optical mapping studies with contracting tissues was the long processing times required for the numerical tracking of the data when using conventional algorithms implemented on the CPU. For example, a single-core CPU algorithm implemented in Matlab (45) was used in Christoph et al. (19), Christoph et al. (6), and Christoph and Luther (16) to track motion and the algorithm required about 10 s per 128×128 pixel video frame to compute a motion-compensated version of that frame. By contrast, here, it was shown that GPU-based implementations in OpenCV can achieve a 5,000-fold increase in processing speed and can process more than 500 frames per second on the same data. FIGS. 65, 66 show comparisons of the processing speeds achieved with the TV-L1, Brox, Lucas-Kanade, NVIDIA, and Farneback GPU algorithms and the Lucas-Kanade CPU (Matlab) and TV-L1 and Farnebäck CPU (both OpenCV with Python) algorithms when applied to different optical mapping data. All processing speeds were obtained with the NVIDIA RTX 3070 GPU (approx. $500). FIGS. 65A-B show a comparison of the processing speeds with low-resolution (128×128 pixels) voltage-sensitive optical mapping data, as is also shown on the very right in FIG. 60. FIG. 65A shows a comparison of the processing speeds of the two slower TV-L1 and Brox algorithms with the reference Matlab CPU implementation of the Lucas-Kanade motion tracking algorithm. While it was found that the reference algorithm provides high tracking accuracies (16), it only provided in its particular Matlab implementation processing speeds of about 0.1 fps. By comparison, the OpenCV implementation of the TV-L1 algorithm achieves 1.6 fps on the CPU (running on 1 core) or 4.56 fps on the GPU, respectively, and is, therefore, about 15 times faster on the CPU and about 50 times faster on the GPU than the reference algorithm on the low resolution video data. The Brox motion tracking algorithm achieves processing speeds of 51 fps on the GPU, which is almost 500 times faster than the reference CPU algorithm. The processing speeds of the Brox algorithm and all other faster GPU algorithms cannot be displayed using the same scale on the y-axis as in FIG. 65A. They are therefore shown in a separate FIG. 65B. The processing times shown in FIGS. 65A, B include the time required for the pre-(blue) and post-processing (green) of the video data (smoothing, contrast-enhancement, and warping). The processing times were calculated and are stated in milliseconds per frame pair for 128×128 pixel video images averaged over 10,000 video image pairs. The processing speeds of the other faster GPU motion tracking algorithms in FIG. 65B are several orders of magnitudes faster than both the TV-L1 and the reference CPU algorithm. Even the Brox algorithm, which is much faster than both the reference and the TV-L1 algorithms and can be barely seen in FIG. 65A, is significantly slower than the other algorithms shown in FIG. 65B. The GPU-based Lucas-Kanade, Farnebäck, and NVIDIA motion tracking algorithms achieve processing speeds of 795, 852, and 1,000 fps, respectively, when tracking only motion. Together with pre- and post-processing, the effective processing speeds reduce to 489 fps, 510 fps, and 560 fps on 128×128 pixel video data, respectively. Since the video data was recorded at 500 fps, the processing can be performed in real-time with the NVIDIA and Farneback algorithms. The fast GPU algorithms are about 5,000 times faster than the CPU reference algorithm used in Christoph et al. (6, 19), Christoph and Luther (16), and Kappadan et al. (20). While the pre- and post-processing significantly contributes to the overall processing times with the fast algorithms shown in FIG. 65B, it does not significantly affect the overall processing speeds with the slower tracking algorithms in FIGS. 65A,B. In FIG. 65A the processing time is dominated by the motion tracking, and the pre- and post-processing is negligible in comparison to the time it takes to compute the optical flow fields. Accordingly, with the Brox algorithm, the overall effective processing speed only slightly decreases from 53 fps to 51 fps when taking into account pre- and post-processing. The pre- and post-processing steps are described in more detail in Section 4.2.4. In FIGS. 65, 66, a kernel diameter size of kₓ=k_{y}=3 pixels was used and kₜ=3 time steps for the spatio-temporal smoothing was used and a kernel diameter size of kₓ=k_{y}=7 was used for the contrast-enhancement.

FIGS. 65A-B show comparison of processing speeds for tracking motion in optical mapping recordings using different CPU- and GPU-based optical flow estimation algorithms. Processing times measured in milliseconds per frame pair for 128×128 pixel video images (averaged over 10,000 video image pairs) including pre- and post-processing to obtain motion-stabilized videos were compared. Processing was performed on a workstation with AMD Ryzen Threadripper 3970X 3.7 GHZ CPU and an NVIDIA Geforce RTX 3070 GPU. FIG. 65A shows comparison of a single-core CPU-based Lucas-Kanade reference algorithm (used in Christoph et al. (6) and Christoph and Luther (16)) implemented in Matlab (0.1 fps) with TV-L1 (1.6 fps on CPU, 4.6 fps on GPU, both implemented in Python with OpenCV). FIG. 65B shows the remaining algorithms provide even higher processing speeds with up to a 5,000-fold increase in processing speed: Brox 51 fps (53 fps), Lucas-Kanade 489 fps (795 fps), Farnebäck 148 fps (176 fps) on CPU and 510 fps (852 fps) on GPU, NVIDIA SDK 560 fps (1,000 fps), stated as effective frames per second (fps) with (and without) pre- and post-processing. Preprocessing: contrast-enhancement and spatiotemporal data smoothing as described in Christoph and Luther (16). Post-processing: spatiotemporal smoothing of the computed flow fields and using the flow fields to obtain motion-compensated images (warping). The pre-(0.14 ms per framepair) and post-prossing (0.65 ms per framepair when using GPU-based warping, 0.88 ms when using CPU-based warping) time is the same for all algorithms and is performed on the CPU with parallelization.

Correspondingly, FIGS. 66A-B show a comparison of the processing speeds with 128×128, 256×256, 512×512, and 1,024×1,024 pixel video images, respectively. The data shows that GPU-based processing [particularly the 3 fastest algorithms NVIDIA (GPU), Farnebäck (GPU), and Lucas-Kanade (GPU)] provides substantial increases in processing speed, particularly with larger video images. The required processing time scales linearly with the video image size. FIG. 66A shows the effective processing speeds including pre- and post-processing and FIG. 66B without pre- and post-processing. FIGS. 65, 66 show that with small video images the Farnebäck CPU algorithm (running on 1 core) is competitive with the GPU-based algorithms (approximately 3× slower). When computing the processing times for the GPU algorithms, the spatio-temporal smoothing was executed on the CPU, while the warping was executed on the GPU. When computing the processing times for the CPU algorithms, all pre- and post-processing was executed on the CPU. Pre- and post-processing on the CPU vs. on the GPU (partially CPU) is about 30% slower and does not lead to a substantial overall reduction in processing speed (for videos with 128×128 pixels). A substantial acceleration was not observed with the more expensive and more performant RTX 3080 or 3090 GPU models over the RTX 3070 GPU.

FIG. 66A-B show comparison of processing speeds achieved with 5 different motion tracking algorithms (5 GPU+1 CPU version): Farneback (GPU), NVIDIA SDK (GPU), Lucas-Kanade (GPU), Farnebäck (CPU), Brox (GPU), and TV-L1 (GPU). Processing speeds for video images with 128×128, 256×256, 512×512, and 1024×1024 pixels, respectively. Results were obtained with an NVIDIA Gefore RTX 3070 GPU (approx. $500). FIG. 66A shows processing speeds (frames per second) with pre- and post-processing (contrast-enhancement, spatio-temporal smoothing, motion tracking, spatio-temporal smoothing of displacement vector fields, and warping of videos) to obtain motion-stabilized videos. Motion-stabilized videos can be produced in real-time at 500 fps with the Farneback, NVIDIA, and Lucas-Kanade GPU algorithms with small videos with 128×128 pixels. FIG. 66B shows processing speeds (frames per second) without pre- and post-processing tracking only motion. The Farneback CPU algorithm (running on 1 core) is competitive with the GPU-based algorithms with small video images (approximately 3× slower).

Numerical motion tracking and motion-stabilization are used during optical mapping experiments. The high processing speeds allow one to immediately assess the quality of the videos in between recordings. The Farnebäck GPU algorithm was previously used to track motion and compensate for motion artifacts in calcium imaging data of isolated stomach strips (36). The high-resolution calcium imaging data was acquired at a spatial resolution of 1,280×1,080 pixels and acquisition speeds of 50 fps using a Zyla camera (Andor, Oxford Instruments, UK). With the GPU-based Farnebäck algorithm it became possible to screen and analyze a large number of 15 s long videos containing 750 high-resolution video images, and to effectively reduce motion artifacts and measure calcium transients at high spatial resolutions across large fields of view. Without the high processing speeds, the data analysis would not have been feasible.

### 4.3.2. Motion Tracking Accuracy

The tracking accuracy of numerical motion tracking algorithms depends on various factors, such as scene and video image properties, as well as the type, magnitude, and complexity of the motion, among other factors. Here, it was determined the effect of fluorescence signal strength, measured as the fractional change in fluorescence intensity AF/F, and image noise o onto the tracking accuracy using the synthetic video data described in Section 4.2.1. It was found that all tested GPU algorithms are more or less sensitive to and adversely affected by fluorescence and noise. The tracking accuracy deteriorates with increasing fluorescent signal strengths and noise levels. However, the first issue related to fluorescent signal strength can be resolved using numerical contrast-enhancement. Overall, the Farnebäck algorithm provides the best performance in terms of accuracy and robustness.

FIGS. 67A-C show accuracy of GPU-based motion tracking algorithms applied to synthetic voltage-sensitive optical mapping data. The synthetic data consists of video image pairs showing the tissue in two different deformed states (I₀ and I₁), and dense 2D displacements (hsv-color-code depicts orientation and magnitude) describing the deformation between the two images. In addition, the synthetic video images include action potential wave patterns, which cause a local decrease in fluorescence (−ΔF/F). The ground truth displacements and tracking outcomes are shown next to each other. FIG. 67A shows tracking accuracy with original video with 0, −3, and −10% fluorescence signal strength, respectively. The increasing fluorescent signal causes tracking artifacts, particularly with the TV-L1 and Lucas-Kanade algorithms. FIG. 67B shows tracking accuracy with contrast-enhanced video images with −3% fluorescence signal strength. The contrast-enhancement reduces tracking artifacts with all algorithms. However, with the TV-L1 and Brox algorithms, the contrast-enhancement generates noisy tracking results (the contrast-enhancement also amplifies noise). FIG. 67C shows tracking accuracy with original video images with 3% noise and −3% fluorescence signal strength. All tracking algorithms are very sensitive to noise: the tracking accuracy deteriorates quickly with noise.

FIGS. 68A-C show optical traces obtained from contracting heart surface during ventricular fibrillation (VF) in voltage-sensitive optical mapping recordings before and after numerical motion tracking with 5 different GPU-accelerated motion tracking algorithms (Lucas-Kanade, TV-L1, Farnebäck, NVIDIA, Brox). FIG. 68A Left: shows raw optical trace (black) obtained without numerical motion tracking exhibiting substantial motion artifacts; Right: shows optical trace (green) after numerical motion tracking using Farneback GPU algorithm and motion-stabilization. The optical trace exhibits a series of action potentials (downstrokes correspond to action potential upstrokes). FIG. 68B shows comparison of numerical motion-stabilization using different GPU algorithms; Left: without contrast-enhancement; Right: with contrast-enhancement. Most of the algorithms are less accurate without contrast-enhancement. With contrast-enhancement, all algorithms behave very similarly and provide similar, sufficiently accurate results. FIG. 68C shows individual comparison of algorithms used with contrast-enhancement (color) and without (black).

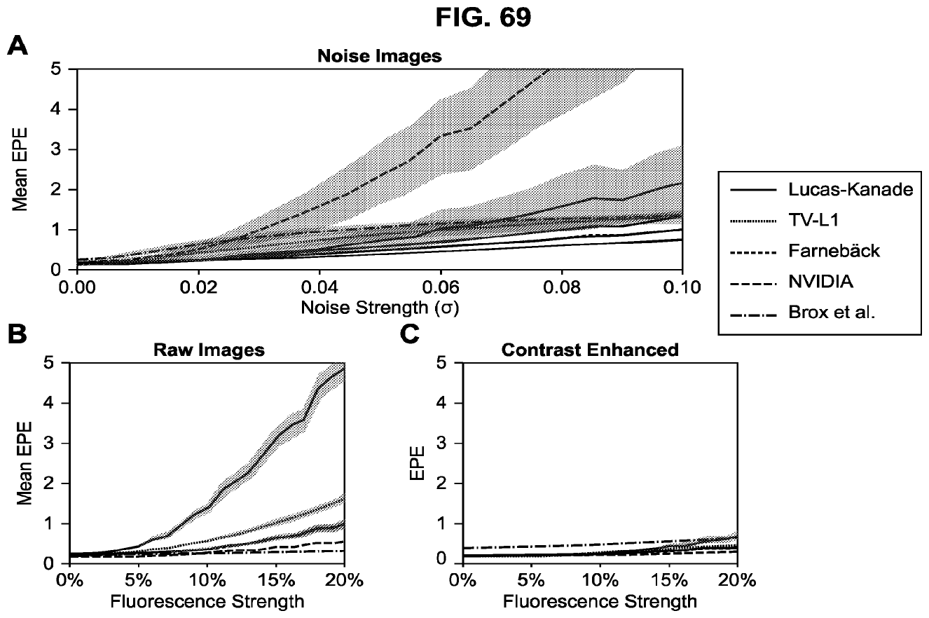

FIGS. 69A-C show comparison of motion tracking error of 5 different GPU-accelerated motion tracking algorithms (Lucas-Kanade, TV-L1, Farnebäck, NVIDIA, and Brox). The error was determined using synthetically generated optical mapping recordings with ground truth displacement data obtained in simulations. The error is measured as the average end-to-end point error (EPE) between the estimated (tracked) optical flow vector field and the ground-truth optical flow vector field. FIG. 69A shows tracking error (EPE) increases with noise standard deviation o. While all algorithms are sensitive to noise, the NVIDIA algorithm is very sensitive to noise and quickly produces larger (than sub-pixel) errors, even at relatively low noise levels. The Farnebäck algorithm is the most robust algorithm against noise. FIG. 69B shows increase in tracking error (EPE error) with increasing fluorescence strength ΔF/F [%]. FIG. 69C shows compensation for steep increase in tracking (EPE) error with increasing fluorescence strength ΔF/F [%] when tracking contrast-enhanced versions of the original videos shown in FIG. 69B.

When tracking motion in videos of fluorescing and contracting tissue, it is crucial to carefully disentangle motion from physiological phenomena encoded within the fluorescence during the processing. The results are otherwise likely to include both motion and tracking artifacts, as described previously in Christoph et al. (16) and already introduced in Section 4.1.1. In the absence of any fluorescent signals or vanishing fractional change in fluorescence (ΔF/F=0%), tissue motion simply corresponds to movements of grayvalue patterns (or optical flow) in the image plane. Most tracking algorithms require or assume brightness constancy when computing displacements of tissue regions in between two grayvalue images. However, because fluorescent indicators produce brightness changes when reporting physiological phenomena, brightness constancy is not fulfilled during optical mapping, see second and third row in FIG. 67A. For example, during the depolarization phase of the action potential the fluorescent dye Di-4-ANEPPS produces a decrease in fluorescence measured behind a near-infrared long-pass filter (because the emission spectrum shifts toward shorter wavelengths during the depolarization). Therefore, action potential waves correspond to grayvalue patterns moving across another moving and deforming grayvalue pattern representing the movements of the tissue, refer to the two left video images in the third row in FIG. 67A with ΔF/F=−10%. In other words, action potential, or calcium waves produce optical flow just as much as movements of the tissue. As a result, tracking algorithms can inadvertently track either or a mix of the two phenomena. This effect was previously described in Christoph and Luther (16) and can be observed when examining the tracking outcome with the TV-L1, Farnebäck, and Lucas-Kanade algorithms in the second and third rows in FIG. 67A. Without any fluorescent signal (|ΔF/F|=0%), all algorithms track the ground truth flow fields sufficiently accurate, refer to the first row in FIG. 67A and the left region in the graph shown in FIG. 69B, which shows the average EPE of each algorithm plotted over fluorescence strength from 0 to 20% absolute fractional change in fluorescence |ΔF/F|. The EPE was determined over a dataset containing 20,000 image pairs. All algorithms exhibit small, sub-pixel tracking errors close to zero at 0% fractional change in fluorescence. However, the EPEs grow quickly with increasing fluorescent strengths: the Lucas-Kanade, TV-L1, and Farnebäck algorithms exceed EPEs larger than 1 pixel with fluorescence strengths above 5, 10, and 20%, respectively. Correspondingly, the 2nd and 3rd rows in FIG. 67A show how the tracked flow fields become less accurate or deteriorate entirely with 3 and 10% fluorescent signal strengths. With the TV-L1 algorithm, the resulting displacement vectors correlate with the shape of the action potential conduction pathway, suggesting that the algorithm inadvertently tracks the action potential. The Brox algorithm is an exception, as it does not appear to be affected by strong fluorescent signals. However, the Brox algorithm produces (too small) displacements, which do not seem to describe the motion of the tissue accurately.

The accidental tracking of action potential or calcium waves can be minimized by generating contrast-enhanced versions of the original videos, as described in Section 4.2 and previously in Christoph and Luther (16) and shown in FIGS. 67B, 68B. In short, the contrast-enhanced videos are calculated by computing the maximal and minimal pixel intensities within a small circular region around each pixel and normalizing that pixel by these two values. As a result, local grayvalue patterns representing the tissue become amplified yielding videos with maximal spatial contrast (contrast-enhancement acts as a spatial high-pass filter while it suppresses temporal fluctuations). At the same time, time-varying signals caused by fluorescent reporters are inhibited in contrast-enhanced videos. The video images in FIG. 67B were calculated using kernels with a diameter of kₓ=k_{y}=11 pixels, also refer to FIG. 70 for a comparison of contrast-enhanced videos with different kernel sizes. As FIG. 67B and the plots in FIG. 69C show, all GPU algorithms produce more reliable tracking results with contrast-enhancement, and the overall EPE becomes substantially reduced with strong fluorescent signals. Mismatches between tracked and ground-truth displacement vectors stay below sub-pixel levels for all algorithms and all fluorescence strengths with contrast-enhancement. In particular, the Farnebäck and Lucas-Kanade algorithms achieve tracking accuracies comparable to when tracking the original videos without fluorescent signals (|ΔF/F=0%|), c.f. row 1 in FIG. 69A. Overall, it appears that contrast-enhancement is a viable approach that works with many different numerical motion tracking algorithms. However. It was found that contrast-enhancement can cause noisier tracking outcomes, particularly with the TV-L1 and Brox algorithms, refer to FIGS. 67B, 68B,C. The tracked displacement vector fields were accordingly smoothed when applying the algorithms to contrast-enhanced video data.

FIGS. 70A-C show contrast-enhancement with different kernel diameters k for three different optical mapping recordings. The kernel size is an important input parameter, which needs to be adjusted to match the properties of the video image. FIG. 70A shows contrast-enhancement with 320×440 video images shown in FIG. 61: the ideal kernel diameter for the contrast-enhancement is about 9 or 11 pixels. FIG. 70B shows contrast-enhancement with 256×256 video image: the ideal kernel diameter is about 7 pixels. FIG. 70C shows contrast-enhancement with 128×128 video image: the ideal kernel diameter is about 3 or 5 pixels. A too small kernel creates noise, whereas a too large kernel reduces the local contrast, enhances large scale gradients, and/or produces too large features.

The sensitivity of the motion tracking algorithms to noise is shown in FIGS. 67C, 69A. The Gaussian noise with zero mean and standard deviation o was added to each pixel of each synthetic video image individually. The amount of noise strength of 3% shown in FIG. 67C corresponds to noise with a standard deviation of σ=0.03 intensity counts added or subtracted on average to or from each pixel. Note that the videos are normalized and contain only pixels with intensity values I_{(x, y)} ∈[0, 1]. The plots in FIG. 69A show how the tracking accuracy of the Lucas-Kanade, TV-L1, Farneback, NVIDIA, and Brox algorithms deteriorates with increasing noise strengths. The EPE was determined over a dataset containing 20,000 image pairs. The NVIDIA and Lucas-Kanade algorithms are the most sensitive to noise: their mean EPE increases steeply with noise levels σ>0.02-0.04. The TV-L1, Brox, and Farneback algorithms are more robust, but their accuracy also degrades significantly with noise. Overall, the Farnebäck algorithm performed best with noise, particularly with noise levels below σ<0.06, and provides sub-pixel accuracies up to noise levels of σ=0.08.

### 4.4. Analysis

The ability to image electrophysiological and mechanical phenomena in contracting tissues simultaneously at high speeds and spatial resolutions is a highly sought-after methodology in physiological research. Optical techniques, such as optical mapping, are in principle ideally suited for this task. However, performing optical mapping with moving tissues is still challenging. In this study, one of the key hurdles in the application of numerical motion tracking to optical mapping data was addressed: identifying motion tracking algorithms that i) are applicable to optical mapping data and ii) provide sufficiently high processing speeds. It was demonstrated that freely-available, open-source motion tracking algorithms implemented on a GPU can be used to track motion in various types of optical mapping data while they also provide substantial increases in processing speeds. High processing speeds are necessary as they open the path for the routine, day-to-day application of numerical motion tracking in optical mapping experiments, open the path for real-time applications, and will allow the high-throughput offline analysis of large video data sets as demonstrated in Vogt et al. (36). In previous studies (6, 16, 19), computations were so time consuming that they prohibited a more systematic and streamlined analysis of optical mapping videos. Now motion artifacts are routinely compensated fully automatically and motion-compensated optical maps can be analyzed on the fly during an optical mapping experiment in a laboratory. The data in this study represent typical use cases, for which it is shown that numerical motion artifact compensation can be performed efficiently suggesting that the methodology could be used routinely by other laboratories. Real-time processing could be utilized to measure and monitor both electrophysiological and mechanical quantities instantaneously, to eventually control experimental parameters in response to the real-time monitoring. It could, furthermore, obviate time-consuming post-processing and the storing of the video data for post-processing. In summary, it was demonstrated that with GPU-based numerical motion artifact compensation it is now possible to perform optical mapping with contracting cardiac tissue with any video camera (with low-cost high-resolution cameras such as the IDS or Basler cameras or more expensive medium to high-resolution cameras such as the Micam N256 or the Andor Zyla cameras, respectively). The high processing rates can be achieved with consumer GPU hardware that costs about $500 (e.g., NVIDIA Geforce RTX 3070 GPU), which could facilitate the more widespread adoption of numerical motion tracking and motion stabilization in basic cardiovascular research.

Overall, the Farneback GPU algorithm is a suitable candidate for tracking motion in optical mapping videos. It provides high processing speeds while being only mildly sensitive to noise and fluorescence artifacts. Among the tested algorithms, it was found to be the most robust algorithm able to handle also large deformations during sinus rhythm. It should be applicable and provide sufficient tracking accuracies in a wide range of optical mapping applications. While the GPU implementations of the Lucas-Kanade and NVIDIA motion tracking algorithms provide similarly high processing rates, they are extremely sensitive to noise, which may limit them to low-noise applications. Furthermore, the Lucas-Kanade algorithm is very sensitive to the fluorescent signal. The Brox algorithm, by comparison, behaves very differently: it is much slower, is robust against fluorescence artifacts, but produces noisy vector fields when tracking contrast-enhanced videos and consistently underestimates motion in the 128×128 pixel video images. Surprisingly, the Lucas-Kanade algorithm is the least robust, most sensitive algorithm of the 5 GPU algorithms. In previous studies (6, 16, 19), a CPU variant of the Lucas-Kanade algorithm (45) was used and achieved high tracking accuracies, which was validated in the same way as in this study with synthetic data. This discrepancy may be related to differences in the implementations of the algorithm ((45) vs. OpenCV). Furthermore, low-light optics and cameras (Teledyne Photometrics Evolve camera) were used and much attention was paid to properly staining and illuminating the tissue to achieve high signal-to-noise ratios. Additionally, the video data was smoothed (using small kernels in the order of 3-5 pixels in diameter) before tracking to suppress residual noise. Therefore, with 5-10% fluorescent signal strength (ΔF/F) and contrast-enhancement, experimental setups were operated in a regime shown on the left in FIG. 69C, where the Lucas-Kanade algorithm still provides high accuracies. This study highlights the importance of the pre-processing (spatio-temporal smoothing) of the video data and confirms the effectiveness of contrast-enhancement to increase the robustness of the tracking. The severe sensitivity of some of the GPU motion tracking algorithms to noise is yet a major limitation, which needs to be addressed in future research.

While there is an abundance of other numerical motion tracking algorithms, the present experimental results were focused on some of the most popular and freely available motion tracking algorithms to establish an overview of the applicability of these ‘classical’ algorithms to optical mapping data. The 5 motion tracking algorithms that were tested are part of OpenCV, an open-source computer vision library, which is freely available on all major operating systems. Therefore, it should be possible to reproduce the findings and use them as a reference and guidance when developing motion tracking software. All algorithms were tested with their default parameters and there may be other parameter regimes that may yet improve the performance of the algorithms. In future research, it is aimed to research other motion tracking techniques and develop custom motion tracking algorithms, which are specifically designed to be used in optical mapping studies.

### 4.1. Limitation: Numerical Motion Compensation Alone Does Not Abolish Motion Artifacts

In these experimental results, a focus was on assessing the performance of different motion tracking algorithms and it was shown that motion tracking can track and stabilize motion in optical mapping videos accurately and can significantly reduce motion artifacts. However, motion tracking alone cannot entirely remove motion artifacts per se. With motion tracking, the optical measurement can be performed in a co-moving frame of reference. This means that the frame of reference is changed from the static laboratory coordinate-based frame measuring the activity pixel-by-pixel to the co-moving frame, in which the motion of the heart is apparently non-existent. However, the relative motion between the (moving) heart and the (static) light sources illuminating the heart is not physically removed by the tracking. Even though the heart's motion is no longer visible in the motion-compensated optical maps, the relative motion persists after motion compensation. Therefore, as the frame of reference changes from static to co-moving, the heart appears to stop moving and instead there is apparent movement in the light sources. The continuing movement of the heart relative to the light sources causes motion artifact. These residual motion artifacts can be seen in FIGS. 61E and 64E. Illumination-related motion artifacts can only be inhibited by combining numerical motion tracking with either i) ratiometric imaging (18, 20, 44), or ii) estimating the motion of the heart in a three-dimensional inhomogeneous light-field and correcting the optical signals for this change in illumination (19). Illumination-related motion artifacts are most critical during sinus rhythm, and less critical during fibrillation, as the illumination varies more strongly with larger amplitudes of motion (20). There are other potential sources for motion artifacts including absorption and reflection changes due to the orientation and compression state of the tissue. However, such effects have never been described in detail.

To date, it is concluded that optical mapping with contracting cardiac tissues remains challenging. Not all measurements which can be performed with pharmacologically contraction-inhibited hearts can equally be performed as easily with contracting hearts. For instance, while it is fair to compute activation maps from sinus rhythm data as shown in FIG. 61C using only numerical motion compensation, or compute dominant frequencies or phase maps from ventricular fibrillation data as shown in FIG. 62E (with the Farneback algorithm) using only numerical motion compensation, it may be necessary to combine numerical motion compensation with ratiometry or numerical light-field correction in order to measure action potential durations accurately from a strongly contracting heart. The future of electromechanical optical mapping is bright. However, further study is necessary until it becomes a widely applicable and easy-to-use imaging technique.

### 4.5. Conclusion

It was demonstrated that it is possible to perform optical mapping with contracting cardiac tissues in real-time and with large video images using GPU-accelerated numerical motion tracking and motion compensation algorithms. Five different motion tracking algorithms were tested on optical mapping data of contracting rabbit, mouse, and pig hearts and cardiac cell cultures imaged with 4 different high-speed cameras as well as on synthetic optical mapping data. It was found that the different motion tracking algorithms can behave very differently. Among the tested algorithms, the Farneback GPU algorithm is one of the fastest and best suited algorithms for optical mapping data providing sufficiently high tracking accuracies and very high processing speeds. Real-time processing speeds were achieved with small videos (128×128 pixels) acquired at 500 fps and with large videos (800×800 pixels) acquired at 40 fps. The high processing speeds provided by GPU-based algorithms could make electromechanical optical mapping more attractive for routine, day-to-day use and open the path for real-time or high-throughput applications.

### 4.6. References

The numbering related to the following references apply with respect to the experimental results presented in Example 4.
[1] Salama G, Lombardi R, Elson J. Maps of optical action potentials and NADH fluorescence in intact working hearts. Am J Physiol. (1987) 252:384-94. doi: 10.1152/ajpheart.1987.252.2.H384
[2] Davidenko J M, Pertsov A V, Salomonsz R, Baxter W, Jalife J. Stationary and drifting spiral waves of excitation in isolated cardiac muscle. Nature. (1992) 355:349-51. doi: 10.1038/355349a0
[3] Efimov I R, Rendt J M, Salama G. Optical maps of intracellular [Ca2+] i transients and action-potentials from the surface of perfused guinea-pig hearts. Circulation. (1994) 90:1469-80. doi: 10.1161/01.CIR.90.3.1469
[4] Witkowski F X, Leon L J, Penkoske P A, Giles W R, Spano M L, Ditto W L, et al. Spatiotemporal evolution of ventricular fibrillation. Nature. (1998) 392:78-82. doi: 10.1038/32170
[5] Gray R A, Pertsov A M, Jalife J. Spatial and temporal organization during cardiac fibrillation. Nature. (1998) 392:75-8. doi: 10.1038/32164
[6] Christoph J, Chebbok M, Richter C, Schröder-Schetelig J, Bittihn P, Stein S, et al. Electromechanical vortex filaments during cardiac fibrillation. Nature. (2018) 555:667-72. doi: 10.1038/nature26001
[7] Hou J H, Kralj J M, Douglass A D, Engert F, Cohen A E. Simultaneous mapping of membrane voltage and calcium in zebrafish heart in vivo reveals chamber-specific developmental transitions in ionic currents. Front Physiol. (2014) 5:344. doi: 10.3389/fphys.2014.00344
[8] Weber M, Scherf N, Meyer A M, Panáková D, Kohl P, Huisken J. Cell-accurate optical mapping across the entire developing heart. eLife. (2017) 6: e28307. doi: 10.7554/eLife.28307
[9] Vandendriessche B, Sieliwonczyk E, Alaerts M, Loeys B L, Snyders D, Schepers D. Optical mapping in hiPSC-CM and Zebrafish to resolve cardiac arrhythmias. Hearts. (2020) 1:181-99. doi: 10.3390/hearts1030018
[10] Rohr S, Salzberg B M. Multiple site optical recording of transmembrane voltage (MSORTV) in patterned growth heart cell cultures: assessing electrical behavior, with microsecond resolution, on a cellular and subcellular scale. Biophys J. (1994) 67:1301-15. doi: 10.1016/S0006-3495 (94) 80602-2
[11] Bub G, Glass L, Publicover N G, Shrier A. Bursting calcium rotors in cultured cardiac myocyte monolayers. Proc Natl Acad Sci USA. (1998) 95:10283-7. doi: 10.1073/pnas.95.17.10283
[12] Entcheva E, Bien H. Macroscopic optical mapping of excitation in cardiac cell networks with ultra-high spatiotemporal resolution. Progr Biophys Mol Biol. (2006) 92:232-57. doi: 10.1016/j.pbiomolbio.2005.10.003
[13] Herron T J, Lee P, Jalife J. Optical imaging of voltage and calcium in cardiac cells and tissues. Circ Res. (2012) 110:609-23. doi: 10.1161/CIRCRESAHA.111.247494
[14] Nanthakumar K, J J, Mass S, Downar E, Pop M, Asta J, et al. Optical mapping of Langendorff-perfused human hearts: establishing a model for the study of ventricular fibrillation in humans. Am J Physiol Heart Circ Physiol. (2007) 293:875-80. doi: 10.1152/ajpheart.01415.2006
[15] Rohde G K, Dawant B M, Lin S F. Correction of motion artifact in cardiac optical mapping using image registration. IEEE Trans Biomed Eng. (2005) 52:338-41. doi: 10.1109/TBME.2004.840464
[16] Christoph J, Luther S. Marker-free tracking for motion artifact compensation and deformation measurements in optical mapping videos of contracting hearts. Front Physiol. (2018) 9:1483. doi: 10.3389/fphys.2018.01483
[17] Swift L M, Kay M W, Ripplinger C M, Posnack N G. Stop the beat to see the rhythm: excitation-contraction uncoupling in cardiac research. Am J Physiol Heart Circ Physiol. (2021) 321: H1005-13. doi: 10.1152/ajpheart.00477.2021
[18] Zhang H, lijima K, Huang J, Walcott G P, Rogers J M. Optical mapping of membrane potential and epicardial deformation in beating hearts. Biophys J. (2016) 111:438-51. doi: 10.1016/j.bpj.2016.03.043
[19] Christoph J, Schröder-Schetelig J, Luther S. Electromechanical optical mapping. Progr Biophys Mol Biol. (2017) 130:150-69. doi: 10.1016/j.pbiomolbio.2017.09.015
[20] Kappadan V, Telele S, Uzelac I, Fenton F, Parlitz U, Luther S, et al. High-Resolution optical measurement of cardiac restitution, contraction, and fibrillation dynamics in beating vs. blebbistatin-uncoupled isolated rabbit hearts. Front Physiol. (2020) 11:464. doi: 10.3389/fphys.2020.00464
[21] Lee P, Calvo C J, Alfonso-Almazn J, Quintanilla J G, Chorro F J, Yan P, et al. Low-cost optical mapping systems for panoramic imaging of complex arrhythmias and drug-action in translational heart models. Sci Rep. (2017) 92:43217. doi: 10.1038/srep43217
[22] Marina-Breysse M, Garca-Escolano A, Vila-Garca J, Reale-Nosei G, Alfonso-Almazn J M, Yan P, et al. A complete and low-cost cardiac optical mapping system in translational animal models. Front Physiol. (2021) 12:696270. doi: 10.3389/fphys.2021.696270
[23] Fan J, Suo J, Wu J, Xie H, Shen Y, Chen F, et al. Video-rate imaging of biological dynamics at centimetre scale and micrometre resolution. Nat Photonics. (2019) 13:809-16. doi: 10.1038/s41566-019-0474-7
[24] Sacconi L, Silvestri L, Rodrguez E C, Armstrong G A B, Pavone F S, Shrier A, et al. KHz-rate volumetric voltage imaging of the whole zebrafish heart. Biophys Rep. (2022) 2:100046. doi: 10.1016/j.bpr.2022.100046
[25] Bradski G. The OpenCV Library. Dr. Dobb's Journal of Software Tools (2000). 26. Raudies F. Optic flow. Scholarpedia. (2013) 8:30724. doi: 10.4249/scholarpedia.30724
[27] Fortun D, Bouthemy P, Kervrann C. Optical flow modeling and computation: a survey. Comput Vis Image Understand. (2015) 134:1-21. doi: 10.1016/j.cviu.2015.02.008
[28] Tu Z, Xie W, Zhang D, Poppe R, Veltkamp R C, Li B, et al. A survey of variational and CNN-based optical flow techniques. Signal Process. (2019) 72:9-24. doi: 10.1016/j.image.2018.12.002
[29] Fleet D, Weiss Y. Optical flow estimation. In: Handbook of Mathematical Models in Computer Vision. Springer-Verlag (2006). doi: 10.1007/0-387-28831-7₋₁₅
[30] Sun D, Roth S, Black M J. Secrets of optical flow estimation and their principles. In: 2010 IEEE Computer Society Conference on Computer Vision and Pattern Recognition. San Francisco, CA: IEEE (2010). p. 2432-9.
[31] Nesmith H W, Zhang H, Rogers J M. Optical mapping of electromechanics in intact organs. Exp Biol Med. (2020) 245:368-73. doi: 10.1177/1535370219894942
[32] Bourgeois E B, Bachtel A D, Huang J, Walcott G P, Rogers J M. Simultaneous optical mapping of transmembrane potential and wall motion in isolated, perfused whole hearts. J Biomed Optics. (2011) 16:096020. doi: 10.1117/1.3630115
[33] Zhang H, Yu H, Walcott G P, Paskaranandavadivel N, Cheng L K, OGrady G, et al. High-resolution optical mapping of gastric slow wave propagation. Neurogastroenterol Motility. (2019) 31: e13449. doi: 10.1111/nmo.13449
[34] Khwaounjoo P, Rutherford S L, Svrcek M, LeGrice I J, Trew M L, Smaill B H. Image-based motion correction for optical mapping of cardiac electrical activity. Ann Biomed Eng. (2015) 43:1235-46. doi: 10.1007/s10439-014-1172-8
[35] Rodriguez M P, Nygren A. Motion estimation in cardiac fluorescence imaging with scale-space landmarks and optical flow: a comparative study. IEEE Trans Biomed Eng. (2015) 62:774-82. doi: 10.1109/TBME.2014.2364959
[36] Vogt M, Schulz B, Wagdi A, Lebert J, van Belle G J, Christoph J, et al. Direct optogenetic stimulation of smooth muscle cells to control gastric contractility. Theranostics. (2021) 11:5569-84. doi: 10.7150/thno.53883
[37] Lucas B D, Kanade T. An iterative image registration technique with an application to stereo vision. In: Proceedings of the 7th International Joint Conference on Artificial Intelligence-Volume 2. San Francisco, CA: Morgan Kaufmann Publishers Inc. (1981). p. 674-9.
[38] Farnebäck G. Two-frame motion estimation based on polynomial expansion. In: Goos G, Hartmanis J, van Leeuwen J, Bigun J, Gustavsson T, editors. Image Analysis. vol. 2749. Berlin; Heidelberg: Springer Berlin Heidelberg (2003). p. 363-70.
[39] Brox T, Bruhn A, Papenberg N, Weickert J. High accuracy optical flow estimation based on a theory for warping. In: Kanade T, Kittler J, Kleinberg J M, Mattern F, Mitchell J C, Nierstrasz O, editors. Computer Vision-ECCV 2004. vol. 3024. Berlin; Heidelberg: Springer Berlin Heidelberg (2004). p. 25-36.
[40] Zach C, Pock T, Bischof H. A duality based approach for realtime TV-L1 optical flow. In: Hamprecht F A, Schnorr C, Jahne B, editors. Pattern Recognition. Berlin; Heidelberg: Springer Berlin Heidelberg (2007). p. 214-23.
[41] Horn B K P, Schunck B G. Determining optical flow. Artif Intell. (1981) 17:185-203. doi: 10.1016/0004-3702 (81) 90024-2
[41] Bouguet J-Y. Pyramidal Implementation of the Lucas Kanade Feature Tracker. Intel Corporation, Microprocessor Research Labs (2000). Available online at: http://robots.stanford.edu/cs223b04/algo_tracking.pdf
Sánchez Pérez J, Meinhardt-Llopis E, Facciolo G. TV-L1 optical flow estimation. Image Process Line. (2013) 3:137-50. doi: 10.5201/ipol.2013.26
[44] Bachtel A D, Gray R A, Stohlman J M, Bourgeois E B, Pollard A E, Rogers J M. A novel approach to dual excitation ratiometric optical mapping of cardiac action potentials with Di-4-ANEPPS using pulsed LED excitation. IEEE Trans Biomed Eng. (2011) 58:2120-6. doi: 10.1109/TBME.2011.2148719
[45] Periaswamy S, Weaver J B, Healy D M, Rockmore D N, Kostelec P J, Farid H. Differential affine motion estimation for medical image registration. In: Aldroubi A, Laine A F, Unser M A, editors. Wavelet Applications in Signal and Image Processing VIII. San Diego, CA (2000). doi: 10.1117/12.408594

### Example 5: Motion Tracking of Fluorescing Tissues using Deep Neural Networks

### 5.1. Introduction

As discussed herein, five classical motion tracking algorithms were evaluated for optical mapping and it was found that, while some of them provide good and better results than others, all require substantial pre-processing of the video data in order to produce reliable results. None of the algorithms could be applied out of the box to optical mapping data, as motion tracking algorithms are susceptible to noise and can inadvertently track action potential waves rather than motion, see FIG. 11b in Christoph and Luther (2018). To remedy the latter issue, a preprocessing filter that amplifies the local contrast in the images is employed. This method, contrast-enhancement, is based on the assumption that the fluorescence changes are small compared to the local contrast. However, based on experience, this contrast-enhancement approach can perform poorly if:
1. Images have insufficient local contrast, leading to areas with a lack of texture. Contrast enhancement relies on the local intensity minimum and maximum within a pixel's vicinity, and if the difference between the two is low, it can lead to salt-and-pepper like noise. This is an issue particularly for cell-cultures, where the change of base fluorescence between nearby pixels is small due to more uniform dye-loading compared to isolated hearts. Consequently, the optical flow algorithm may struggle to accurately estimate motion in these areas.
2. Recordings have a high level of noise. Contrast-enhancement can strongly amplify the noise if it is on the same order of magnitude as the local contrast. As in the previous case, this can lead to tracking errors as the motion tracking algorithm is unable to match the noisy areas of the images.
3. Occlusions occur, such as when part of the tissue or heart moves outside the image frame, rotation or twisting of the heart, or complex three-dimensional deformation. In these cases, the optical flow algorithm may be unable to find sufficient correspondences between the two images for those areas. Boundaries between foreground and background are strongly amplified by the contrast-enhancement step, but they do not necessarily correspond to the same areas of the tissue or heart. It was observed that optical flow algorithms often fail catastrophically in these situations, resulting in large tracking errors that extend over a significant area of the image, which might be due to the smoothness constraints of the motion vector field that the optical flow algorithms attempt to impose.

These issues are regularly encountered, which can limit analyzing the optical mapping recordings in some situations. Ideally, one would want an optical flow algorithm which is robust to these issues and can be applied to optical mapping data without the need for smoothing the data or for contrast-enhancement.

In recent years, the field of computer vision has experienced a paradigm shift in optical flow research, transitioning from classical traditional approaches to deep learning approaches (Zhai et al., 2021; Shah & Xuezhi, 2021). These data-driven techniques have shown remarkable improvements in performance and robustness when applied to a wide variety of complex and challenging scenarios, see e.g. the Sintel (Butler et al., 2012) and KITTI 2015 (Geiger et al., 2012) benchmarks. FlowNet (Dosovitskiy et al., 2015) pioneered the use of deep learning in optical flow estimation, using a encoder-decoder approach similar to the neural network architectures discussed herein. Subsequent convolutional neural networks, such as PWC-Net (Sun et al., 2018) and RAFT (Teed & Deng, 2020), have outperformed classic flow techniques by incorporated domain knowledge and principles from classical optical flow methods into their network architecture. This is remarkable because large-scale datasets with real world imagery and known ground truth dense optical flow fields simply do not exist, as dense flow fields are neither measurable with a sensor nor trivial to annotate by humans. Instead, these supervised models are primarily trained on synthetic optical flow datasets which are generated by rendering 3D scenes with known optical flow, such as FlyingChairs (Dosovitskiy et al., 2015) and FlyingThings3D (Mayer et al., 2016). This is another example of a synthetic-to-real approach discussed herein.

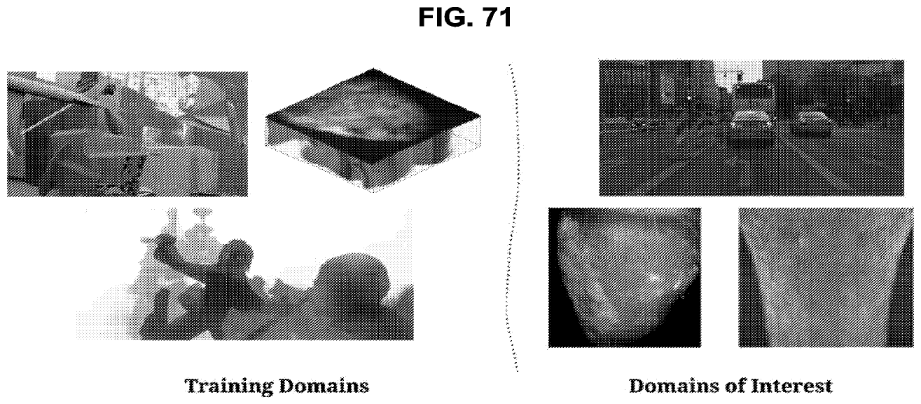

FIG. 71 shows an illustration of the synthetic-to-real approach. Optical flow neural networks are trained on synthetic data (left), with the aim of applying them to real-world (right). Examples for training data are the FlyingThings3D dataset (top left), or the Sintel dataset (bottom), which are generated by rendering 3D scenes. Here, synthetic optical mapping data generated using experimental images and numerical simulations is used (top right of the left panel).

These experimental results present preliminary work on training a deep learning model for optical flow estimation using a synthetic optical mapping image dataset and test it on both synthetic and experimental optical mapping data. The results indicate that this model outperforms classical algorithms in estimating optical flow and can be applied to optical mapping data without requiring contrast enhancement. In effect, the neural network learns to disregard the fluorescence signals and instead focus on the motion of the tissue or heart, creating its own form of contrast-enhancement and other pre- or post-processing (e.g. smoothing). In doing so, it overcomes the limitations of contrast-enhancement discussed herein and the limitations of the brightness-constancy assumption of classical optical flow algorithms.

### 5.2. Methods

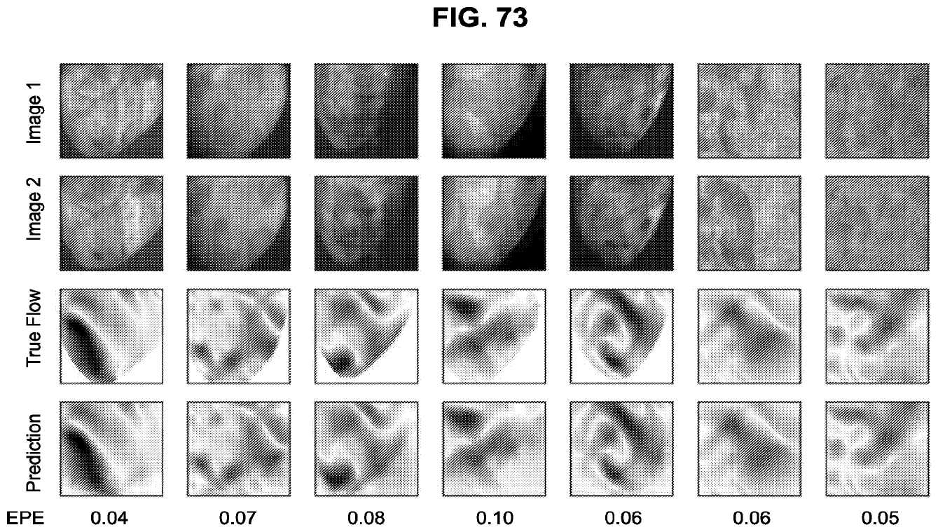

The synthetic training data was generated with the same approach used to evaluate classical optical flow algorithms, see Lebert et al. (2022). In essence, experimental image data is combined with data from numerical simulation of cardiac tissue to generate synthetic image pairs with known deformation and fluorescence. First, a ‘texture dataset’ is generated consisting of single video frames Iₜₑₓₜᵤᵣₑ from various recordings obtained in different voltage-sensitive optical mapping studies, including whole isolated rabbit hearts and hiPSC-derived cardiomyocyte monolayer cultures. If necessary, the images were segmented into foreground and background, which are referred to as the mask in the following. In total, recordings from eight different heart views and four cell cultures were used for the training texture dataset, the validation texture dataset was generated from six separate heart views and four separate cell cultures. Second, a ‘simulation dataset’ was generated consisting of two electrical wave patterns v1, v₂ and a displacement vector field ~u sampled from various simulations of cardiac tissue generated for different studies, e.g. Lebert and Christoph (2019) and Christoph and Lebert (2020). Around 300 different simulations were used, which were split into a training and validation dataset. The simulations are diverse with various different electrical and mechanical parameters and exhibit electrical dynamics ranging from focal, traveling, spiraling to chaotic waves. For each dataset sample, a random simulation and two random simulation time points t1, t2 within it were picked, and the electical excitation states at those two times were used as fluorescence wave dyanmics images. For 3D bulk numerical simulations one layer was chosen at random. The displacement vector field ~u (x, y) was computed as the displacement between the mechanical configuration of the simulation ~d (x, y, t) between these two time points: ~u (x, y)=~ d (x, y, t2)−~ d (x, y, t1). Because the two time points are chosen at random and can be many spiral rotations apart, the displacement ~u does typically not correspond to the electrical dynamics but is a more complicated deformation. In the final step, the ‘texture dataset’ and ‘simulation dataset’ are combined to create the final training and validation dataset of synthetic optical mapping images. In this step, an image Iₜₑₓₜᵤᵣₑ from the texture dataset and an excitation image v (x, y) and displacement field ~u (x, y) from the simulation dataset was chosen at random. A synthetic image I₁ is then generated using:

$${I}_{i} ( x , y ) = ( 1 - f \cdot {v}_{i} ( x , y ) ) \cdot {I}_{texture} ( x , y )$$

where f=ΔF/F is a randomly chosen fluorescence strength between 0% to 30%. Additionally, the mask of the texture image was used to set the v (x, y)=0 for the background. The image I₂ was generated using the same texture image and then deformed by the displacement vector field ~u (x, y), a process commonly known as warping. In total 50,000 training samples and 20,000 validation samples were independently generated for the synthetic optical mapping datasets, one sample consists of the two images I₁, I₂, the ground truth optical flow ~u (x, y), and a mask of the foreground. FIG. 73 shows examples of the generated synthetic optical mapping images. As in Lebert et al. (2022), the spatial resolution of the images is 128×128 pixels. If necessary, e.g. for the cell culture imaging data with larger spatial resolution, a random 128×128 region was used for the texture.

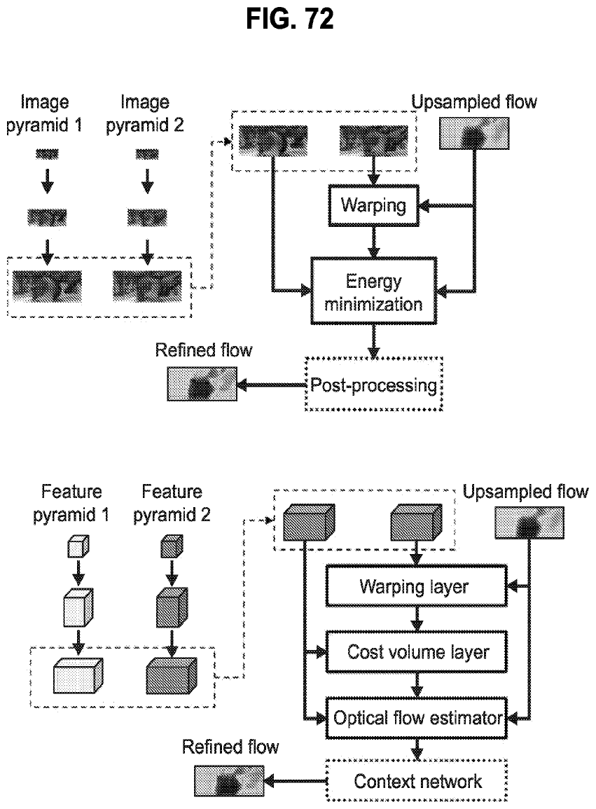

FIG. 72 shows a comparison of PWC-Net architecture and a classical coarse-to-fine approach. Left: Image pyramid with energy minimization-based refinement at each pyramid level. Right: Feature pyramid and refinement at each level by PWC-Net, involving feature warping, cost volume computation. At each step convolutional layers are used, overall the architecture is similar to the other fully convolutional networks discussed herein. Reproduced from Sun et al. (2018).

FIG. 73 shows results on the validation dataset using PWC-Net: each column is a different sample, the first five columns show whole hearts, the last two are images generated from cell culture monolayers. The top two rows show the two grayscale input images, the third row shows the ground truth motion vector field and the forth row shows the predicted optical flow with the end-to-end point error (EPE) displayed below. The optical flow vector fields are HSV color-coded, orientation as hue and magnitude of the displacements as saturation.

PWC-Net (Sun et al., 2018) was selected as the neural network for this study for these experimental results. Its architecture is inspired by three classical optical flow principles: pyramids, warping, and cost volumes, as illustrated in FIG. 72. Classical algorithms often employ image pyramids to address motion across various scales and progressively refine flow estimates. Here, however, the network operates on feature maps generated by the convolutional layers instead of the image data. The network incorporates the physical knowledge optical flow by warping the feature maps with optical flow estimates, and by computing the similarity of features at different potential displacements, known as a cost volume. The implementation of PWC-Net by Ferriere (2018) was the basis for this code, the most notable modifications performed are: 1) the multi-scale L2 loss function was modified so that loss values outside of the texture mask are ignored; 2) the network was trained directly on floating-point grayscale images, instead of 8-bit RGB images; 3) the number of pyramid levels was reduced from 6 to 2 due to the small image size based on initial evaluation. During training, Gaussian white noise was added to the input images as data augmentation, and samples were randomly flipped horizontally and/or vertically. A cyclical learning rate schedule (Smith, 2015) with a minimum learning rate of 0.0001 and a maximum learning rate of 0.0005 and the Adam optimizer (Kingma & Ba, 2015) was used for training. The network with 4,384,088 trainable parameters was trained for 50,000 steps with a batch size of 32 on a Nvidia RTX 2080 GPU with TensorFlow (Martín Abadi et al., 2015) version 1.15.

The tracking accuracy, or tracking error, of the estimated flow fields was evaluated by computing the average end-to-end point error (EPE). EPE is the average Euclidean distance between the displacement vectors estimated by motion tracking algorithms and the ground-truth or reference displacement vectors, which are derived from simulations or a reference algorithm, respectively. EPE is calculated for every displacement vector, averaged over all foreground pixel locations, and then further averaged across all 20,000 video images of the validation dataset. The optical flow vector fields are represented as color-coded HSV maps with hue indicating orientation and saturation denoting magnitude of displacements. When displaying flow vector field maps, the maximum magnitude of the ground-truth flow field is used as a normalization factor for the estimated flow fields.

### 5.3. Analysis

It was found that deep learning-based motion tracking outperforms conventional motion tracking algorithms in terms of accuracy and robustness when tracking motion in fluorescence videos, while having processing speeds comparable to the fastest conventional algorithms. One of the main challenges faced by classical optical flow algorithms is the need for substantial preprocessing, such as contrast-enhancement, to produce reliable results. PWC-Net, on the other hand, was able to accurately estimate optical flow without the need for such pre-processing steps.

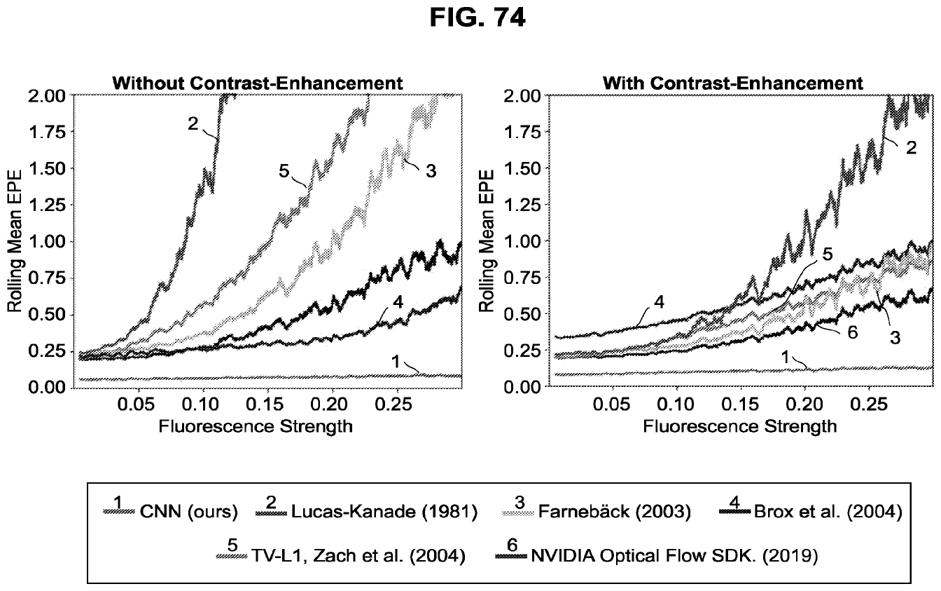

FIGS. 74A-B show tracking accuracy comparison between deep learning-based (brown) and conventional motion tracking algorithms as fluorescent signal strength increases (fractional change in fluorescence ΔF/F). Evaluation on the entire validation dataset by computing the rolling mean EPE using a window size of 500 samples. For the conventional algorithms, errors are computed a) without and b) with the use of contrast-enhancement as preprocessing. The deep neural network provides high accuracies over all signal strengths, while conventional motion tracking algorithms deteriorate quickly with increasing signals without contrast-enhancement. Even with contrast-enhancement the classical algorithms are not as resilient to an increase of fluorescence strength as the CNN.

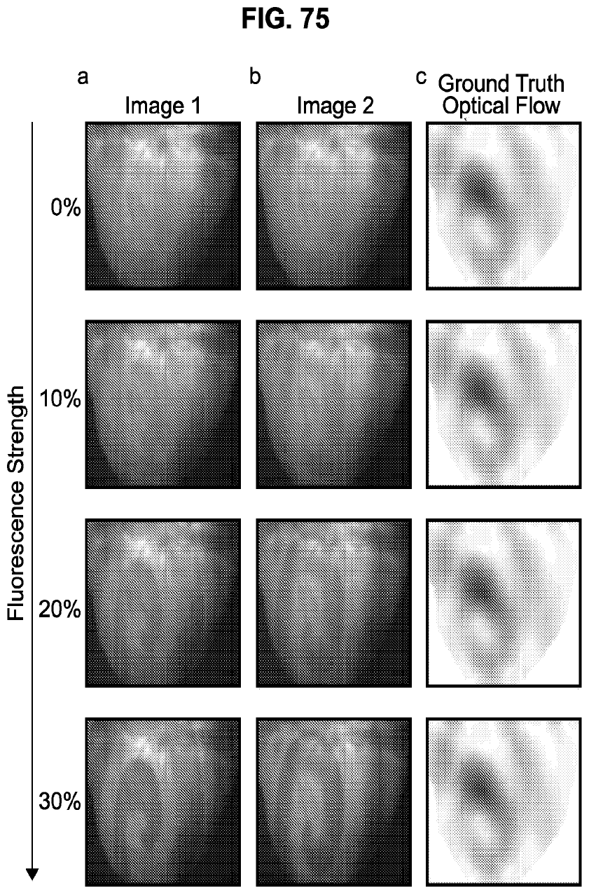

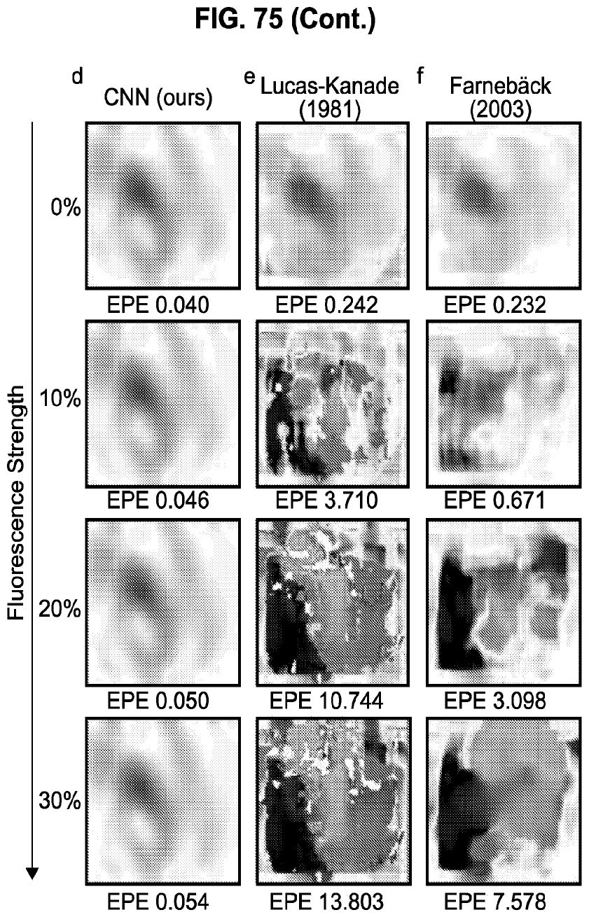

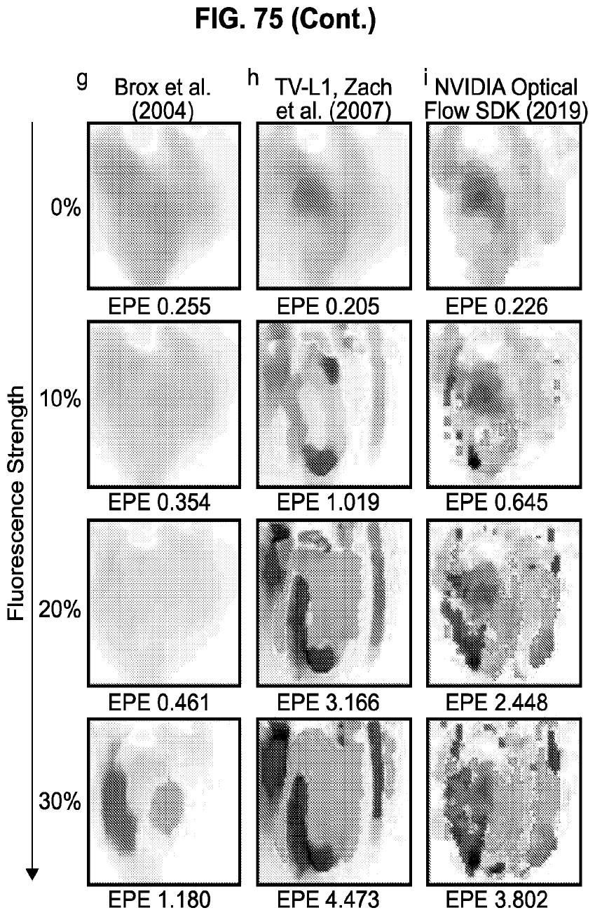

FIG. 75 shows seven samples of the validation dataset and the predicted optical flow vector fields by the neural network. Examples with a high fluorescence strength were chosen for the figure so that the fluorescent wave patterns are clearly visible. The neural network was able to accurately predict the motion fields for each sample. Visually there is little difference between the ground-truth and the predicted optical flow vector maps. Note that the end-to-end point error (EPE) was computed for the foreground pixels only, see ground truth optical flow maps. During training, the loss for the background pixels was set to zero, which is why there is no clear separation between foreground and background in the prediction flow maps. The reason behind this training choice is that the masks are not perfect and the background pixels are not always correctly identified, and for certain applications optical flow in the background pixels is not relevant.

FIG. 75 shows a comparison of deep learning-based motion tracking (column d) with conventional motion tracking algorithms as fluorescence strength increases. Columns a and b show the two input images, column c shows the ground truth optical flow field (identical for each row). Along the vertical direction, the fluorescent signal strength ΔF/F was varied from 0% to 30% for the same sample. While the conventional algorithms (columns e to i) are highly sensitive to the fluorescence, the CNN is robust to the strength of the fluorescence signal.

Across the entire validation dataset consisting of 20,000 samples, the average EPE prediction error for neural network was 0.10±0.07. FIGS. 74A-B show the error in relation to fluorescence strength intensity, comparing PWC-Net (brown) to conventional optical flow algorithms: Lucas and Kanade (1981) (pink), Farnebäck (2003) (red), Brox et al. (2004) (green), TV-L1 Pérez et al. (2013) (orange), and NVIDIA Optical Flow SDK (2019) (blue). In this figure, a rolling mean average EPE using a window size of 500 samples is shown, as the fluorescence strength is randomly distributed across the validation dataset. For the neural network the EPE does not increase with stronger fluorescence, while conventional optical flow algorithms deteriorate quickly. This suggests that the network has learned to ignore the fluorescence wave patterns and instead focuses on the deformation of the tissue. In effect, the network has learned a better version of contrast enhancement and is also more accurate at estimating the motion fields. The performance against classical algorithms with contrast-enhancement pre-processing (kernel size 7) is shown in FIG. 74B. While the conventional optical flow algorithms perform better, high fluorescence strengths still affect them as contrast-enhancement does not completely remove the fluorescence wave pattern. In contrast, for PWC-Net the prediction error shows no dependence on the fluorescence strength. FIG. 75 shows a comparison of the predicted flow fields for different fluorescence strengths for all methods. Columns a and b show the synthetic input images. The fluorescence strength is increased by 10% for each row. The ground-truth in column c is identical for each row. The predictions of PWC-Net (column d) are most accurate. The prediction error increases slightly for this sample as fluorescence strength increases. The predictions of the conventional algorithms (columns e to i) was computed without contrast enhancement, the accuracy of the predictions deteriorates quickly with increasing fluorescence strength.

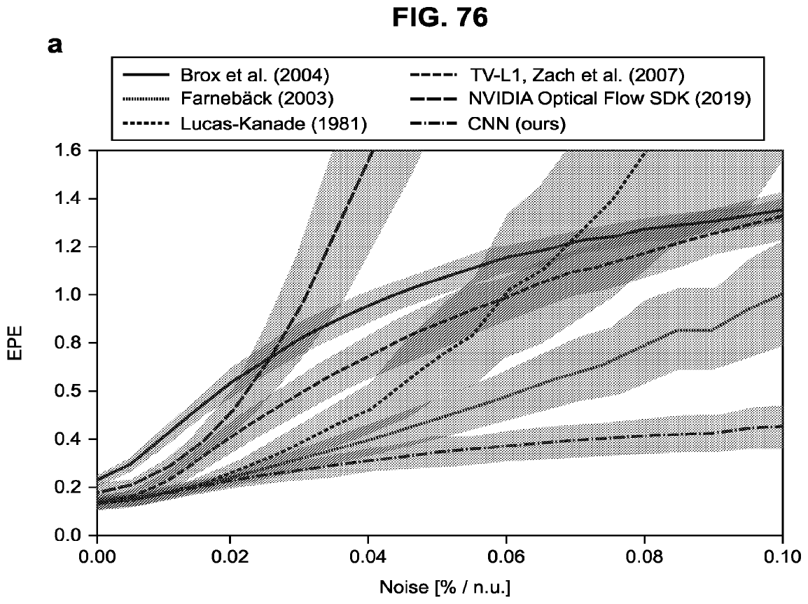

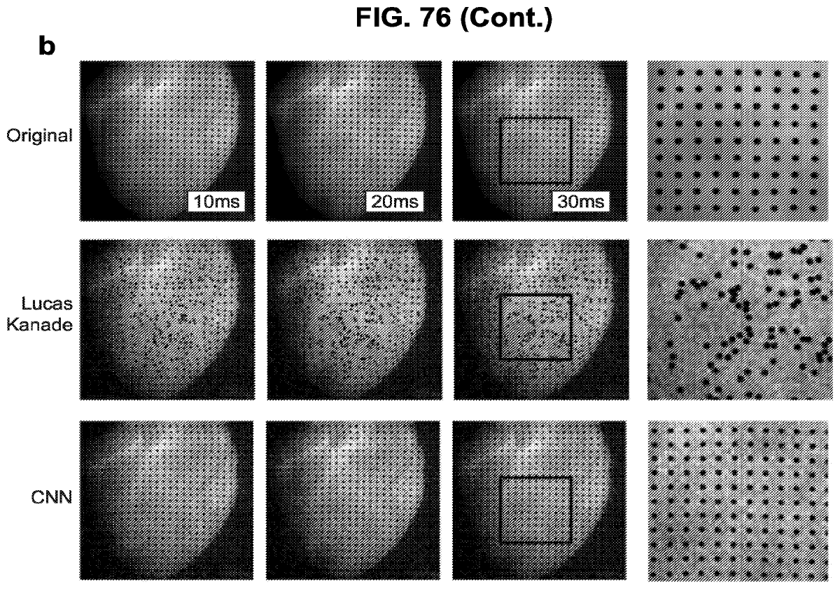

The neural network is also applied to experimental data directly. However, since the ground truth optical flow fields are unknown, reference optical flow fields computed by the methods from Christoph and Luther (2018) were used to evaluate the performance. In particular, the resilience of the network to noise was evaluated by adding Gaussian white noise with standard deviation of up to 0.1 to the normalized video images. A noise level of 0.1 is much stronger than is seen in optical mapping experiments. FIG. 76A shows the comparison of the optical flow methods. The EPE evaluation was computed against a combination of six optical mapping recordings of isolated hearts. Compared to the conventional algorithms, the network was more robust against noise, as the EPE did only increase mildly. For the conventional optical flow algorithms, the pre- and postprocessing step that usually were perform were used, including contrast-enhancement and smoothing of the flow fields. FIG. 76B shows the tracking results for Lucas Kanade and PWC-Net on such a noisy recording (=0.08). Black dots represent the location of material points being tracked throughout the video, see also Christoph and Luther (2018). The first shows the original recording with the reference flow field, the second row Lucas Kanade and third row the tracking from PWC-Net. Lucas Kanade is unable to track the recording, the tracking points do not line up with with the tissue locations. In contrast, PWC-Net shows good tracking results as the tracking locations closely match the reference flow field.

FIGS. 76A-B show tracking accuracy of deep learning-based motion tracking versus conventional motion tracking on experimental video data with added gaussian noise. FIG. 76A shows tracking accuracy declines significantly for conventional tracking algorithms as noise increases, but only mildly with the deep learning-based tracking. Here, the end-to-end point error (EPE) is computed against a reference set of known good flow fields. Contrast-enhanced and other processing steps were used for the for the conventional tracking algorithms. FIG. 76B shows tracking results with substantial noise (σ=0.08) in the video images, three representative frames of a recording. First row shows reference tracking on the original recording with no additional noise, second and third row show the results for Lucas Kanade and PWC-Net respectively. Images on the left show an inset of the tracking results from the third frame.

In terms of processing speed, PWC-Net was comparable to the GPU-accelerated Farneback (2003) algorithm, which were currently predominantly used here and which is characterized in Lebert et al. (2022). For 128×128 pixel images on a Nvidia 2080 GPU both can process around 500 frames per second, when taking into account that Farnebäck requires additional processing steps such as contrast-enhancement.

### 5.4. Analysis

These experimental results demonstrate that utilizing deep learning for motion tracking of moving tissues with fluorescent wave patterns offers a significant advantage over traditional optical flow algorithms in optical mapping data. Tested on both synthetic and experimental data, this approach proved more robust to noise and has the potential to be a powerful solution for motion analysis in optical mapping studies, addressing many of the limitations encountered with traditional optical flow techniques. The PWC-Net architecture effectively managed the challenges posed by optical mapping data and tracked motion even in the presence of strong fluorescence signals. Notably, the tracking accuracy did not deteriorate with increasing fluorescence strength, a factor to which conventional optical flow algorithms are highly sensitive, necessitating preprocessing techniques such as contrast-enhancement to eliminate wave patterns. The network does not require such preprocessing, as it learns its own version of contrast-enhancement to ignore fluorescent wave patterns.

However, these experimental results are preliminary and further work is needed to ensure the network's generalizability. Other aspects to consider include lighting inhomogeneities, blurred or out-of-focus areas in the image, partial obstructions of the image, and other artifacts. One additional challenge is that the network is currently trained to 128×128 pixel images, which are smaller than the imaging data that can currently be acquired. Some embodiments have moved to higher resolution cameras and new deep learning optical flow architectures, which may be more capable. Efforts to extend the network to larger images and more diverse datasets are ongoing, as well as comparisons against other model architectures, such as RAFT (Teed & Deng, 2020).

In these experimental results, 2D deformations of computer simulations were employed to generate optical flow maps. However, the motion of isolated hearts during optical mapping is complex, involving twisting and rotation. The optical flow represents the apparent tissue motion on the imaging sensor, which does not necessarily directly correspond to the displacements obtained from simulations. Other embodiments generate the synthetic optical mapping data by simulating the surface deformation of a 3D heart model as captured by a virtual camera. This would enable the creation of optical flow maps that are more realistic and closely resemble the optical mapping data collected here. Also discussed herein is panoramic optical mapping, where the heart's surface geometry is reconstructed as it deforms. This data could be utilized to train a more advanced optical flow neural network.

### Example 6: Panoramic Voltage-Sensitive Optical Mapping of Contracting Hearts Using Cooperative Multi-View Motion Tracking with 12 to 24 Cameras

### 6.1. Introduction

Imaging action potential waves in contracting beating hearts is a long-standing technical challenge in cardiovascular research [1, 2, 3, 4, 5, 6, 7, 8]. The action potential waves can be measured optically across the surface of isolated hearts using optical mapping, which employs voltage-sensitive fluorescent indicators, high-speed cameras and optical filters. Nevertheless, the optical signals which propagate rapidly across the heart muscle are very small and in order to be able to measure action potential waves it is necessary to combine sensitive optical equipment with precise numerical post-processing [5, 7]. Electrical mapping techniques, such as catheter-based electrode mapping or multi-electrode arrays [9], are a viable option to map electrical impulse phenomena in the heart, but they require physical contact with the tissue, do not allow simultaneous mechanical measurements of tissue strain, and do not achieve the same high spatial resolutions as optical mapping. Optical mapping of action potential waves is routinely done with pharmacologically contraction-inhibited hearts [10, 11, 12] and was even performed on the entire non-moving heart surface using panoramic multi-camera optical mapping [13, 14, 15, 16]. However, optical mapping of action potential waves on the contracting heart surface is much more challenging than in non-contracting hearts. It has been demonstrated in only a small number of studies [2, 3, 4, 5, 6, 7] and in even fewer studies when the measurements were performed on the deforming three-dimensional heart surface using multi-camera optical mapping [2, 3].

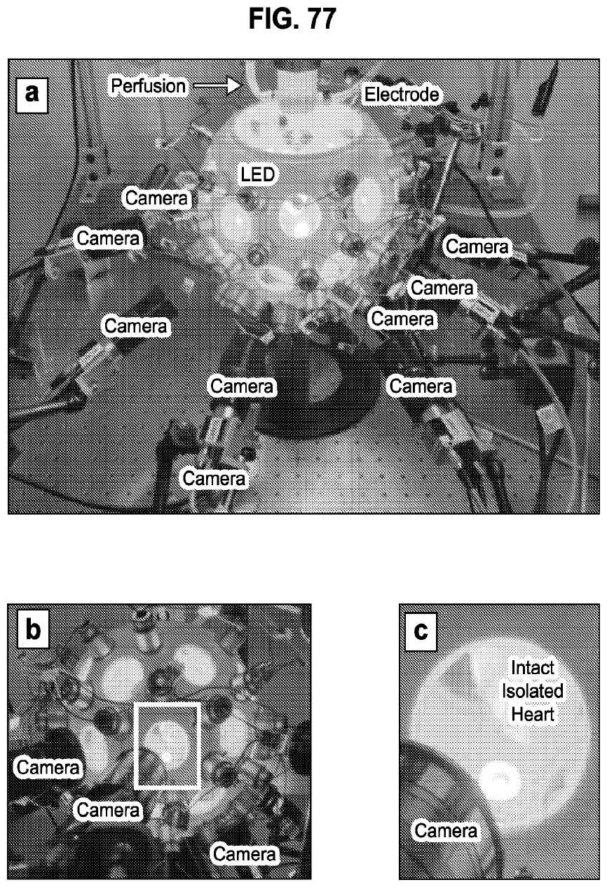

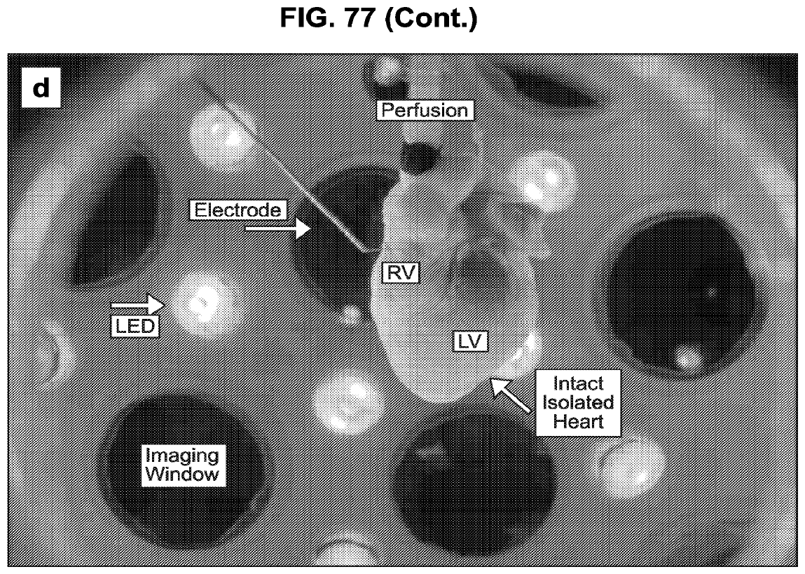

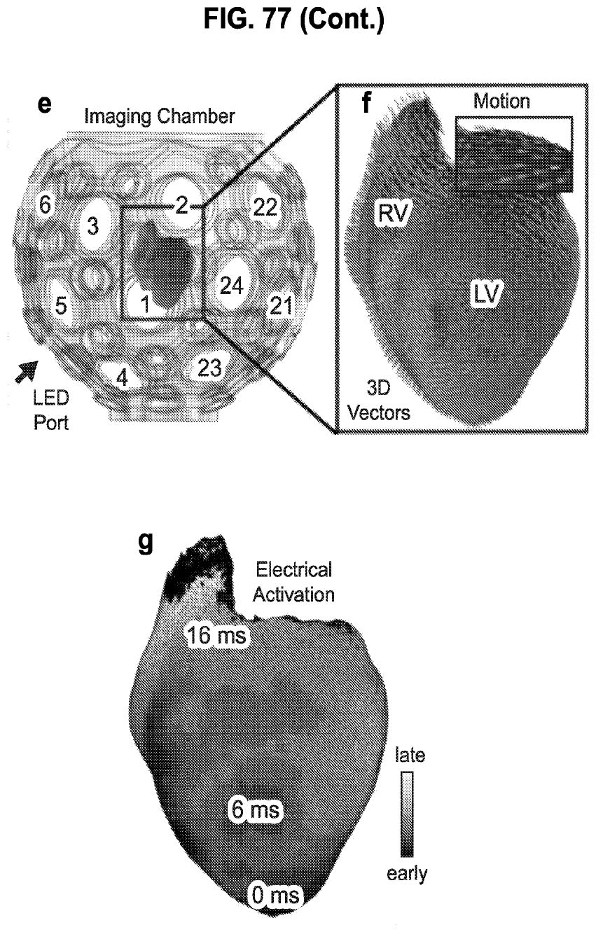

FIGS. 77A-G show an imaging setup for panoramic voltage-sensitive optical mapping of action potential waves on the entire surface of contracting isolated hearts. FIGS. 77A-B show imaging setup consisting of a soccer ball-shaped imaging chamber with 24 windows, a constant-pressure Langendorff-perfusion (tubing and glassware) system, and a multi-camera optical mapping system with 12 high-speed video cameras (acA720-520 μm, Basler). FIG. 77C shows a close-up of illuminated heart inside imaging chamber. FIG. 77D shows an isolated rabbit heart (New Zealand White) immersed in Tyrode solution inside imaging chamber, as seen through one of the 24 windows. The heart can be stimulated using a micro-electrode (optionally). FIG. 77E shows a technical drawing of imaging chamber with 24 large windows for imaging and 48 ports for illuminating the heart with light-emitting diodes (LEDs, 532 nm green center wavelength). The LEDs are positioned around each of the large imaging windows for even illumination of the heart surface. FIG. 77F shows a reconstructed three-dimensional ventricular surface with grayscale video texture map and three-dimensional time-varying displacement vector field (red) indicating motion. FIG. 77G shows an electrical activation map measured on moving three-dimensional ventricular surface.

In the experimental results described here, a panoramic multi-camera optical mapping system is presented, with which action potential waves were imaged, for the first time, across the entire ventricular surface of a fully contracting, strongly deforming heart. The embodiment of the system comprises a custom-designed soccerball-shaped imaging chamber, with which it is possible to position up to 24 high-speed cameras around the heart and simultaneously evenly illuminate the tissue surface with up to 48 light-emitting diodes (LEDs). Using 12 cameras, action potential waves were imaged propagating across the entire contracting ventricular surface of isolated intact rabbit hearts during sinus rhythm, paced rhythms, and ventricular fibrillation. The electrical activation was mapped and epicardial deformation and tissue strain was measured at high spatial (0.5-1.0 million pixels, 150 μm per pixel) and temporal (2 ms) resolutions. The embodiment of the imaging system is not only the first fully panoramic electromechanical optical mapping system covering the entire 360° heart surface, but it also provides unprecedented spatial resolutions and has several key improvements and practical advantages over previously reported three-dimensional electromechanical optical mapping systems [2, 3].

### 6.2. Technical Background

Optical mapping is a highly sensitive measurement, which involves measuring small fractional changes in fluorescence exhibited by fluorescent dyes or genetic indicators in response to a physiological change, such as a change in the transmembrane potential during an action potential. Motion in optical mapping studies is typically associated with so-called motion artifacts, which arise quickly even with the slightest motion [17, 18, 5]. With the normal motion of the heart, and without numerical motion correction, motion artifacts completely compromise the measurements as they override the fractional change in fluorescence, and prohibit further analysis of the data. Numerical motion tracking can effectively suppress motion artifacts [2, 3, 4, 5, 6, 7, 19, 20], and is particularly effective when further combined with ratiometric imaging [2, 6]. Nevertheless, eliminating motion artifacts and measuring artifact-free action potential waves on the contracting heart surface is a non-trivial task and yet a challenge that needs to be solved.

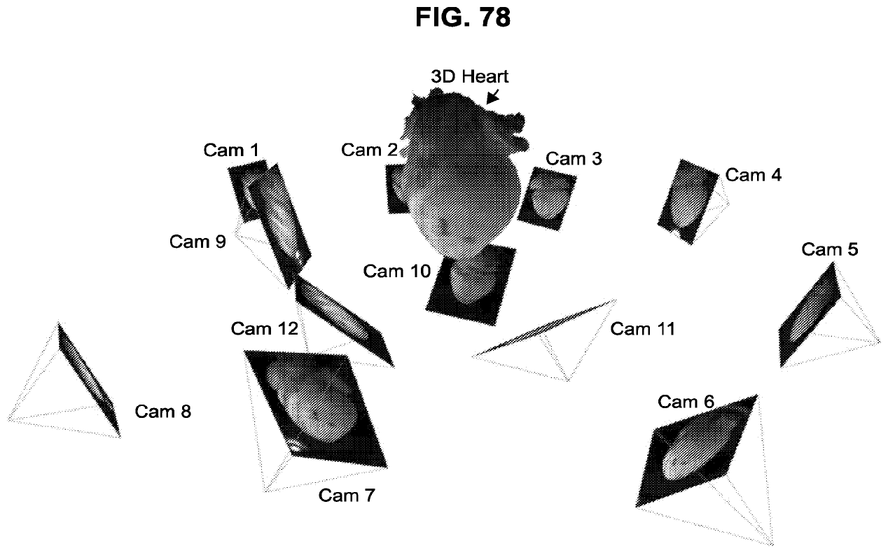

FIG. 78 shows camera poses and three-dimensional reconstruction of an isolated rabbit heart obtained with 12 high-speed cameras (acA720-520 μm, Basler). The video data was used to automatically generate a dynamic reconstruction of the ventricles at 500 frames per second. The heart was stained with voltage-sensitive fluorescent dye (Di-4-ANEPPS) to measure action potential waves across the deforming ventricular surface. Pharmacological excitation-contraction uncouplers, such as Blebbistatin, were not applied in order to image the beating heart. To image the entire ventricular surface, 9 cameras were used horizontally (cameras 1-9, all 12 mm focal length except camera 8), and 3 cameras were used from below (cameras 10-12, 8 mm focal length) to image the ventricles and apex, respectively. The angle between the cameras varied and was roughly 35-45°. The heart was illuminated evenly from the sides and from below with 40 light-emitting diodes (LEDs) positioned around the heart in the LED-ports which are part of the imaging chamber.

Numerical motion tracking and three-dimensional reconstructions of objects are routine tasks in the field of computer vision. However, computer vision techniques are still not extensively utilized in the field of cardiac optical mapping, likely because they require particular technical expert knowledge and are challenging to adopt by cardiovascular researchers. Moreover, three-dimensional optical mapping of contracting hearts is a more challenging problem than most other computer vision tasks, which may have also hindered computer vision experts from entering the field. The technical challenges include: The tissue needs to be tracked precisely, with sub-pixel precision, to be able to measure the optical signals accurately per material coordinate over time and even slight tracking errors can lead to motion artifacts [5]. The optical signals are small and the spatial resolution of the videos is low, typically ranging in the order of 100×100 to 300×300 pixels. The fluorescence changes rapidly over time as it reports physiological function, which can introduce tracking artifacts [5, 7]. Noise, inhomogeneous illumination and other factors can cause further tracking and measurement artifacts. Overall, the video data comprises very particular image features largely determined by the properties of the involved fluorescent indicators and filters. Lastly, the heart's motion and deformation is large, with the heart potentially moving out of the field of view. Together, these factors make optical mapping of contracting tissues a uniquely challenging problem. By default and without further customization, stereoscopic imaging or photogrammetry techniques, which are frequently used for shape reconstruction, lack the precision required for physiological measurements. Accordingly, attempts to reconstruct and optically map the three-dimensional surface of beating hearts were demonstrated in only two studies thus far [2, 3].

Zhang et al. [2] demonstrated for the first time that action potential waves can be imaged on the contracting three-dimensional left ventricular surface using multi-camera optical mapping. The imaging, motion tracking and three-dimensional surface reconstruction was performed with 4 cameras and required fiducial markers glued to the epicardial surface, which were manually identified and then tracked through the video images in a semi-automatic fashion, as described in [21]. Subsequently, the reconstructed three-dimensional heart surface consisted of a low-resolution polygon surface comprising about 20-30 polygons with vertices defined by the fiducial markers used for tracking. Action potential measurements were averaged within each of the polygons without exploiting the full 128×128 pixel resolution of the video images. The cameras were positioned at an angle of 45° to allow sufficient overlap between their fields of view.

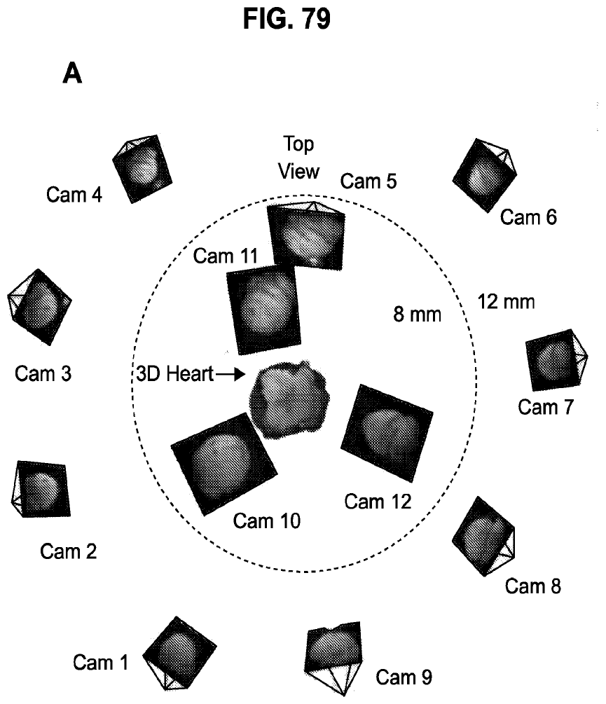

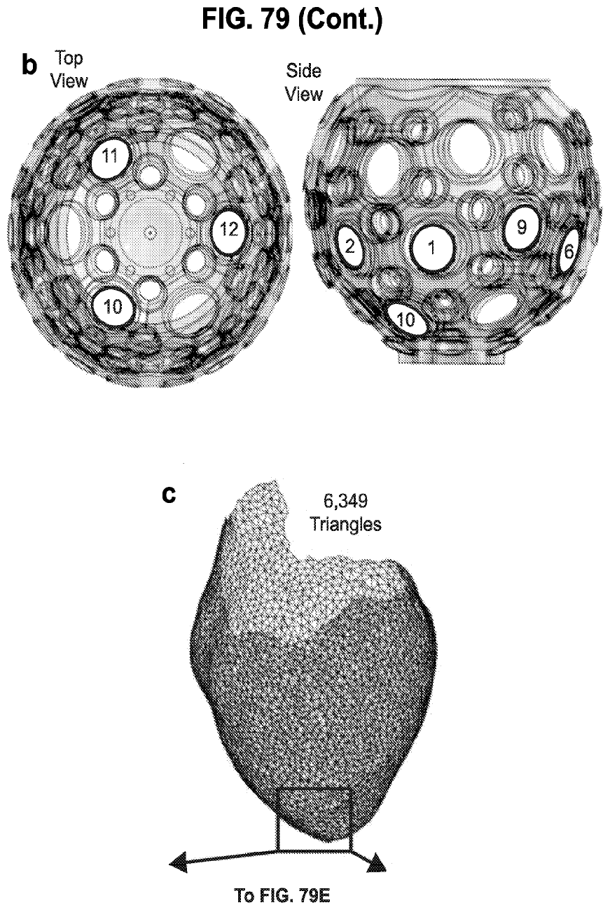

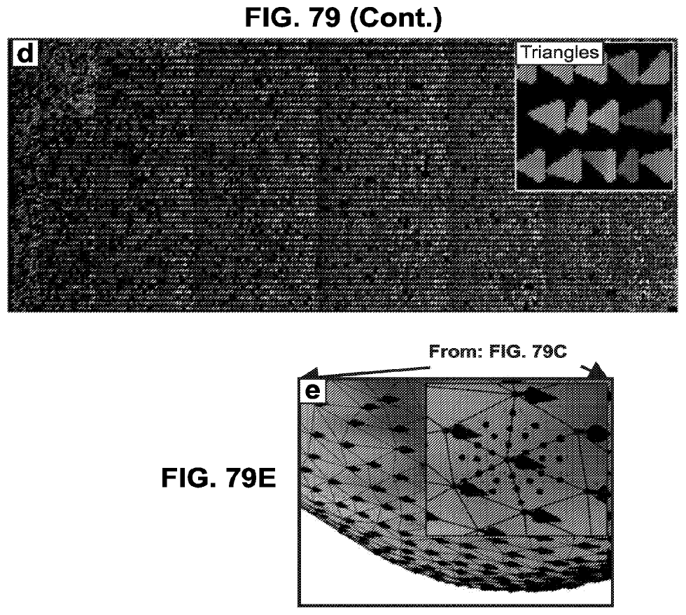

FIGS. 79A-E show an imaging configuration for panoramic voltage-sensitive optical mapping of the entire contracting ventricular surface with 12 cameras. FIG. 79A shows camera poses and 3D reconstruction of rabbit heart (top view). Imaging was performed with 8 mm and 12 mm lenses with shorter and longer working distances, respectively. FIG. 79B shows technical drawing (top & side views) of soccerball-shaped imaging chamber. Horizontal imaging was performed through windows 1-9 and apical imaging through windows 10-12. FIG. 79C shows reconstructed ventricular epicardial surface displayed as a wireframe mesh consisting of 3,235 vertices and 6,349 polygon triangles, respectively. FIG. 79D shows all 6,349 polygon triangles and corresponding pixel textures comprising the ventricular surface, which is resolved by 668,793 pixels. Each polygon triangle consists on average of about 100 pixels. The spatial resolution is about 150 μm per pixel. FIG. 79E shows polygon triangles mapped onto mesh and three-dimensional motion tracking. The tracking scheme involves sampling intensity values at points (red) which surround each vertex from the corresponding pixels in the cameras. Red vectors indicate computed local displacements of the vertices defining the epicardial surface.

Christoph and Schröder-Schetelig et al. [3] demonstrated that action potential waves can be imaged on the contracting three-dimensional left ventricular surface at higher spatial resolutions and without markers attached to the heart surface. The imaging and three-dimensional reconstruction was also performed with 4 cameras at 45°, but the heart's motion was tracked automatically in every pixel in each of the 4 videos, subsequently yielding a high-resolution polygon surface (4,000-5,000 polygons) with a spatial resolution comparable to the resolution of the video images (128×128 pixels). With the 4 cameras, it was possible to reconstruct nearly one half of the ventricular surface of the beating heart. However, one major drawback was that the approach required a static shape reconstruction of the pharmacologically uncoupled contraction-inhibited heart following each experiment. After the optical mapping of the contracting heart was completed, the heart was uncoupled with Blebbistatin. Once the heart was completely quiescent (15-30 minutes), it was rotated once around its long axis using a stepping motor while 72 images of its silhouette were taken every 5° throughout the 360° rotation. The silhouettes were then used to reconstruct the three-dimensional geometry of the quiescent heart using a shape-from-contour algorithm. Finally, the three-dimensional mesh showing the static heart was deformed using the two-dimensional motion tracking data obtained with each of the video cameras showing the beating heart. While this approach provided three-dimensional reconstructions of the shape of the deforming heart over time, it did not provide these reconstructions immediately during imaging. The required additional experimental part at the end with Blebbistatin was difficult to execute, and added post-processing steps and potential complications to each experiment that could prohibit further analysis. Most critically, the time available for imaging the beating heart was limited to a few minutes.

In the experimental results presented here, the previous shortcomings were address by introducing a stereoscopic multi-camera optical mapping approach according to an embodiment, which uses multi-view imaging and three-dimensional cooperative motion tracking of the heart surface with many low-cost cameras. This approach neither requires fiducial markers as in [2], nor a complicated static shape reconstruction obtained with Blebbistatin as in [3], is fully automatic, can be used to immediately reconstruct the heart surface and image action potential waves at high resolutions.

### 6.3. Methods

### 6.3.1. Soccerball-Shaped Imaging Chamber

A soccerball-shaped imaging chamber according to an embodiment was designed, which facilitates panoramic optical mapping of contracting hearts with up to 24 cameras simultaneously. The chamber allows a dense coverage of the heart with overlapping fields of views, while also providing an evenly distributed, unobstructed illumination of the heart with up to 48 light-emitting diodes (LEDs). The chamber's geometry is based on a truncated icosahedron or soccerball geometry. The soccerball is open at the top, has 24 penta- or hexagonal surfaces and an outflow at the bottom. Imaging can be performed through round windows in each of the 24 surfaces. The angle of the optical axes between two adjacent windows is 37.4° or 41.8°, respectively. Illumination can be provided through 48 small round windows (LED-ports) located at the vertices between the penta- or hexagonal surfaces. The volume of the chamber is about 5 liters. The size of the chamber is determined by the working distance of the cameras and lenses, the size of the heart and the size of the windows through which imaging is performed. Hearts were positioned at the center of the imaging chamber, such that they can be seen through all windows without any obstructions. The chamber was 3D-printed (3D printer: Creality CR-10 V2, material: white PLA+ by Overture). The plexiglas windows were glued into the window openings using silicone.

6.3.2. Langendorff-Perfusion of isolated Hearts

New Zealand White rabbits (N=3) were anaesthetized and hearts were quickly excised and transferred into ice-cold cardioplegic solution. All procedures were in accordance with animal welfare law and were approved by the Instutitional Animal Care and Use Committe (IACUC) at the University of California, San Francisco. Hearts were prepared and subsequently placed inside the imaging chamber filled with warm, oxygenated Tyrode solution (5 L) and attached to retrograde Langendorff-perfusion within 15-20 minutes after excision. Hearts were perfused in constant-pressure mode. The Langendorff-perfusion system comprised a 5 L water-jacketed reservoir (Radnoti, model no. 120142-5, USA) that was positioned about 0.6-0.7 m above the heart and connected via tubing to a heating coil with integrated bubble trap (Radnoti, model no. 158840, 10 ml, USA) positioned directly above the heart. The perfusion pressure was regulated by adjusting the height of the water reservoir to create perfusion pressures of 50±5 mm Hg. Perfusion pressure was monitored using a digital manometer (Hti-Xintai Instrument, HT-1890, China). The Tyrode was oxygenated and its pH-level was kept constant at 7.4 by bubbling Carbogen into the reservoir throughout the experiment. The pH-level was monitored using a digital pH meter (Extech PH220, Taiwan). An immersion circulator-heater (Polystat, Cole-Palmer, USA) was used to pre-heat water to 37° C.±0.5° C., which was then pumped through the water-jacketed reservoir and heating coil to pre-heat the Tyrode solution. The resulting temperature of the Tyrode flowing out of the heating coil into the heart was 36.75° C.±0.25° C. Hearts were stained with two 250 μl bolus injections of voltage-sensitive dye (Di-4-ANEPPS, Biotium, Germany) into the bubble trap. The second bolus was injected 5 minutes after the first bolus and imaging was started after about 10⁻¹⁵ minutes after the first injection. In this study, all hearts were imaged without pharmacological uncoupling agents, such as Blebbistatin.

### 6.3.3. Voltage-sensitive Multi-Camera Optical Mapping

Optical mapping was performed using up to 12 high-speed CMOS cameras (acA720-520 μm, Basler, Germany), which were positioned around the imaging chamber on an optical table (TMC, USA). The cameras were aligned and fixed in place using articulator arms (Noga, Japan). The average angle between neighboring cameras was approx. 35-45°. The cameras produce 12-bit (dynamic range: 0-4,096) greyscale video images with a maximal spatial resolution of 720×540 pixels. Imaging was performed at speeds of 500 fps with a spatial resolution of 440×320 pixels. The cameras were triggered using a signal generator (FY8300, FeelElec, China) to synchronize the video acquisition. Emission light was collected through machine vision lenses (3× CF8ZA-1s from below, 9× CF12ZA-1s horizontal, Fujinon, Japan) mounted onto each of the cameras together with long-pass emission filters (Dark Red #29, Heliopan, Germany), which block green excitation light and transmit red and near-infrared fluorescent light. With the magnification of the lenses, the video images covered a field of view of about 4×5 cm at working distances of about 10⁻¹⁵ cm). Green excitation light was provided by up to 40 high-power light-emitting diodes (Cree XLamp XP-E2 High Power LED Star Single 1 Up, 530 nm center wavelength, USA), which were directed at the heart through the small LED-ports between the imaging windows of the imaging chamber). The videos from the cameras were streamed via USB onto a computer (AMD Ryzen Threadripper 3960X CPU, 2 TB SSD working memory, Gigabyte Technology Co. TRX40 mainboard) using the 9 integrated USB ports and additional external USB hubs (10 Gbps, USB 3.2 Gen 2).

### 6.3.4. Rhythm Control and Other Data Acquisition

Paced rhythms were induced using a custom-made micro-electrode (FHC Inc., USA), which was connected to an isolated pulse stimulator (A-M Systems, model 2100, USA), which was externally triggered using custom-made software. Regular ventricular pacing was induced using 8 ms long biphasic pulses applied at 3-8 Hz. Ventricular fibrillation was induced using burst pacing at frequencies ranging from 15-50 Hz and terminated using a custom-made defibrillator. Camera triggers and stimulation pulses were recorded during the experiment using an analog-to-digital data acquisition system (PowerLab 16/35, ADInstruments, Australia).

### 6.3.5. Camera Calibration

Camera calibration refers to the process of determining extrinsic and intrinsic camera parameters with the goal of undistorting the cameras and establishing the relative positioning and alignment of the cameras with respect to each other. Camera calibration was performed after each imaging experiment using an 8×10 ChArUco calibration target with a 4 mm checkerboard pattern and ArUco fiducial markers in each of the black squares [22]. The calibration target was manually positioned and rotated within the imaging chamber and a full set of images was taken per pose of the target with all cameras simultaneously. The target was typically observable by at least 2-3 cameras. The calibration procedure required typically 30-100 of image sets in total, while at least 2-3 sets needed to be taken for each camera. With both the extrinsic and intrinsic camera parameters, it is possible to transform a 3D world coordinate to an image coordinate, but for the transformation to be accurate it must account for camera distortion. In addition to the camera's inherent distortion, the refraction from the light passing from water to glass to air introduces significant additional distortion. Kwon et al. showed that the refraction error can be accounted for using standard camera distortion calibration [23]. multical was used to perform the camera calibrations [24].

6.3.6. Three-Dimensional Motion Tracking and Surface Reconstruction using Cooperative Multi-View Tracking

Using the camera calibration, COLMAP was used to produce raw, static 3D reconstructions of the heart for each frame over time. A frame corresponds to a set of video images acquired at the same time by the different cameras. The static reconstructions were performed independently from each other frame-by-frame over the sequence of frames, producing a sequence of unique, uncorrelated raw meshes with different numbers of vertices and topologies. Visually, these meshes reflect the motion of the heart when texturized, but they cannot be used to measure action potential waves or strain.

To compute a single moving mesh that represents a dynamic reconstruction of the moving heart surface, a three-dimensional mesh tracking technique by Klaudiny et al. was used [26]. This cooperative patch-based mesh tracking technique produces a single moving mesh with fixed topology and the same number of vertices over time from the individual meshes from the static reconstructions. It does so by tracking the vertices of a template mesh, searching for the corresponding tissue segments in both the other already pre-computed raw meshes and in the different camera images. In embodiments, the template mesh corresponds to one of the raw meshes obtained during the static reconstructions used as a reference frame. The reference frame shows either the heart shortly before the depolarization of the ventricles during sinus rhythm, or is an arbitrary frame in case of arrhythmias. To produce the next mesh in the sequence of frames, the algorithm uses first the reference mesh and then each previous mesh as a starting point and then computes and minimizes an error function for each vertex ve using a patch-match inspired approach [27]. This results in a new time-varying mesh with vertices moving in a manner such that its positions have moved in accordance with the video data from the different cameras.

In more detail, the vertex error function is comprised of an image-based component and a mesh-based component. For the image component, a set of sample points surrounding the vertex on the mesh's surface were created, as shown in FIG. 1 in [26]. This is done for the vertex in the previous mesh and vertex in the new mesh, which is initially a replication of the previous mesh. These sample points are projected into the camera images in which they are visible in. The pixel values of the previous mesh's vertex sample points are compared with the new mesh's vertex sample points using a normalized cross-correlation similarity measure. This ensures that the area around the vertex in the new mesh is similar to the corresponding vertex in the previous mesh. For the mesh component, it computes the distance of the vertex from the surface of the raw, static mesh. This distance is passed through the Tukey biweight function to reduce the effect of outliers. The image error iₑ and mesh error me components are summed with a weighting term w for the mesh error: vₑ=iₑ+w·mₑ. The mesh error reduces the search space and drift over time. The vertex error was optimized using a cooperative optimization algorithm that optimizes all vertices together [27]. It begins by randomly translating each vertex multiple times and then keeping track of the displacement that produces the lowest error. In the next step, each vertex applies their neighboring vertices best displacements to itself and checks if it improves the error. The random search and neighbor searching stages are repeated in succession 4-5 times to find the vertex positions for the new mesh that minimize the error. Subsequently, the displacements of the new mesh's vertices were taken with respect to the previous mesh and a weighted smoothing was applied to the displacements based on the vertex error. This results in a completed mesh tracking step for one frame, which is then repeated sequentially for all frames to produce the time-varying mesh over the entire sequence of frames.

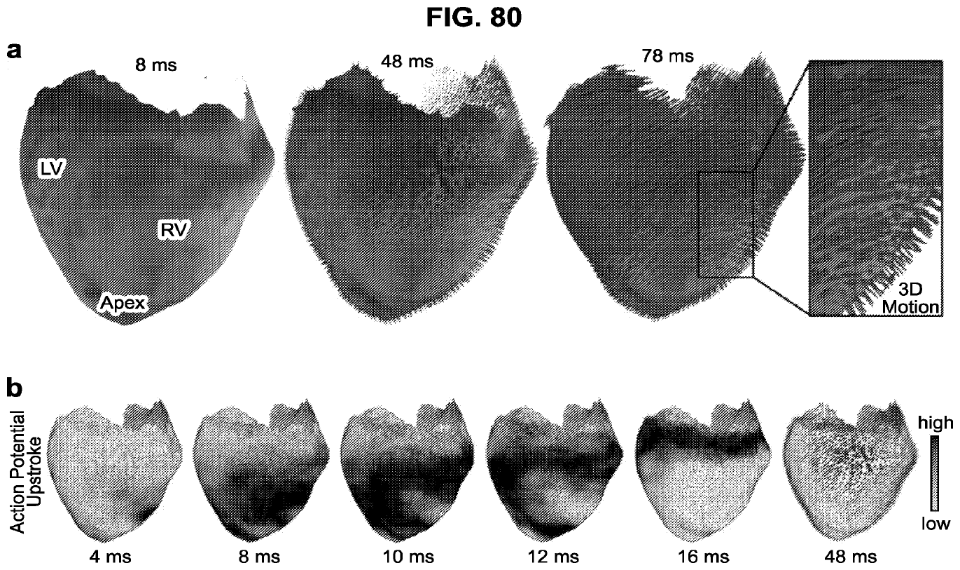

FIGS. 80A-B show sinus rhythm imaged in a contracting rabbit heart with voltage-sensitive multi-camera optical mapping consisting of 12 cameras. Using three-dimensional motion tracking and shape reconstruction techniques, action potential wave fronts were imaged across the entire contracting and strongly deforming ventricular surface (LV/RV: left/right ventricle). FIG. 80A shows raw grayscale video data mapped onto three-dimensional reconstructed ventricular surface together with motion vectors (red) indicating motion and deformation of the ventricular surface with respect to the mechanical configuration at 0 ms (beginning of depolarization). FIG. 80B shows action potential wave front (dark: upstroke, high negative rate of change in intensity) propagating across ventricular surface triggering the contraction and deformation of the heart. Noticeable contraction sets in about 30 ms after the beginning of the depolarization of the ventricles.

### 6.3.7. Post-Processing

The vertices of the time-varying mesh describe the motion of the heart surface. With that information, signal analysis can be performed in a co-moving frame of reference. Accordingly, the video images were warped using the two-dimensional projections of the movements of the three-dimensional mesh in the camera images to be able to further process the data in an image-based co-moving frame of reference. This step significantly reduced motion artifacts. However, because the resolution of the mesh is lower than the resolution of the video images, optical flow-based motion tracking was further performed as described in Lebert et al. [7] using a GPU-accelerated version of the Farneback algorithm [28] on the already warped video images and warped them further with respect to the reference video images. This last step significantly reduced residual motion and motion artifacts in the triangles between the vertices and is included in all motion-compensated optical maps shown throughout this study. Otherwise, the raw grayscale video images were texture-mapped showing the moving heart onto the moving mesh to produce texturized meshes. Similarly, the warped, processed and then unwarped videos were projected onto the moving mesh. Projections were performed using a weighted average based on the angle between the mesh's surface normal and the camera's direction. The texture data was saved in 8-bit format as a PNG file, so the textures could be processed by general mesh viewing tools. Each camera had 2-4 neighboring cameras at a time depending on their overlap. The average angle between cameras used for depth-map creation was 54±18°, which includes nearest-neighbouring cameras as well as second-nearest cameras and cameras further away.

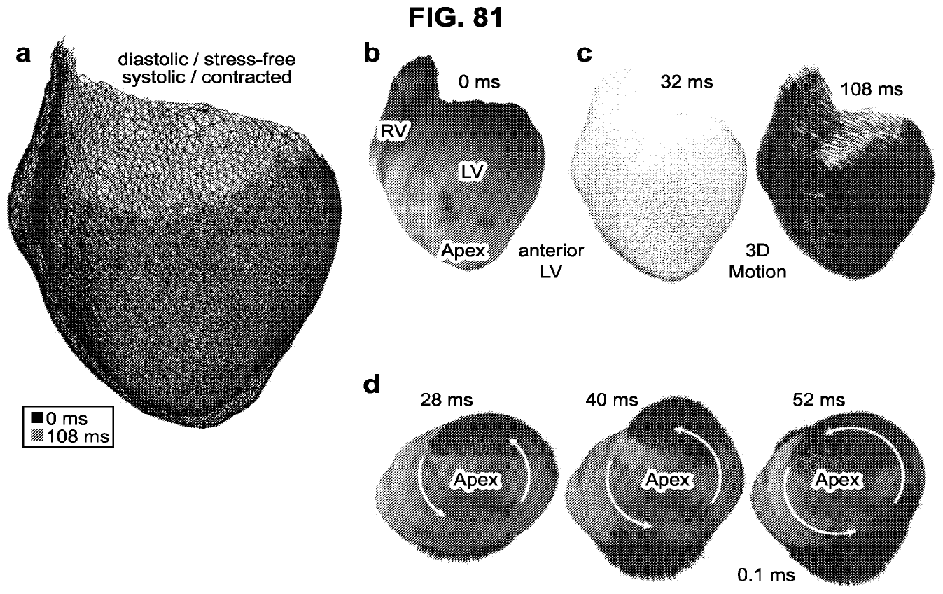

FIGS. 81A-D show ventricular wall motion during sinus rhythm imaged with a 12 camera optical mapping system. FIG. 81A shows wireframe meshes of epicardial surface in diastolic, stress-free (black) and deformed, contracted (red) mechanical configuration. The mesh consists of 3,325 vertices or 6,349 polygon triangles, respectively. Average nearest-neighbour distance: 0.85±0.38 mm (black mesh). FIG. 81B shows raw video images of left ventricle (LV) projected onto mesh. FIG. 81C shows three-dimensional displacement vector field (red) indicating motion at 32 ms and 108 ms, respectively, with respect to diastolic, stress-free mechanical configuration at Oms. FIG. 81D shows an apical view with displacement vectors (red) indicating motion with respect to the previous frame. The fastest contractile motion occurs between 30 and 50 ms, and the ventricles exhibit rotational, torsional motion around apex.

All post-processing of the video data was performed in a co-moving frame of reference as well as in an image-based fashion in the individual warped video images. The three-dimensional post-processed (e.g. normalized) optical maps are the average of multiple post-processed videos and are combined using a weighted average with the weights being the angle between the triangle normals and camera's view direction. To visualize action potential waves, the optical signals representing action potentials were normalized in the video data per pixel over time. A pixelwise normalization was used with a temporal sliding window (30-50 frames length), or a framewise difference computation with subsequent temporal normalization to amplify the optical signal. Spatial or temporal smoothing was not applied throughout.

### 6.3.8. Phase Maps and Phase Singularity Calculation

Phase maps were computed from the motion-stabilized and pixel-wise normalized optical mapping videos in each camera individually. The phase angle was computed for each time-series using the Hilbert transform. The resulting phase maps were smoothed using a complex order parameter filter as described in [29]. The smoothed phase maps were then projected onto the three-dimensional mesh and averaged from multiple camera perspectives if the respective pixels overlapped. Phase singularities were computed on the three-dimensional mesh surface by computing the circular integral of the gradient of the phase, as described in [30].

### 6.4. Results

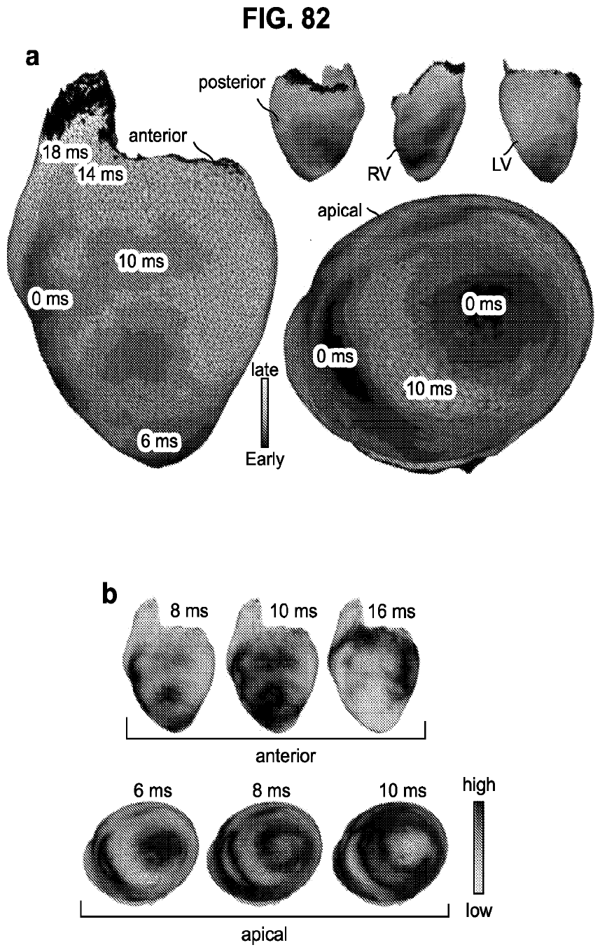

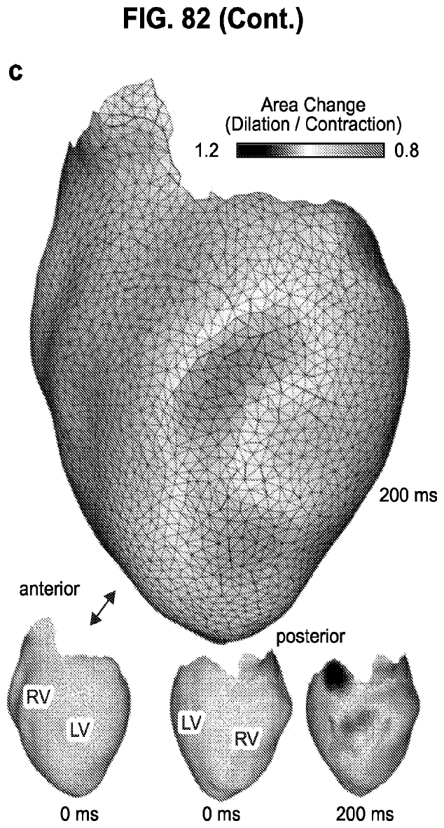

FIGS. 82A-C show panoramic electrical activation and strain maps in a contracting isolated rabbit heart. FIG. 82A shows electrical activation map with local activation times (black: early/0 ms, yellow/white: late/20 ms; anterior, posterior, apical view, etc.) computed form the upstroke of the action potential in FIG. 82B. The entire ventricles get activated within 20 ms. The activation map indicates multiple epicardial break-through sites in the apical and septal regions. FIG. 82B shows action potential wave front (black: upstroke, high negative rate of change in intensity) and electrical activation of ventricles (0 ms: beginning depolarization) used to compute activation map in FIG. 82A as seen from anterior and apical perspectives, respectively. FIG. 82C shows local area change of epicardial surface (blue: dilated, red: contracted, 1.2/0.8=20% area increase/decrease) computed for each triangle as an approximation for epicardial strain (from anterior perspective). Bottom left: Undeformed, stress-free epicardial surface (0 ms: beginning depolarization) from anterior perspective, respectively. Bottom center and right: Stress-free and deformed epicardial surface (0 vs. 200 ms), respectively, from posterior perspective.

Fully panoramic voltage-sensitive optical mapping with contracting isolated hearts was performed, imaging action potential waves across the entire three-dimensional deforming ventricular surface during sinus rhythm, ventricular pacing and ventricular fibrillation.

### 6.4.1. Sinus Rhythm

Sinus rhythm poses the most challenging imaging condition when imaging contracting hearts in optical mapping studies. During sinus rhythm, the heart contracts and deforms much stronger than during most paced rhythms or during arrhythmias. Accordingly, it is more challenging to track the tissue, as it is likely to move out of the field of view or deform so strongly that optical flow algorithms cannot correlate two video images when imaging with a single camera. Likewise, motion artifacts can become very strong during sinus rhythm. Results show that, using an embodiment of a multi-view cooperative tracking approach, it is possible to track the strongly deforming ventricles during sinus rhythm and subsequently map action potential wave fronts propagating across the entire three-dimensional ventricular surface at unprecedented resolutions. FIGS. 80A and 81B-D show the reconstructed three-dimensional ventricular surface together with displacement vectors (red, size of arrows scales with length) indicating the motion and deformation of the heart as it contracts in response to the depolarization of the ventricles. The vectors in FIGS. 80A and 81C indicate displacements of the epicardial surface with respect to its stress-free unloaded mechanical configuration during diastole just before the onset of the depolarization (at 0 ms), while the vectors in FIG. 81D indicate instantaneous displacements between subsequent frames with respect to the previous frame acquired 2 ms earlier. The instantaneous displacements therefore correlate with contractile speed. The mechanical measurements allow one to analyze the intensity fluctuations across the heart surface in a co-moving frame of reference and compensate motion artifacts. Correspondingly, FIGS. 80B and 82B show the action potential wave that triggers the contraction as it propagates across the left and right ventricles (LV, RV). The black wave corresponds to the action potential wave front or upstroke, which was computed as the difference between pixel intensities in subsequent frames in the co-moving frame. The high negative rate of change in intensity (negative ΔF/F) is associated with Di-4-ANEPPS reporting the rapid depolarization and change in transmembrane voltage. The action potential wave originates in several locations in the apical and septal regions in both the left and right ventricles and propagates upwards towards the base of the heart, activating the entire ventricular muscle within less than 20 ms, as also depicted by the electrical activation map shown FIG. 82A. The ventricles begin to contract and deform shortly thereafter. However, noticeable contraction sets in only about 30 ms after the onset of the depolarization of the ventricles. The ventricles exhibit maximal contraction velocities of about 0.1 m/s about 40-50 ms after the onset of the depolarization with peak velocities at 48 ms. These high contraction speeds then lead to the large deformations depicted by the wireframe mesh in FIG. 81A and the corresponding displacements shown in FIG. 81C. The experimental results show that the depolarization phase of the action potential and the onset of contraction are clearly separated in time. Overall, the heart exhibits an intricate and large deformation including torsional motion around the apex. It moves upwards as it contracts, while its base remains roughly in its vertical position. The epicardial surface experiences mostly contractile strain with a local area decrease of about 10⁻²⁰%, however, some regions experience tensile strain with a local area increase of about 5-15%.

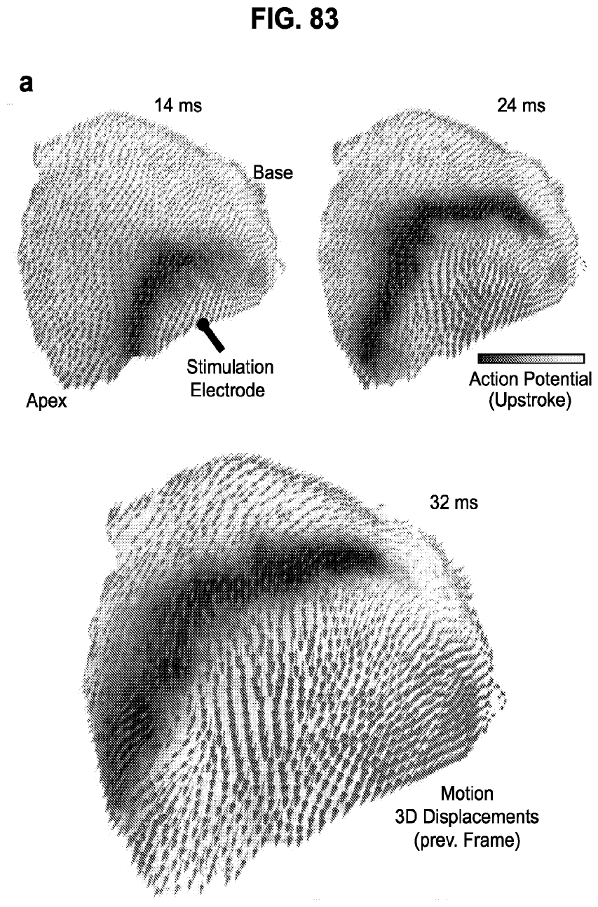

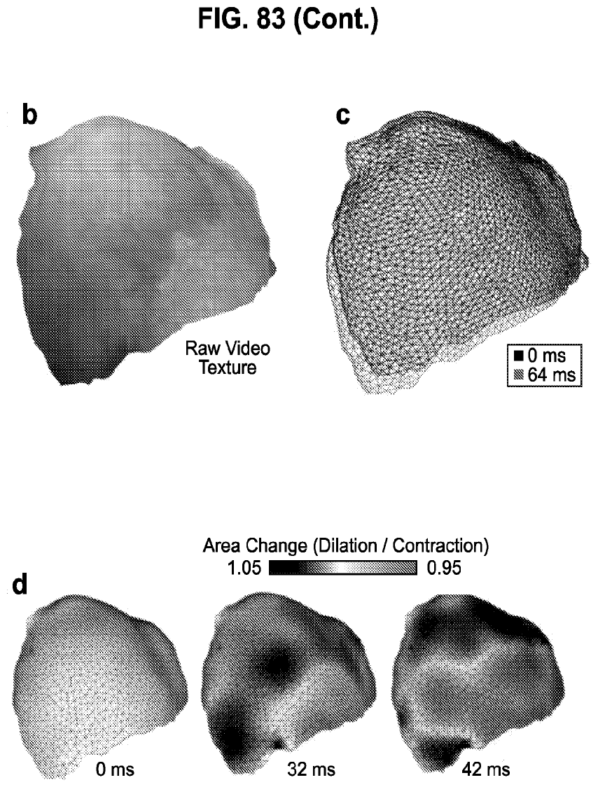

FIGS. 83A-D show action potential wave front (black) propagating across the left ventricular (LV) surface of a rabbit heart following a pacing stimulus. Three-dimensional reconstruction obtained with 5 cameras. FIG. 83A shows action potential wave front (black: upstroke, ∂tI(t)) propagating across strongly deforming LV surface (red: three-dimensional displacement vectors indicating motion with respect to previous frame, acquired 4 ms earlier). The tissue moves down and towards the approaching wave and then contracts noticeably when the action potential wave front has passed. FIG. 83B shows raw video texture. FIG. 83C shows wireframe mesh before stimulation (black) and 64 ms after stimulation (red). Tissue moved down and left. FIG. 83D shows area change (per triangle) in response to action potential wave (blue: dilation/area increase, red: contraction/area decrease). The area change was computed with respect to the mechanical configuration at 0 ms. The ventricular surface contracts behind the action potential wave front as expected.

These experimental results demonstrate that it is possible to measure both the electrical and mechanical dynamics of the heart during sinus rhythm simultaneously at very high spatial and temporal resolutions. In this example, both the wireframe mesh and the vector field consist of 3,235 vertices or displacement vectors, respectively, the vertices forming 6,349 polygon triangles. The average distance between two neighbouring vertices is 0.85±0.38 mm and the average area of a polygon triangle is about 0.53±0.17 mm² (at Oms). The entire ventricular surface is resolved by 668,793 pixels, where each polygon triangle consists on average of about 100 pixels. The spatial resolution of the texture map is so high that it is possible to identify individual small vessels and other fine-scaled structures across the ventricular surface. Per pixel, the spatial resolution is about 150 μm. The horizontal (short-axis) diameters of the heart are 2.89 cm and 3.36 cm along the shorter and longer axes of the elliptical shape of the heart, respectively.

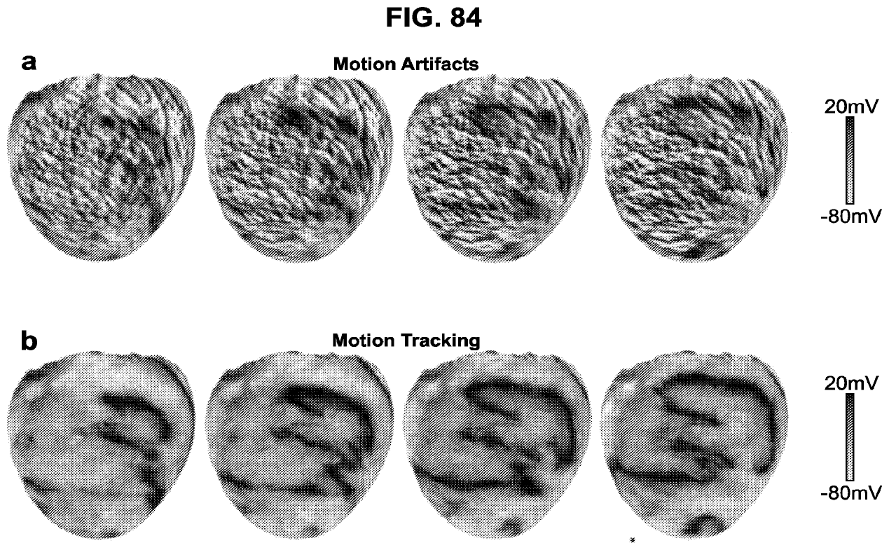

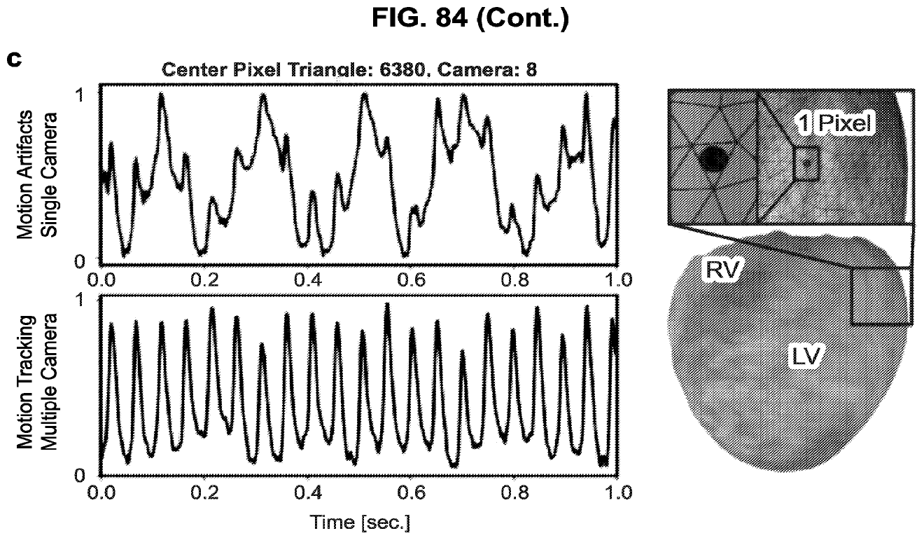

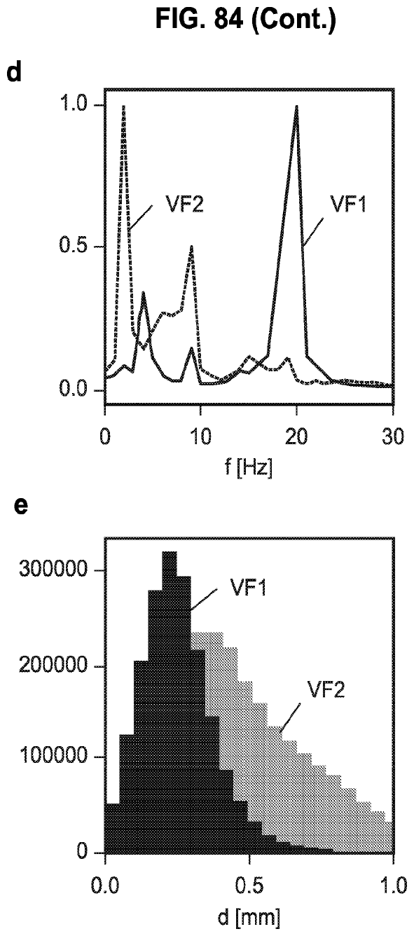

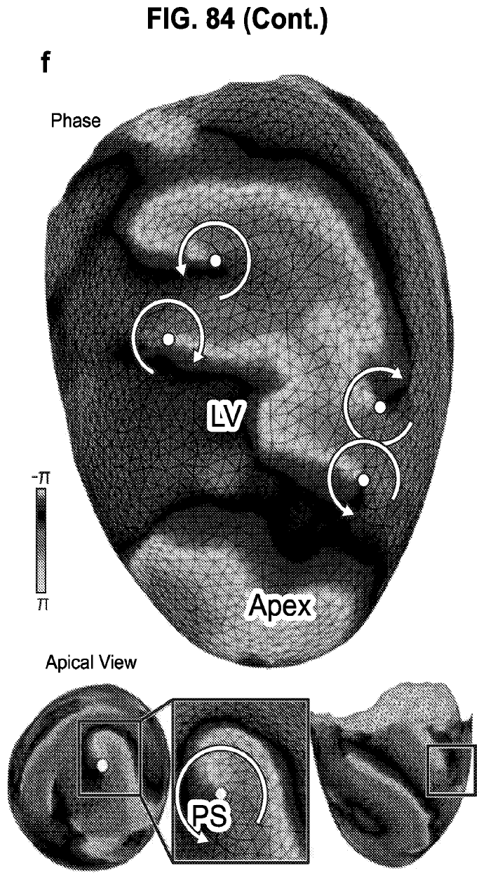

FIG. 84A-F show ventricular fibrillation (VF) imaged across the entire surface of a contracting isolated rabbit heart using voltage-sensitive 3D optical mapping with 12 cameras. FIG. 84A shows optical maps before motion tracking with motion artifacts. FIG. 84B shows optical maps after motion tracking showing action potential vortex waves on left ventricular (LV) surface (black: depolarized tissue, light gray: repolarized tissue, pixel-wise normalized video data, 30-50 frames sliding window). FIG. 84C shows optical traces before (top) and after (bottom) motion tracking and co-moving analysis measured in one pixel (marked by red sphere). FIG. 84D shows frequency spectra (cumulative) indicating fast VF1 episode (black, dominant peak at 20 Hz) shown in a-c,f) and ‘slow’ VF2 episode (light gray, dominant peak at 10 Hz) computed from (all) motion-compensated traces. FIG. 84E shows episode VF2 (light gray) exhibits larger contractile motion than episode VF1 (black): distribution of displacement magnitudes d [mm] with respect to first frame (1 sec. long video). FIG. 84F shows corresponding phase maps and phase singularities (PS) computed from motion-compensated optical maps shown in FIG. 84B with subsequent spatio-temporal smoothing of the phase maps.

### 6.4.2. Paced Rhythms

To investigate the relationship between electrical waves and mechanical deformation in more detail, a planar action potential wave was imaged as it propagates across and deforms the left ventricle (LV) during pacing. Pacing stimuli were applied using the micro-electrode shown in FIG. 77D. FIG. 83A shows an action potential wave front (black) originating from the stimulation site (bottom) and propagating upwards across the deforming left ventricle. As in FIGS. 80B and 82B, the wave corresponds to the action potential wave front or upstroke (normalized temporal derivative of intensity ∂tI(t) with dark regions emphasizing a high negative rate of change in intensity). The motion and deformation of the epicardial surface is indicated by three-dimensional displacement vectors (red), which indicate shifts with respect to the previous frame acquired 4 ms earlier. The tissue moves down and towards the approaching wave and then contracts noticeably and more quickly when the action potential wave front has passed. Accordingly, the area change across the epicardial surface indicates dilated areas (blue) before the action potential wave front approaches and contracting areas (red) within the action potential wave, which is further experimental evidence for electromechanical waves consisting of closely correlated electrical and mechanical wave phenomena, as described in [4, 31]. The data was generated using 5 cameras, which were positioned on one side of the heart, using the top, center and bottom windows. Imaging was performed with 8 cameras, but camera data that was partially obstructed by the pacing electrode was discarded. The imaged area corresponds to the anterior LV.

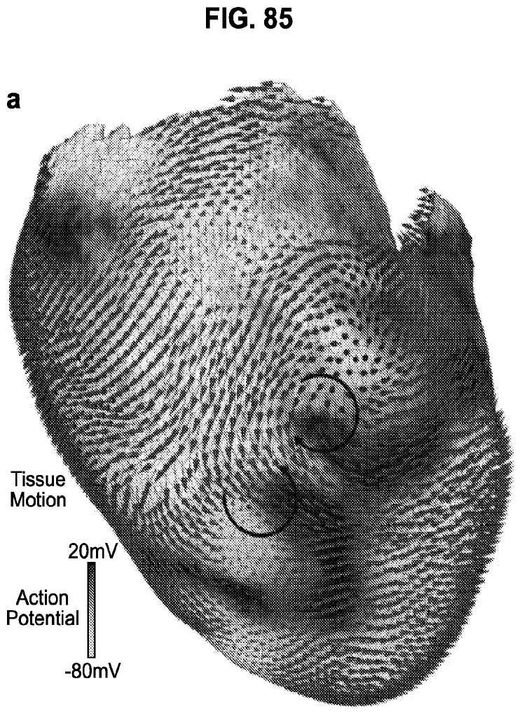

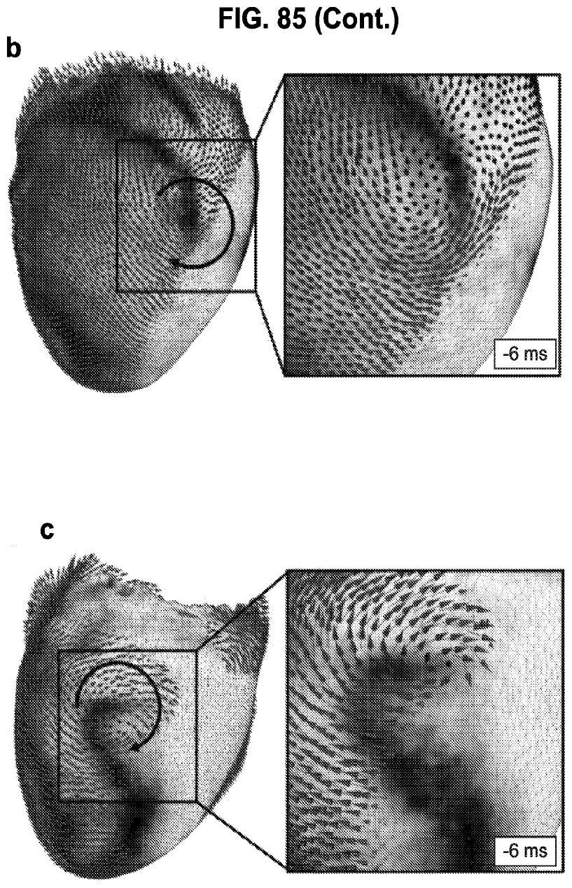

FIGS. 85A-C shows vortex-like rotating action potential waves (black: depolarized tissue, light gray: repolarized tissue) cause rotating mechanical motion of the ventricular surface in a fibrillating and contracting rabbit heart. The vectors (red) indicate three-dimensional tissue displacement with respect to the ventricles's mechanical configuration 20 ms earlier. In order to highlight curls in the displacement field, the vectors are normalized and do not encode magnitude but only direction. In some regions the vectors point into the heart muscle and are therefore not visible. FIG. 85A shows figure-of-eight or double rotor action potential wave pattern consisting of a pair of clockwise and counter-clockwise rotating waves, which induces two mechanical curls or vortex-like structures in the displacement vector field nearby. FIG. 85B shows clockwise rotating action potential wave and clockwise rotating curl in the displacement vector field in the left ventricle. FIG. 85C shows clockwise rotating action potential wave and clockwise rotating curl in the displacement vector field in the right ventricle. Inserts show the same area 6 ms earlier to demonstrate that the phenomenon evolves smoothly over a brief period.

### 6.4.3. Ventricular Fibrillation

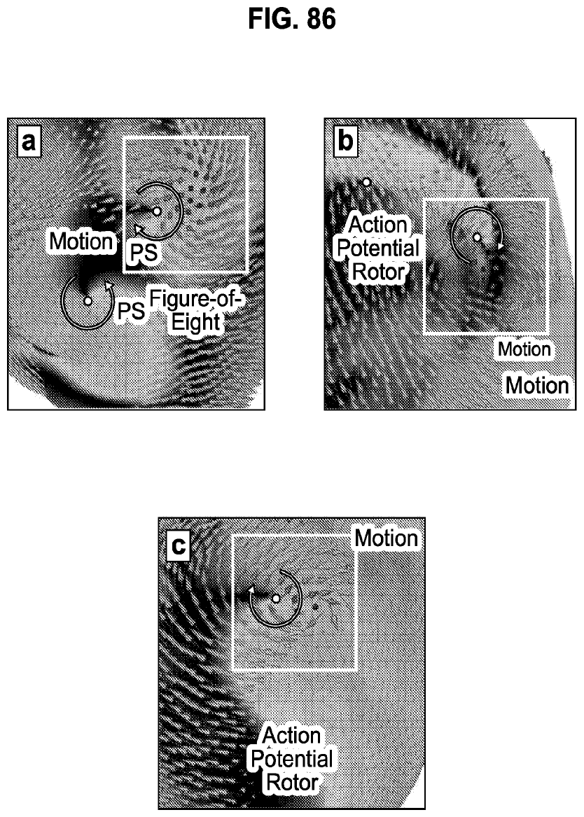

Hearts contract only minimally during ventricular fibrillation. However, the residual contractile motion during fibrillation is strong enough to produce considerable motion artifacts in optical mapping studies. In these experimental results, two episodes of ventricular fibrillation (VF) are discussed: one with very little to almost no motion (VF1), shown in FIGS. 84A-F, and one with finite, moderate contractile motion (VF2), shown in FIGS. 85 and 86. The different contractile strengths between the two VF episodes can be associated with a much shorter action potential duration, which leads to accelerated fibrillatory dynamics (20 Hz activation frequencies) and very minimal motion in episode VF1, versus a longer action potential duration which leads to slower fibrillatory dynamics (10 Hz activation frequencies) and moderate contractile motion in episode VF2. While episode VF1 is an interesting example to discuss motion artifacts, in episode VF2 electrical wave patterns could be correlated with mechanical deformation patterns. Both episodes were recorded in the same heart.

FIGS. 86A-C show action potential vortex waves and mechanical vortices on the three-dimensional, deforming ventricular surface during ventricular fibrillation (VF) in a rabbit heart. The action potential vortex waves are displayed as pinwheel-shaped phase patterns with lines of equal phase merging at the rotational centers of the action potential waves, which were computed and displayed as phase singularities (PS, white dots). Curl-shaped mechanical displacement vectorfields (red vectors) emerge in the vicinity to electrical PS. As in FIGS. 85A-C, the vectors indicate the direction, not the magnitude of the epicardial displacements (all vectors normalized) to emphasize curls in the vectorfield. Displacements were calculated with respect to the tissue's mechanical configuration 20 ms earlier.

FIG. 84A-B show that even with very little to almost no motion the tissue needs to be tracked, because otherwise motion artifacts become so strong that they prohibit further analysis of the data. FIG. 84A shows pixel-wise normalized optical maps, which were not tracked and subsequently not analyzed in a co-moving frame. Motion artifacts manifest as the high-frequency dark-bright pattern, which superimposes the action potential waves, see [5] for an in-depth characterization and explanation of their origin. By contrast, FIG. 84B shows that the pixel-wise normalized optical maps that were computed in the co-moving frame after motion tracking do not exhibit any significant motion artifacts, but instead show multiple reentrant vortex-like action potential wave patterns. Note that here and in FIGS. 85A-C, the action potential wave were displayed and not the action potential wave front as in FIGS. 80-83. The dark area corresponds to depolarized tissue, the bright area to repolarized tissue (normalized units [0,1]). In this episode VF1, the action potential duration is extremely short in the order of 30-40 ms and the wavelength is in the order of 1-3 mm, respectively. In both FIGS. 84A-B and 85, intensity fluctuations were amplified numerically using pixel-wise normalization with a sliding window (with a length of 50 frames). The tissue only moves on average by about 0.2-0.3 mm. The fact that the strong motion artifacts in FIG. 84A occur at all with this little motion highlight the necessity to track the tissue and analyze the video data in a co-moving frame of reference more generally. All other examples in this study, particularly the sinus and pacing data, exhibit much stronger motion and it is therefore absolutely required to employ motion tracking and motion artifact compensation in general with moving tissue preparations. FIG. 84C shows optical traces before and after motion tracking with and without motion artifacts, respectively. The top trace shows a measurement without tracking obtained from a single pixel of one of the cameras (red dot, the pixel is located at the center of the respective polygon triangle). The time-series shows an unrecognizable, distorted sequence of action potential waves superimposed by strong intensity modulations, which are likely caused by the back-and-forth movement of the darker nearby vessel through the area. The bottom trace shows the corresponding measurement obtained in a co-moving frame, again from a single pixel. As the measurement was obtained from the same area of the tissue (the center pixel of the polygon triangle) due to the tracking, the motion artifacts from above are largely compensated and the trace shows instead a sequence of action potentials. The residual baseline modulations are residual illumination-related motion artifacts likely caused by relative motion between the tissue and the LEDs. As this relative motion cannot be removed by motion tracking per se, it would be required to employ ratiometric imaging [2, 6] or numerical light-field correction methods [3] to suppress these residual motion artifacts. However, it was found that the tracking as well as the averaging of data from multiple cameras significantly enhances the imaging quality.

Lastly, FIG. 84F displays phase maps, which were computed from the motion-stabilized optical maps and traces shown in FIGS. 84A-C. The phase maps emphasize the vortex-like structure and topological organization of the action potential wave pattern, and indicate the rotational centers of the electrical vortex waves as phase singular points (PS, white dots). In this example, there are several rotating vortices including a figure-of-eight pattern in the anterior left ventricle (LV) and a persistent rotating vortex close to the apex. The phase singularities allow us to automatically track electrical vortex waves across the moving three-dimensional ventricular surface.

With the larger contractions in episode VF2, it is possible to now investigate the relationship between reentrant action potential vortex waves and the three-dimensional deformation of the epicardial ventricular surface. FIGS. 85A-C show three examples of reentrant action potential waves (black: depolarized tissue, light gray: repolarized tissue, normalized units [0,1]): FIG. 85A shows a figure-of-eight pattern and FIGS. 85B-C show two clockwise rotating action potential waves in the left and right ventricle, respectively. In this slower episode, the action potential waves induce noticeable contractions, which lead to macroscopic deformations of the ventricles in response to the electrical activations. The dominant frequency of the action potential vortex wave dynamics is about 10 Hz. At these slower activation frequencies, the contractions are much stronger than at the higher frequencies in episode VF1 in FIGS. 84A-F, which is an observation that was also made in [4]. The tissue moves on average by at least about 0.5-1.0 mm and in many instances even stronger than that. The larger deformations produce smoothly varying vector fields in space and time and it is possible to identify curls in these deformation patterns. Note that the displacement vectors are scaled differently than in FIGS. 80-83 and 85. Here they display only the direction of the movements of the vertices and not the magnitude of these movements. This visualization was chosen to highlight vortices or curls in the displacement vector fields, which emerge repeatedly close to electrical phase singularities (PS). Displacement vectors which point into the heart muscle are not visible in FIGS. 85-86. FIG. 86A-C show the corresponding phase maps and the mechanical curls in the vicinity of electrical PS. This data confirms a strong correlation between action potential waves and tissue deformation during ventricular fibrillation, and hints at topological abnormalities in the form of mechanical vortices/curls that emerge in the deformation of the heart wall during ventricular fibrillation. The strong correlation was first described in [4] analyzing single-camera optical mapping data. Here, these experimental results present for the first time, three-dimensional measurements of reentrant action potential waves and co-localized vortex-like tissue deformation across the ventricular surface.

### 6.5. Analysis

In these experimental results, it was demonstrated that it is possible to image action potential waves at high resolutions across the entire deforming ventricular surface of contracting isolated hearts using voltage-sensitive multi-camera optical mapping and three-dimensional numerical motion tracking. With such an embodiment, it is possible to measure electrical and mechanical phenomena in the heart at very high spatial and temporal resolutions, and measure typical quantities, such as electrical activation times and tissue strain, simultaneously in three dimensions across the moving and deforming heart surface. The measurement data is invaluable as it can be used to study the heart's electromechanics in unprecedented detail and to inform and calibrate electromechanical computer models. While 12 cameras were used in obtaining the presented experimental results, an embodiment of an imaging chamber fits up to 24 cameras and other embodiments are configured to image the whole heart including the atria with more cameras. It is anticipated that 2 sets of up to 12 cameras can be used to image action potential and calcium waves simultaneously across the entire surface of the contracting heart to study the three main physiological players underlying the heartbeat: voltage, calcium and contraction.

### 6.5.1. Practical Considerations

An overall approach and design of the embodiment of the imaging setup utilizes the recent availability of industrial-grade low-cost cameras [32]. These cameras produce lower quality video images, but it was found that averaging the overlapping video images across the three-dimensional surface produces high signal qualities. The lower costs enabled the integration of far more cameras into the setup than previously possible with more expensive scientific cameras. This enabled the pursuit of a more robust panoramic optical mapping approach. In the past, multi-camera optical mapping systems consisted of up to 4 scientific cameras [2, 3], the main limiting factor being cost. While 3-4 cameras are sufficient for mapping the entire surface of non-moving hearts [13, 14, 15, 16], and low-cost cameras were already used for that purpose [32], it was estimated in [3] that at least 8 cameras would be necessary to image the entire ventricular surface of a contracting heart.

In these experimental results, it was shown that operating 12 or more cameras is technically and practically feasible. The video data from 12 cameras was streamed onto a single computer using USB, and major issues were not experienced with camera failures, the high data rates, or calibrating the cameras. Nevertheless, the high data rates and large amounts of data that need to be captured, stored and processed during and after each experiment do pose a challenge. With 24 cameras and video image resolutions of 440×320 pixels, data rates of 3 gigabytes per second would be obtained. Accordingly, a 10 second long recording with 24 cameras would create 30 gigabytes of data. In the case of the embodiment used in connection with these experimental results, with 12 cameras, a single experiment with 25-50 recordings produced on average about 0.5-1.0 terabytes of data. While the video images were cropped and unnecessary data was discarded during the experiment to save storage space, further work is needed to compress the raw data into a suitable format for long-term storage.

Despite the many cameras, the embodiment of the imaging setup is cost-effective with an overall approximate cost of φ20,000 for 24 cameras (φ400 each) including the respective lenses ($500-800 each). By comparison, a single scientific camera costs φ10,000-φ50,000 (e.g. Brainvision Scimedia MiCAM Ultima/N256, Teledyne Photometrics Evolve 128, Oxford Instruments Andor) and a setup of 4-10 scientific cameras would correspondingly cost S100,000-$500,000, which is prohibitively expensive for most laboratories. Adopting a low-cost design with industrial-grade cameras could enable more researchers to venture into the world of panoramic three-dimensional optical mapping.

### 6.5.2. Advantages

An advantage of an embodiment of the present invention is that it does not rely on fiducial markers [2] or any additional shape reconstruction methods [3]. As already mentioned herein, the method described in Zhang et al. [2] required fiducial markers, which impose lower spatial resolutions and may affect the tissue's mechanics. In Christoph and Schröder-Schetelig et al. [3] it was necessary to perform a static shape reconstruction with the excitation-contraction uncoupling agent Blebbistatin. This additional step limited the time that was available for optical mapping to 5-15 minutes, because swelling of the heart, photo-bleaching or wash-out of the fluorescent dye would compromise the motion tracking and shape reconstructions. With the approach of embodiments of the present invention, there are no such constraints. It is possible to perform measurements over 1-2 hours, then calibrate the cameras, track the tissue in the recorded data and generate three-dimensional dynamic reconstructions in a fully automatic fashion post-acquisition. Currently, the only manual step during post-processing comprises selecting a region of interest in the raw reconstructions and cleaning the mesh before continuing with the dynamic reconstructions.

Another advantage is that the soccerball-shaped imaging chamber allows one to image and illuminate the heart from as many as 24 and 48 pre-defined locations, respectively, which are evenly spaced and distributed around the heart. This circumvents several issues with regard to inhomogeneous illumination, space restrictions and other complications that arose with previous experimental setups. A crucial element in optical mapping studies with contracting hearts is to illuminate the heart as homogeneously as possible. The homogeneous illumination minimizes illumination-related motion artifacts, which are caused by relative motion between the tissue and the light sources, see limitations section in the discussion in [7]. Even though it is impossible to illuminate the heart perfectly homogeneously, embodiments' illumination scheme produces smaller illumination gradients than if illumination occurred from fewer sites. For instance, in octagonal baths with 8 glass walls, which were used in [3] and [4], the fewer windows restrict the positioning of the cameras, which need to image perpendicularly through the windows, and require careful positioning of the light sources to avoid back-reflections from the glass surfaces in the camera images or shadows on the heart surface. These issues were circumvented with embodiments' chamber design, which allows the unobstructed illumination and imaging of the heart. A similar approach was described for an optical fiber-based imaging setup reported in [33].

Lastly, very high spatial resolutions were achieved thanks to the many cameras and the Sony IMX287 CMOS sensor, which produces images in the order of 300×300 pixels at 500 fps. Electrical wave phenomena were measured on the entire ventricular surface with about 0.5-1 million pixels (about 150 μm per pixel). At these resolutions, it was possible to identify single small vessels across the ventricular surface. Thus far, optical mapping was performed only on parts of the contracting ventricular surface, with video images in the order 100×100 pixels. The Sony sensor comprises a lower dynamic range and smaller pixels (6.9 μm×6.9 μm) and is associated with lower light-efficiency and signal-to-noise ratio (than e.g. Teledyne Photometrics Evolve 128), see also [32]. However, it was demonstrated that the signal quality is excellent in a multi-camera setting with the averaging of the signals from multiple cameras. Previously, it was determined that the video data resulting from these cameras can be tracked [7].

### 6.5.3. Limitations

A limitation in certain cases is that it is challenging to fully compensate residual illumination-related motion artifacts, see also section 4.1 in [7]. The physical motion between the tissue and the light sources persists even though the motion of the heart is apparently non-existent in the co-moving frame of reference after motion tracking. Regardless of the tracking, in physical space the tissue moves from slightly brighter to darker areas and vice versa, which leads to modulations of the baseline of the optical signals. While these baseline modulations can be compensated during post-processing, in other embodiments, ratiometric imaging and numerical methods are integrated to compensate illumination-related motion artifacts more thoroughly. Currently, data in which the heart surface is occluded (e.g. by the micro-electrode) is discarded. Another challenging technical aspect during the experiments is associated with the low dynamic range of the cameras (12-bit: 4,096 counts): the apertures of each of the cameras may be manually adjusted throughout the experiment to ensure that the brightnesses of the video images would match and that the full dynamic range of the sensor was utilized. These adjustments could only be made after staining and eventually needed to be repeated with each restraining for each camera individually. Even though GPU-accelerated motion tracking [7] was used in the 2D part of a hybrid 3D/2D tracking approach, the 3D part is implemented on the CPU and it takes several hours to track a few hundred frames. Other embodiments implement fully GPU-accelerated tracking to increase processing speeds. Lastly, still other embodiments have a direct method to validate reconstructions and further work is required to cross-register and compare the heart shape with the heart shape generated with a different imaging modality, such as ultrasound or computerized tomography. However, the minimal amount of residual dissociation-related motion artifacts, as described in [5], and the minimal blurring of the averaged video images projected onto the reconstructed heart surface, are indirect measures and indicators for the high precision and accuracy of embodiments of the invention.

### 6.6. Appendix

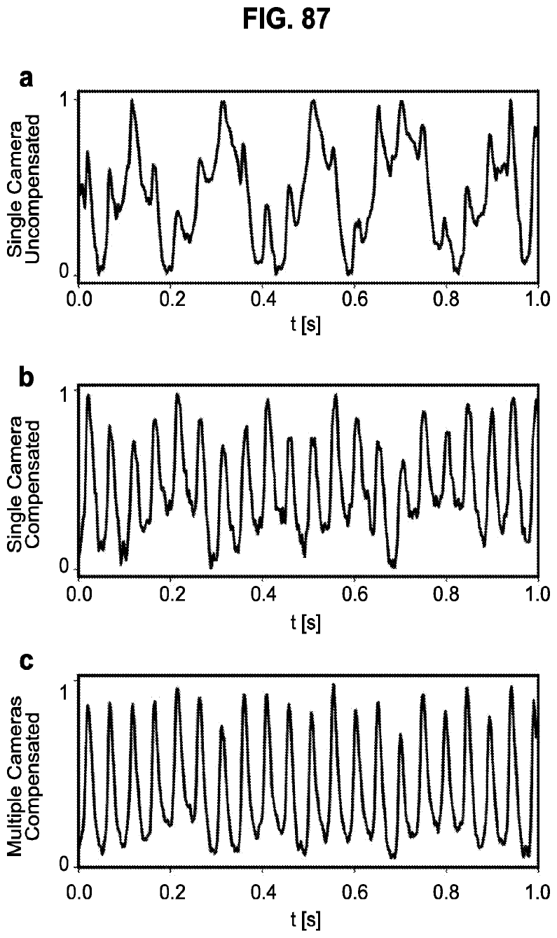

FIGS. 87A-C show optical traces from the ventricular surface of a fibrillating, contracting rabbit heart measured with a multi-camera optical mapping system. FIG. 87A shows uncompensated optical trace from a single pixel in the raw video data. The trace shows largely motion artifacts. FIG. 87B shows optical trace after motion compensation from a single pixel in the motion-stabilized video data by one camera (cam 11). The trace shows a sequence of action potentials and residual motion artifacts. FIG. 87c shows optical trace after motion compensation from a single pixel on the surface of the three-dimensional mesh (triangle 6380) averaged from multiple motion-stabilized videos by different cameras with overlapping fields of view. The trace shows a sequence of action potentials and significantly reduced residual motion artifacts.

### 6.7. Conclusions

For over 30 years, optical mapping of action potential or calcium waves has been performed in contraction-inhibited hearts. As a result, studying the interplay of cardiac bioelectricity and biomechanics, two of the main players involved in the heartbeat, has been limited. Here, these experimental results demonstrated, for the first time, panoramic optical mapping of action potential waves across the entire, three-dimensional deforming ventricular surface of beating hearts. Optical mapping system of embodiments provides high-resolution imaging data, which can be used to study both the electrical and mechanical heart muscle physiology in unprecedented detail, and could allow new insights into the mechanisms of mechano-electric feedback.

### 6.8. References

The numbering related to the following references apply with respect to the experimental results presented in Example 6:
[1] Salama et al. G. Salama, R. Lombardi, and J. Elson, Maps of optical action potentials and NADH fluorescence in intact working hearts, American Journal of Physiology-Heart and Circulatory Physiology 252, H384 (1987).
[2] Zhang et al. H. Zhang, K. lijima, J. Huang, G. P. Walcott, and J. M. Rogers, Optical mapping of membrane potential and epicardial deformation in beating hearts, Biophysical Journal 111, 438 (2016).
[3] Christoph et al. J. Christoph, J. Schröder-Schetelig, and S. Luther, Electromechanical optical mapping, Progress in Biophysics and Molecular Biology 130, 150 (2017).
[4] Christoph et al. J. Christoph, M. Chebbok, C. Richter, J. Schröder-Schetelig, P. Bittihn, S. Stein, I. Uzelac, F. H. Fenton, G. Hasenfuss, R. J. Gilmour, and S. Luther, Electromechanical vortex filaments during cardiac fibrillation, Nature 555, 667 (2018).
[5] Christoph and Luther J. Christoph and S. Luther, Marker-free tracking for motion artifact compensation and deformation measurements in optical mapping videos of contracting hearts, Frontiers in Physiology 9, 1483 (2018).
[6] Kappadan et al. V. Kappadan, S. Telele, I. Uzelac, F. Fenton, U. Parlitz, S. Luther, and J. Christoph, High-resolution optical measurement of cardiac restitution, contraction, and fibrillation dynamics in beating vs. Blebbistatin-uncoupled isolated rabbit hearts, Frontiers in Physiology 11, 10.3389/fphys.2020.00464 (2020).
[7] Example 4: Real-Time Optical Mapping of Contracting Cardiac Tissues With GPU-Accelerated Numerical Motion Tracking (provided above).
[8] V. Kappadan, A. Sohi, U. Parlitz, S. Luther, I. Uzelac, F. Fenton, N. S. Peters, J. Christoph, and F. S. Ng, Optical mapping of contracting hearts, The Journal of Physiology n/a, https://doi.org/10.1113/JP283683.
[9] Chen et al. Z. Chen, K. Nguyen, G. Kowalik, X. Shi, J. Tian, M. Doshi, B. R. Alber, X. Guan, X. Liu, X. Ning, M. W. Kay, and L. Lu, Transparent and stretchable au-ag nanowire recording microelectrode arrays, Advanced Materials Technologies 8, 2201716 (2023).
[10] Gray et al. R. A. Gray, J. Jalife, A. Panfilov, W. T. Baxter, C. Cabo, J. M. Davidenko, and A. M. Pertsov, Nonstationary vortexlike reentrant activity as a mechanism of polymorphic ventricular tachycardia in the isolated rabbit heart, Circulation 91, 2454 (1995).
[11] Swift et al. L. M. Swift, M. W. Kay, C. M. Ripplinger, and N. G. Posnack, Stop the beat to see the rhythm: excitation-contraction uncoupling in cardiac research, American Journal of Physiology-Heart and Circulatory Physiology 321, H1005 (2021).
[12] Efimov et al. I. R. Efimov, V. P. Nikolski, and G. Salama, Optical imaging of the heart, Circ. Res. 95, 21 (2004).
[13] Bray et al. M.-A. Bray, S.-F. Lin, and J. Wikswo, Three-dimensional surface reconstruction and fluorescent visualization of cardiac activation, IEEE Transactions on Biomedical Engineering 47, 1382 (2000).
[14] Kay et al. M. Kay, P. Amison, and J. Rogers, Three-dimensional surface reconstruction and panoramic optical mapping of large hearts, IEEE Transactions on Biomedical Engineering 51, 1219 (2004).
[15] Kay et al. M. W. Kay, G. P. Walcott, J. D. Gladden, S. B. Melnick, and J. M. Rogers, Lifetimes of epicardial rotors in panoramic optical maps of fibrillating swine ventricles, American Journal of Physiology-Heart and Circulatory Physiology 291, H1935 (2006).
[16] Rogers et al. J. M. Rogers, P. G. Walcott, J. D. Gladden, S. B. Melnick, and M. W. Kay, Panoramic optical mapping reveals continuous epicardial reentry during ventricular fibrillation in the isolated swine heart, Biophysical Journal 92, 1090 (2007).
[17] Rohde et al. G. K. Rohde, B. M. Benoit, and S. Lin, Corrections of motion artifacts in cardiac optical mapping using image registration, IEEE Trans. Biomed. Eng. 52, 338 (2005).
[18] Svrcek et al. M. Svrcek, S. Rutherford, and B. Smaill, Characteristics of motion artifacts in cardiac optical mapping studies, Conference Proceedings, 31st Annual International Conference of the IEEE EMBS, Minneapolis, USA 31, 3240 (2009).
[19] Khwaounjoo et al. P. Khwaounjoo, S. L. Rutherford, M. Svrcek, I. J. LeGriece, M. L. Trew, and B. H. Smaill, Image-based motion correction for optical mapping of cardiac electrical activity, Ann. Biomed. Eng. 43, 1235 (2015).
[20] Rodriguez and Nygren M. P. Rodriguez and A. Nygren, Motion estimation in cardiac fluorescence imaging with scale-space landmarks and optical flow: a comparative study, IEEE Trans. Biomed. Eng. 62, 774 (2015).
[21] Bourgeois et al. E. B. Bourgeois, A. D. Bachtel, J. Huang, G. P. Walcott, and J. M. Rogers, Simultaneous optical mapping of transmembrane potential and wall motion in isolated, perfused whole hearts, Journal of Biomedical Optics 16, 096020 (2011).
[22] Garrido-Jurado et al. S. Garrido-Jurado, R. Muñoz-Salinas, F. Madrid-Cuevas, and M. Marín-Jimenez, Automatic generation and detection of highly reliable fiducial markers under occlusion, Pattern Recognition 47, 2280 (2014).
[23] Kwon Y.-H. Kwon, Object plane deformation due to refraction in two-dimensional underwater motion analysis, Journal of Applied Biomechanics 15, 396 (1999).
[24] Oliver Batchelor Oliver Batchelor, multical (2023).
[25] Schönberger et al. J. L. Schönberger, E. Zheng, M. Pollefeys, and J.-M. Frahm, Pixelwise view selection for unstructured multi-view stereo, in European Conference on Computer Vision (ECCV) (2016).
[26] Klaudiny and Hilton M. Klaudiny and A. Hilton, Cooperative patch-based 3d surface tracking, in 2011 Conference for Visual Media Production (IEEE, 2011).
[27] Barnes et al. C. Barnes, E. Shechtman, A. Finkelstein, and D. B. Goldman, PatchMatch: A randomized correspondence algorithm for structural image editing, ACM Transactions on Graphics (Proc. SIGGRAPH) 28 (2009).
[28] Farnebäck G. Farneback, Two-Frame Motion Estimation Based on Polynomial Expansion, in Image Analysis, Vol. 2749, edited by G. Goos, J. Hartmanis, J. van Leeuwen, J. Bigun, and T. Gustavsson (Springer Berlin Heidelberg, Berlin, Heidelberg, 2003) pp. 363-370.
[29] Lebert et al. J. Lebert, N. Ravi, F. H. Fenton, and J. Christoph, Rotor localization and phase mapping of cardiac excitation waves using deep neural networks, Frontiers in Physiology 12, 10.3389/fphys.2021.782176 (2021).
[30] Li et al. X. Li, T. P. Almeida, N. Dastagir, M. S. Guillem, J. Salinet, G. S. Chu, P. J. Stafford, F. S. Schlindwein, and G. A. Ng, Standardizing single-frame phase singularity identification algorithms and parameters in phase mapping during human atrial fibrillation, Frontiers in Physiology 11, 10.3389/fphys.2020.00869 (2020).
[31] Lebert and Christoph J. Lebert and J. Christoph, Synchronization-based reconstruction of electromechanical wave dynamics in elastic excitable media, Chaos: An Interdisciplinary Journal of Nonlinear Science 29, 10.1063/1.5101041 (2019).
[32] Lee et al. P. Lee, C. J. Calvo, J. M. Alfonso-Almazin, J. G. Quintanilla, F. J. Chorro, P. Yan, L. N. Loew, D. Filgueiras-Rama, and J. Millet, Low-cost optical mapping systems for panoramic imaging of complex arrhythmias and drug-action in translational heart models, Scientific Reports 7, 10.1038/srep43217 (2017).
[33] Rieger et al. M. Rieger, C. Dellenbach, J. vom Berg, J. Beil-Wagner, A. Maguy, and S. Rohr, Enabling comprehensive optogenetic studies of mouse hearts by simultaneous opto-electrical panoramic mapping and stimulation, Nature Communications 12, 10.1038/s41467-021-26039-8 (2021).

### Example 7: Combined Optical Mapping and 4D Ultrasound

### 7.1. Introduction

In addition to the optical mapping setup described in Example 6, the imaging setup also incorporates an ultrasound system for the simultaneous acquisition of volumetric ultra-sound (US) images of the beating heart. In this section, I describe this part of the experimental setup, provide a more detailed description of some of the electronic parts of the setup, describe the processing of the ultrasound data to extract the mechanical deformation of the heart, and apply the above discussed neural network to this data.

### 7.2. Experimental Setup

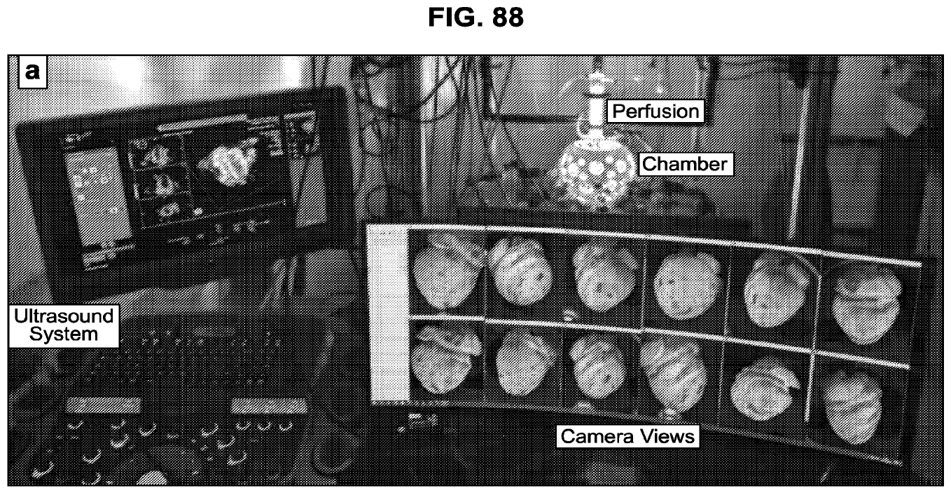

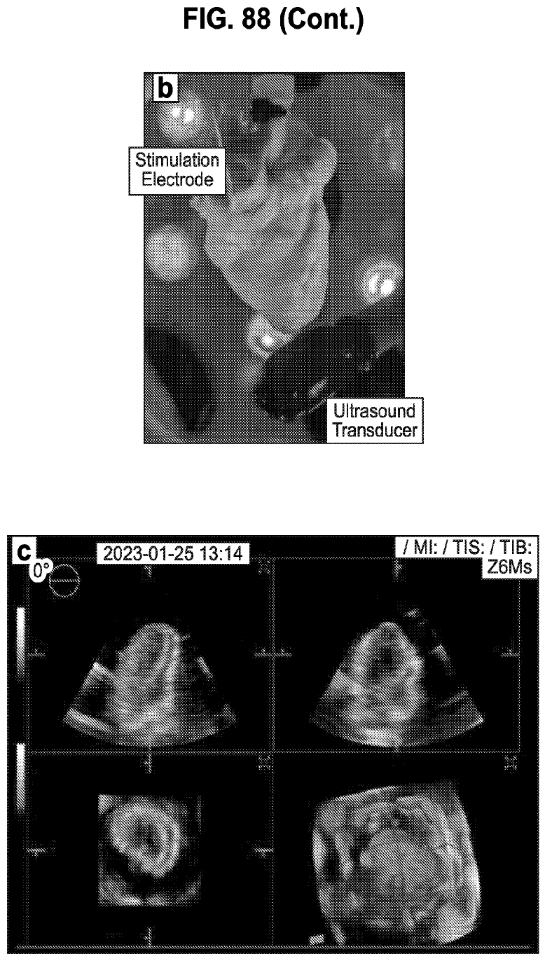

FIG. 88A presents an overview of the complete panoramic imaging setup according to an embodiment. The acquisition of ultrasound data is enabled by an ultrasound transducer, which is carefully positioned within the perfusion chamber as depicted in FIG. 88B. Piezoelectric elements within the probe generate ultrasound waves by converting electrical signals into mechanical waves, and vice versa. As these ultrasound waves penetrate the tissue, they reflect back based on the density and composition of the structures within the heart. The Siemens Acuson SC2000 ultrasound system (Siemens Healthineers, Germany) is employed with the Z6Ms phased array transducer, which has 48×48 piezoelectric elements operating at a 2.5 MHz center frequency. This two-dimensional matrix of piezoelectric elements generates a diverging, pyramidal-shaped beam through electronic scanning. The received echoes are processed to create real-time 3D ultrasound images, commonly known as 4D ultrasound. For details on the principles behind volumetric ultrasound imaging, refer to Prager et al. (2010) and Huang and Zeng (2017). The Z6Ms is a transesophageal echocardiography (TEE) transducer, allowing one to operate it fully submerged in the perfusion chamber. The generated B-mode (brightness mode) volumetric ultrasound recordings are shown in FIG. 88C. The TEE transducer can image at speeds between 10 and 134 volumes per second (vps) using the T1 imaging mode, depending on the selected field of view and depth. The maximum acoustical field of view for the Z6Ms is 90°×90° with a depth of 260 mm. Embodiments typically set a field of view of about 70°×62° with a depth of around 70 mm to record rabbit hearts with an acquisition rate of around 26 vps. This is slower than one would like for the timescales of the heart's contraction. E.g., during pacing it takes about 90 ms for the electrical propagation to envelop the complete ventricles, in which time embodiments can capture only 2.3 volumes. One could image the heart at a field of view of 42°×42° to achieve imaging speeds of 46 vps, which gives embodiments more than 4 volumes during the activation. However, this currently captures only a portion of the heart, posing challenges for segmentation and motion tracking as parts of the heart move in and out of the field of view, as described herein. Other embodiments aim to capture the entire heart in the field of view at imaging speeds of about 40 vps through careful positioning of the transducer and choice of the imaging parameters.

FIG. 88A shows an overview of the panoramic optical mapping setup with volumetric ultrasound. FIG. 88B shows a transesophageal (TEE) matrix-array ultrasound transducer (Z6Ms, Siemens) imaging the rabbit heart from below, LEDs in the background. FIG. 88C shows cross-sectional views of the B-mode (brightness mode) volumetric ultrasound recording, volumetric rendering in the bottom right.

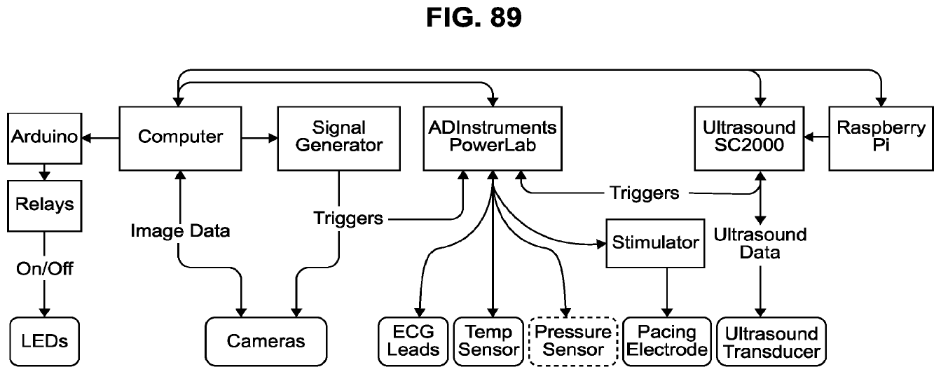

FIG. 89 shows a schematic representation of the computer-controlled optical mapping system's electrical components, enabling synchronized acquisition of camera data, ultrasound data, and time-series measurements including ECG and temperature readings.

The frame acquisition of embodiments' cameras (500 fps) is synchronized using a TTL (transistor-transistor logic) trigger signal. However, the acquisition of ultrasound data cannot be controlled with a TTL signal. To address this issue, the ultrasound system was modified to obtain a TTL signal indicating when it is recording. This signal, along with the camera triggers, are recorded using the ADInstruments PowerLab acquisition system. The ultrasound recordings have a maximum duration of 10 seconds, utilizing a ring buffer that continuously retains the most recent 10 seconds of data in memory. To stop the acquisition and save the data automatically, a Raspberry Pi (Model 3 A+, Raspberry Pi Foundation) was used to emulate a USB footswitch connected to the US system. During the experiment, one can communicate with the ultrasound system using the DICOM (Digital Imaging and Communications in Medicine) standard to log the corresponding camera and ultrasound recordings. This communication is facilitated by the pynetdicom software library (scaramallion et al., 2022).

A significant portion of obtaining the presented experimental results involved advancing and developing software tools essential to various aspects of embodiments. This is also the case for experimental setups, where these tools streamline experimental procedures, ensure seamless integration of equipment, and facilitate data analysis. The following list outlines software tools developed for acquiring and processing optical mapping and ultrasound data:
ExpCoordinator: This software suite orchestrates and coordinates the various components of embodiments' experimental setup. ExpCoordinator coordinates the camera data acquisition, the signal generator for camera synchronization, LEDs, ADInstruments LabChart for time-series acquisition, the ultrasound machine through DICOM and a Raspberry Pi, and logging of the steps taken during the experiment, e.g. dye infusion. ExpCoordinator is designed to be modular and extensible.
Multi-Camera Recording: Recording software for multi-camera acquisition during embodiments' optical mapping experiments. Supports recording of monochromatic 8 to 16-bit image data for the Basler pylon and IDS μEye CMOS cameras. Written in C++.
Component Software: Software for various experimental components with which the ExpCoordinator coordinates, e.g. a Python library to communicate with the signal generator, software for the Arduino which controls the LEDs via electromechanical relays, and software for the Raspberry Pi.
Monochrome: Viewer for scientific monochromatic video data, such as optical mapping recordings and simulation data. Monochrome is designed for ease of use and versatility to view multiple recordings at once, in particular scientific high-dynamic-range recordings, such as embodiments' 12-bit camera recordings or floating-point data. Monochrome offers playback at very high frame rates, along with the capability to add layers for enhanced analysis and visualizing motion tracking information. Written in C++, Python bindings are available.
Optimap: A Python software library for analysis of optical mapping recordings. It encompasses various algorithms and computational methods for processing optical mapping recording, in particular for motion tracking and compensation. Written in Python with some of backend computation in C++.
Ultrasound Toolbox: An internal Python library for processing, converting, and motion tracking volumetric ultrasound data. The toolbox combines various tools for visualizing, tracking, masking, processing of segmentation, and converting between data formats of volumetric data. Additionally, it can analyze the ultrasound and camera TTL signals recorded by LabChart to synchronize the recordings.
   7.3. Cardiac Deformation from 4D Ultrasound

For the inverse-mechano electrical problem, there is a need to extract the deformation of the heart muscle from the recorded B-mode volumetric ultrasound data. In particular, there is a need for a particle based representation of moving and deforming ventricles to apply a neural network which was trained on SPH simulations, as described herein. Accomplishing this task is challenging due to the presence of acoustic interferences (speckle noise) and artifacts, including shadows, reverberations, and attenuation, in the recorded B-mode volumetric ultrasound data. Speckle noise, a well-known characteristic of ultrasound imaging, results from interactions between emitted waves and small anatomical structures that are significantly smaller than the ultrasound wavelength. These interactions give rise to scattering phenomena that create either destructive or constructive interference patterns, which manifest as granular, textured patterns. Although speckle noise is commonly considered an undesirable feature that negatively impacts the quality of ultrasound images, speckles do follow the motion of underlying tissues, making them reliable spatial markers for tracking moving tissues, such as the heart. Other ultrasound artifacts, particularly reverberations from the fluid surface and bath walls, acoustic shadows, and attenuation, can cause significant information loss and make the segmentation and motion tracking of ventricles in the imaging setup challenging. In this section, a processing pipeline is described, which comprises four steps: preprocessing, ventricular segmentation, dense motion tracking of the ultrasound data using optical flow, and postprocessing to generate a particle-based representation of the ventricles.

In the first step, the recorded volumetric ultrasound data is converted into a uniform Cartesian coordinate system to facilitate further processing. The ultrasound system stores the recorded B-mode data in DICOM format as a series of 3D volume arrays using a manufacturer-specific spherical coordinate system (range, azimuth, elevation). The spatial resolution of the raw ultrasound data decreases with distance from the transducer. This data is resampled into a uniform Cartesian coordinate system using cubic spline interpolation.

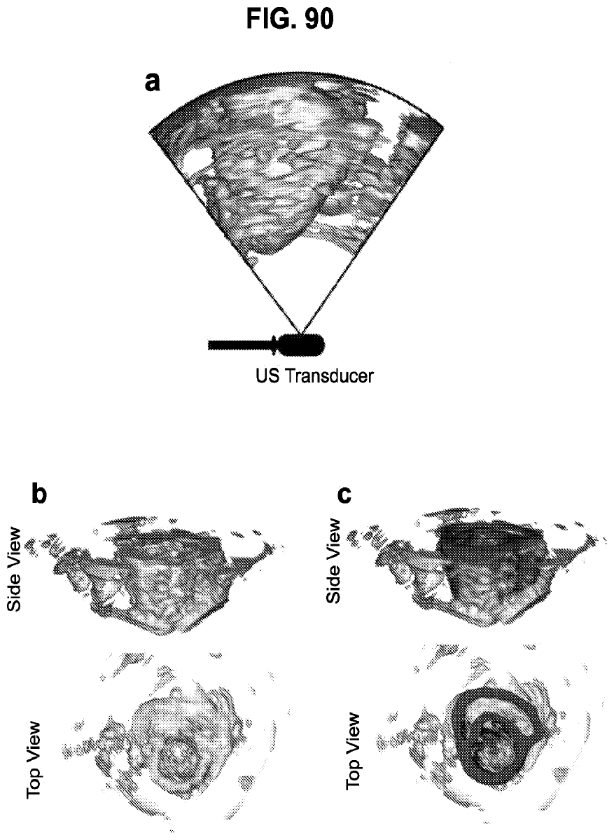

In the next step, a segmentation mask of the ventricles for one reference volume of the recording is created. The segmentations were performed manually and 3D Slicer (Fedorov et al., 2012) was used for the 3D segmentation of the ventricles and left and right chambers. The segmentation of volumetric ultrasound data is challenging: the contrast between areas of interest is often low and open contours and ill-defined boundaries frequently occur (Noble & Boukerroui, 2006; Mozaffari & Lee, 2016). FIG. 90 shows an example volume rendering of the ultrasound data and the segmented ventricles. In some cases, the complete ventricles were not segmented, as some of the upper parts are often obscured by reverberation and other artifacts and could not be motion tracked reliably. Additionally, if pacing through the stimulation electrode was used, the position of the electrode was segmented either directly from the recording or by using a separate recording where the electrode position was marked in the ultrasound images using a hex key.

FIGS. 90A-C show ventricular segmentation from B-mode ultrasound data. FIG. 90A shows a frontal view of a volume rendering of the pyramidal-shaped ultrasound data. FIG. 90B shows a side and top view with the upper portion of the pyramid removed for improved visualization of the heart. FIG. 90C shows manually segmented ventricles shown in red.

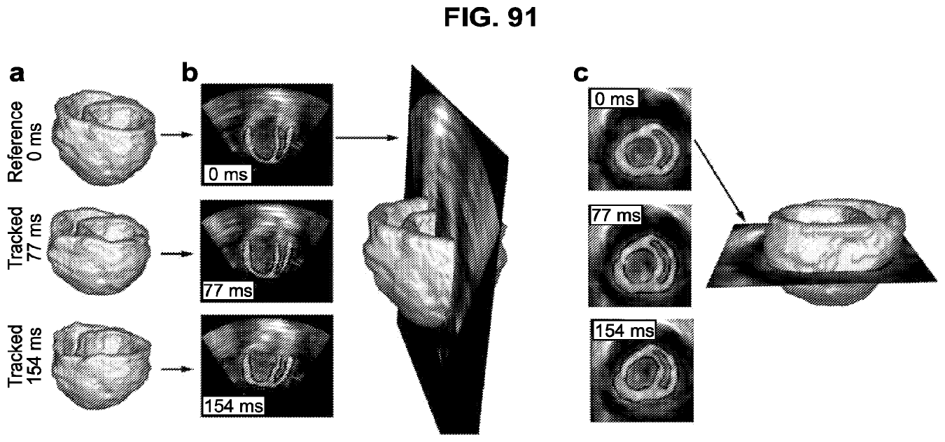

Most of the techniques used for cardiac motion estimation from 3D ultrasound fall into three main categories: block-matching, optical flow and elastic image registration (Alessandrini et al., 2016). Building on experience in tracking optical mapping data, it was chosen to utilize a 3D implementation of the Farneback (2003) optical flow method to track the volumetric ultrasound data, which has been also used in other 2D and 3D echocardiography studies (Danudibroto et al., 2016; Ostvik et al., 2021). A GPU-accelerated implementation of 3D Farnebäck by Seitz (2018) was used, which was slightly modified (to fix some bugs). The motion between the segmented reference volume to all other volumes in the recording was computed. FIGS. 91A-C demonstrate the results of the dense motion tracking by warping the reference segmentation using the optical flow fields. Overall, it was observed that the motion tracking works well. The warped segmentations correspond closely to the motion of the heart observed in the ultrasound data. However, tracking errors for areas with artifacts in the ultrasound recording, as well as temporal inconsistencies occur frequently. Spatio-temporal smoothing filters of the computed optical flow fields were employed to reduce these errors. However, it is unclear how strong this smoothing should be as it could also remove the fine-scaled motion of the heart. The optical flow computation is demanding, requiring about 6 hours of computation time per recording, or about 1.3 minutes per volume, on a Nvidia RTX 24000 GPU, depending on the resolution. A faster alternative implementation of 3D Farnebäck and 3D Lucas and Kanade (1981) by Huang et al. (2023) was evaluated. However, after a systematic parameter search, suitable parameters that yielded results as good as those obtained with the Seitz (2018) implementation could not be found. To search for suitable parameters, the Optuna hyperparameter optimization library (Akiba et al., 2019) was used and the computed optical flow was evaluated by warping the reference volume and computing the root mean squared error against the target volume on either the volume as a whole or on the segmented ventricles. However, this optimization did not yield better optical flow based on visual comparison than those obtained with the Seitz (2018) implementation when using 6 pyramid levels and default parameters otherwise.

FIGS. 91A-C show optical flow-based motion tracking of the ventricles in 4D ultrasound data. Segmentation of the ventricles was performed for one volume (reference volume), and subsequently deformed using the dense optical flow between the reference volume and the other time points. FIG. 91A shows reference segmentation (t=0 ms) and estimated segmentation at t=77 ms and t=154 ms using motion tracking. FIGS. 91B-C show cross-sectional views of the ultrasound data for these three time points with segmentation boundaries shown in red. Note that these cross-sectional views may not depict the identical sections of the heart, as the heart moves out-of-plane.

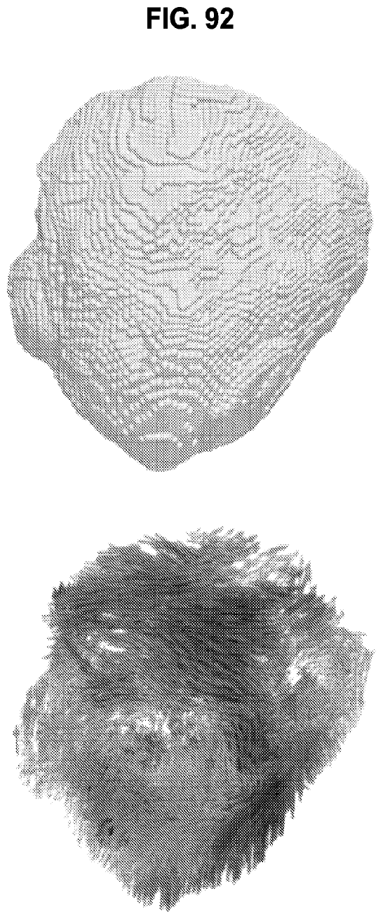

In the final step, the segmentation on the reference volume and the optical flow motion maps are combined to generate the sparse particle-based representation. Each voxel was represented inside the segmented ventricles as a particle with coordinates corresponding to the center of the voxel. Particles for which the brightness in the B-mode ultrasound are below a certain threshold are removed to reduce tracking errors. The trajectory of these particle over time is then given by the computed optical flow maps from the reference volume to the other volumes. FIG. 92 shows an example of the particle-based representation of the ventricles in the 4D ultrasound data.

FIG. 92 shows particle-based representation of ventricles from 4D ultrasound data, generated by combining the reference volume segmentation and optical flow motion maps. Right: View of the particle-based representation. Left: Displacement vectors from one volume to the next.

### 7.4. Inverse Mechano-Electrical Predictions

This section describes how the inverse mechano-electrical neural network described herein can be applied on the experimental data of beating rabbit hearts. The neural network was trained exclusively on the bi-ventricular SPH simulations, and was evaluated on the numerical simulation data described herein, where it is shown that the neural network could predict the electrical excitation from the mechanical deformation with high accuracy for numerical simulations, with predictions for simulations featuring focal dynamics proving to be the most accurate and robust.

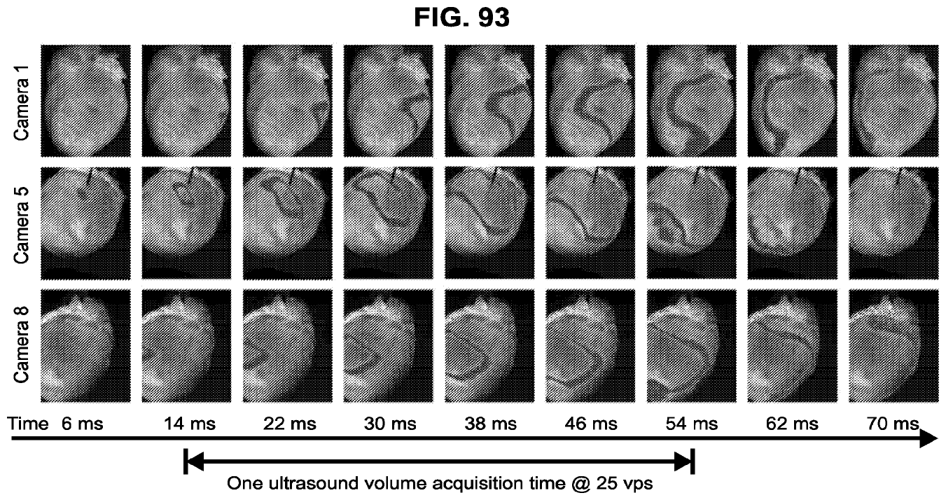

These focal dynamics were replicated in voltage-sensitive optical mapping experiments of beating rabbit hearts using local pacing with a platinum-iridium micro electrode (FHC, USA) at different heart locations. FIG. 93 shows a sample propagation of the action potential wave front (false colors, purple) across the heart following a stimulation. For this visualization, the embodiment motion tracked and compensated the fluorescent recordings, and then computed the frame difference using a window size of 5 frames and used a pixel-wise normalization. This extracts the front of the action potential wave, which is then warped back into the moving frame and overlaid on top of the original recordings. The wave originates from the tip of the electrode which is in direct contact with the heart, see the images of camera 5.

FIG. 93 shows action potential wavefront propagation across a beating rabbit heart over time following electrical stimulation. Optical mapping with voltage-sensitive staining, the wavefront was extracted from the change in fluorescence signal and is shown in false color (purple). Views from three cameras, camera 1 (top), 5 (middle), and 8 (bottom), out of an embodiment's 12-camera setup are shown. Camera 1 is positioned to the left of camera 5, and camera 8 to the right. Stimulation by the pacing electrode occurred at t=0 ms.

As illustrated in FIG. 93, the volumetric ultrasound imaging rate of around 25 vps is slow in comparison to the timescale of the electrical wave dynamics in the rabbit heart. The neural network of embodiments requires five displacement vector fields as input and was trained on simulation data with a synthetic imaging speed of 31 Hz to 77 Hz, as described herein.

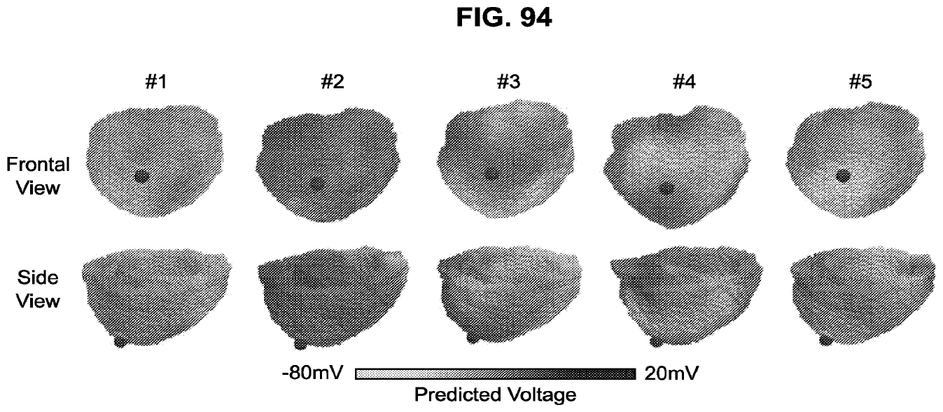

This section presents the preliminary results of applying the inverse mechano-electrical neural network described herein to pacing data of a rabbit heart using the particle-based representation of the ultrasound data. The embodiment used a spatio-temporal smoothing filter for the computed 3D optical flow fields to reduce tracking errors. The neural network model was trained on the SPH simulation dataset described herein using four all electrophysiology models, e.g. it is the model with which predictions were generated. The primary aim of this initial validation step was to determine whether the location of the pacing sites could be identified. However, the neural network predictions were not accurate and robust enough to locate the pacing sites reliably. Instead, spurious excitation patterns are predominant, which make an identification of a pacing site difficult. FIG. 94 shows predictions of the electrical excitation for five pacing stimulations in a rabbit heart from 4D ultrasound. The stimulation electrode was placed on the right ventricle and stimulation was performed with a frequency of 4 Hz. The true action potential wave dynamics is a focal wave emanating from the electrode location. The predictions are all from a single 10 second long 4D ultrasound recording which was acquired at 25.8 vps. The position of the electrode was identified from the ultrasound recording and is marked in the visualization with a red circle. The timing of the stimulation pacing and the ultrasound acquisition were recorded using ADInstruments PowerLab acquisition system, the predictions for the volume acquired after the stimulation occurred are shown. The input of the neural network is the heart's motion for five displacement frames subsequent to the stimulation.

FIG. 94 shows inverse mechano-electrical prediction with the neural network for 5 pacing stimulations in a rabbit heart from 4D ultrasound (shown are the individual predictions for the next volume acquired after the stimulation occurred). The red circle marks the location of the pacing electrode. After each stimulation a focal wave emanates away from this location and the prediction should show a focal pattern. The pacing site could not be reliably identified from the predicted excitation state. However, some individual predictions show focal patterns, which is promising and motivates further work. The neural network was only trained on SPH simulation data and predicts the three-dimensional transmembrane voltage from the mechanical motion obtained by motion tracking the 4D ultrasound data. The imaging rate of the ultrasound was 25.8 vps.

### 7.5. Conclusions

Over the past 30 years, the field of cardiac optical mapping has predominantly steered clear of studying contracting hearts due to the technique's high sensitivity to motion. Throughout the experimental results presented here, however, embodiments have been used to image beating mice hearts, rabbit hearts, and cardiac cell cultures using optical mapping. In this section, the innovative methods that enabled these achievements are described; the first fully panoramic electromechanical optical mapping system is presented; and the integration of volumetric ultrasound is demonstrated. This experimental platform is a powerful tool for studying the interplay between electrical excitation and tissue deformation, and will be used to further investigate the inverse mechano-electrical problem. In the following, the implications for the inverse mechano-electrical problem and the field of cardiac optical mapping are summarized.

These sections focused on the motion tracking and compensation of optical mapping data. The methods discussed in these sections are not only applicable to cardiac optical mapping, but also extend to various other fluorescence imaging data of moving samples in general. For instance, embodiments have successfully applied these methods to calcium imaging data of isolated stomach strips (Vogt et al., 2021). In Lebert et al. (2022), it was demonstrated that real-time motion tracking and compensation of optical mapping data is achievable using GPU-accelerated algorithms. Five optical flow algorithms implemented by the open-source OpenCV library (Bradski, 2000) were evaluated on optical mapping data of contracting rabbit, mouse, and pig hearts and cardiac cell cultures imaged with four different high-speed cameras as well as on synthetic optical mapping data. Based on this evaluation, Farnebäck (2003) was selected as a standard motion tracking algorithm for optical mapping data. One of the reasons for this choice was the very high processing speeds, which have enabled embodiments to efficiently process the video data from embodiments' panoramic multi-camera optical mapping system with up to 16 cameras. All evaluated conventional optical flow algorithms were highly sensitive to a change of the fluorescence wave patterns between the tracked images, requiring preprocessing in form of contrast-enhancement to remove them. The limitations of contrast-enhancement were discussed and a deep learning approach for motion tracking that is robust to the fluorescent wave patterns was presented. The deep learning approach does not require contrast-enhancement; instead, the model learns on its own to prevent cross-talk between motion and physiological function encoded in the fluorescence signal. The model is trained on synthetic optical mapping data and has been evaluated on both synthetic and experimental optical mapping data. The network outperforms conventional methods both in tracking accuracy as well as robustness to noise, and in contrast to conventional algorithms showed no decrease in tracking accuracy as fluorescence signal strength increases. However, additional work is required to ensure the network's ability to process larger images and its generalization to image data from different experimental setups. One of the key challenges for training any supervised neural network is the generation of training data in sufficient quantity and diversity. Here the training data needs to be expanded to data from more cameras and tissues, as well as simulations which cover more diverse scenarios. Embodiments of the panoramic optical mapping setup could be a valuable tool for this purpose, as it generates large quantities of video data and reconstructs 3D deforming and moving meshes of the heart which could be used to generate synthetic optical mapping data with a higher degree of realism.

Also described is the first fully panoramic electromechanical optical mapping system covering the entire 360° heart surface and that provides several key improvements over previously reported 3D electromechanical optical mapping setups (Zhang et al., 2016; Christoph et al., 2017). Using 12 calibrated high-speed cameras, the entire 3D deforming ventricular surface was reconstructed with corresponding high-resolution voltage-sensitive optical measurements. To achieve this, multi-view stereo reconstruction using COLMAP (Schönberger et al., 2016) was used to reconstruct a deformable mesh for each recording. This mesh was then fitted to the video data over time to accurately capture the deformation of the heart (Klaudiny & Hilton, 2011; Furukawa & Ponce, 2010). With this advanced setup, an embodiment was able to measure action potential waves at unprecedented resolutions on the contracting 3D surface of rabbit hearts during sinus rhythm, paced rhythms, and during ventricular fibrillation. This setup allows embodiments to capture the complete electrical surface dynamics and investigate the interplay between electrical excitation and tissue deformation. While conventional computer vision and stereo vision techniques were used primarily, in recent years, deep learning methods have increasingly been used for multi-view 3D reconstruction (Wang et al., 2021). The use of these techniques could further improve the accuracy of reconstructed meshes and their deformation over time, allowing for even more precise measurements of the heart's electrical and mechanical dynamics. Overall, an embodiment comprising a fully panoramic electromechanical optical mapping system offers an innovative and highly effective approach to studying the electromechanical behavior of the heart under normal and abnormal conditions.

Finally, the integration of optical mapping and 4D ultrasound is presented, which is the final step in realizing an experimental setting that could eventually provide a proof-of-principle that electrophysiologial wave phenomena can be reconstructed from cardiac deformation. A volumetric transesophageal transducer was used to image isolated rabbit hearts with simultaneous and synchronized panoramic optical mapping. While some embodiments focus on rabbit hearts, others focus on pig hearts and utilize a transthoracic echocardiography (TTE) transducer (4Z1c, Siemens) for higher temporal resolution and improved image quality. To measure the deformation of the ventricles in the volumetric ultrasound data, a GPU-accelerated 3D version of the Farnebäck (2003) optical flow algorithm was used and manual segmentation of the ventricles in 3D Slicer (Fedorov et al., 2012). The segmentation and motion tracking of 4D ultrasound data pose significant challenges. The segmentation process is laborious and time-consuming due to high noise levels and other artifacts, which result in open contours and ill-defined boundaries of the heart in the volumetric ultrasound data. These issues stem in part from choices of ultrasound imaging parameters, which prioritize imaging speed at the expense of image quality. Other embodiments could involve streamlining the segmentation process by employing automatic segmentation techniques and reducing spurious echoes and reverberations by using ultrasound-absorbing materials at the fluid surface and the chamber walls.

An optical flow-based motion tracking approach for the volumetric ultrasound demonstrates satisfactory to good results with the potential for further refinement. The ultrasound data is difficult to track due to the same problems encountered during segmentation. The approach works well for the large-scale motion, however tracking errors persists in some regions of the ultrasound image, in particular in the upper part towards the base of the heart, where spatial resolution decreases and artifacts are more frequent. Validating the accuracy of motion tracking for small-scale motion, particularly for intramural deformation, is challenging due the three-dimensional nature of data acquired using embodiments of the invention. Further research and exploration are necessary to identify the optimal optical flow algorithm parameters for accurately capturing these intricate movements. Some embodiments improve the tracking accuracy by switching to a volume-to-volume approach, tracking the motion from one time point to the next one, instead of from a reference volume to all other volumes. However, ultimately, there is a need to transform the motion-tracking data into a particle-based representation for the electrical reconstruction neural network, which is not directly possible in the same fashion when using a volume-to-volume tracking approach. Similar to tracking the optical mapping data, embodiments use an optical flow neural network trained on synthetic ultrasound data with known ground truth deformation. Ostvik et al. (2021) and Evain et al. (2022) have successfully demonstrated such an approach by training a modified PWC-Net neural network for optical flow on synthetic 2D ultrasound data. Several frameworks exist for simulating ultrasound data, such as SIMUS (Garcia, 2022) and Field II (Jensen, 2004). Embodiments comprising Field II were used to simulate ultrasound data using SPH simulations of embodiments. By directly using the SPH particles as scatter points, it is possible to generate realistically looking 2D ultrasound data. Ultimately, training the inverse mechano-electrical neural network directly on synthetic 3D ultrasound data generated from the SPH simulations could improve the accuracy of the predictions and bypass the challenges of accurately segmenting and motion-tracking the ultrasound data.

The successful integration of panoramic optical mapping and volumetric ultrasound enable the ability to study the interplay between electrical excitation and tissue deformation in the heart. Embodiments used in connection with extracting heart muscle deformation from ultrasound recordings will continue to advance understanding and capabilities in this area. Embodiments of a neural network for predicting the electrical wave dynamics from the mechanical deformation data have been applied to predict the electrical wave pattern when tested on recordings in which a rabbit heart was paced. Such were used to identify the pacing site from which the focal wave originates in the predictions. Such applications are challenging for several reasons. First, the ultrasound imaging speed was too low to capture the fast electrical wave dynamics. With the around 26 volumes per second (vps), embodiments captured only on average 2.3 volumes in the about 90 ms it takes for the electrical propagation to propagate across the ventricles. For the next experiments, embodiments are configured to achieve acquisition speeds of about 46 vps by further restricting the field of view of the ultrasound transducer and careful positioning of it. Embodiments of a neural network developed for the in silico application uses five volumes as input and is trained on more temporally dense training data. Second, the motion tracking of the ultrasound data contains tracking errors, which can manifest as spurious large deformation of certain areas. These need to be replicated as a data augmentation step in the training process to improve the robustness of the neural network against it. Currently, spatio-temporal smoothing of the estimated motion fields is used to reduce these artifacts. However, this could also remove important fine-scaled intramural deformation. Fourth, the neural network is currently trained on the SPH simulation which are not specially targeting rabbit hearts, but rather are much more similar to human-sized heart. Moreover, more work is needed to establish a precise calibration between the coordinate systems of the ultrasound and the panoramic mesh reconstruction from the cameras, e.g. using 3D-printed phantoms (Ronchetti et al., 2022).

### Example 8: Further Development of Mechano-Electrical Deep Learning Models

Although the deep learning-based inverse mechano-electrical reconstruction approach was successfully used to simulate data discussed above, the neural network was solely trained on synthetic data generated with SPH simulations. Further investigation and optimization of the approach with experimental data may be necessary. The immediate next steps are to improve the ultrasound imaging acquisition rate and the motion tracking of the volumetric ultrasound data (see Example 7), as well as to improve the robustness of the neural network with respect to noise, in particular the noise characteristics of the tracking data. Additionally, characterizing how well the mechanical dynamics of the experimental data are represented in the simulation training dataset is needed. In the medium term, the network could be finetuned on the experimental surface electrical data using the reconstructed surface meshes of the panoramic imaging setup. In the long term, one could train the inverse mechano-electrical neural network or a 3D optical flow neural network on synthetic ultrasound data generated using cardiac electromechanically coupled simulations.

New echocardiography technologies such as ultrafast ultrasound imaging, which allow acquisition rates up to 5200 volumes per second (Papadacci et al., 2019), could also be used to resolve the three-dimensional motion of the heart with higher accuracy and spatio-temporal resolution. Ultimately, the next validation steps after a successful validation in a Langendorff-perfused heart model would be a working heart model, which would allow the investigation of the impact of preload and after load on the inverse mechano-electrical reconstruction, or in-vivo models such as clinically acquired catheter mapping data. A key concern regarding the use of fully convolutional neural networks in clinical applications is the difficulty in assessing their true limitations. Combining deep learning with physics-based modeling could potentially better address these issues. Physics-informed neural networks (PINNs) (Raissi et al., 2019; Martinet al., 2022) present a promising approach in this regard. This work demonstrates that the inverse mechano-electrical approach will likely ultimately prove to be successful in overcoming the obstacles associated with clinical translation and adoption, and will make a meaningful impact in the field of cardiac imaging. This disclosure provides a first proof-of-principle and lays out the groundwork for the clinical development of the approach in the future.

In at least some of the previously described embodiments, one or more elements used in an embodiment can interchangeably be used in another embodiment unless such a replacement is not technically feasible. It will be appreciated by those skilled in the art that various other omissions, additions, and modifications may be made to the methods and structures described above without departing from the scope of the claimed subject matter. All such modifications and changes are intended to fall within the scope of the subject matter, as defined by the appended claims.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

What is claimed is:

1. A method of configuring a model to reflect electrical and mechanical behavior of a tissue, the method comprising:
collecting initial data comprising corresponding electrophysiological activation patterns of a tissue and mechanical movement patterns of the tissue; and
configuring a model of the tissue based on the corresponding electrophysiological activation patterns of the tissue and mechanical movement patterns of the tissue.

2. The method of claim 1, wherein the initial data comprises dynamic structural imaging data.

3. The method of claim 1 or 2, wherein the initial data comprises model training data.

4. The method of any one of one the previous claims, wherein the initial data comprises model configuration data.

5. The method of any one of the previous claims, wherein the initial data reflects an initial state of tissue electrophysiology and mechanical movement.

6. The method of any one of the previous claims, wherein the electrophysiological activation patterns comprise one or more of: electrical, optical, acoustical, and/or photo-acoustical measurements of electrical excitation of the tissue and/or structure of the tissue.

7. The method of any one of the previous claims, wherein the initial data is obtained from a subject.

8. The method of claim 7, wherein the initial data comprises subject-specific data.

9. The method of claim 8, wherein the subject-specific data comprises one or more of: demographic data, genetic information, patient history data, structural imaging data, and/or quantitative medical data.

10. The method of claim 8 or 9, wherein the method further comprises configuring the model of the tissue based on the subject information.

11. The method of claim 10, wherein the model of the tissue is configured based on the subject information after the model is configured based on the electrophysiological activation patterns and mechanical movement patterns of the tissue.

12. The method of any one of the previous claims, wherein the dynamic structural imaging data comprises ultrasound imaging data, magnetic resonance imaging (MRI) data, or computed tomography (CT) imaging data.

13. The method of claim 12, wherein the ultrasound imaging data reflects mechanical movement patterns.

14. The method of any one of the previous claims, wherein the dynamic structural imaging data comprises optical data.

15. The method of claim 14, wherein the optical data reflects electrophysical activation patterns.

16. The method of any one of the previous claims, wherein the dynamic structural imaging data comprises fused imaging and optical data.

17. The method of any one of the previous claims, wherein the dynamic structural imaging data comprises imaging data over time.

18. The method of any one of the previous claims, wherein the initial data comprises synthetic data.

19. The method of any one of the previous claims, wherein the initial data comprises data generated using a numerical model configured to represent intra- and/or extra-cellular processes of the tissue.

20. The method of any one of the previous claims, wherein the initial data comprises data generated using a computational technique.

21. The method of claim 20, wherein the computational technique comprises one or more of: finite-difference methods (FDM), finite element method (FEM), finite volume method (FVM), smoothed-particle hydrodynamics (SPH), and boundary-element method (BEM), and/or a differentiable version of such technique, optionally comprising a differential SPH simulation fitted to data.

22. The method of any one of the previous claims, wherein the model is trained using training data comprising electrophysiological data and mechanical deformation data, or fitted to such data using gradient descent techniques.

23. The method of any one of the previous claims, wherein electrophysiological activation patterns comprise one or more of: action potential (AP) waves or wave fronts, calcium waves or wave fronts, AP activation maps, calcium activation maps, calcium concentrations or changes in calcium concentrations, oxygenation or changes in oxygenation, and/or pH or changes in pH.

24. The method of any one of the previous claims, wherein the electrophysiological activation patterns comprise intramural electrical activity of the tissue.

25. The method of any one of the previous claims, wherein the electrophysiological activation patterns comprise electrical activity on the tissue surface.

26. The method of any one of the previous claims, wherein the electrophysiological activation patterns comprise a sinus wave pattern, a focal wave pattern, and/or a reentrant wave pattern.

27. The method of any one of the previous claims, wherein the electrophysiological activation patterns comprise a three-dimensional wave pattern.

28. The method of any one of the previous claims, wherein the mechanical movement patterns comprise tissue deformation.

29. The method of any one of the previous claims, wherein the mechanical movement patterns comprise tissue deformation triggered by the electrical activation patterns.

30. The method of any one of the previous claims, wherein the method further comprises applying the model to estimate an electrophysiological activation pattern of a tissue of a subject by:
inputting experimental data to the model, wherein the experimental data comprises one or more mechanical movement patterns; and
receiving estimated electrophysiological activation patterns output from the model.

31. The method of any one of the previous claims, wherein the model infers electrical activity of the tissue from mechanical deformation of the tissue.

32. The method of any one of the previous claims, wherein the model infers internal electrical activity of the tissue from mechanical deformation of the tissue.

33. The method of any one of the previous claims, wherein the method further comprises applying the model to estimate a mechanical movement pattern of a tissue of a subject by:
inputting experimental data to the model, wherein the experimental data comprises one or more electrophysiological activation patterns of the tissue; and
receiving estimated mechanical movement patterns output from the model.

34. The method of any one of the previous claims, wherein the method further comprises applying the model to estimate electrophysiological activation patterns of a tissue of a subject by:
inputting data to the model, wherein the input data comprises one or more electrophysiological activation patterns of the tissue; and
receiving estimated subsequent electrophysiological activation patterns output from the model.

35. The method of claim 34, wherein the electrophysiological activation pattern input to the model comprises experimental data.

36. The method of claim 34 or 35, wherein the electrophysiological activation pattern input to the model comprises electrode-recordings.

37. The method of any one of the previous claims, wherein the electrophysiological activation pattern input to the model comprises an incomplete measurement of electrical activity of the tissue.

38. The method of any one of the previous claims, wherein the electrophysiological activation pattern input to the model comprises sparse measurement data or data with low spatial resolution.

39. The method of any one of the previous claims, wherein the electrophysiological activation pattern input to the model comprises simulated data.

40. The method of any one of the previous claims, wherein the estimated subsequent electrophysiological activation patterns comprise chaotic wave phenomena.

41. The method of any one of the previous claims, wherein the method further comprises applying the model to estimate a mechanical movement pattern of a tissue of a subject by:
inputting experimental data to the model, wherein the experimental data comprises one or more mechanical movement patterns of the tissue; and
receiving estimated subsequent mechanical movement patterns output from the model.

42. The method of any one of the previous claims, wherein the method further comprises applying the model to estimate electrophysiological activation patterns and mechanical movement patterns of a tissue of a subject by:
inputting experimental data to the model, wherein the experimental data comprises one or more electrophysiological activation patterns of the tissue or mechanical movement patterns of the tissue; and
receiving estimated electrophysiological activation patterns and mechanical movement patterns output from the model.

43. The method of any one of the previous claims, wherein the method further comprises applying the model to estimate the presence of abnormal electrophysiological activation patterns of a tissue of a subject by:
inputting experimental data to the model, wherein the experimental data comprises one or more electrophysiological activation patterns of the tissue or mechanical movement patterns of the tissue; and
receiving an estimate of a presence of abnormal electrophysiological activation patterns of the tissue output from the model.

44. The method of claim 43, wherein the abnormal electrophysiological activation patterns comprise electrophysiological activation patterns associated with an arrythmia, tachycardia, and/or the presence of scar tissue.

45. The method of any one of the previous claims, wherein the method further comprises:
obtaining ultrasound imaging data of the tissue;
applying the model to the obtained ultrasound imagining data; and
receiving an estimate from the model of the presence of an abnormal physiological symptom.

46. The method of claim 45, wherein the abnormal physiological symptom comprises one or more of: an abnormal electrical circuit, an abnormal tissue characteristic, scar tissue, diseased tissue, tissue comprising a discontinuity, and/or fibrotic tissue.

47. The method of any one of the previous claims, wherein the method further comprises applying the model to estimate a location of, or particular morphology associated with, an electrical circuit causing an abnormal electrophysiological activation pattern.

48. The method of any one of the previous claims, wherein the method further comprises applying the model to identify tissue associated with an abnormal electrophysiological activation pattern.

49. The method of any one of the previous claims, wherein the method further comprises applying the model to identify a location for tissue ablation or other tissue modification to remove an abnormal electrophysiological activation pattern.

50. The method of any one of the previous claims, wherein the model is further configured based on subject information comprising one or more of: demographic data, genetic information, patient history data, structural imaging data, and/or quantitative medical data.

51. The method of any one of the previous claims, wherein data input to and/or data output from the model comprises spatio-temporal data.

52. The method of any one of the previous claims, wherein data input to and/or data output from the model comprises a sequence of images.

53. The method of any one of the previous claims, wherein data output from the model comprises a single image.

54. The method of any one of the previous claims, wherein data input to the model and data output from the model each comprise three-dimensional representations of the tissue over time.

55. The method of any one of the previous claims, wherein the initial data comprises the experimental data.

56. The method of any one of the previous claims, wherein the experimental data is distinct from the initial data.

57. The method of any one of the previous claims, wherein the model is first configured using the initial data before being subsequently trained using experimental data.

58. The method of any one of the previous claims, wherein the experimental data comprises dynamic structural imaging data.

59. The method of claim 58, wherein the dynamic structural imaging data comprises ultrasound imaging data.

60. The method of claim 58 or 59, wherein the dynamic structural imaging data comprises optical data.

61. The method of any one of claims 58-60, wherein the dynamic structural imaging data comprises fused imaging and optical data.

62. The method of any one of claims 58-61, wherein the dynamic structural imaging data comprises imaging data over time.

63. The method of any one of the previous claims, wherein the experimental data comprises one or more of: ultrasound, MRI, and/or CT scan data.

64. The method of any one of the previous claims, wherein the method further comprises generating synthetic data for use configuring or training the model.

65. The method of any one of the previous claims, wherein the initial data comprises synthetic data.

66. The method of any one of the previous claims, wherein the model is trained using synthetic data.

67. The method of any one of claims 64-66, wherein the synthetic data comprises data generated based on a mechanical model of the tissue or an electrophysiological model of the tissue.

68. The method of any one of claims 64-67, wherein generating the synthetic data comprises:
generating variations of tissue models in silico; and
applying such variations of tissue models to generate synthetic data.

69. The method of any one of claims 64-68, wherein the synthetic data comprises data generated by applying a mass-spring model (MSM), a finite-element model (FEM), and/or a smoothed-particle hydrodynamics (SPH) framework, or differentiable versions thereof, optionally comprising a differentiable simulation version of MSM, SPH, and/or FEM).

70. The method of any one of claims 64-69, wherein the synthetic data comprises data generated by applying an electromechanically coupled simulation.

71. The method of any one of the previous claims, wherein the method further comprises:
generating a plurality of models of structures of the tissue, wherein each of the structures varies in at least one aspect; and
applying the plurality of models to generate synthetic data comprising electrophysiological activation patterns of the tissue and/or mechanical movement patterns of the tissue.

72. The method of claim 71, wherein each of the structures varies in one or more of: tissue shape, tissue thickness, tissue stiffness, contractile force, fiber orientations, interconnections between regions of tissues, velocities of electrical activation patterns, durations of electrical activation waves, and/or spatial heterogeneity.

73. The method of any one of the previous claims, wherein the method further comprises using numerical simulations of tissue behavior to generate synthetic data.

74. The method of any one of the previous claims, wherein the synthetic data is configured to mimic experimental data.

75. The method of any one of the previous claims, wherein data used to configure or train the model is augmented by one or more of: adding random noise to the data, randomly rotating or flipping the data, removing a random section of the data, randomly scaling the data, blurring the data, and/or adding artifacts to the data.

76. The method of any one of the previous claims, wherein the synthetic data comprises electrophysiological activation patterns exhibiting a sinus wave, a focal wave, and/or a reentrant wave.

77. The method of any one of the previous claims, wherein data used to configure or train the model comprises electrophysiological activation patterns exhibiting a sinus wave, a focal wave, and/or a reentrant wave.

78. The method of any one of the previous claims, wherein the method further comprises:
generating ground-truth electrical activation patterns of the tissue;
using the ground-truth electrical activation patterns to estimate mechanical movement patterns of the tissue;
applying the model to the estimated mechanical movement patterns to estimate the electrical activation patterns.

79. The method of any one of the previous claims, wherein the model is trained using training data generated from a plurality of electrophysiological models and mechanical deformation models.

80. The method of any one of the previous claims, wherein the data generated by the plurality of electrophysiological models is applied to the plurality of mechanical deformation models to generate data comprising corresponding electrical activation and mechanical movement patterns.

81. The method of any one of the previous claims, wherein the model comprises a model of electrophysiological activation patterns of the tissue.

82. The method of any one of the previous claims, wherein the model comprises a model of calcium concentrations, oxygenation, and/or pH of the tissue or changes in one or more of calcium concentrations, oxygenation, and/or pH of the tissue.

83. The method of any one of the previous claims, wherein the model comprises a model of mechanical movement of the tissue.

84. The method of any one of the previous claims, wherein the model comprises a model of three-dimensional images or three-dimensional motion vectors of the tissue.

85. The method of any one of the previous claims, wherein mechanical movement of the tissue is represented based on three-dimensional images or three-dimensional motion vectors.

86. The method of any one of the previous claims, wherein the model is trained to learn mechanical movement of the tissue based on three-dimensional images or three-dimensional motion vectors.

87. The method of any one of the previous claims, wherein the method comprises estimating electrical activity of the tissue based on three-dimensional images or three-dimensional motion vectors representing tissue movement using the model.

88. The method of any one of the previous claims, wherein the model comprises a deep learning model.

89. The method of any one of the previous claims, wherein the model comprises one or more of: a convolutional neural network, a U-Net, a sparse Res U-Net, a denoising diffusion neural network, a sparse denoising diffusion neural network, a transformer, a graph neural network, and/or a large language model.

90. The method of claim 89, wherein the model comprises convolutional or attention mechanisms.

91. The method of any one of the previous claims, wherein the model comprises a sparse encoding-decoding convolutional neural network.

92. The method of any one of the previous claims, wherein the model comprises a virtual vector model.

93. The method of any one of the previous claims, wherein the model comprises a numerical simulation.

94. The method of any one of the previous claims, wherein the model comprises a mass-spring model (MSM), a smoothed-particle hydrodynamics (SPH) model, or a finite element method (FEM) model.

95. The method of any one of the previous claims, wherein the model comprises a differentiable electrical, mechanical, or electromechanical tissue model.

96. The method of any one of the previous claims, wherein the model comprises a simulation core component.

97. The method of any one of the previous claims, wherein the initial data comprises tissue motion data, tissue geometry data, and/or other subject-specific data.

98. The method of any one of the previous claims, wherein the model is trained using gradient descent optimization.

99. The method of any one of the previous claims, wherein the model comprises a numerical model configured to represent intra- and extra-cellular processes of the tissue.

100. The method of any one of the previous claims, wherein the model comprises a computational technique comprising one or more of: finite-difference methods (FDM), finite element method (FEM), finite volume method (FVM), smoothed-particle hydrodynamics (SPH), and/or boundary-element method (BEM).

101. The method of any one of the previous claims, wherein the model comprises an idealized model of the tissue.

102. The method of any one of the previous claims, wherein the model comprises a subject-specific model of the tissue.

103. The method of any one of the previous claims, wherein the model comprises a model of the tissue comprising one or more of: a fiber-architecture model of the tissue, scar placement within the tissue, and/or fibrosis placement within the tissue.

104. The method of any one of the previous claims, wherein the method further comprises applying the model for drug screening.

105. The method of any one of the previous claims, wherein the method further comprises of applying the model for treatment of a subject.

106. The method of any one of the previous claims, wherein the model comprises a digital twin of a subject.

107. The method of any one of the previous claims, wherein the method further comprises generating a virtual simulation of the tissue using the model.

108. The method of any one of the previous claims, wherein the configuring comprises simulating a tissue function as the model learns the contractile motion and electrophysiological wave pattern associated with the tissue.

109. The method of any one of the previous claims, wherein the configuring comprises simulating a pumping heart as the model learns the contractile motion and electrophysiological wave pattern associated with the tissue.

110. The method of any one of the previous claims, wherein the method further comprises using the model to evolve the behavior of a simulated tissue over time.

111. The method of any one of the previous claims, wherein the electrical behavior of the tissue is integrated with the mechanical behavior of the tissue in the model.

112. The method of any one of the previous claims, wherein the method further comprises applying the model to estimate a sequence in which different tissue locations are electrically activated.

113. The method of any one of the previous claims, wherein the method further comprises applying the model to estimate an electrical activity activation map of the tissue.

114. The method of any one of the previous claims, wherein the method further comprises applying the model to estimate a location of scar tissue on the tissue.

115. The method of any one of the previous claims, wherein the method further comprises applying the model to estimate a location of tissue that exhibits an abnormal electrical property.

116. The method of claim 115, wherein the abnormal electrical activity comprises one or more of abnormal conductivity and/or abnormal conduction speed for the tissue.

117. The method of any one of the previous claims, wherein the method further comprises applying the model to estimate a location of tissue that exhibits an abnormal mechanical property.

118. The method of claim 117, wherein the abnormal mechanical property comprises abnormal contractility for the tissue.

119. The method of any one of the previous claims, wherein the method further comprises validating the model based on clinical treatment of a subject.

120. The method of any one of the previous claims, wherein the method further comprises validating the model, wherein validating the model comprises:
using the model to estimate an electrical activation pattern of the tissue based on an observed movement pattern of the tissue; and
confirming the electrical activation pattern is associated with the observed movement pattern of the tissue.

121. The method of any one of the previous claims, wherein the method further comprises validating the model, wherein validating the model comprises:
obtaining an electrical activation pattern of a tissue or a mechanical activation pattern of a tissue;
estimating an electrical activation pattern or a mechanical activation pattern by applying the model to the electrical activation pattern or a mechanical activation pattern;
using a second model to estimate an electrical activation pattern or a mechanical activation pattern by applying the second model to the electrical activation pattern or a mechanical activation pattern, wherein the second model comprises a physics-based model; and
assessing the accuracy of the model by comparing the estimates from the model against the estimates from the second model.

122. The method of any one of the previous claims, wherein the method further comprises collecting ultrasound imaging data of an in vivo tissue of a subject;
applying the collected imaging data to the model to estimate electrical activation patterns within the tissue of the subject; and
identifying one or more aspects of an abnormal electrophysiological activation pattern of the tissue of the subject.

123. The method of any one of the previous claims, wherein the method is a method of non-invasively analyzing electrophysiological processes of the tissue.

124. The method of any one of the previous claims, wherein the method is a method of non-invasively mapping a heart's electrical activity.

125. A method of observing mechanical movement patterns and corresponding electrophysiological activation patterns of a tissue, the method comprising:
introducing a tissue into an imaging apparatus configured to image electrophysiological activation patterns and mechanical movement patterns of the tissue, wherein the imaging apparatus comprises:
an enclosed interior volume into which the tissue is introduced;
a substrate on which the tissue is mounted;
an electrode for electrically stimulating the tissue;
a perfusion subsystem configured to support physiological conditions of the tissue;
a plurality of light sources configured for optically illuminating the tissue;
an imaging system comprising an optical imaging subsystem configured for obtaining optical images of the tissue; and
a plurality of mounts configured for mounting the light sources and aspects of the imaging system such that the tissue is evenly illuminated, and the imaging system obtains optical images of the tissue from a plurality of different perspectives;
electrically stimulating the tissue with the electrode;
imaging of the tissue by applying the imaging system in response to a stimulation of the tissue, wherein tissue stimulation comprises one or more of electrical, optical, and/or pharmacological stimulation of the tissue;
imaging electrophysiological activation patterns of the tissue by applying the imaging system to obtain voltage- or calcium-sensitive optical mapping images in response to the stimulation of the tissue; and
combining the obtained images of mechanical movement patterns and electrophysiological activation patterns to reconstruct movement of the tissue in response to the stimulation.

126. The method of claim 125, wherein the imaging system of the imaging apparatus further comprises an ultrasound imaging subsystem configured for obtaining ultrasound images of the tissue.

127. The method of claim 126, wherein the mechanical movement patterns of the tissue are imaged by applying the ultrasound imaging subsystem in response to a stimulation of the tissue.

128. The method of claims 125 to 127, wherein the imaging apparatus further comprises a plurality of electrodes configured to detect electrophysiological activation patterns of the tissue.

129. The method of claim 128, wherein the plurality of detection electrodes are configured to measure electrocardiograms from the tissue.

130. The method of claims 125 to 129, wherein the imaging apparatus further comprises a power source configured to supply power to the imaging system and/or the plurality of light sources.

131. The method of claims 125 to 130, wherein the plurality of mounts are configured for mounting aspects of the imaging system such that every point of the surface of the tissue is imaged from two or more perspectives.

132. The method of claims 125 to 131, wherein the method further comprises optically stimulating the tissue using the plurality of light-sources.

133. The method of any one of claims 125 to 132, wherein the method further comprises pharmacologically stimulating the tissue using pharmacological compounds or drugs.

134. The method of any one of claims 125 to 133, wherein the method further comprises pharmacologically immobilizing aspects of the tissue using pharmacological compounds or drugs.

135. The method of any one of claims 125 to 134, wherein the imaging apparatus comprises an imaging chamber or an imaging rig.

136. The method of claim 135, wherein the imaging chamber or rig comprises a plurality of windows configured for illuminating the tissue and obtaining images of the tissue from a plurality of different perspectives.

137. The method of any one of claims 125 to 136, wherein the optical imaging subsystem comprises a multi-camera high-speed imaging system.

138. The method of any one of claims 125 to 137, wherein the method comprises obtaining panoramic images of the tissue.

139. The method of claim 138, wherein every point on the surface of the tissue is imaged.

140. The method of any one of claims 125 to 139, wherein the method further comprises:
repeatedly obtaining representations of corresponding electrophysiological activation patterns of the tissue and mechanical movement patterns of the tissue; and
combining such representations into a data set for configuring a model according to claims 1 to 124.

141. The method of claim 140, wherein the method further comprises:
collecting electrical activation patterns and associated mechanical movement patterns for the tissue present in the imaging chamber; and
applying the electrical activation patterns and associated mechanical movement patterns as training data for the model.

142. The method of any one of claims 125 to 141, wherein the method further comprises applying a numerical motion compensation technique to images of the tissue.

143. The method of any one of claims 125 to 142, wherein the method further comprises applying an optical mapping technique combined with a numerical motion tracking technique to measure an electrical activation pattern in the tissue moving in the imaging apparatus.

144. The method of any one of claims 125 to 143, wherein every point of the surface of the tissue is imaged from two or more perspectives and the method further comprises applying a stereoscopic motion tracking technique to measure an electrical activation pattern and/or a mechanical movement pattern in the tissue moving in the imaging apparatus.

145. The method of any one of claims 125 to 144, wherein the method comprises applying a GPU-based motion tracking algorithm to process imaging data of the tissue.

146. The method of any one of claims 125 to 145, wherein the method further comprises:
collecting a plurality of raw images of the tissue;
applying an optical flow estimation technique or a differentiable rendering technique to the raw images to compensate for tissue movement; and
generating a plurality of compensated images of the tissue to estimate tissue deformation.

147. The method of claim 146, wherein every point of the surface of the tissue is imaged from two or more perspectives and the optical flow estimation technique is a stereoscopic optical flow estimation technique or a multi-view differentiable rendering technique used for tracking three-dimensional motion.

148. The method of any one of claims 125 to 147, wherein the method further comprises applying one or more optical flow estimation techniques or differentiable motion estimation techniques or motion compensation techniques to images of the tissue moving to mitigate motion artifacts.

149. The method of any one of claims 125 to 148, wherein the method comprises imaging the tissue without applying a pharmacological excitation-contraction uncoupling agent to the tissue.

150. The method of any one of claims 125 to 149, wherein the method comprises imaging electrophysiological activation patterns of the tissue or mechanical movement patterns of the tissue without a fiducial marker.

151. The method of any one of claims 125 to 150, wherein the method comprises imaging electrical activation patterns across a tissue surface as the tissue deforms.

152. The method of any one of claims 125 to 151, wherein the method comprises imaging electrical activation patterns across a surface of the tissue (optionally comprising an atrial or a ventricular surface) as the tissue contracts.

153. A method comprising validating a model according to any one of claims 1 to 124 based on imaging obtained using the imaging apparatus according to any one of claims 125 to 152.

154. The method of any one of the previous claims further comprising:
validating the model, wherein validating the model comprises:
using the model to estimate an electrical activation pattern of the tissue based on an observed movement pattern of the tissue; and
confirming the electrical activation pattern is associated with the observed movement pattern of the tissue.

155. The method of any one of the previous claims further comprising validating the model, wherein validating the model comprises:
obtaining an electrical activation pattern of a tissue or a mechanical activation pattern of a tissue;
estimating the electrical activation pattern or the mechanical activation pattern by applying the model to the electrical activation pattern or a mechanical activation pattern;
using a second model to estimate the electrical activation pattern or the mechanical activation pattern by applying the second model to the electrical activation pattern or a mechanical activation pattern, wherein the second model comprises a physics-based model; and
assessing the accuracy of the model by comparing the estimates from the model against the estimates from the second model.

156. The method of any one of the previous claims further comprising:
collecting ultrasound imaging data of an in vivo tissue of a subject;
applying the collected imaging data to the model to estimate electrical activation patterns within the tissue of the subject; and
identifying one or more aspects of an abnormal electrophysiological activation pattern of the tissue of the subject.

157. The method of any one of the previous claims, wherein the method is a method of non-invasively analyzing electrophysiological processes of the tissue.

158. The method of any one of the previous claims, wherein the method is a method of non-invasively mapping a heart's electrical activity.

159. The method of any one of the previous claims, wherein the tissue comprises electrically excitable tissue.

160. The method of any one of the previous claims, wherein the tissue comprises anisotropic tissue.

161. The method of any one of the previous claims, wherein the tissue comprises cardiomyocytes.

162. The method of any one of the previous claims, wherein the tissue is cardiac tissue, lung tissue, intestinal tissue, or uterine tissue.

163. The method of claim 162, wherein the tissue is cardiac tissue.

164. The method of any one of the previous claims, wherein the tissue comprises one or more of: left ventricle tissue, right ventricle tissue, left atrium tissue, and/or right atrium tissue.

165. The method of claim 164, wherein the tissue comprises left and right ventricles.

166. The method of any one of the previous claims, wherein the tissue comprises a biventricular heart geometry.

167. The method of any one of the previous claims, wherein the tissue is human tissue, rabbit tissue, pig tissue, sheep tissue, dog tissue, mouse tissue, rat tissue, or guinea pig tissue.

168. The method of claim 167, wherein the tissue is human tissue or pig tissue.

169. The method of any one of the previous claims, wherein the tissue comprises a discontinuity.

170. The method of claim 169, wherein the discontinuity is scar tissue or fibrotic tissue.

171. The method of any one of the previous claims, wherein the tissue comprises an isolated organ.

172. The method of any one of the previous claims, wherein the tissue comprises cultured tissue.

173. The method of any one of the previous claims, wherein the tissue comprises elastic excitable media.

174. The method of any one of the previous claims, wherein the tissue exhibits muscle fiber anisotropy.

175. A method of assessing a model, the method comprising:
generating ground-truth electrical activation patterns of a tissue;
using the ground-truth electrical activation patterns to estimate mechanical movement patterns of the tissue;
applying a model of the tissue to the estimated mechanical movement patterns of the tissue to estimate associated electrical activation patterns;
comparing the estimated associated electrical activation patterns with the ground-truth electrical activation patterns; and
assessing the accuracy of the model's predictions based on results of comparing the estimated and ground-truth electrical activation patterns.

176. A method of training a model to estimate electrophysiological activation patterns based on mechanical movement patterns, to estimate mechanical movement based on electrophysiological activation patterns, and/or to estimate a future electromechanical state based on electrophysiological activation patterns or mechanical movement patterns, the method comprising:
collecting a plurality of training data comprising corresponding electrophysiological activation patterns of a tissue and mechanical movement patterns of the tissue;
representing the electrophysiological activation patterns of the tissue as voltages, calcium concentrations, oxygenation, pH, and/or a temporal change thereof over a volume representing the tissue;
representing the mechanical movement of the tissue as three-dimensional images and/or motion vectors over the volume representing the tissue;
training a model using the electrophysiological activation patterns and corresponding mechanical movement.

177. The method of claim 176, wherein the model is trained to estimate electrophysiological activation patterns, given a representation of mechanical movement of the tissue.

178. The method of any one of claims 176 to 177, wherein the model is trained to estimate electrophysiological activation patterns, including abnormal patterns and locations, over a time period.

179. The method of any one of claims 176 to 178, wherein the training data comprises a sequence of images of corresponding electrophysiological activation patterns of the tissue and mechanical movement patterns of the tissue at consecutive times.

180. The method of any one of claims 176 to 179, wherein the training data comprises simulation data.

181. The method of claim 180, wherein the simulation data comprises results of electromechanical simulations.

182. The method of any one of any of claims 176 to 181, further comprising augmenting the training data with simulation data.

183. The method of claim 182, wherein augmenting the training data with simulation data comprises:
generating a plurality of computer-based models of structures of the tissue, wherein each of the structures varies in at least one aspect; and
using the plurality of tissue models to generate simulated results of corresponding electrical activation patterns of the modeled tissue and mechanical movement of the modeled tissue.

184. The method of claim 183, wherein each of the structures varies in one or more of: tissue shape, tissue thickness, tissue stiffness, contractile force, fiber orientations, interconnections between regions of tissues, velocities of electrical activation patterns, durations of electrical activation waves, and/or spatial heterogeneity.

185. The method of claim 184, wherein each of the structures varies in spatial heterogeneity, the spatial heterogeneity varied comprising one or more of: tissue shape, tissue thickness, tissue stiffness, contractile force, fiber orientations, interconnections between regions of tissues, velocities of electrical activation patterns, and/or durations of electrical activation waves.

186. The method of any one of claims 183 to 185, wherein the plurality of computer-based models of structures of the tissue comprise numerical simulations comprising one or more of: finite-difference methods (FDM), finite element method (FEM), finite volume method (FVM), smoothed-particle hydrodynamics (SPH), and/or boundary-element method (BEM).

187. The method of any one of claims 183 to 186, wherein augmenting the training data with simulation data further comprises:
simulating the introduction of electrophysiological activation patterns, action potential waves, or calcium waves into the modeled tissue.

188. The method of claim 187, wherein the electrophysiological activation patterns comprise one or more of a sinus wave, focal wave, and/or reentrant wave.

189. The method of any one of claims 176 to 188, wherein the volume representing the tissue comprises an idealized or patient-specific computer-based model of the tissue, optionally comprising one or more of a fiber-architecture model of the tissue, scar or fibrosis placement within the tissue of the tissue.

190. An imaging apparatus for imaging electrophysiological activation patterns and mechanical movement patterns of a tissue, wherein the imaging apparatus comprises:
an enclosed interior volume configured to receive biological tissue;
a perfusion subsystem configured to support physiological conditions of the tissue;
a substrate on which the tissue is mounted;
a plurality of light sources configured for illuminating the tissue;
an imaging system comprising an optical imaging subsystem configured for obtaining optical images of the tissue; and
a plurality of mounts configured for mounting the light sources and aspects of the imaging system such that the tissue is evenly illuminated, and the imaging system obtains optical images of the tissue from a plurality of different perspectives.

191. The imaging apparatus of claim 190, wherein the imaging system further comprises an ultrasound imaging subsystem configured for obtaining ultrasound images of the tissue.

192. The imaging apparatus of claim 190 or 191, wherein the imaging apparatus comprises a plurality of windows configured for illuminating the tissue and obtaining images of the tissue from a plurality of different perspectives.

193. The imaging apparatus of any one of claim 190 or 192, wherein the imaging apparatus further comprises a plurality of electrodes configured to detect electrophysiological activation patterns of the tissue.

194. The imaging apparatus of claim 193, wherein the plurality of detection electrodes are configured to measure electrocardiograms from the tissue.

195. The imaging apparatus of any one of claims 190 to 194, wherein the imaging apparatus further comprises a power source configured to supply power to the imaging system and/or the plurality of light sources.

196. The imaging apparatus of any one of claims 190 to 195, wherein the imaging apparatus comprises a truncated icosahedron shape.

197. The imaging apparatus of any one of claims 190 to 196, wherein the imaging apparatus comprises a soccer ball geometry.

198. The imaging apparatus of any one of claims 190 to 197, wherein the imaging apparatus comprises an open top and an open bottom.

199. The imaging apparatus of any one of claims 190 to 198, wherein the open top and open bottom comprise fluidic pathways.

200. The imaging apparatus of any one of claims 190 to 199, wherein the imaging apparatus comprises 24 penta- or hexagonal surfaces.

201. The imaging apparatus of any one of claims 190 to 200, wherein the imaging apparatus comprises a shape with angles of optical axes between two adjacent surfaces of 37.4° or 41.8°.

202. The imaging apparatus of any one of claims 190 to 201, wherein the surfaces comprise windows configured for mounting cameras.

203. The imaging apparatus of any one of claims 190 to 202, wherein the imaging apparatus comprises a plurality of windows present at vertices between surfaces of the imaging apparatus and configured to receive LEDs to illuminate the interior of the imaging apparatus.

204. The imaging apparatus of any one of claims 190 to 203, wherein the interior of the imaging apparatus is configured to be uniformly illuminated.

205. The imaging apparatus of any one of claims 190 to 204, wherein the optical imaging subsystem comprises six or more cameras.

206. The imaging apparatus of any one of claims 190 to 205, wherein the optical imaging subsystem comprises 12 or more cameras.

207. The imaging apparatus of any one of claims 190 to 206, wherein an angle between neighboring cameras is between 35 and 45°.

208. The imaging apparatus of any one of claims 190 to 207, wherein the optical imaging subsystem comprises a plurality of CCD or CMOS cameras.

209. The imaging apparatus of any one of claims 190 to 208, wherein the optical imaging subsystem is configured for measuring changes in fluorescence of aspects of the tissue.

210. The imaging apparatus of any one of claims 190 to 209, wherein the optical imaging subsystem is configured for measuring changes in fluorescence of aspects of the tissue in response to a physiological change of the tissue.

211. The imaging apparatus of claim 210, wherein the physiological change comprises a change in transmembrane potential of the tissue.

212. The imaging apparatus of any one of claims 190 to 211, wherein the optical imaging subsystem is configured to image from the plurality of cameras simultaneously.

213. The imaging apparatus of any one of claims 190 to 212, wherein the optical imaging subsystem comprises a plurality of filters configured to block excitation light originating from the plurality of light sources.

214. The imaging apparatus of any one of claims 190 to 213, wherein the perfusion subsystem comprises a constant-pressure Langendorff-perfusion subsystem.

215. The imaging apparatus of any one of claims 190 to 214, wherein the volume of the imaging apparatus is approximately 5 L.

216. The imaging apparatus of any one of claims 190 to 215, wherein the imaging apparatus further comprises an electrode configured for electrically stimulating the tissue.

217. The imaging apparatus of claim 216, wherein the stimulation electrode is configured to introduce regular pacing of electrical stimulation to the tissue.

218. The imaging apparatus of claim 216 or 217, wherein the stimulation electrode is configured to introduce burst pacing of electrical stimulation to the tissue.

219. The imaging apparatus of any one of claims 190 to 218, wherein the imaging apparatus is configured to obtain 360° imaging of the tissue.

220. The imaging apparatus of any one of claims 190 to 219, wherein the imaging apparatus is configured to simultaneously obtain images of every point on the surface of the tissue from two or more perspectives.

221. The imaging apparatus of any one of claims 190 to 220, wherein the imaging apparatus is configured to simultaneously obtain images of every point on the surface of the tissue from four or more perspectives.

222. The imaging apparatus of any one of claims 190 to 221, wherein the imaging apparatus is configured to obtain images of the tissue while the tissue moves.

223. The imaging apparatus of any one of claims 190 to 222, wherein the imaging apparatus is configured to obtain images of the tissue while the tissue deforms.

224. The imaging apparatus of any one of claims 190 to 223, wherein the imaging apparatus comprises a 3D-printed substrate.

225. The imaging apparatus of any one of claims 190 to 224, wherein the imaging apparatus further comprises a temperature-regulation subsystem.

226. The imaging apparatus of any one of claims 190 to 225, wherein the imaging apparatus a processor comprising memory operably coupled to the processor, wherein the memory comprises instructions stored thereon, which, when executed by the processor, cause the processor to control the imaging apparatus.

227. A system for utilizing a model of a tissue, the system comprising:
a processor comprising memory operably coupled to the processor, wherein the memory comprises instructions stored thereon, which, when executed by the processor, cause the processor to implement the steps of a method of any one of claims 1 to 189.

228. The system of claim 227, wherein the system further comprises an imaging apparatus according to any of claims 190 to 226, wherein the imaging apparatus is operably connected to the processor and memory.

229. The system of claim 227 or 228, wherein the memory further comprises instructions store thereon, which, when executed by the processor cause the processor to:
control the imaging apparatus to collect images; and
apply a numerical motion tracking algorithm to mitigate motion artifacts present in the images.

230. A non-transitory computer readable storage medium comprising instructions stored thereon, the instructions comprising an algorithm configured to implement the steps of a method of any one of claims 1 to 189.

231. A method for identifying a subject as having a heart rhythm disorder, the method comprising:
imaging the heart of the subject using 4D ultrasound in order to generate spatio-temporal mechanical deformation data of the subject's heart during one or more contractions; and
inputting the mechanical deformation data into a model according to any one of claims 1 to 124, wherein the output of the model is used to determine if the subject has a heart rhythm disorder.

232. A method for performing catheter ablation on the heart of a subject having a heart rhythm disorder, the method comprising:
imaging the heart of the subject using 4D ultrasound in order to generate spatio-temporal mechanical deformation data of the subject's heart during one or more contractions;
inputting the mechanical deformation data into a model according to any one of claims 1 to 124, wherein the output of the model is used to locate tissue associated with an abnormal electrical wave, an abnormal electrical pathway, or scar tissue; and
ablating tissue associated with the abnormal electrical wave, the abnormal electrical pathway, or the scar tissue in order to disrupt formation or propagation of the abnormal electrical wave.
